(19) United States
(12) Patent Application Publication
(10) Pub. No.: US 2014/0315834 A1
(43) Pub. Date: Oct. 23, 2014

(54) COMPOUNDS AND COMPOSITIONS FOR USE IN THE PREVENTION AND TREATMENT OF INFLAMMATION-RELATED DISORDERS, PAIN AND FEVER, SKIN DISORDERS, CANCER AND PRECANCEROUS CONDITIONS THEREOF

(71) Applicant: Medicon Pharmaceuticals, Inc., Setauket, NY (US)

(72) Inventor: Basil RIGAS, Setauket, NY (US)

(73) Assignee: Medicon Pharmaceuticals, Inc., Setauket, NY (US)

(21) Appl. No.: 14/034,421

(22) Filed: Sep. 23, 2013

Related U.S. Application Data
(60) Provisional application No. 61/703,980, filed on Sep. 21, 2012.
(60) Provisional application No. 61/704,021, filed on Sep. 21, 2012.

(51) Int. Cl. C07F 9/572; C07C 235/84; C07F 9/141

(52) U.S. Cl. CPC C07F 9/5728; C07F 9/141; C07C 235/84
USPC 514/25; 514/80; 514/107; 514/119; 514/120; 514/166; 536/18.2; 548/414; 558/159; 558/170; 558/179; 560/129

(57) ABSTRACT

The present invention provides novel compounds and pharmaceutical compositions for the prevention and/or treatment of cancer and precancerous conditions thereof, for the treatment of pain and fever, for the treatment of skin disorders, and for treating and/or preventing inflammation-related diseases and/or cardiovascular diseases. The compounds of the invention also have analgesic properties and anti-platelet properties. The compounds of the invention may be provided to animals, including mammals and humans, by administering a suitable pharmaceutical dose in a suitable pharmaceutical dosage form. The compounds of the invention have improved efficacy and safety, including higher potency and/or fewer or less severe side effects, than conventional therapies. The compounds of the invention comprise a biologically active moiety or portion (A) that has, or is modified to have at least one carboxyl group. The moiety A is preferably an aliphatic, aromatic or alkylaryl group, preferably derived from a non-steroidal anti-inflammatory drug or NSAID (A). The moiety A is bound to a linker moiety (B) via the carboxyl of group A and a linking atom that is selected from oxygen, nitrogen, and sulphur, to form a carboxylic ester, and amide, or a thioester, bond (X¹) between groups A and B. Moiety B is a single bond, an aliphatic group, a substituted benzene, or an alkylene substituted hydrocarbon chain, which in turn is bound to functional moiety Z, which facilitates access of the compound into cells. The moiety Z can comprise, for example, a phosphorous-containing group, a nitrogen-containing group, or a folic acid residue.

### CROSS REFERENCE TO RELATED APPLICATIONS

This application claims benefit of U.S. Provisional Application Nos. 61/703,980 and 61/704,021, which were both filed on Sep. 21, 2012, and each of which is hereby incorporated by reference in its entirety.

The invention relates to compounds and pharmaceutical compositions for the prevention and/or treatment of cancer and precancerous conditions thereof, for inhibition of platelet aggregation, for the treatment of pain and fever, for the treatment of skin disorders, and for treating and/or preventing other inflammation-related diseases.

### BACKGROUND OF THE INVENTION

Cancer remains a major cause of mortality in the industrial world. Despite significant advances in early detection and treatment, the management of several widespread types of cancer, e.g. lung and pancreatic cancer, remains difficult—and patient survival is poor. Widespread metastases often render surgery ineffectual, leaving chemotherapy as the treatment of choice. Thus there is a clear and pressing need for the development of new agents that are characterized by higher efficacy and lower toxicity. Important in this effort would be the ability to target compounds to cancer cell e.g., by designing compounds with properties that promote binding to the cancer cell, thus maximizing efficacy and minimizing toxicity (lower drug doses will be needed, with less side effects)

Inflammation is a common theme in cancer pathogenesis that has emerged in recent years. This observation provided the basis for exploring and enhancing the chemopreventive properties of anti-inflammatory drugs, including the so-called nonsteroidal anti-inflammatory drugs (NSAIDs). Generally, chemoprevention, or chemoprophylaxis, is a therapy aimed at preventing a disease or infection, such as cancer, as by administering a medication or chemical composition.

NSAIDs generally have anticancer effects restricted to cancer prevention (i.e., chemoprevention) (J. A. Baron, J Natl Cancer Inst 2004, 96, 4-5; E. J. Jacobs et al. J Natl Cancer Inst 2005, 97, 975-80; M. J. Thun et al., Novartis Foundation Symposium 2004, 256, 6-21; discussion 2-8, 49-52, 266-9). The chemopreventive properties of NSAIDs have been established through epidemiological studies and interventional trials. For example, a meta-analysis of 91 epidemiological studies showed a significant exponential decline with increasing intake of NSAIDs in the risk for 7-10 malignancies including the four major types: colon, breast, lung, and prostate cancer (R. E. Harris et al., Oncology Reports 2005, 13, 559-83; T. L. Ratliff, J Urology 2005, 174, 787-8).

However, the application of NSAIDs to chemoprevention is hampered by two serious limitations: (1) suboptimal efficacy, which is less than 50% (K. Kashfi, B. Rigas, Biochem Soc Trans 2005, 33, (Pt. 4), 724-727); and (2) side effects, which makes their long-term use problematic. For example, significant side effects are seen with sulindac, including gastrointestinal (up to 20% of patients), CNS (up to 10%), skin rash and pruritus (5%), and transient elevations of hepatic enzymes in plasma (J. II Roberts, J. Morrow (2001), in: J. G. Flardman, L. E. Limbird (eds); Goodman and Gilman's The Pharmacological Basis of Therapeutics, 10th edn. McGraw-Hill: New York, pp. 687-731).

These observations suggest: (1) the intrinsic anticancer properties of NSAIDs need enhancement if they are to be used successfully in the prevention and/or treatment of various types of cancer; and (2) the considerable side effects of NSAIDs need to be reduced, in particular if a chemoprevention application is contemplated, i.e. for long-term administration to subjects at risk of cancer.

There have been two main approaches to address these limitations. First, several groups have tried to chemically modify NSAIDs to enhance their efficacy and/or safety, e.g., nitric oxide-donating NSAIDs (B. Rigas Curr Opin Gastroenterol 2007, 23, 55-59; B. Rigas, J. L. Williams Nitric Oxide, 2008, 19, 199-204). Second, the development of combination strategies that include conventional NSAIDs. The most successful combination includes sulindac and difluoromethylornithine (DFMO), which reduced by 69% the recurrence of adenomas in humans at risk for sporadic colon adenomas (F. L. Meyskens et al. Cancer Prey Res (Phila) 2008, 1, 9-11). DFMO inhibits ornithine decarboxylase, which catalyses the rate limiting step in polyamine synthesis, whereas sulindac stimulates polyamine acetylation and export from the cell. This results in reduced intracellular polyamine levels leading to suppressed growth of cancer cells (E. W. Gerner, F. L. Meyskens Jr Nat Rev Cancer 2004, 4, 781-792; E. W. Gerner et al. Amino Acids 2007, 33, 189-195). The efficacy and side effects of known chemically modified NSAIDs can still bear improvement, particularly for long-term clinical applications.

There is a need to develop compounds and pharmaceutical compositions with improved efficacy and safety profiles for the treatment and/or prevention of various types of cancer and precancerous conditions.

During the course of work disclosed here, the applicant made the unexpected observation that the chemical modification of NSAIDs could also be applied to other biologically active compounds having a carboxylic moiety or made to acquire such a moiety. Indeed, such chemical modification renders additional compounds other than NSAIDs both efficacious, as described herein, and safe.

The subject matter of this application is also directed at the prevention and/or treatment of pain and fever. Pain is the most common symptom for which patients seek medical assistance. In the case of incurable diseases, treatment for pain may last for extended periods of time. Although subjective, most pain is associated with inflammation and tissue damage, thus having a physiological basis.

Analgesics are drugs used to decrease pain without causing loss of consciousness or sensory perception. There are two general classes of analgesics: (1) anti-inflammatory, routinely prescribed for short-term pain relief and for modest pain; and (2) opioids, for either short-term or long term relief of severe pain.

The opioid analgesics, or narcotics, include all natural or synthetic chemical compounds closely related to morphine and are thought to activate one or more receptors on brain neurons. Opioid analgesics have serious side effects and are to be used with caution. Side effects include: (1) tolerance, which requires gradually increasing doses to maintain analgesia; (2) physical dependence, which means that the narcotics must be withdrawn gradually if they are discontinued after prolonged use; (3) constipation, which requires careful attention to bowel function, including use of stool softeners, laxatives, and enemas; and (4) various degrees of somnolence, or drowsiness, which requires adjustments in dosages and dose scheduling, or possibly varying the type of narcotic to find one better tolerated by the patient.

The role of platelets in thrombus formation is well established. Hence, antiplatelet agents find wide application in the control of thrombosis. For example, rupture of an atherosclerotic plaque is the usual initiating event in an acute coronary syndrome, often leading to thrombus formation. Persistent thrombotic occlusion results in acute myocardial infarction. Antiplatelet agents are extensively used in such clinical circumstances, e.g., in unstable angina and non-ST elevation myocardial infarction.

Antiplatelet agents in clinical use include 1) aspirin; 2) the P2Y12 receptor blockers, clopidogrel, ticlopidine, prasugrel ticagrelor, and cangrelor, which block the binding of adenosine diphosphate to a platelet receptor P2Y12; and 3) anti-GP llb/llla antibodies and receptor antagonists.

All of these agents can cause significant side effects. For example, and mentioning only the main ones: 1) aspirin causes gastrointestinal intolerance or bleeding, allergy (primarily manifested as bronchospasm or asthma), and worsening of pre-existent bleeding. 2) P2Y12 receptor blocker therapy also causes bleeding (its most significant side effect). The common combination of clopidogrel plus aspirin further enhances the incidence of bleeding episodes. Life-threatening bleeding with these agents has been reported. Other important side effects: neutropenia and thrombotic thrombocytopenia purpura/hemolytic uremic syndrome. 3) Major bleeding can occur after the administration of a GP llb/llla inhibitor.

Overall, there is a need to develop compounds with improved efficacy and safety profiles for the treatment and/or prevention of various types of cancer and precancerous conditions, inhibition of platelet aggregation, pain and fever, as well as for the treatment of other inflammation-related disorders and inflammatory skin disorders (e.g., genital warts).

Aspirin (acetylsalicylic acid) is a derivative of salicylic acid invented over a century ago and remains the prototypical NSAID. The anti-inflammatory properties of aspirin are firmly established and explained, in part, by its ability to inhibit the enzyme cyclooxygenase. The anticancer properties of aspirin are also known, but are restricted to cancer prevention. This effect is thought to be mediated through multiple mechanisms.

Despite the fact that aspirin and other related NSAID compounds (e.g., 2-mercaptobenzoic acid and anthranilic acid) have been known in the art for many years, unfortunately, their usefulness as a chemopreventive agent has always been limited by the shortcomings described above (e.g., suboptimal efficacy and side effects). In particular embodiments this application discloses structural derivatives of NSAIDs (e.g., aspirin, 2-mercaptobenzoic acid and anthranilic acid) as well as structural derivatives of other compounds having a carboxylic moiety or made to acquire such a moiety which demonstrate enhanced efficacy and safety as described herein (e.g., higher potency and reduced side-effects) compared to their parent compounds.

### SUMMARY OF THE INVENTION

The present invention provides novel compounds and pharmaceutical compositions for the prevention and/or treatment of cancer and precancerous conditions thereof, for the treatment of pain and fever, for the treatment of skin disorders, and for treating and/or preventing inflammation-related diseases and/or cardiovascular diseases. The compounds of the invention also have analgesic properties and antiplatelet properties. The compounds of the invention may be provided to animals, including mammals and humans, by administering a suitable pharmaceutical dose in a suitable pharmaceutical dosage form. The compounds of the invention have improved efficacy and safety, including higher potency and/or fewer or less severe side effects, than conventional therapies.

The compounds of the invention comprise a biologically active moiety or portion (A) that has, or is modified to have at least one carboxyl group. The moiety A is preferably an aliphatic, aromatic or alkylaryl group, preferably derived from a nonsteroidal anti-inflammatory drug or NSAID (A). The moiety A is bound to a linker moiety (B) via the carboxyl of group A and a linking atom that is selected from oxygen, nitrogen, and sulphur, to form a carboxylic ester, and amide, or a thioester, bond (X¹) between groups A and B. Moiety B is a single bond, an aliphatic group, a substituted benzene, or an alkylene substituted hydrocarbon chain, which in turn is bound to functional moiety Z, which facilitates access of the compound into cells. The moiety Z can comprise, for example, a phosphorous-containing group, a nitrogen-containing group, or a folic acid residue. Suitable choices for moieties A, X¹, B and Z are given herein. Further, it will be understood that each specific moiety A, X¹, B and Z in each of the specific examples herein is representative, and may be used in other combinations with other exemplified moieties, i.e. any A, X¹, B or Z moiety in any exemplary compound disclosed herein may potentially be substituted by any other A, X¹, B or Z moiety disclosed herein.

In one embodiment, the invention comprises a compound of Formula I:

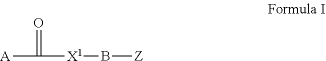

or an enantiomer, diastereomer, racemate, tautomer, salt, hydrate or cocrystal thereof, for use in the treatment and/or prevention of cancer, e.g. lung and brain cancer and precancerous conditions thereof, wherein said treatment and/or prevention comprises administering a pharmaceutically effective dose of the Formula I compound to a human or animal in need thereof. In Formula I:

X¹ is selected from the group consisting of —O—, —S— and —NR¹—,

R¹ is hydrogen or C₁₋₁₀₀-alkyl, preferably C-₁₋₂₂-alkyl, particularly preferred C₁₋₁₀-alkyl.

A is an optionally substituted aliphatic, heteroaliphatic, aromatic, hetero-aromatic substituent or alkylaryl substituent having in a preferred embodiment 1 to 100, and even more preferably 1 to 42 carbon atoms.

Preferably, A is derived from among NSAIDs. In one of the preferred embodiments, A is selected from the group consisting of:

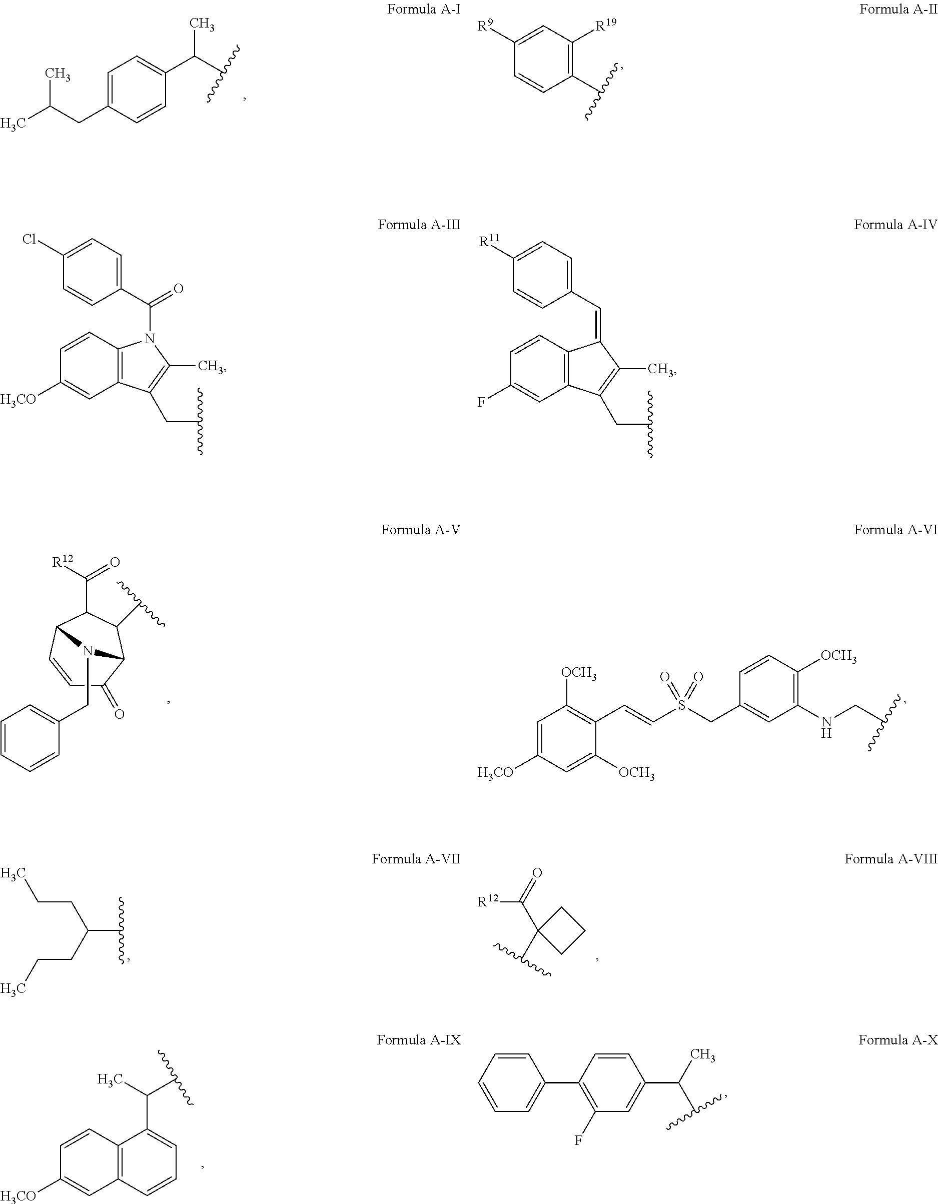

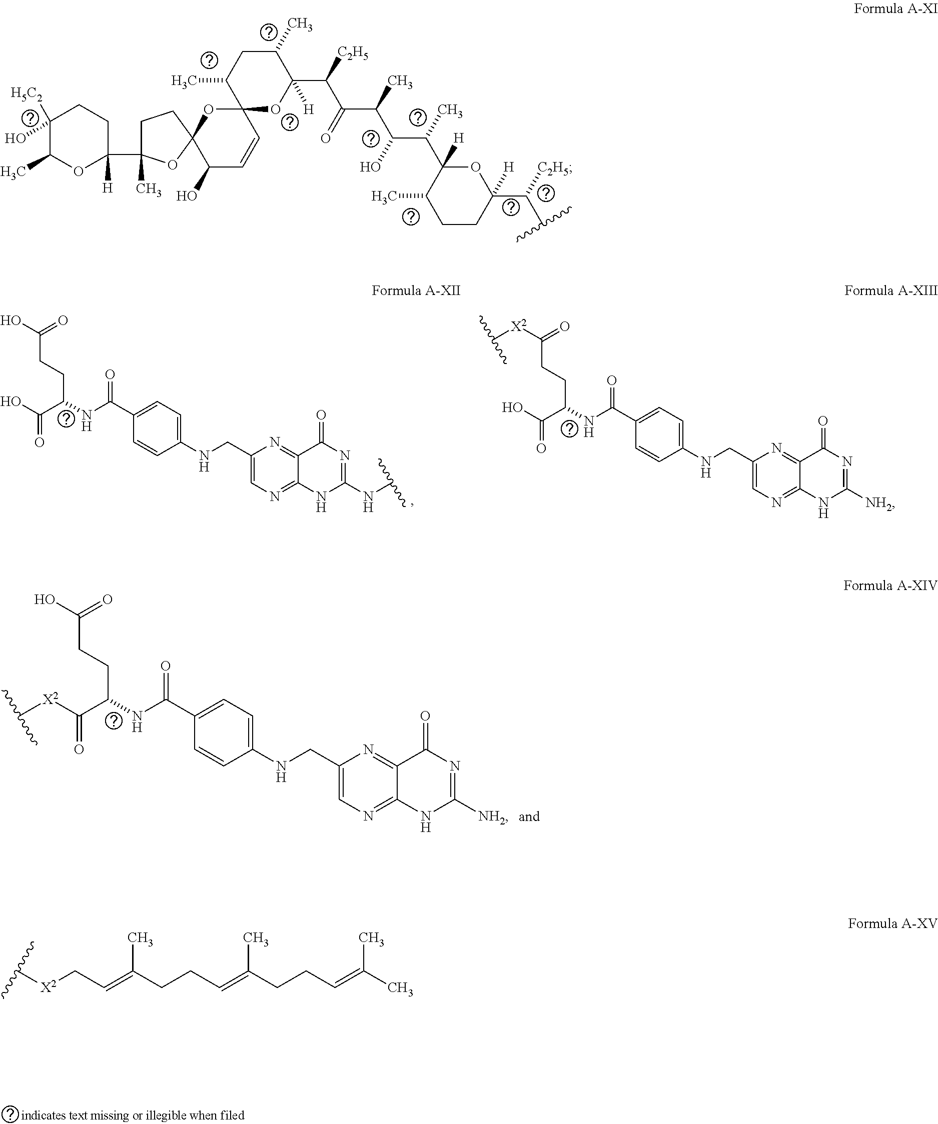

R⁹ is selected from hydrogen and trifluoromethyl;

R¹⁰ is selected from —X₂—C(O)—CH₃,

R¹¹ is selected from —SCH₃, —S(O)CH₃ and —S(O)₂CH₃;

R¹² is selected from hydroxy, —B—Z and Formula A-XII,

whereby X² is selected from the group consisting of —O—, —S— and —NR¹³—

—R¹³ is hydrogen or C₁₋₆-alkyl.

B is selected from the group consisting of

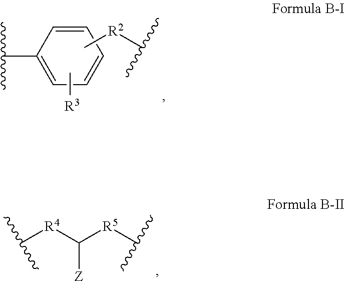

a single bond, and

an aliphatic substituent, preferably with 1 to 100, more preferred with 1 to 42, and particularly preferred with 1 to 22 carbon atoms,

R², R⁴ and R⁵ is the same or different C₁₋₃-alkylene,

R₃ is hydrogen, C₁₋₆-alkyl, halogenated C₁₋₆-alkyl; C₁₋₆-alkoxy, halogenated C₁₋₆-alkoxy, —C(O)—C₁₋₆-alkyl, —C(O)—C₁₋₆-alkyl, —OC(O)C-₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxyl.

Z is selected independently from the group consisting of

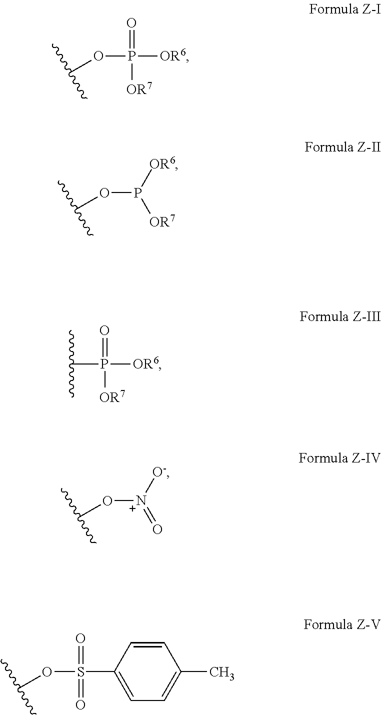

and a folic acid residue;

R⁶ is independently selected from hydrogen, C₁₋₁₀₀-alkyl, preferably C₁₋₆-alkyl, and polyethylene glycol residue,

R⁷ is selected from hydrogen, C₁₋₁₀₀-alkyl, preferably C₁₋₆-alkyl, and polyethylene glycol residue; or

B together with Z forms a structure:

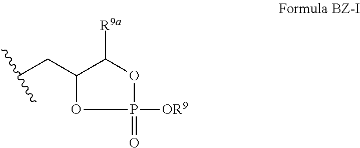

R⁶ is defined as above,

R⁸ is independently selected from hydrogen, an aliphatic substituent, preferably with 1 to 22 carbon atoms, more preferred C₁₋₆-alkyl, and a polyethylene glycol residue.

Preferably, the folic acid residue is selected from the group consisting of

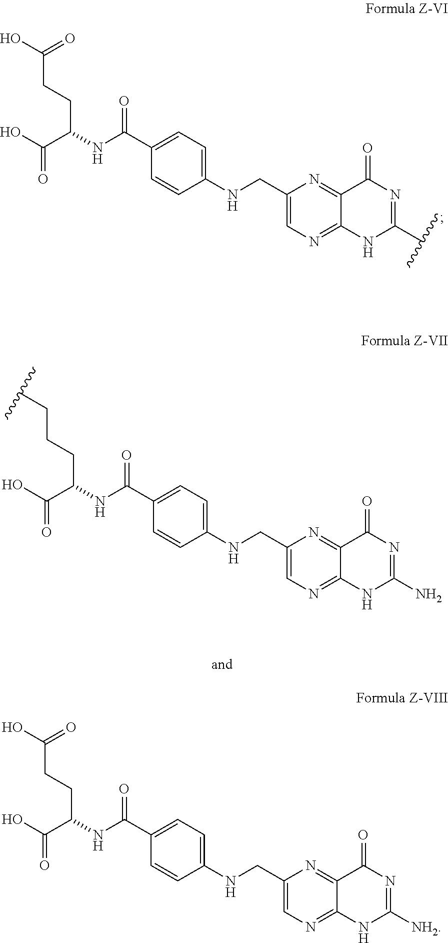

In one embodiment, A is represented by Formula A-I or A-IV, X¹ is —O— and —B—Z is not —CH₂)₄—O—P(O)(OC₂H₅)₂. In another embodiment, A is represented by Formula A-II and X¹ is not —O— and/or —B— is an aliphatic substituent with 1 to 100, preferably with 1 to 42 carbon atoms. In another embodiment, A is selected from the group consisting of:

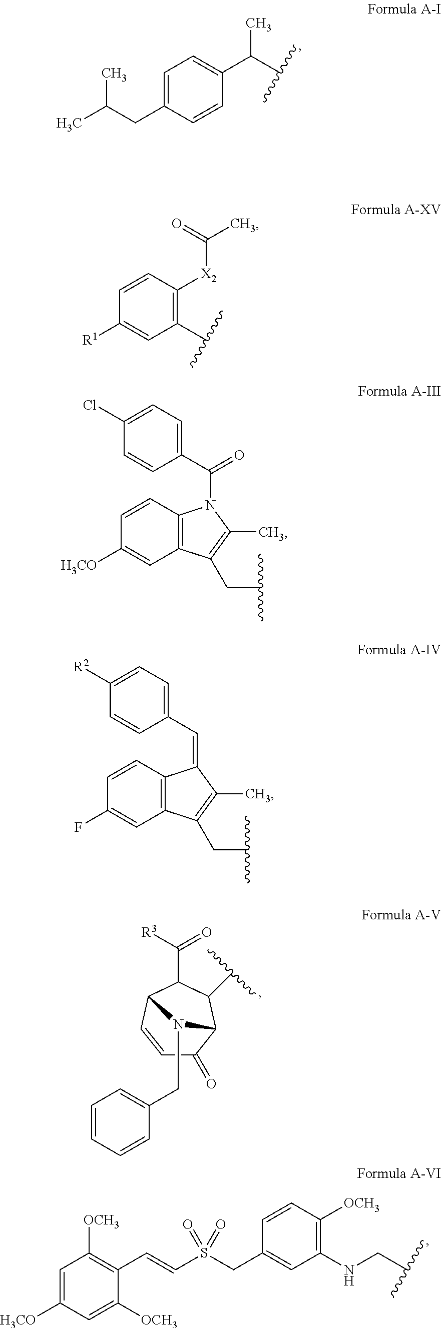

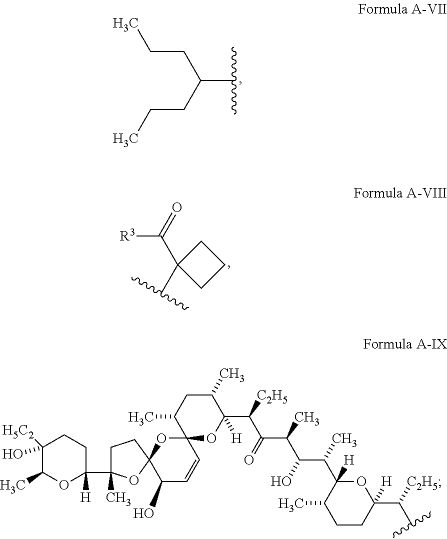

R¹ is selected from hydrogen and trifluoromethyl;

R² is selected from —SCH₃, —S(O)CH₃ or —S(O)₂CH₃;

R³ is selected from hydroxy, —B—Z and

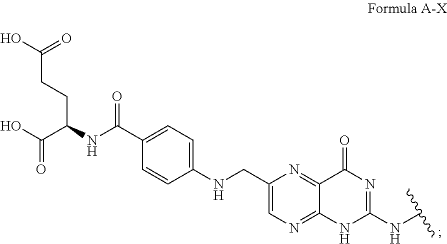 [SMILES: *Nc1nc(=O)c2nc(CNc3ccc(C(=O)N[C@H](CCC(=O)O)C(=O)O)cc3)cnc2[nH]1]

X¹ and X² are independently selected from the group consisting of —O—, —S— and NR⁴—, R⁴ being hydrogen or C₁₋₆-alkyl;

B is selected from the group consisting of

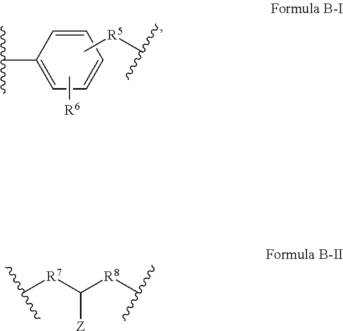

and an aliphatic group, preferably with 1 to 22 carbon atoms,

R⁵, R⁷ and R⁸ is the same or different _{C1-3}-alkylene,

R⁶ is hydrogen, C₁₋₃-alkyl, halo or methoxy,

Z is selected independently from the group consisting of

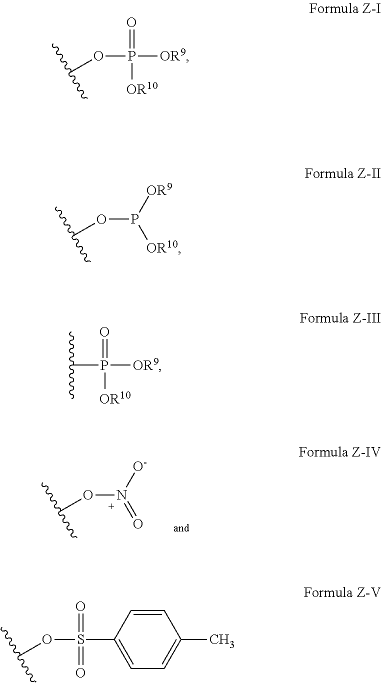

R⁹ is independently selected from hydrogen, C₁₋₆-alkyl or polyethylene glycol residue,

R¹⁰ is selected from C₁₋₆-alkyl or polyethylene glycol residue;

or B together with Z forms a structure

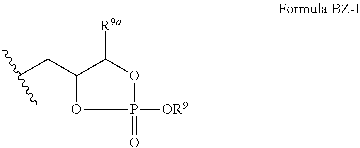

R⁹ being defined as above,

R^{9a} being independently selected from hydrogen, an aliphatic group, preferably with 1 to 22 carbon atoms more preferred C₁₋₆-alkyl or polyethylene glycol residue, provided that if A is represented by Formula A-I or A-IV and X¹ is —O— then —B—Z is not —(CH₂)₄—O—P(O)(OC₂H₅)₂; further provided that if A is represented by Formula A-II—then X¹ is not —O— and/or —B— is an aliphatic group with 1 to 22 carbon atoms.

In one embodiment, the present invention relates to a compound of Formula I for use in the treatment and/or prevention of pain. In a further embodiment, the present invention relates to a compound of Formula I for use in the treatment and/or prevention of cancer and/or precancerous conditions thereof. In another embodiment, the present invention relates to a compound of Formula I for use in the treatment and/or prevention of inflammation-related diseases. In another embodiment, the present invention relates to a compound of Formula I for use as an antipyretic (i.e., fever-reducing) agent. In yet another embodiment, the present invention relates to a compound of Formula I for the prevention of thrombus formation, the dissolution of thrombus formation, or the inhibition of platelet aggregation in general or more specifically in the cardiovascular system or even more specifically in coronary arteries.

In a further aspect, the present invention is directed to a pharmaceutical composition comprising a compound of Formula I, as described generally herein, and a pharmaceutically acceptable excipient. Pharmaceutical compositions of the present invention can comprise one or more further pharmaceutical agents in addition to one or more compounds of Formula I. Each compound of Formula I can be administered alone or in combination with other active agents.

A further embodiment of the present invention provides pharmaceutical compositions for prevention and/or treatment of cancer or precancerous conditions, including but not limited to precancerous conditions, such as benign prostatic hypertrophy, colon adenomas, actinic keratosis and various premalignant conditions of the lung, breast, oral cavity, cervix, and pancreas, and also cancer of the mouth, stomach, colon, rectum, lung, prostate, liver, breast, pancreas, skin, brain, head and neck, bones, ovaries, testicles, uterus, small bowel, lymphoma and leukemia.

A further embodiment of the present invention provides pharmaceutical compositions for the prevention of arterial or venous thrombosis or for the dissolution of thrombi or early blot clots or for the prevention of platelet aggregation.

A further embodiment of the present invention provides pharmaceutical compositions for the prevention of arterial or venous thrombosis, or for the dissolution of thrombi or early blot clots, or for the prevention of platelet aggregation. In another embodiment, the composition is useful in the treatment of human and animal inflammation related diseases, including, but not limited to neoplasms (i.e., tumors), rheumatologic diseases such as rheumatoid arthritis and Sjogren's syndrome; cardiovascular diseases, such as coronary artery disease, peripheral vascular disease and hypertension; neurodegenerative diseases, such as Alzheimer's disease and its variants or cerebrovascular diseases; and autoimmune diseases for example lupus erythematosus.

In a further embodiment, the invention is directed to a method for inhibiting chronic inflammation in a subject in need thereof, by administering to the subject an amount of the compound or composition of the present invention effective to inhibit inflammation. The subject may be a human patient or animal, for instance a mammal.

In yet another embodiment, the present invention provides methods for treating any disorder related to undesirable inflammation comprising administering to a subject (e.g., human patient or animal) in need thereof a therapeutically effective amount of a compound of Formula I of the invention or a pharmaceutical composition comprising a compound of the invention. In a preferred embodiment, the disorder includes, but is not limited to rheumatologic diseases such as for example rheumatoid arthritis and Sjogren's syndrome; cardiovascular diseases, such as, for example, coronary artery disease, peripheral vascular disease and hypertension; neurodegenerative diseases, such as, for example, Alzheimer's disease and its variants or cerebrovascular diseases; autoimmune diseases such as for example lupus erythematosus; and other conditions characterized by chronic inflammation of organs such as for example the lung, such as chronic bronchitis or the sinuses, such as chronic sinusitis, and the skin, including eczema or atopic dermatitis, dryness of the skin and recurring skin rashes, contact dermatitis and seborrhoeic dermatitis, neurodermatitis and discoid and venous eczema.

In another embodiment, the present invention provides methods for treating pain. The invention further pertains to a method for alleviating pain, comprising administering to a subject in need thereof a pharmaceutically effective amount of a compound of the present invention or of a pharmaceutical composition of the present invention.

In another embodiment, the present invention pertains to a method for treating fever, comprising administering to a subject in need thereof a pharmaceutically effective amount of a compound of the present invention or of a pharmaceutical composition of the present invention.

A further embodiment of the present invention provides methods for the prevention and treatment of venous thrombosis and arterial thrombosis including but not limited to deep vein thrombosis, portal vein thrombosis, renal vein thrombosis, jugular vein thrombosis, Budd-Chiari syndrome, Paget-Schroetter disease, cerebral venous sinus thrombosis, arterial thrombosis, stroke, myocardial infarction, angina, unstable angina, mural thrombus, hepatic artery thrombosis and arterial embolization.

A further embodiment of the present invention provides methods for the prevention and treatment of venous thrombosis and arterial thrombosis including but not limited to deep vein thrombosis, portal vein thrombosis, renal vein thrombosis, jugular vein thrombosis, Budd-Chiari syndrome, Paget-Schroetter disease, cerebral venous sinus thrombosis, arterial thrombosis, stroke, myocardial infarction, angina, unstable angina, mural thrombus, hepatic artery thrombosis and arterial embolization.

The compounds represented by Formula I may be used for the manufacture of pharmaceutical compositions for treatment of a disease listed above.

In addition to one or more compounds of Formula I, the pharmaceutical compositions of the present invention can comprise one or more further pharmaceutical agents, for instance, compounds having anti-cancer activity. The compound of Formula I can be administered alone or in combination with other active agents.

### BRIEF DESCRIPTION OF THE FIGURES

FIG. 1 shows a nose-only aerosol exposure system.

FIGS. 2A-2F show modes of administration of a compound of Formula I.

FIG. 3 shows the biodistribution of liposomal phospho-ibuprofen amide 1 in mice after i.v. administration at 200 mg/kg.

FIG. 4 shows, in addition to representative fluorescence images of lungs from control (left), ibuprofen (center) and phospho-ibuprofen amide 1 (right) treated mice, the amount of lung tumor per group (based on fluorescence intensity).

FIG. 5 shows inhibition of human lung cancer by phospho-ibuprofen amide 1.

FIG. 6 shows pharmacokinetic study of PTI in mice. Following a single i.p. dose of 100 mg/kg PTI 93 (FIG. 6A) or 58 mg/kg indomethacin (127) (equimolar to PTI) (Indo; FIG. 6B) the plasma levels of intact PTI 93 and indomethacin (127) (hydrolysis product of PTI) were determined at the indicated time points.

FIG. 7 shows effective inhibition of human cancer cell xenograft tumor growth by phospho-tyrosyl-indomethacin (PTI).

FIG. 8 shows levels of phospho-sulindac (PS) 96 and its metabolites in the lungs (A) and plasma (B) of mice subjected to aerosol administration of PS.

FIG. 9 shows survival rates of control and aerosolized-PS treated groups of mice implanted orthotopically with human A549 lung cancer cells (hereinafter “A549 cells”).

FIG. 10 shows reduced tumor load in response to aerosol administration of PS in mice. Tumor size is visualized by Green Fluorescent Protein (GFP) fluorescence in A549 cells expressing recombinant GFP.

FIG. 11 demonstrates tumor shrinkage in response to aerosol administration of PS. 11A demonstrates tumor size as quantified by GFP fluorescence (i.e., luminosity). 11B shows tumor size as determined by overall weight of lung tissue in milligrams; lung weight tends to underestimate the effect of the drug as it includes normal tissue as well.

FIG. 12 shows lung level of PS after inhalation and oral administration in mice.

FIG. 13 shows plasma level of PS after inhalation and oral administration to mice.

FIG. 14 shows the synergistic effect of phosphovalproic acid (PV, 116) and ibuprofen phospho-glycerol amide (PGIA, 4) on the growth of glioblastoma and lung cancer cells (14A). As demonstrated (14B), administering a combination of PV and PGIA significantly enhances the number of Annexin V (+) cells (i.e., cells undergoing apoptosis), as compared to administration of PV monotherapy, PGIA monotherapy, or the sum effect of PV monotherapy and PGIA monotherapy combined.

FIG. 15 shows HPLC chromatograms of extracts from A431 cells treated with ibuprofen, phospho-ibuprofen (PI) 2 bearing phosphate and phospho-diethylphosphate. The vertical lines indicate the respective position in the chromatograms of the peaks of authentic compounds. PI phosphate and ibuprofen generated no discernible peaks.

FIG. 16 shows a pharmacokinetic study of phosphosulindac amide (PSA) 95 and sulindac. 100 mg/kg PSA 95 or 62 mg/kg sulindac (equimolar to PSA, 95) were administered to mice as a single oral gavage dose in corn oil and blood samples were collected at the indicated time points starting at 15 minutes post injection. Plasma levels of the PSA 95 or sulindac metabolites (sulindac sulfide and sulindac sulfone) were determined. Values are the average of duplicate samples (all within 12% of each other).

FIG. 17 shows colon cancer growth inhibition by PSA 95. 17A: PSA 95 inhibited human colon cancer cell xenograft tumor growth. Mice with SW480 human colon cancer xenografts were treated with PSA 95 100 mg/kg/day or vehicle (corn oil) by oral gavage. 17B: Effect of PSA 95 on tumor multiplicity in Apc^{4Min/±} mice. The total number of tumors per animal was reduced after PSA 95 treatment by 85%. Values are Mean±SEM.

FIG. 18 shows the toxicity assessment of phospho-tyrosyl-indomethacin (PTI). 18A: Representative H&E stained gastric tissue sections from control, indomethacin (Indo) or PTI treated mice. Indomethacine caused gastric damage but not PTI. The numerical results are shown below. 18B: PTI shows no genotoxicity; TA98. Salmonella typhimurium strain.

FIG. 19 shows a pharmacokinetic study of PTI in mice. Following a single i.p. dose of 100 mg/kg PTI (19A) or 58 mg/kg indomethacin (equimolar to PTI) (Indo; 19B) the plasma levels of intact PTI and indomethacin (hydrolysis product of PTI) were determined at the indicated time points. The AUCₜₒₜₐₗ of PTI is about 3.5 times higher than that of indomethacin.

FIG. 20 shows effective inhibition of human cancer cell xenograft tumor growth by PTI. Mice with A549 human non-small cell lung cancer (20A) or SW480 human colon cancer (20B) xenografts were treated with PTI 10 or 15 mg/kg/day or vehicle (corn oil) by oral gavage as indicated. Mice with lung cancer xenografts followed a treatment protocol (treatment started when xenografts reached an average volume of 100 mm³ whereas those with SW480 xenografts followed a prevention protocol (drug administration started 1 week prior to cell implantation). Values are Mean±SEM.

FIG. 21 shows a pharmacokinetic study of PEGylated phospho-ibuprofen (PI-PEG) and phospho-ibuprofen (PI) 2 in mice, x-axis: time, hours; y-axis: PI-PEG concentration, μM.

FIG. 22 shows tumor volumes of SW-480 xenografts in nude mice treated with PBS (control) or with PI-PEG (n=9 tumors/group).

FIG. 23 shows the growth of orthotopic MDA-MB231 human breast cancer xenografts in nude mice treated with phospho-aspirin (PA) or acetylsalicylic acid (ASA), starting 1 week prior to cell implantation.

FIG. 24 shows phospho-aspirin (PA) metabolites in plasma (24A) and tumors (24B) and the effect of cytochrome P450 (CYP) isoforms (24C).

FIG. 25 shows a time course of the levels of PA and its metabolites in PA-treated microsomes.

FIG. 26 shows that phospho farnesylthiosalicylic acid (P-FTS) inhibits pancreatic cancer cell growth in vitro in a concentration-dependent manner.

FIG. 27 shows that P-FTS inhibits the growth of MIA PaCa-2 human pancreatic cancer xenografts.

FIG. 28 shows that xenograft tumors treated with either vehicle (control) or P-FTS and stained for Ki-67 expression (proliferation marker) (28A) or by the TUNEL method (apoptosis) (28B).

FIG. 29 shows that P-FTS inhibits Ras activation and reduces ERK1/2 and AKT activation in pancreatic cancer cells. A: Immunoblots of Ras-GTP (active-Ras) and total K-Ras in Panc-1 cells treated without or with P-FTS, as indicated, for 24 hours. B: Immunoblots of Ras-GTP (active Ras) in cell protein extracts from Panc-1 cells treated with 50 μM P-FTS, or 50 μM FTS for 24 hours. *P<0.05 vs. control. C: Immunoblots of p-c-RAF, c-RAF, p-MEK, MEK, p-AKT and AKT in whole cell protein extracts from Panc-1 cells treated with P-FTS as indicated. Loading control: β-actin.

FIG. 30 shows that P-FTS inhibits Ras activation and reduces ERK1/2 and AKT activation in pancreatic xenografts. A: Immunoblots of Ras-GTP (active-Ras) and total K-Ras. B: Immunoblots of p-ERK, ERK, p-AKT and AKT in whole cell protein extracts from MIA PaCa-2 xenografts. Loading control: β-actin. C: Growth of xenograft tumors treated without or with P-FTS and stained for p-ERK expression. The percentage of p-ERK positive cells/field were determined and expressed as the mean±SEM (*P<0.03).

FIG. 31 shows that phospho-valproic acid 116 enhances P-FTS-induced inhibition of pancreatic cancer cell growth. A: Cell viability was determined in MIA PaCa-2 after 24 h of incubation with P-V, P-FTS or both. Results are expressed as % control. B: In this isobologram the additivity line connects the IC₅₀ value of each compound used alone. A and B in the isobolograms represent two different dose pairs of each compound (their respective concentrations are shown in parentheses). The location of both A and B below the additivity line signifies a synergistic effect over the sum of their individual effects combined. C: The percentages of apoptotic cells determined by flow cytometry using the dual staining (Annexin V and PI). The percent of Annexin V(+) cells was calculated and results expressed as % control.

FIG. 32 shows the antithrombotic effect of PS. Carotid artery blood flow following injury with FeCl₃ is examined. In FIG. 32A, a control animal the flow is markedly reduced to near obliteration. In FIG. 32B, a phospho-sulindac 96 treated animal the flow rate is maintained establishing the antithrombotic effect of the test agent.

FIG. 33 shows inhibition of platelet aggregation by various concentrations of phospho-sulindac amide 95.

FIG. 34 shows ex vivo platelet aggregometry in blood obtained from mice treated with phospho-sulindac 96. Maximum inhibition of platelet aggregation was at 1 hr.

FIG. 35 shows that phospho-sulindac (PS) is more efficacious against Lewis lung carcinoma (LLC) in Ces −/− mice. Efficacy of PS (150 mg/kg, i.p.) in wild type (FIG. 35A) and Ces1c −/− mice (FIG. 35B) bearing subcutaneous LLC tumors is shown.

### DETAILED DESCRIPTION OF THE INVENTION

The present invention provides novel compounds and pharmaceutical compositions for the prevention and/or treatment of cancer and precancerous conditions thereof, for the treatment of pain and fever, for the treatment of skin disorders, and for treating and/or preventing inflammation-related diseases and/or cardiovascular diseases. The compounds of the invention also have analgesic properties and antiplatelet properties. The compounds of the invention may be provided to animals, including mammals and humans, by administering a suitable pharmaceutical dose in a suitable pharmaceutical dosage form. The compounds of the invention have improved efficacy and safety, including higher potency and/or fewer or less severe side effects, than conventional therapies.

The compounds of the invention comprise a biologically active moiety or portion (A) that has, or is modified to have at least one carboxyl group. The moiety A is preferably an aliphatic, aromatic or alkylaryl group, preferably derived from a nonsteroidal anti-inflammatory drug or NSAID (A). The moiety A is bound to a linker moiety (B) via the carboxyl of group A and a linking atom that is selected from oxygen, nitrogen, and sulphur, to form a carboxylic ester, and amide, or a thioester, bond (X¹) between groups A and B. Moiety B is a single bond, an aliphatic group, a substituted benzene, or an alkylene substituted hydrocarbon chain, which in turn is bound to functional moiety Z, which facilitates access of the compound into cells. The moiety Z can comprise, for example, a phosphorous-containing group, a nitrogen-containing group, or a folic acid residue. Suitable choices for moieties A, X¹, B and Z are given herein. Further, it will be understood that each specific moiety A, X¹, B and Z in each of the specific examples herein is representative, and may be used in other combinations with other exemplified moieties, i.e. any A, X¹, B or Z moiety in any exemplary compound disclosed herein may potentially be substituted by any other A, X¹, B or Z moiety disclosed herein.

Defined Terms

The term “aliphatic substituent”, as used herein, includes saturated or unsaturated, branched or unbranched aliphatic univalent or bivalent substituents. In the present application, “aliphatic substituent” is intended to include, but is not limited to, alkyl, cycloalkyl, alkylene, alkenylene, alkynylene and alkadienylene substituents. According to the present invention, the aliphatic substituent has 1 to 100, preferably 1 to 42 carbon atoms, preferably 1 to 22 carbon atoms, more preferred 1 to 15 carbon atoms, further preferred 1 to 10 carbon atoms, even more preferred 1 to 6 carbon atoms, for instance 4 carbon atoms. Most preferably, the aliphatic substituent is C₁₋₆-alkylene, e.g. methylene, ethylene, trimethylene and tetramethylene.

The term “alkyl” used in the present application refers to a saturated branched or unbranched aliphatic univalent substituent. Preferably, the alkyl substituent has 1 to 100 carbon atoms, more preferred 1 to 22 carbon atoms, further preferred 1 to 10 carbon atoms, yet more preferred 1 to 6 carbon atoms, even more preferred 1 to 3 carbon atoms. Accordingly, examples of the alkyl substituent include methyl, ethyl, n-propyl, isopropyl, n-butyl, isobutyl, sec-butyl, tert-butyl, n-pentyl and n-hexyl and preferable examples include methyl, ethyl, n-propyl and isopropyl, whereby ethyl and isopropyl are particularly preferred.

As used herein, the term “cycloalkyl” refers to a monocyclic, bicyclic, or tricyclic substituent, which may be saturated or partially saturated, i.e. possesses one or more double bonds. Monocyclic substituents are exemplified by a saturated cyclic hydrocarbon group containing from 3 to 8 carbon atoms. Examples of monocyclic cycloalkyl substituents include cyclopropyl, cyclobutyl, cyclopentyl, cyclopentenyl, cyclohexyl, cyclohexenyl, cycloheptyl and cyclooctyl. Bicyclic fused cycloalkyl substituents are exemplified by a cycloalkyl ring fused to another cycloalkyl ring. Examples of bicyclic cycloalkyl substituents include, but are not limited to decalin, 1,2,3,7,8,8a-hexahydronaphthalene, and the like. Tricyclic cycloalkyl substituents are exemplified by a cycloalkyl bicyclic fused ring fused to an additional cycloalkyl substituent.

The term “alkylene” used in the present application refers to a saturated branched or unbranched aliphatic bivalent substituent. Preferably, the alkylene substituent has 1 to 6 carbon atoms, more preferred 1 to 3 carbon atoms. Accordingly, examples of the alkylene substituent include methylene, ethylene, trimethylene, propylene, tetramethylene, isopropylidene, pentamethylene and hexamethylene. Preferable examples of the alkylene substituent include methylene, ethylene, trimethylene and tetramethylene, whereby tetramethylene is particularly preferred.

The term “alkenylene” as used in the present application is an unsaturated branched or unbranched aliphatic bivalent substituent having a double bond between two adjacent carbon atoms. Preferably, the alkenylene substituent has 2 to 6 carbon atoms, more preferred 2 to 4 carbon atoms. Accordingly, examples of the alkenylene substituent include but are not limited to vinylene, 1-propenylene, 2-propenylene, methylvinylene, 1-butenylene, 2-butenylene, 3-butenylene, 2-methyl-1-propenylene, 2-methyl-2-propenylene, 2-pentenylene, 2-hexenylene. Preferable examples of the alkenylene substituent include vinylene, 1-propenylene and 2-propenylene, whereby vinylene is particularly preferred.

The term “alkynylene” as used in the present application is an unsaturated branched or unbranched aliphatic bivalent substituent having a triple bond between two adjacent carbon atoms. Preferably, the alkynylene substituent has 2 to 6 carbon atoms, more preferred 2 to 4 carbon atoms. Examples of the alkynylene substituent include but are not limited to ethynylene, 1-propynylene, 1-butynylene, 2-butynylene, 1-pentynylene, 2-pentynylene, 3-pentynylene and 2-hexynylene. Preferable examples of the alkenylene substituent include ethynylene, 1-propynylene and 2-propynylene, whereby ethynylene is particularly preferred.

The term “alkadienylene” as used in the present application is an unsaturated branched or unbranched aliphatic bivalent substituent having two double bonds between two adjacent carbon atoms. Preferably, the alkadienylene substituent has 4 to 10 carbon atoms. Accordingly, examples of the alkadienylene substituent include but are not limited to 2,4-pentadienylene, 2,4-hexadienylene, 4-methyl-2,4-pentadienylene, 2,4-heptadienylene, 2,6-heptadienylene, 3-methyl-2,4-hexadienylene, 2,6-octadienylene, 3-methyl-2,6-heptadienylene, 2-methyl-2,4-heptadienylene, 2,8-nonadienylene, 3-methyl-2,6-octadienylene, 2,6-decadi-enylene, 2,9-decadienylene and 3,7-dimethyl-2,6-octadienylene substituents, whereby 2,4-pentadienylene is particularly preferred.

The term “heteroaliphatic substituent”, as used herein, refers to a monovalent or a bivalent substituent, in which one or more carbon atoms have been substituted with a heteroatom, for instance, with an oxygen, sodium, sulfur, nitrogen, phosphorus or silicon atom, wherein the nitrogen and sulfur atoms may optionally be oxidized, and the nitrogen heteroatom may optionally be quaternized. The heteroatom(s) O, N and S may be placed at any interior position of the heteroaliphatic substituent. Examples include —CH₂—CH₂—O—CH₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —CH═CH—O—CH₃, —CH₂—CH═N—OCH₃, and —CH═CH—N(CH₃)—CH₃. A heteroaliphatic substituent may be linear or branched, and saturated or unsaturated.

In one preferred embodiment, the heteroaliphatic substituent has 1 to 100, preferably 1 to 42 carbon atoms. In another preferred embodiment, the heteroaliphatic substituent is a polyethylene glycol (PEG) residue. In another preferred embodiment, the heteroaliphatic substituent is a phosphate residue. In yet another preferred embodiment, the heteroaliphatic substituent is a sodium phosphate residue.

The term “polyethylene glycol” (PEG) refers to a compound of formula H—(OCH₂CH₂)ₙOH in which n has a value typically from 21 to 135, but which is not restricted to this range. Commercial polyethylene glycols having number average molecular weights of 1,000, 1,500, 1,540, 4,000 and 6,000 are exemplary PEGs which are useful in this invention. These solid polyethylene glycols have melting points of 35° C. to 62° C. and boiling or flash points ranging from 430° C. to over 475° C. Preferred polyethylene glycol residues falling within the definition of the present invention are those having the formula —(OCH₂CH₂)ₙOCH₃ in which n is from 21 through 135, and preferably from 40 to 50.

As used herein, “aromatic substituent” is intended to mean any stable monocyclic, bicyclic or polycyclic carbon ring of up to 10 atoms in each ring, wherein at least one ring is aromatic, and may be unsubstituted or substituted. Examples of such aromatic substituents include phenyl, p-toluenyl (4-methylphenyl), naphthyl, tetrahydronaphthyl, indanyl, biphenyl, phenanthryl, anthryl or acenaphthyl. In cases where the aromatic substituent is bicyclic and one ring is non-aromatic, it is understood that attachment is via the aromatic ring.

The term “arylalkyl substituents” refers to alkyl substituents as described above wherein one or more bonds to hydrogen contained therein are replaced by a bond to an aryl substituent as described above. It is understood that an arylalkyl substituent is connected to the carbonyl group in the compound of Formula I through a bond from an alkyl substituent; that is, the substitution of alkyl substituents by one or more arylalkyl substituents is such that at least one alkyl substituent is available for attachment to a carbonyl group. Examples of arylalkyl substituents include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaromatic substituent” as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic heteroaromatic substituents include phenyl, pyridine, pyrimidine or pyridizine rings that are

a) fused to a 6-membered aromatic (unsaturated) heterocyclic ring having one nitrogen atom;

b) fused to a 5- or 6-membered aromatic (unsaturated) heterocyclic ring having two nitrogen atoms;

c) fused to a 5-membered aromatic (unsaturated) heterocyclic ring having one nitrogen atom together with either one oxygen or one sulfur atom; or

d) fused to a 5-membered aromatic (unsaturated) heterocyclic ring having one heteroatom selected from O, N or S.

Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline.

In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The aliphatic, heteroaliphatic, aromatic and heteroaromatic substituents can be optionally substituted one or more times, the same way or differently with any one or more of the following substituents including, but not limited to: aliphatic, heteroaliphatic, aromatic and heteroaromatic substituents, aryl, heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; NaH₂PO₄; Na₂HPO4; —N(Rₓ)₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, hetero-aromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, (alkyl)aryl or (alkyl)heteroaryl substituents described above and herein may be substituted or unsubstituted. Additionally, it will be appreciated, that any two adjacent substituents taken together may represent a 4, 5, 6, or 7-membered substituted or unsubstituted alicyclic or heterocyclic substituent. Additional examples of generally applicable substituents are illustrated by the specific embodiments shown below.

The terms “halo” and “halogen” refer to a halogen atom selected from the group consisting of F, Cl, Br and I. Preferably the halogen atom is Cl or Br, whereby Cl is particularly preferred.

The term “halogenated alkyl substituent” refers to an alkyl substituent as defined above which is substituted with at least one halogen atom. In a preferred embodiment, the halogenated alkyl substituent is perhalogenated. In a more preferred embodiment, the halogenated alkyl substituent is a univalent perfluorated substituent of formula CₙF2ₙ₊₁. Preferably, the halogenated alkyl substituent has 1 to 6 carbon atoms, even more preferred 1 to 3 carbon atoms. Accordingly, examples of the alkyl group include trifluoromethyl, 2,2,2-trifluoroethyl, n-periluoropropyl, n-perfluorobutyl and n-perfluoropentyl. Preferable examples of halogenated alkyl substituents include trifluoromethyl and 2,2,2-trifluoroethyl, whereby trifluoromethyl is particularly preferred.

The phrase, “pharmaceutically acceptable derivative”, as used herein, denotes any pharmaceutically acceptable salt, ester, or salt or cocrystal of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others prodrugs. A prodrug is a derivative of a compound, usually with significantly reduced pharmacological activity, which contains at least one additional moiety, which is susceptible to removal in vivo yielding the parent molecule as the pharmacologically active species. An example of a prodrug is an ester, which is cleaved in vivo to yield a compound of interest. Prodrugs of a variety of compounds, and materials and methods for derivatizing the parent compounds to create the prodrugs, are known and may be adapted to the present invention.

The term “smoking” as used herein, refers to the action of inhaling or tasting the smoke of burning plant material, preferably of tobacco leaves. Smoking further includes a process wherein the smoking composition is heated but not pyrolysed, and the heated vapors are inhaled or tasted by the smoker.

The Compounds of the Invention

One aspect of the present invention relates to the compound of Formula I above,

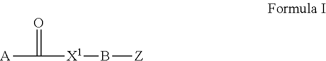

or an enantiomer, a diastereomer, a racemate, a tautomer, salt or hydrate or cocrystal thereof, in which A is an optionally substituted aliphatic, heteroaliphatic, aromatic, heteroaromatic substituent or alkylaryl substituent having 1 to 100, preferably 1 to 42 carbon atoms. Preferably, A is derived from among non-steroidal anti-inflammatory drugs (NSAIDs) having a carboxylic acid moiety in the structure, whereby the carbonyl group of said carboxylic acid moiety corresponds to the carbonyl group in the Formula I.

In some particularly preferred embodiments, the substituent A is derived from among the NSAIDs ibuprofen (Formula A-I), Aspirin® (Formula A-II), indomethacin (Formula A-III) or sulindac (Formula A-IV):

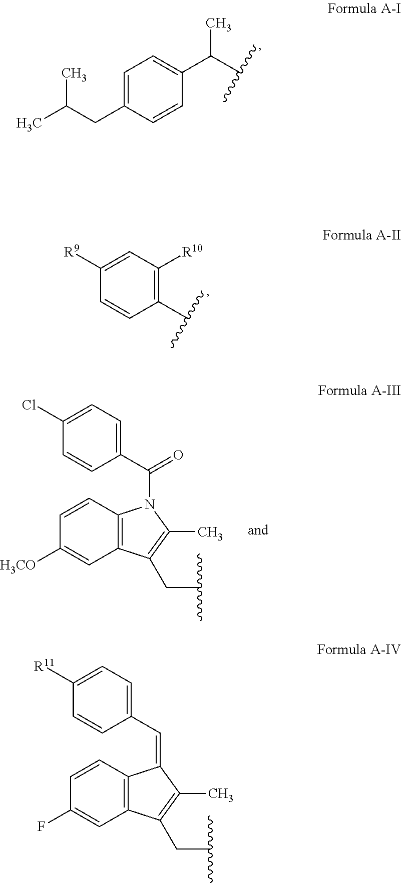

R⁹ being selected from hydrogen and trifluoromethyl,

R¹⁰ being selected from selected from —X²—C(O)—CH₃,

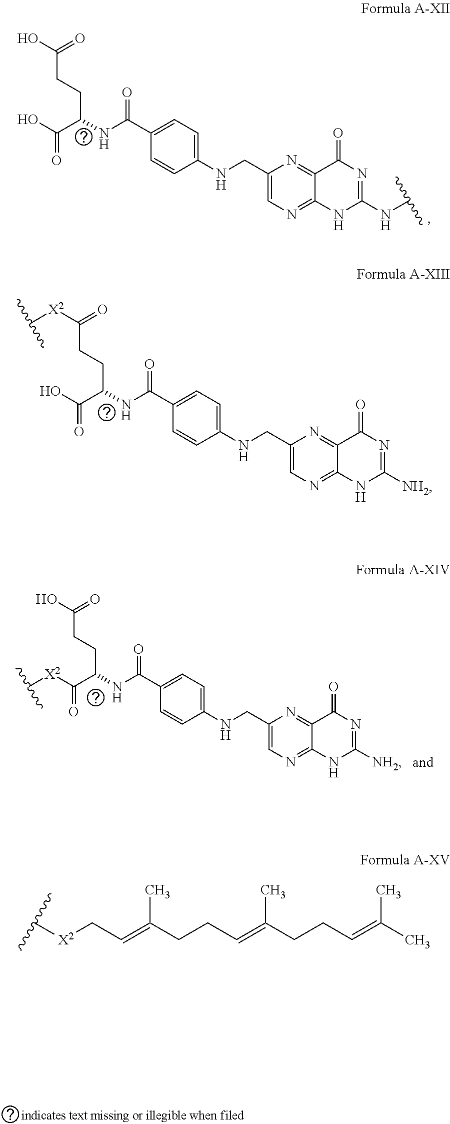

R¹¹ being selected from —SCH₃, —S(O)CH₃, —S(O)₂CH₃ and

X² being selected from the group consisting of —O—, —S— and —NR¹³—, whereby R¹³ is hydrogen or C₁₋₆-alkyl.

The substituent R¹¹ in Formula A-IV is selected from the group consisting of —SCH₃, —S(O)CH₃, —S(O)₂CH₃. Preferably R11 in Formula A-IV is —S(O)CH₃.

In some other embodiments the substituent A is represented by Formulas A-V to A-XI shown below:

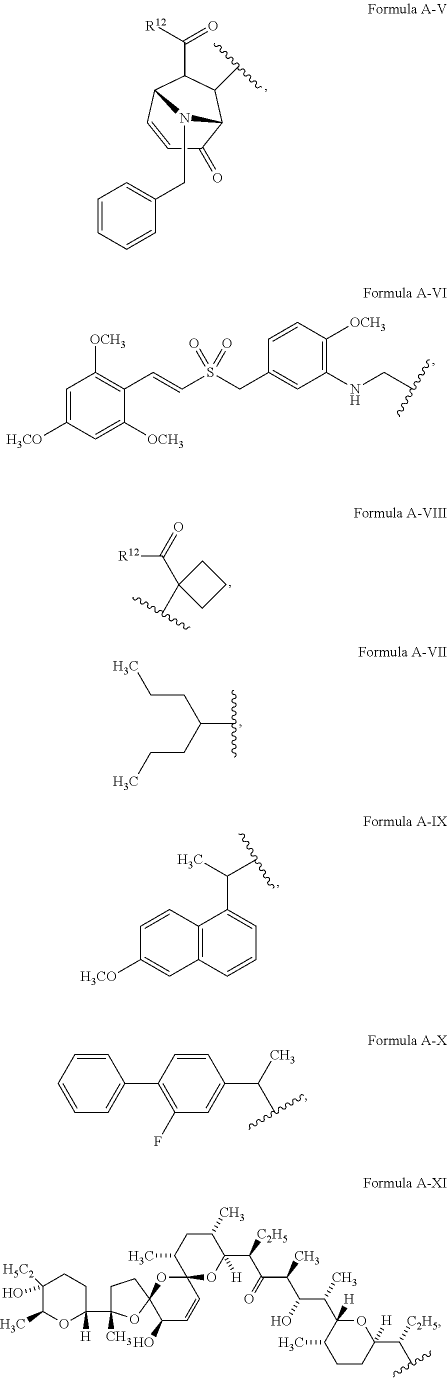

The substituent X¹ in Formula I can be —O—, —S— or —NR¹—, R¹ being hydrogen or an alkyl group having 1 to 100, preferably 1 to 22 carbon atoms, more preferred 1 to 10 carbon atoms, yet even more preferred 1 to 6 carbon atoms and particularly preferred 1 to 3 carbon atoms, such as for instance methyl or ethyl, preferably methyl.

In one of the preferred embodiments, the substituent X¹ in Formula I is —NR¹— and R¹ is hydrogen, the compounds of the present invention being represented by:

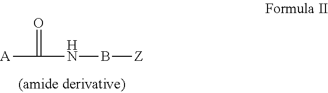

In other preferred embodiments, X¹ in Formula I is —O—. In these embodiments, the compounds of the present invention are represented by:

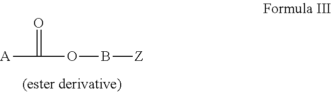

In yet another preferred embodiment, X¹ in Formula I is —S— and the compounds of the present invention are thus represented by:

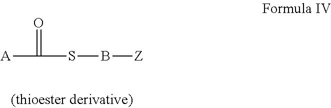

The substituent X² in R¹⁰ of Formula A-II can be —O—, —S— or —NR¹³—, R¹³ being hydrogen or an alkyl substituent having 1 to 3 carbon atoms. Preferably, R¹³ is hydrogen.

In some embodiments, A is represented by

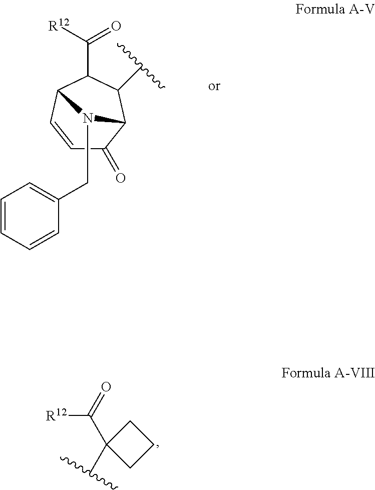

In these embodiments, R¹² is represented by hydroxy, —B—Z or

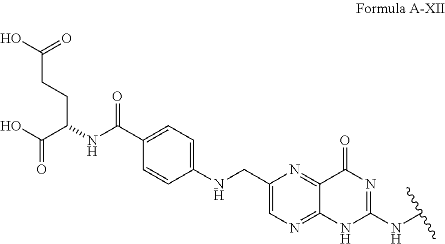

whereby —B— and —Z are as specified for Formula I.

The substituent represented by Formula A-XII is a folic acid residue. Without wishing to be bound by any theory, it is believed that compounds of the invention wherein R¹² is Formula A-XII have a particularly strong activity against lung and brain cancer. In particular, the activity against lung and brain cancer of compounds in which R¹² is represented by Formula A-XII is usually higher than activity against lung cancer of corresponding compounds in which R¹² is hydroxyl substituent.

In one embodiment A is represented by Formula A-1 or A-IV, X¹ is —O— and —B—Z is not —(CH₂)₄—O—P(O)(OC₂H₅)₂. In another embodiment A is represented by Formula A-II and X¹ is not —O— and/or —B— is an aliphatic substituent with 1 to 100, preferably with 1 to 42 carbon atoms.

The substituent B in any of Formulas I, II, III, and IV is

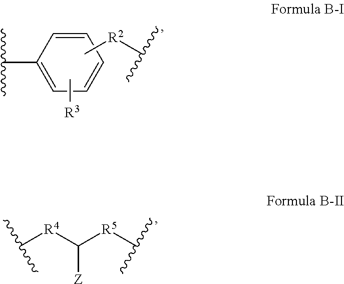

a single bond,

or an aliphatic substituent, preferably with 1 to 100, more preferred with 1 to 42 and particularly preferred with 1 to 22 carbon atoms, preferably with 1 to 15 carbon atoms, more preferred with 1 to 10 carbon atoms and particularly preferred with 1 to 6 carbon atoms.

Substituents R², R⁴, and R⁵ can be the same or different alkylene substituent having 1 to 3 carbon atoms. Preferably the substituent R² in Formula B-1 is selected from the group consisting of methylene, ethylene and trimethylene; in a more preferred embodiment, R² is methylene or ethylene, whereby methylene is particularly preferred.

In yet another preferred embodiment, the substituent B is represented by Formula B-II and R⁴, and R⁵ are identical alkylene substituent having 1 to 3 carbon atoms. In a particularly preferred embodiment, R⁴ and R⁵ are both methylene substituents so that the substituent B in Formula I is represented by Formula B-IV:

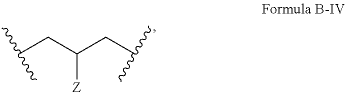

Z is defined below.

In a particularly preferred embodiment, the substituent B forms a glycerol ester residue together with X¹ and Z.

R³ in Formula B-1 can be hydrogen, C₁₋₆-alkyl, halogenated C₁₋₆-alkyl, C-₁₋₆-alkoxy, halogenated C₁₋₆-alkoxy, —C(O)—C₁₋₆-alkyl, —C(O)0-C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy substituents. In some preferred embodiments, R³ in Formula B-I is selected from hydrogen, an alkyl having 1 to 3 carbon atoms, halo and methoxy.

Preferably, R³ is selected from the group consisting of hydrogen, methyl, fluoro, chloro, bromo and methoxy, preferably from the group consisting of hydrogen, methyl, chloro and fluoro. In a particularly preferred embodiment, R³ represents hydrogen so that the substituent B is represented by Formula B-III:

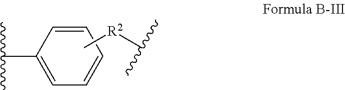

The substitution pattern of the substituent B in Formulas B-I and B-III is not particularly limited. When the substituent B is represented by Formula B-I II the aromatic moiety of the substituent B can be 1,2- or 1,3- or 1,4-substituted. Preferably, the aromatic moiety of B is 1,4-substituted so that B is represented by Formula B-V:

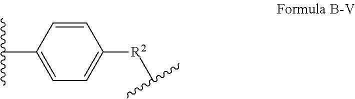

In yet another embodiment of the present invention the substituent B is an aliphatic substituent, preferably having 1 to 22 carbon atoms, more preferred 1 to 6 carbon atoms. In a particularly preferred embodiment, B is selected from the group consisting of alkylene substituents with 1 to 6 carbon atoms, alkenylene substituent having 2 to 6 carbon atoms and alkynylene substituent having 2 to 6 carbon atoms. In a more preferred embodiment, the substituent B is an alkylene substituent with 1 to 6 carbon atoms, preferably an alkylene substituent with 1 to 4 carbon atoms, even more preferably a tetramethylene substituent.

The substituent Z in any of Formulas I, II, III, and IV is selected from the group consisting of

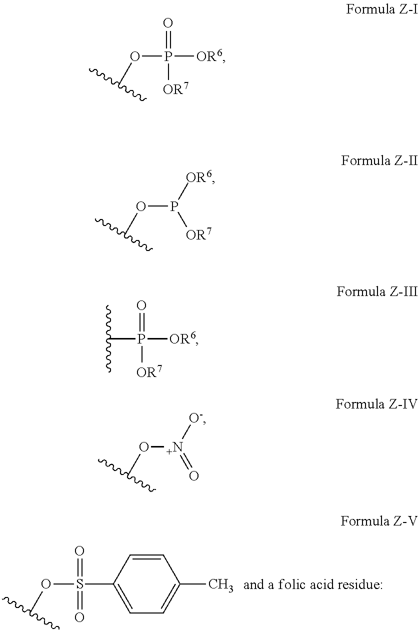

R⁶ is independently selected from hydrogen, C₁₋₁₀₀-alkyl, preferably C₁₋₆-alkyl, and polyethylene glycol substituent,

R⁷ is independently selected from hydrogen, C₁₋₁₀₀-alkyl, preferably C₁₋₆-alkyl, and polyethylene glycol substituent.

The folic acid residue is preferably selected from the one of the following:

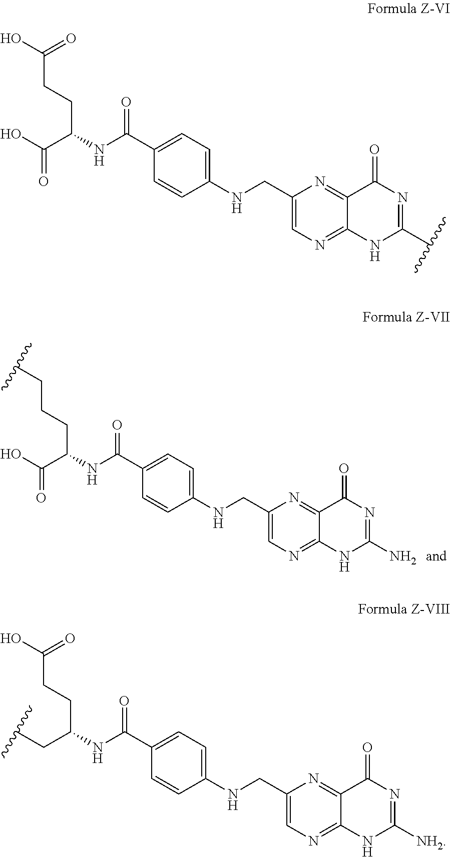 [SMILES: *c1nc(=O)c2nc(CNc3ccc(C(=O)N[C@@H](CCC(=O)O)C(=O)O)cc3)cnc2[nH]1 ; *CCC[C@H](NC(=O)c1ccc(NCc2cnc3[nH]c(N)nc(=O)c3n2)cc1)C(=O)O ; *C[C@H](CCC(=O)O)NC(=O)c1ccc(NCc2cnc3[nH]c(N)nc(=O)c3n2)cc1]

In a particularly preferred embodiment of the present invention, X¹ is —N— and Z is represented by Formula Z-VI. Accordingly, an exemplary compound of the present invention is represented by:

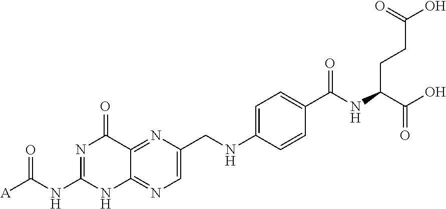

Preferably, the substituent Z is represented by Formula Z-I. In preferred embodiments, R⁶ and R⁷ are identical. Preferably, R⁶ and R⁷ are alkyl substituents having 1 to 42 carbon atoms, more preferred 1 to 22 carbon atoms, even more preferred 1 to 6 carbon atoms, yet even more preferred 1 to 3 carbon atoms, whereby it is most preferred that R⁶ and R⁷ are ethyl substituents. In yet another preferred embodiment, R⁶ is represented by hydrogen and R⁷ is polyethylene glycol residue, for instance (OCH₂CH₂)ₙOCH₃, whereby n is from 40 to 50.

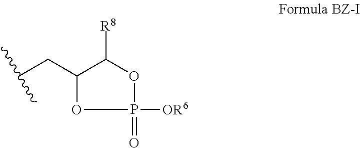

R⁶ is defined as above,

R⁸ is independently selected from hydrogen, an aliphatic substituent, preferably with 1 to 22 carbon atoms, more preferred C₁₋₆-alkyl, and a polyethylene glycol residue.

In yet another preferred embodiment, R⁸ is hydrogen and the substituent B together with the substituent Z forms a structure

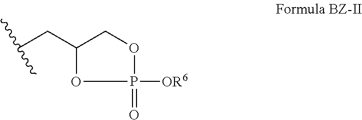

Thus, in some preferred embodiments of the present invention, X¹ is —NR¹—, R¹ is hydrogen;

the substituent B is selected from the group consisting of

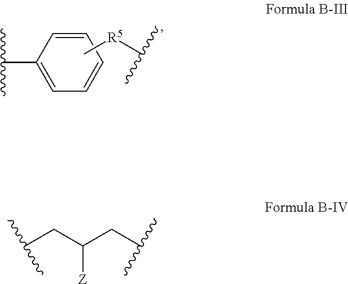

C₁₋₆-alkylene, C₂₋₆-alkenylene and C₂₋₆-alkynylene,

the substituent Z is represented by Formula Z-I and

R⁹ is independently selected from hydrogen, C₁₋₃-alkyl and (OCH₂CH₂)ₙOCH₃,

and

R¹⁰ is independently selected from C₁₋₃-alkyl and (OCH₂CH₂)ₙOCH₃, whereby n is from 40 to 50.

In another preferred embodiment, X¹ is —NR¹—, R¹ is hydrogen; the substituent B is selected from the group consisting of C-₁₋₄-alkylene and

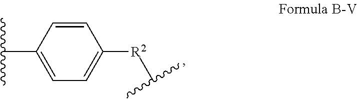

R² is methylene or ethylene; and

R² is methylene or ethylene; and

Z is represented by Formula Z-I, R⁶ and R⁷ being identical C₁₋₃-alkyl substituents.

In another preferred embodiment of the invention, X¹ is —O—; the substituent B is selected from the group consisting of C₁₋₄-alkylene and

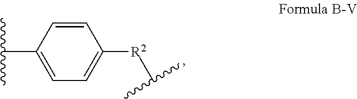

R² is methylene or ethylene; and,

the substituent Z is represented by Formula Z-I, R⁶ and R⁷ being identical C₁₋₃-alkyl substituents.

In yet another preferred embodiment, X¹ is —NR¹—, R¹ is hydrogen; the substituent B is —(CH₂)₄—; and the substituent Z is represented by Formula Z-I, R⁶ and R⁷ being identical C₁₋₃-alkyl substituents.

In yet another preferred embodiment, X¹ is —NH—, —S— or —O—; B is —(CH₂)₄—; and the substituent Z is represented by Formula Z-I, R⁶ and R⁷ being identical C₁₋₃-alkyl substituents.

If the substituent A is represented by Formula A-IV, then R² is preferably S(O)CH₃.

In one embodiment of the invention, the substituent A is represented by Formula A-I. The corresponding compounds are structurally related to ibuprofen. Accordingly, the compounds of Formula I include but are not limited to compounds 1 to 8, the structures of which are shown below:

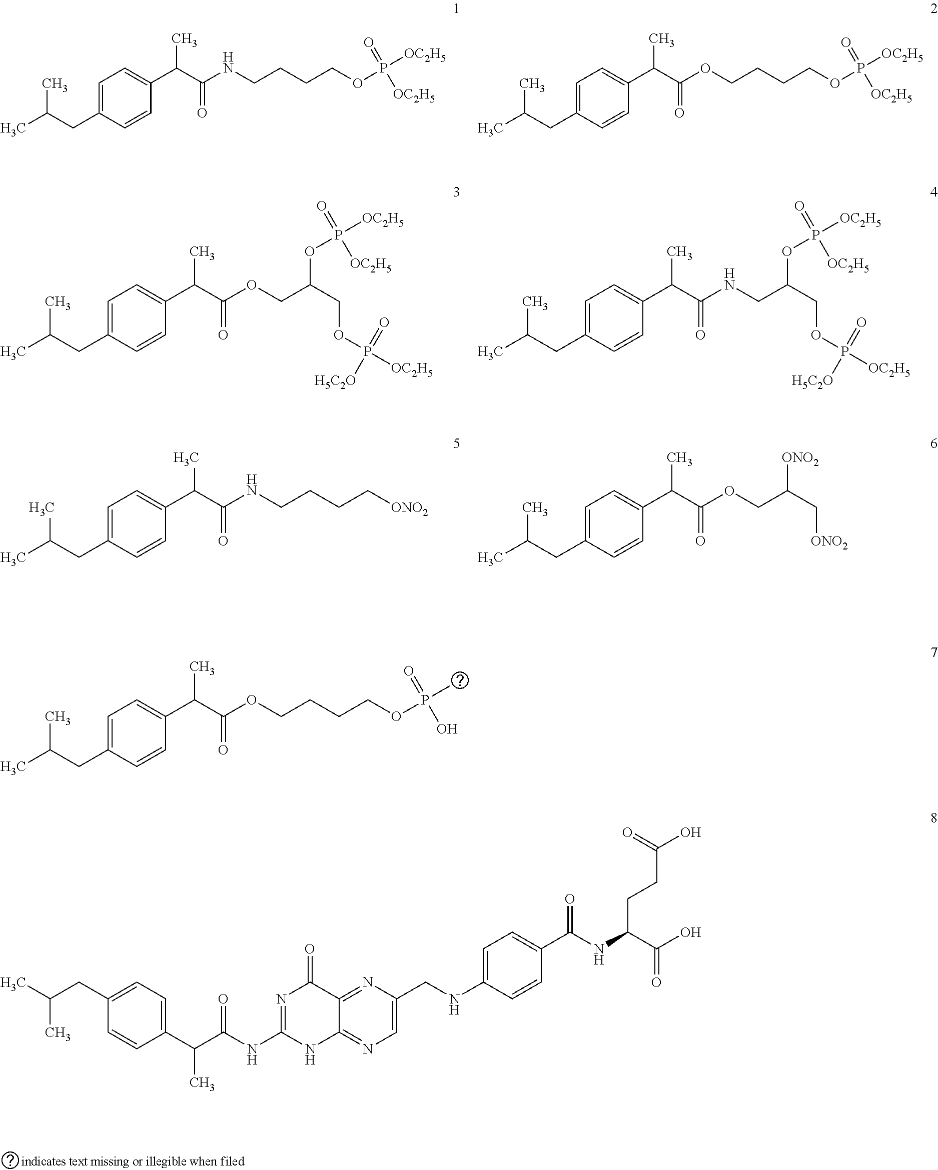

In another embodiment of the invention, the substituent A is represented by Formula A-II, R⁹ is hydrogen and R¹⁰ is —X²—C(O)—CH₃. Thus, the corresponding compounds are structurally related to acetylsalicylic acid (Aspirin®). The corresponding compounds of Formula I include but are not limited to the following compounds 9 to 32:

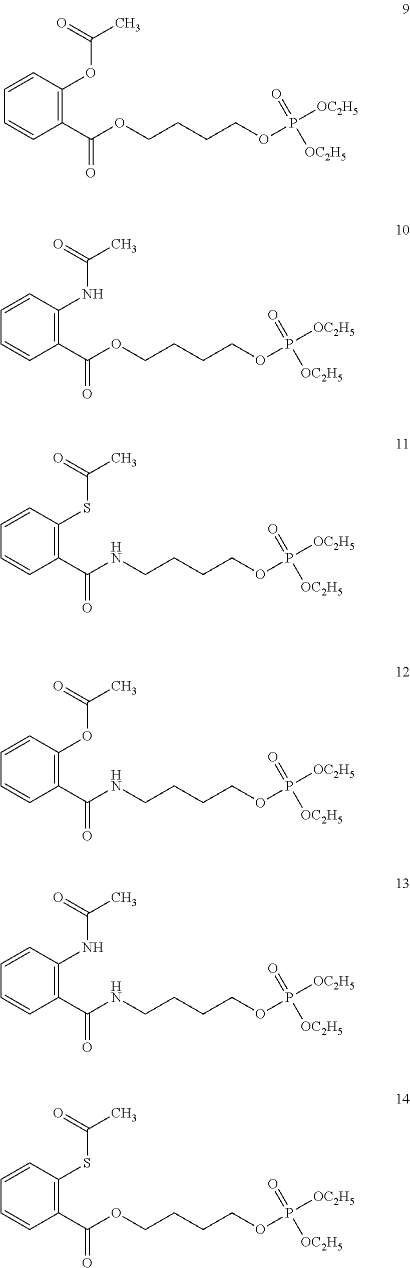 [SMILES: CCOP(=O)(OCC)OCCCCOC(=O)c1ccccc1OC(C)=O ; CCOP(=O)(OCC)OCCCCOC(=O)c1ccccc1NC(C)=O ; CCOP(=O)(OCC)OCCCCNC(=O)c1ccccc1SC(C)=O ; CCOP(=O)(OCC)OCCCCNC(=O)c1ccccc1OC(C)=O ; CCOP(=O)(OCC)OCCCCNC(=O)c1ccccc1NC(C)=O ; CCOP(=O)(OCC)OCCCCOC(=O)c1ccccc1SC(C)=O]

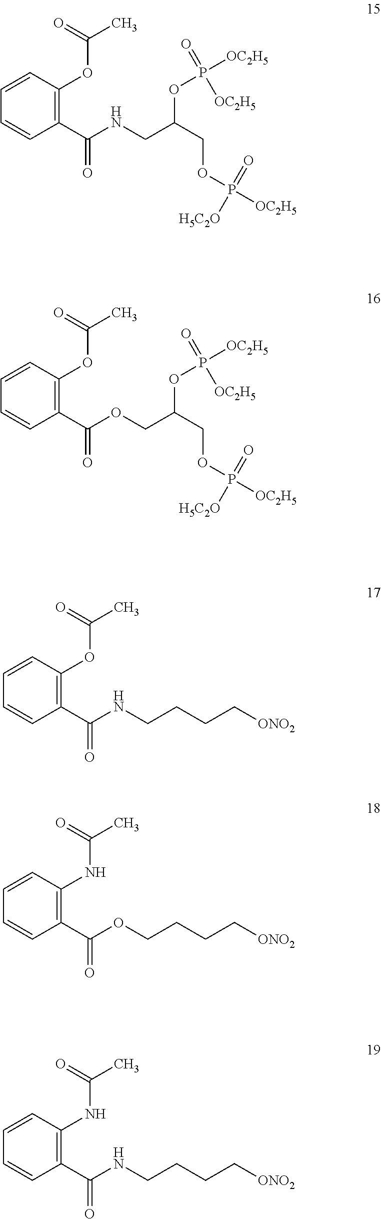

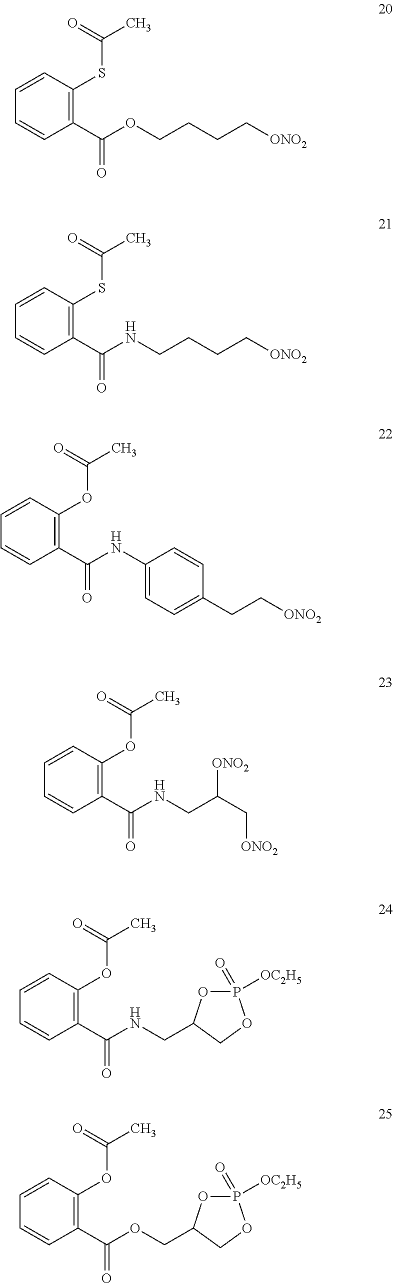

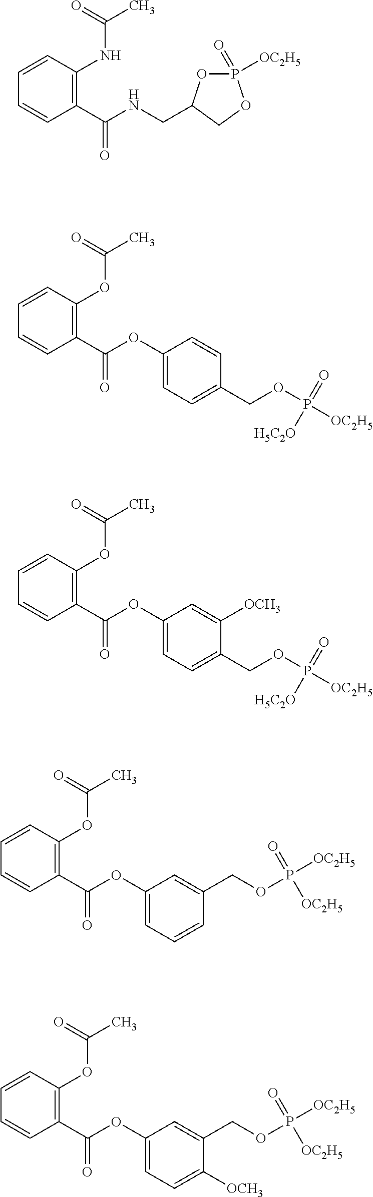

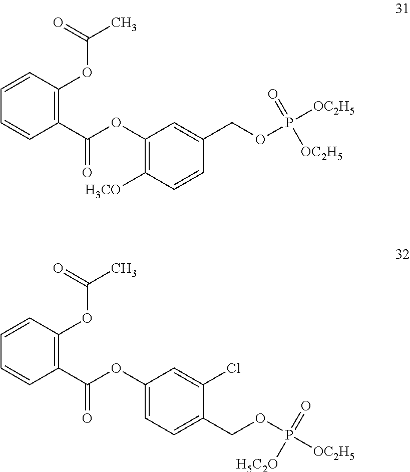

In a further embodiment of the invention, the substituent A is represented by Formula A-II, R⁹ is hydrogen and R¹⁰ is represented by Formula A-XII, Formula A-XIII or Formula A-XIV. The corresponding compounds of Formula I include but are not limited to the following compounds 33 to 50:

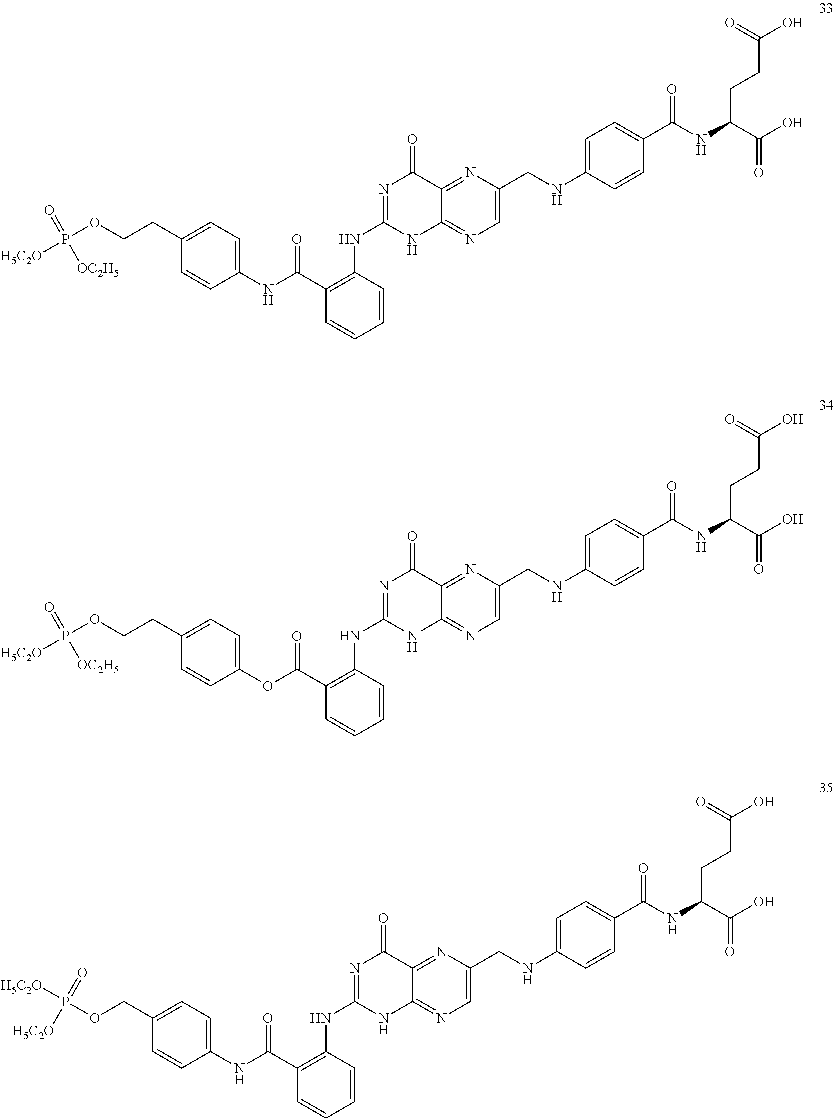

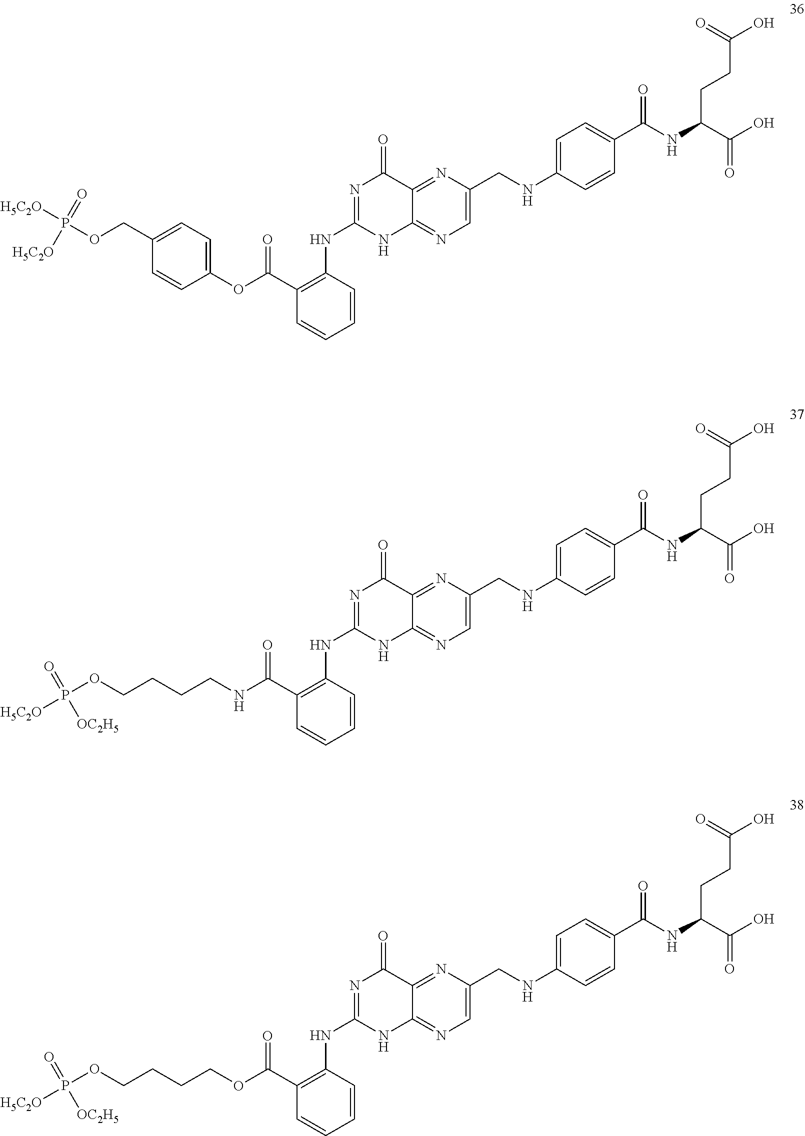

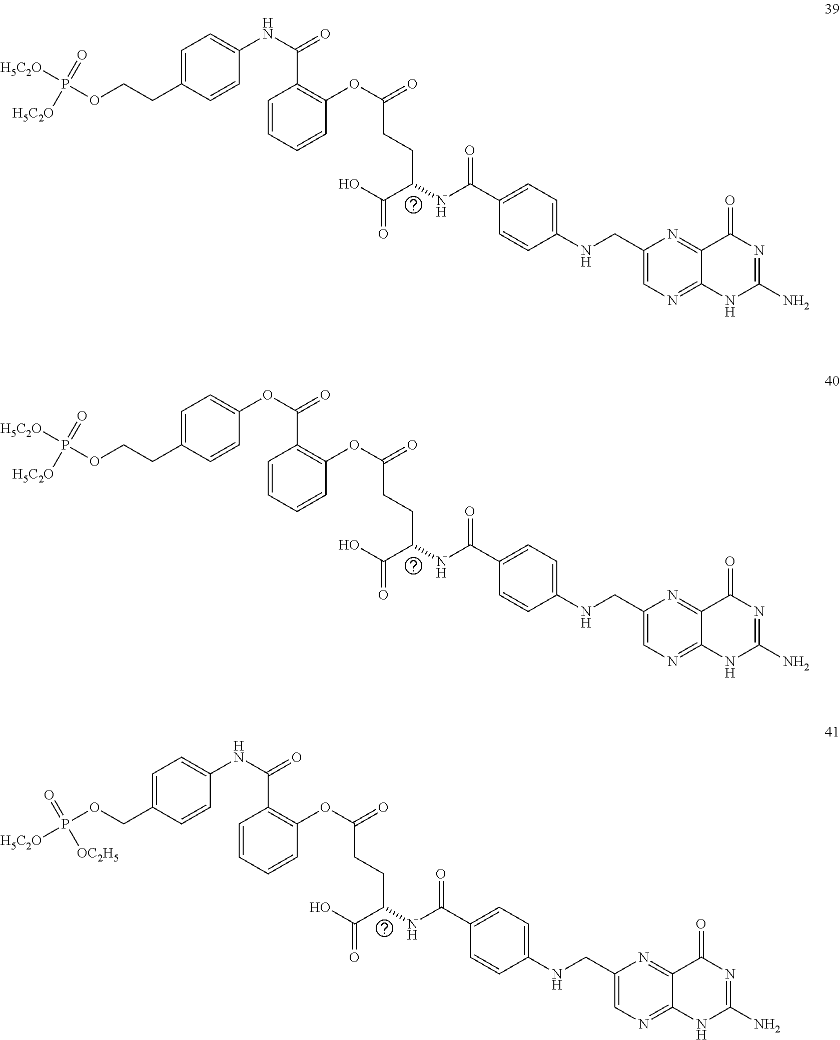

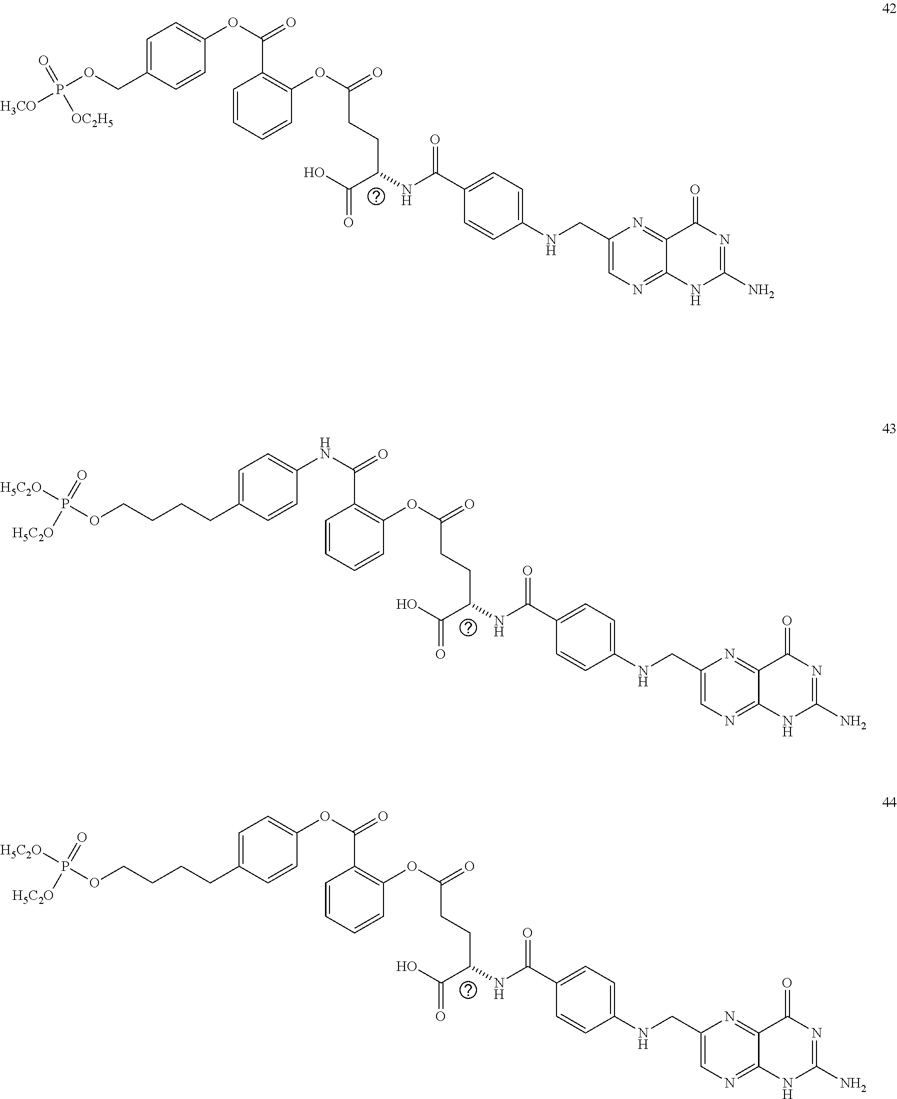

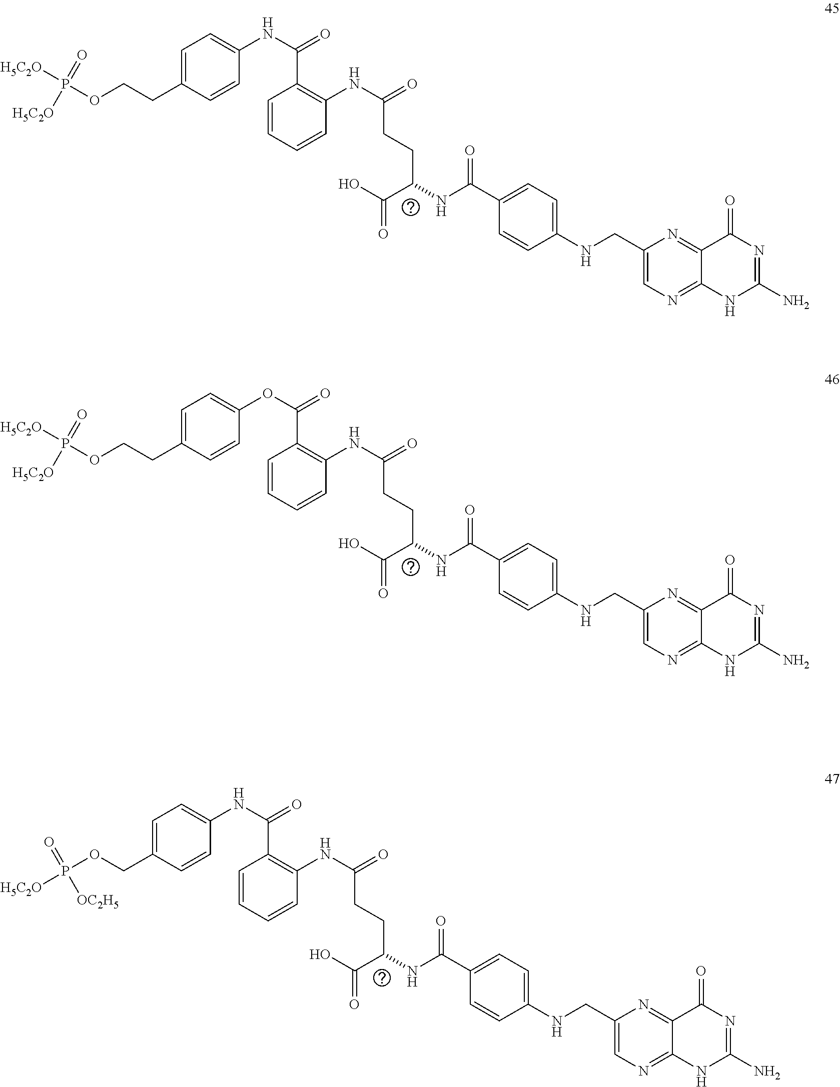

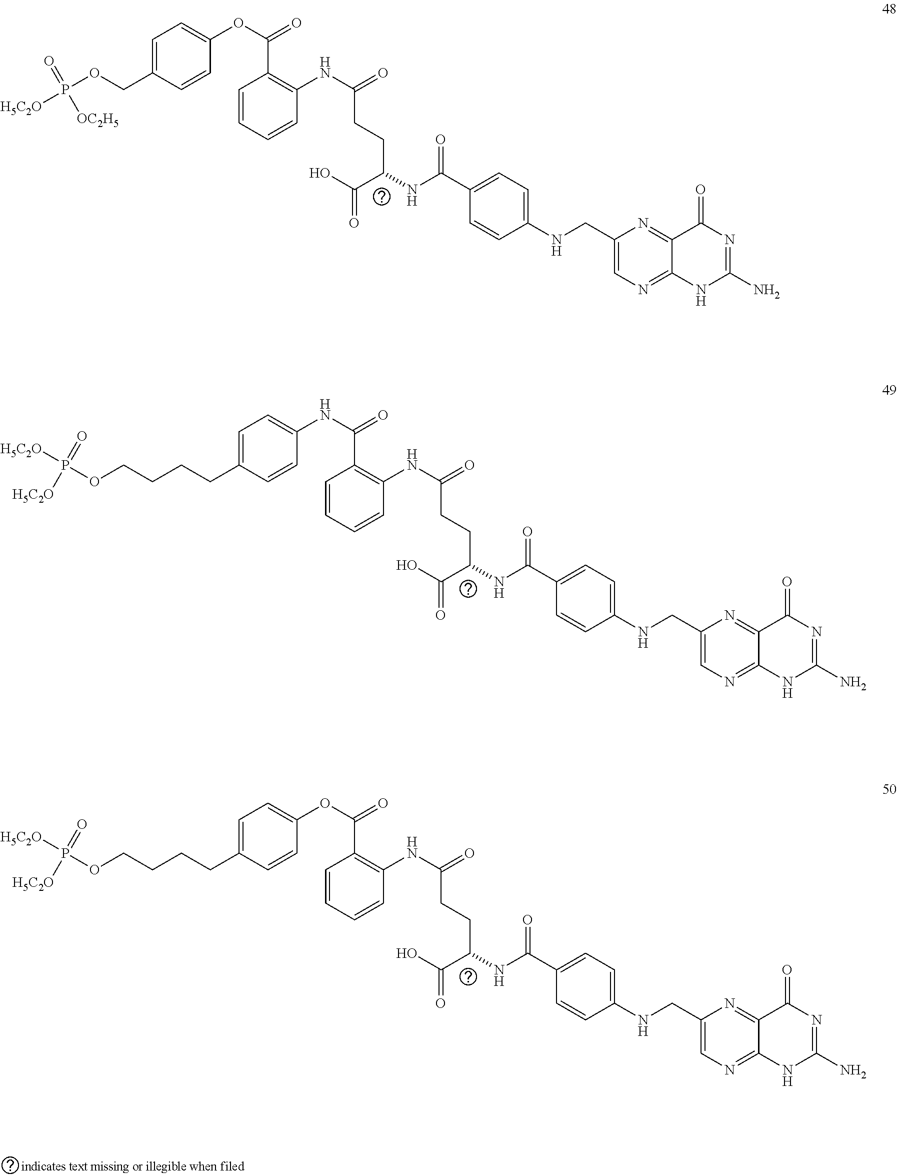

In a further embodiment of the invention, the substituent A is represented by Formula A-II, R⁹ is hydrogen and R¹⁰ is represented by Formula A-XV. The corresponding compounds of Formula I include but are not limited to the following compounds 51 to 68:

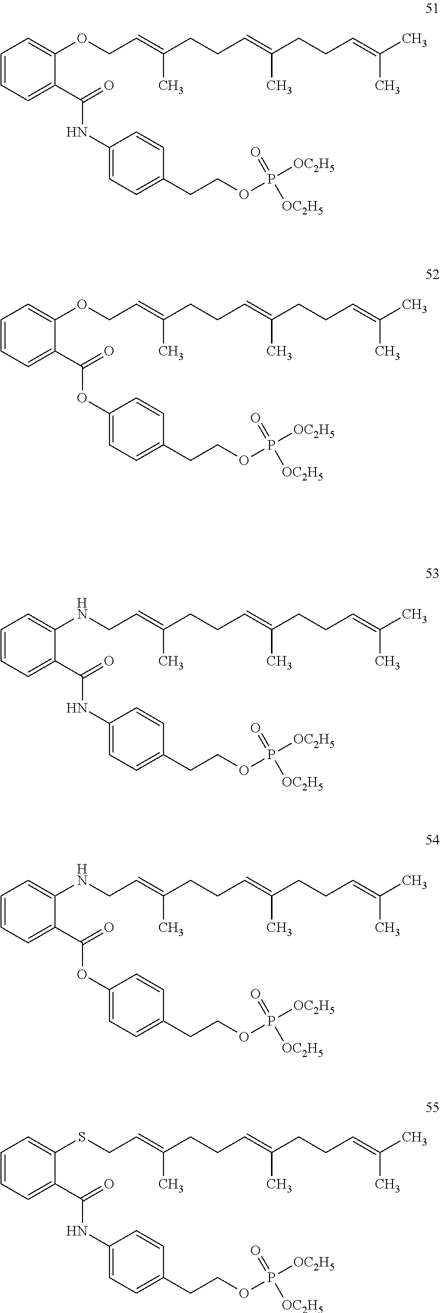 [SMILES: CCOP(=O)(OCC)OCCc1ccc(NC(=O)c2ccccc2OC/C=C(\C)CC/C=C(\C)CCC=C(C)C)cc1 ; CCOP(=O)(OCC)OCCc1ccc(OC(=O)c2ccccc2OC/C=C(\C)CC/C=C(\C)CCC=C(C)C)cc1 ; CCOP(=O)(OCC)OCCc1ccc(NC(=O)c2ccccc2NC/C=C(\C)CC/C=C(\C)CCC=C(C)C)cc1 ; CCOP(=O)(OCC)OCCc1ccc(OC(=O)c2ccccc2NC/C=C(\C)CC/C=C(\C)CCC=C(C)C)cc1 ; CCOP(=O)(OCC)OCCc1ccc(NC(=O)c2ccccc2SC/C=C(\C)CC/C=C(\C)CCC=C(C)C)cc1]

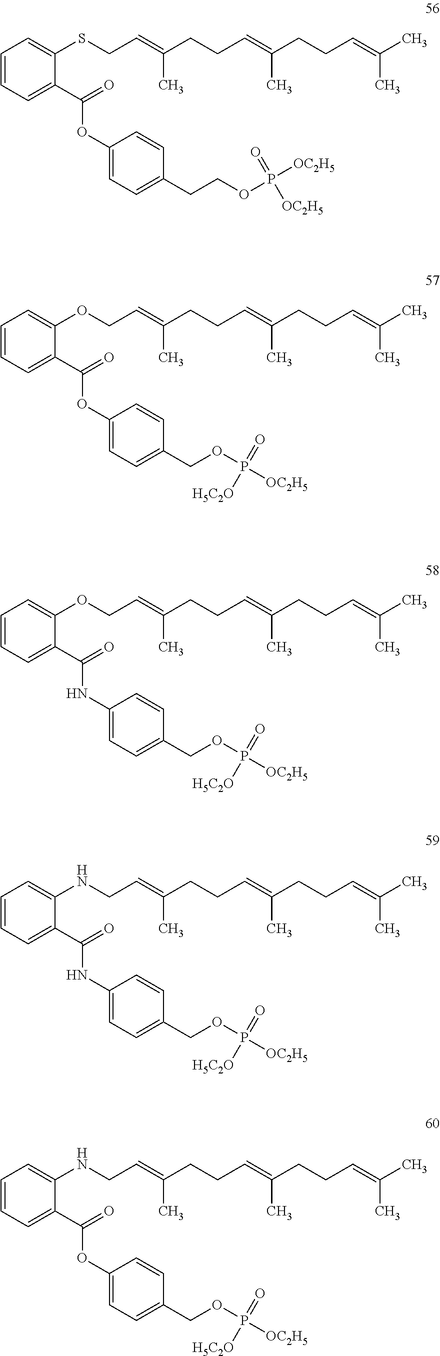

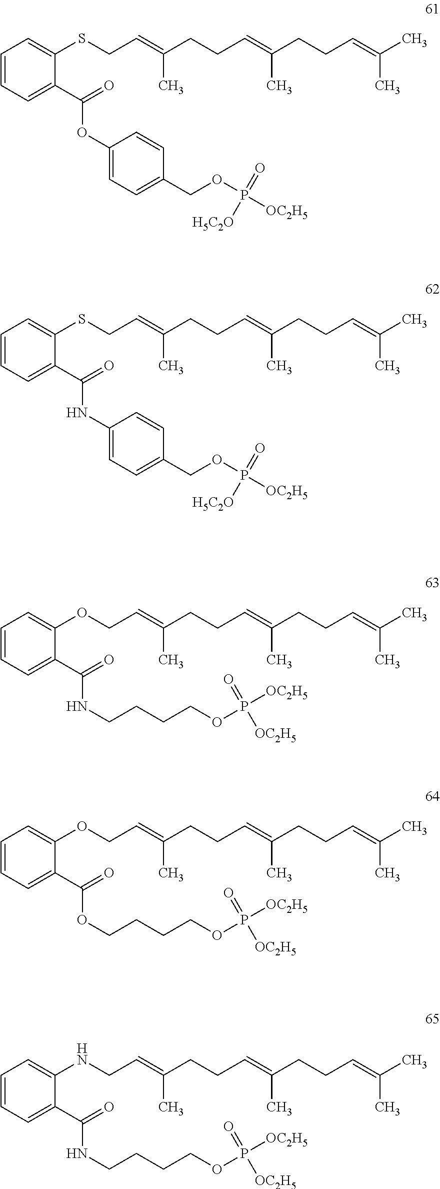

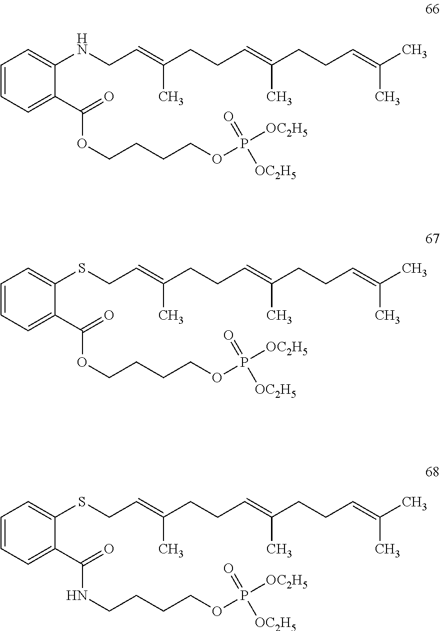 [SMILES: CCOP(=O)(OCC)OCCCCOC(=O)c1ccccc1NC/C=C(\C)CC/C=C(\C)CCC=C(C)C ; CCOP(=O)(OCC)OCCCCOC(=O)c1ccccc1SC/C=C(\C)CC/C=C(\C)CCC=C(C)C ; CCOP(=O)(OCC)OCCCCNC(=O)c1ccccc1SC/C=C(\C)CC/C=C(\C)CCC=C(C)C]

In another embodiment of the invention, the substituent A is represented by Formula A-II, R⁹ is trifluoromethyl and R¹⁰ is —X2-C(O)—CH₃. The corresponding compounds are structurally related to triflusal. According to this embodiment, the compounds of Formula I include but are not limited to the compounds 69 to 74 listed below:

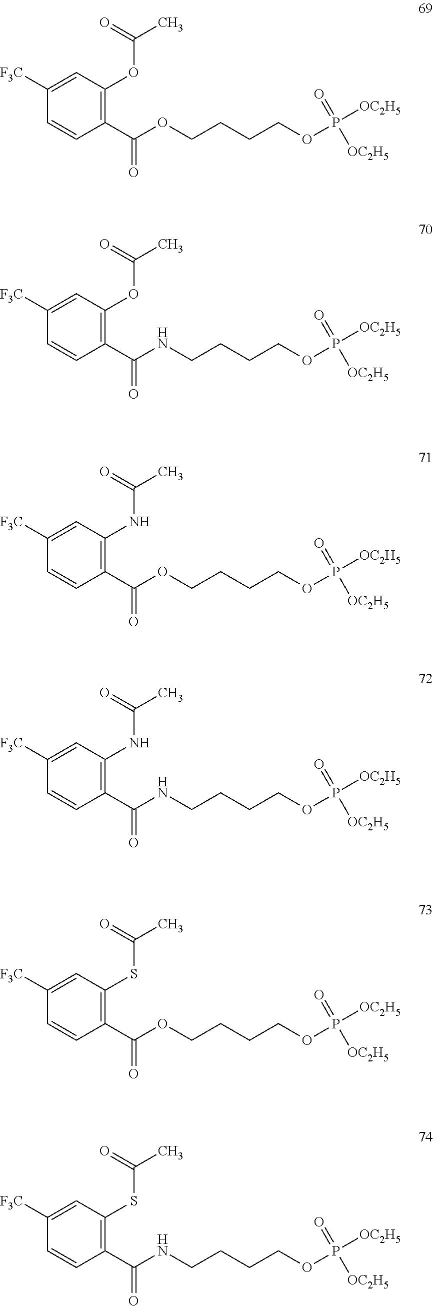 [SMILES: CCOP(=O)(OCC)OCCCCOC(=O)c1ccc(C(F)(F)F)cc1OC(C)=O ; CCOP(=O)(OCC)OCCCCNC(=O)c1ccc(C(F)(F)F)cc1OC(C)=O ; CCOP(=O)(OCC)OCCCCOC(=O)c1ccc(C(F)(F)F)cc1NC(C)=O ; CCOP(=O)(OCC)OCCCCNC(=O)c1ccc(C(F)(F)F)cc1NC(C)=O ; CCOP(=O)(OCC)OCCCCOC(=O)c1ccc(C(F)(F)F)cc1SC(C)=O ; CCOP(=O)(OCC)OCCCCNC(=O)c1ccc(C(F)(F)F)cc1SC(C)=O]

In a further embodiment of the invention, the substituent A is represented by Formula A-II, R⁹ is trifluoromethyl and R¹⁰ is represented by Formula A-XII, Formula A-XIII or Formula A-XIV. The corresponding compounds of Formula I include but are not limited to the following compounds 75 to 92:

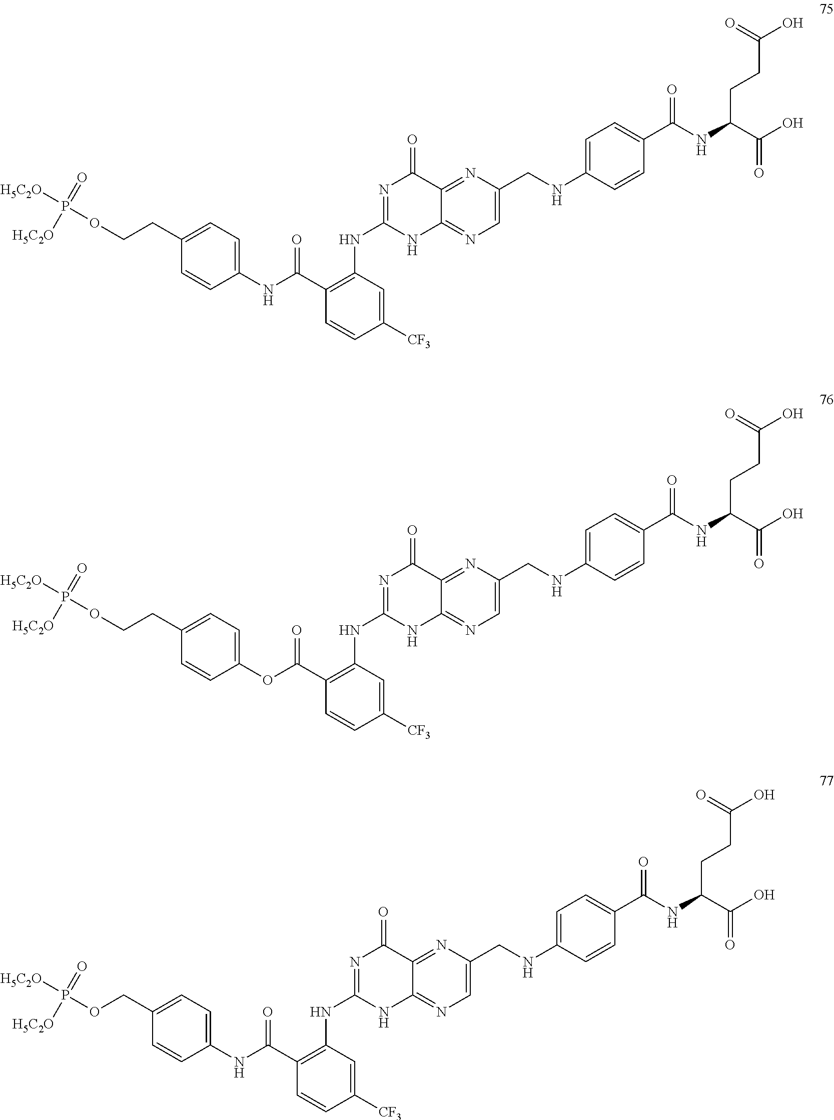

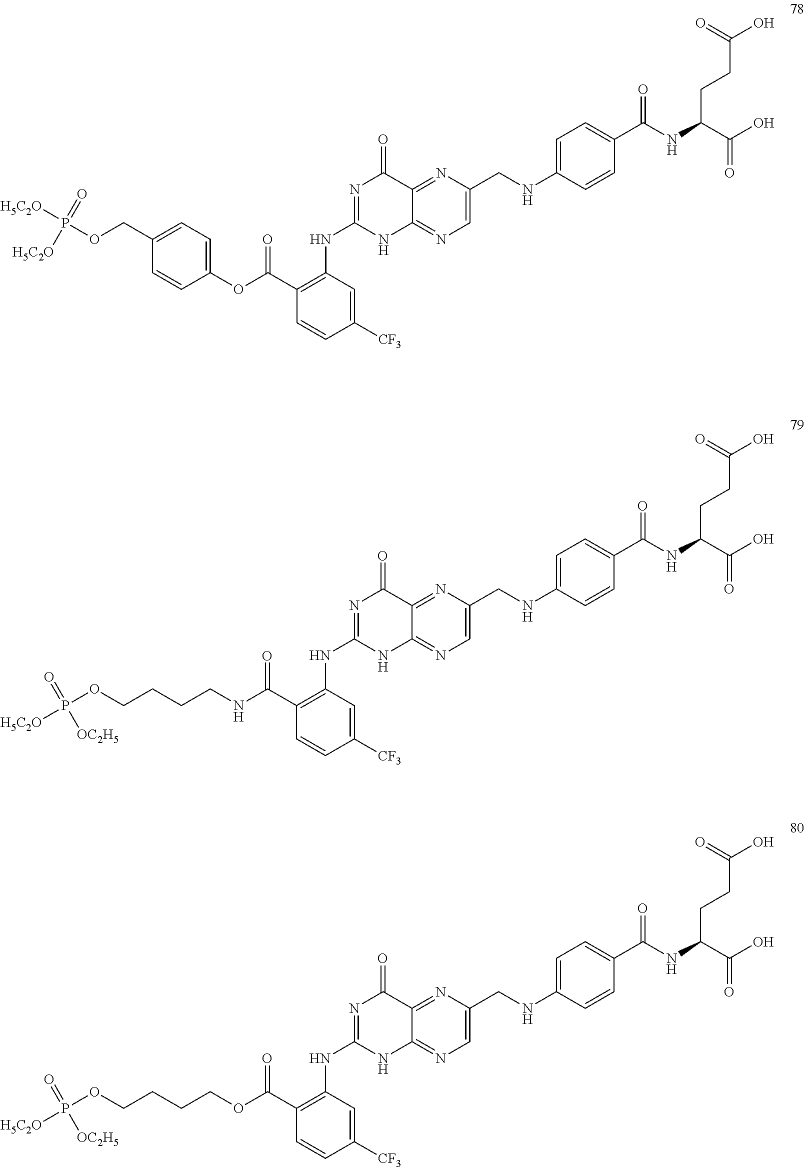

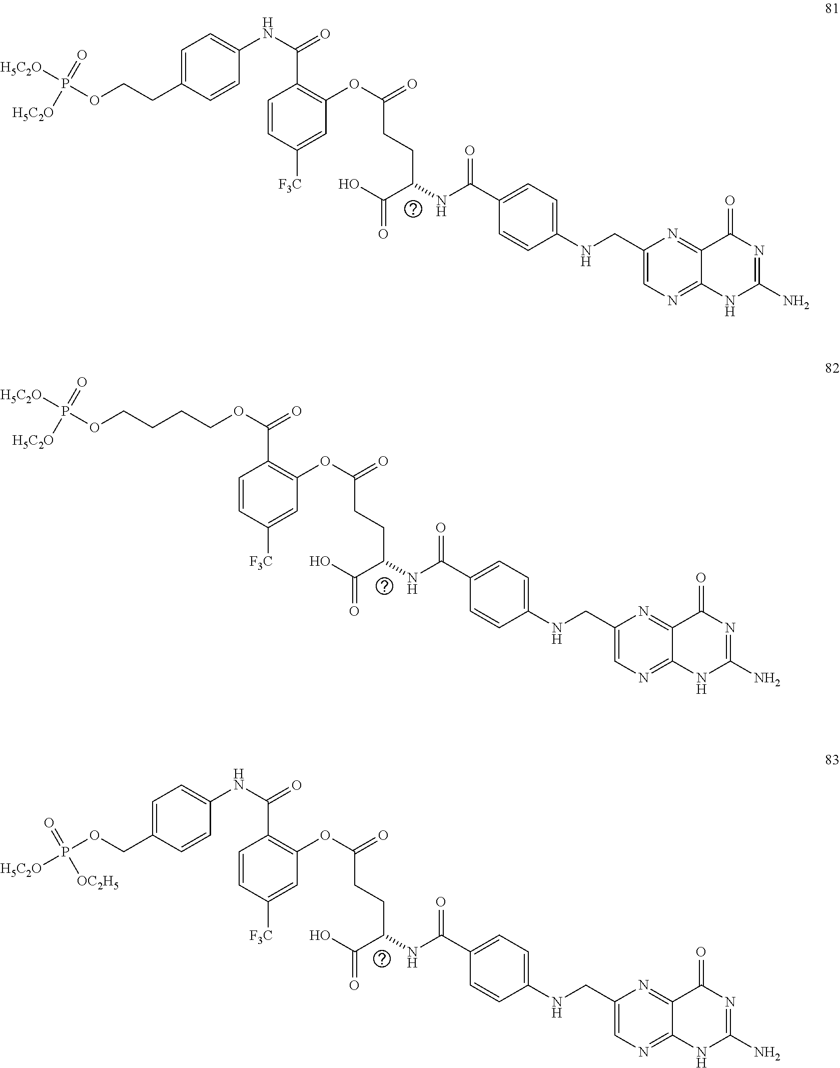

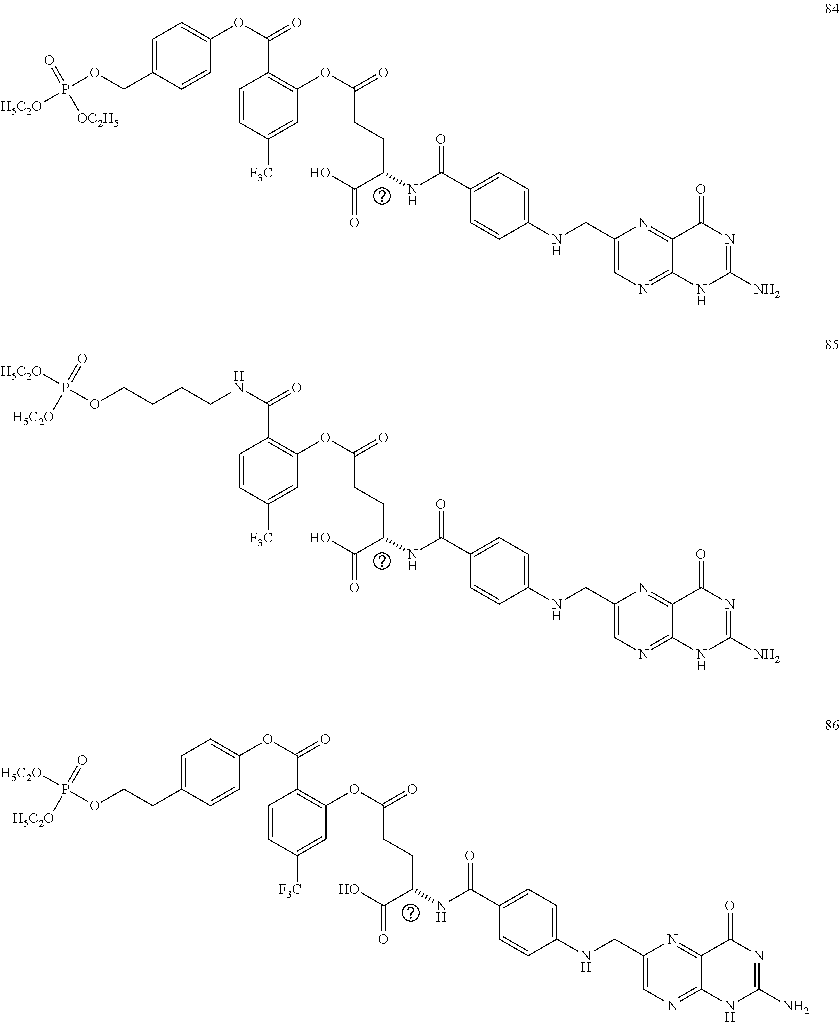

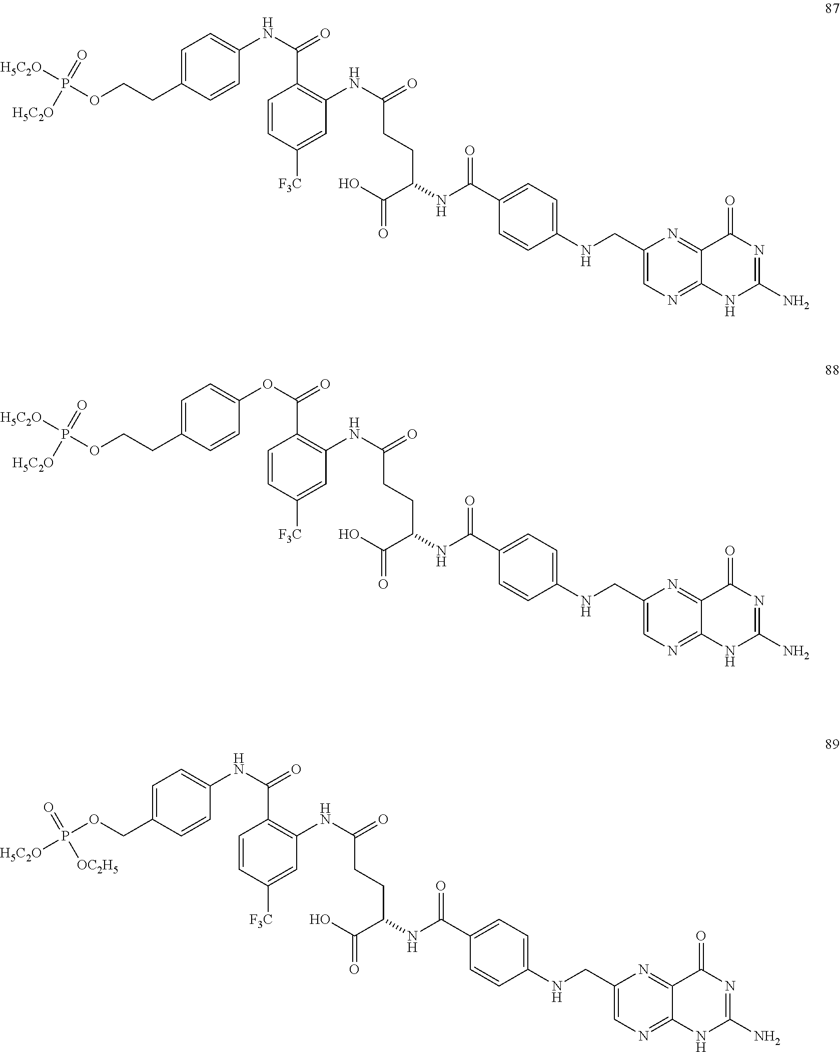

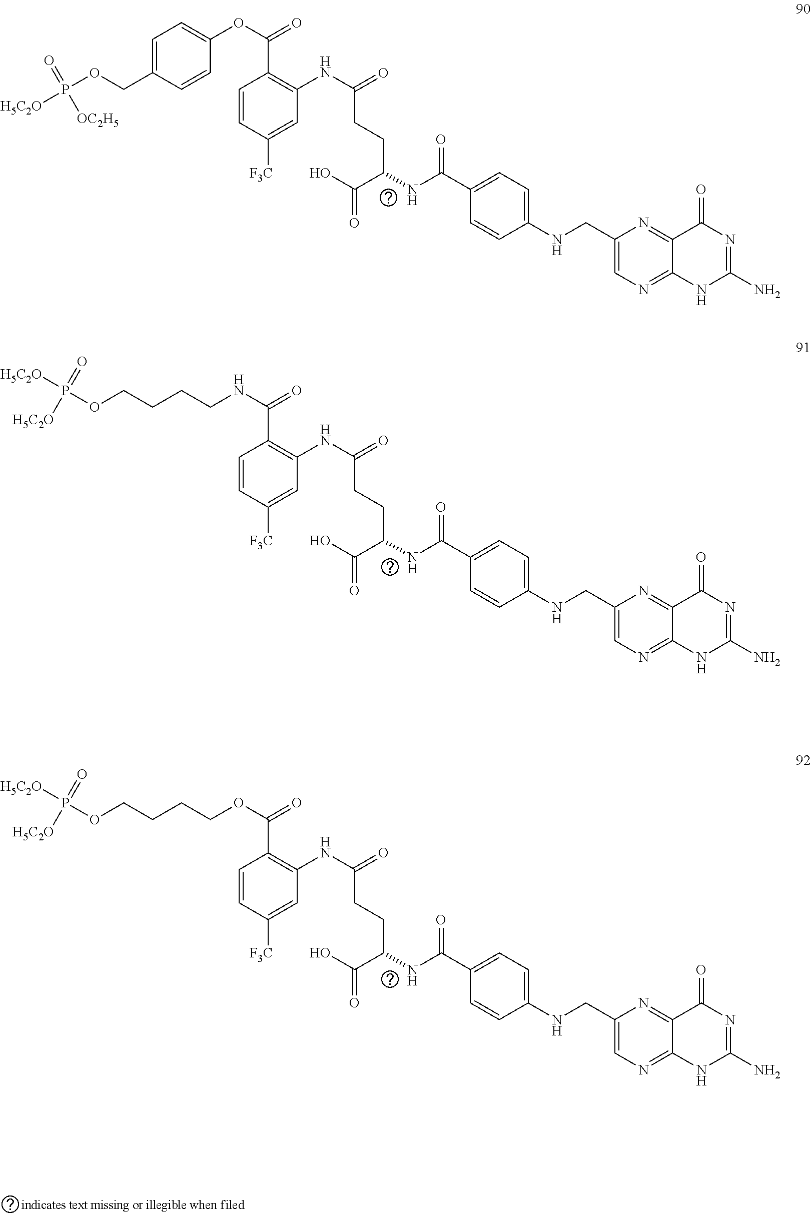

In a further embodiment of the invention, the substituent A is represented by Formula A-III. The corresponding compounds are structurally related to indomethacin. In this embodiment, the compounds of Formula I include but are not limited to the compounds 93 and 94 shown below:

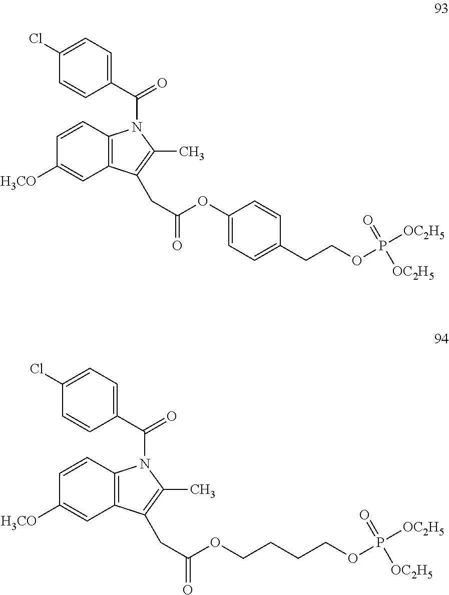 [SMILES: CCOP(=O)(OCC)OCCc1ccc(OC(=O)Cc2c(C)n(C(=O)c3ccc(Cl)cc3)c3ccc(OC)cc23)cc1 ; CCOP(=O)(OCC)OCCCCOC(=O)Cc1c(C)n(C(=O)c2ccc(Cl)cc2)c2ccc(OC)cc12]

Yet another embodiment of the present invention provides compounds of Formula I in which the substituent A is represented by Formula A-IV. Preferably, R¹¹ is S(O)CH₃. The corresponding compounds are structurally related to sulindac. These compounds include but are not limited to the compounds 95 to 100 listed below:

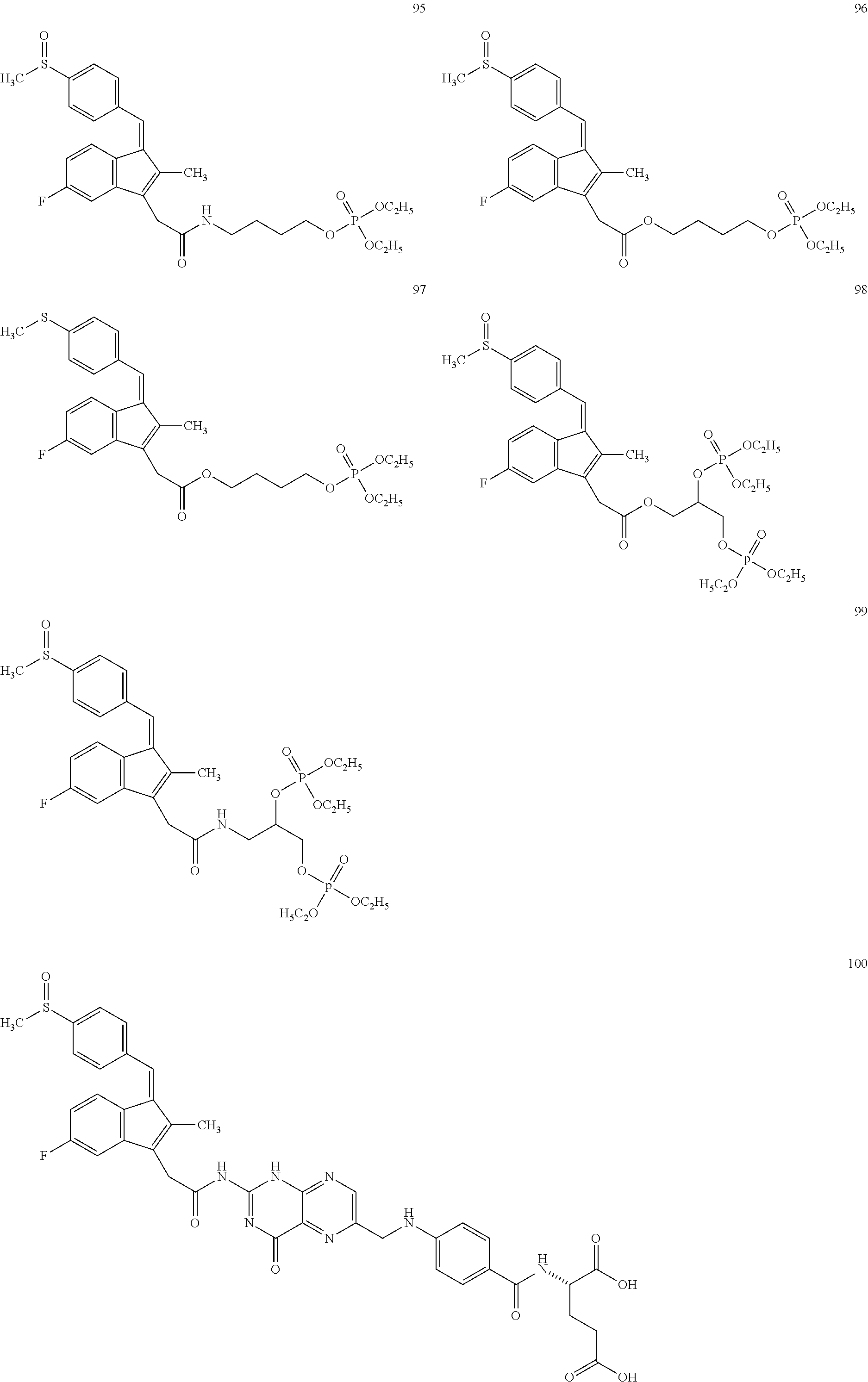

Compounds 96 (phospho-sulindac, hereinafter “PS”) and 97 (phospho-sulindac hereafter “PS-II”) have a strong activity against lung and brain cancer and can be administered to humans by the respiratory route for the purpose of treatment and/or prevention of lung and brain cancer and precancerous conditions thereof.

In a further embodiment of the invention, the substituent A is represented by Formula A-V. The corresponding compounds of Formula I include but are not limited to the compounds 101 to 112 shown below:

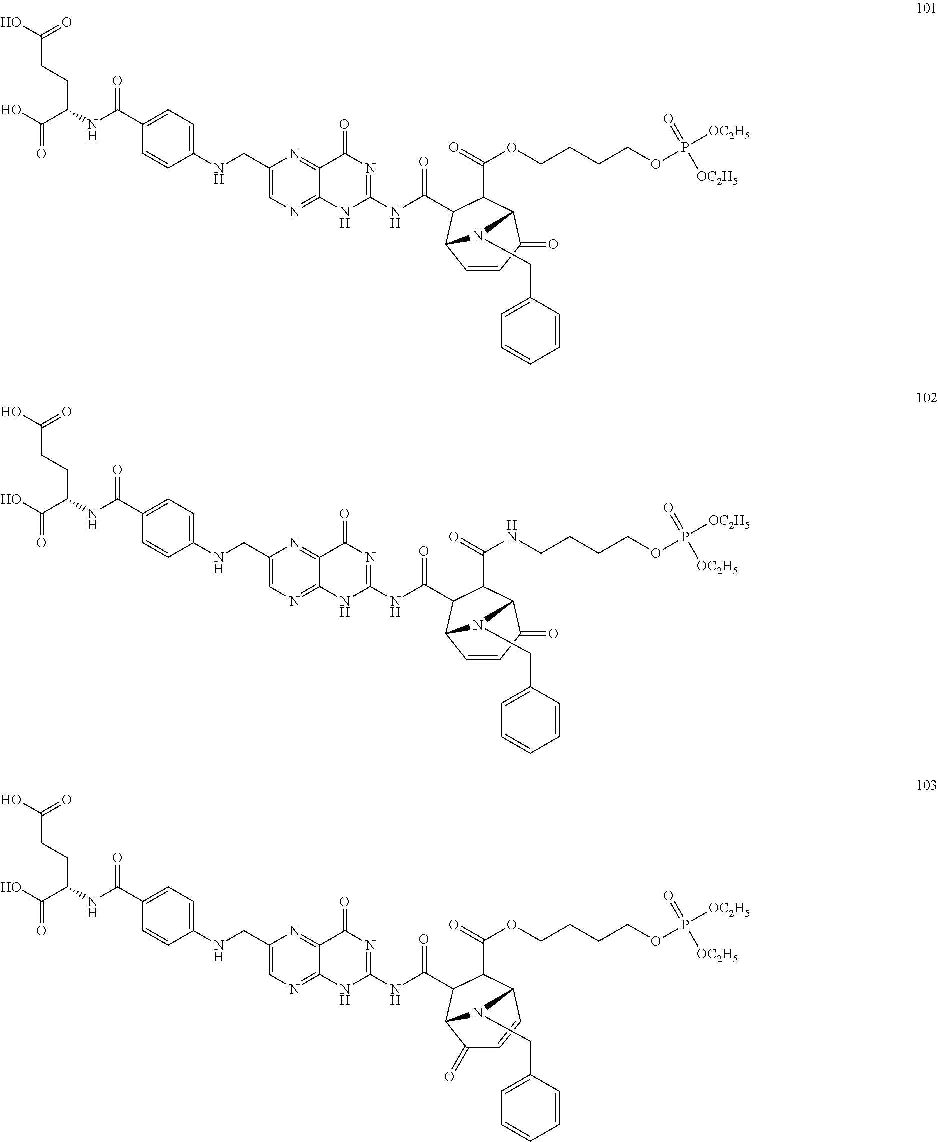 [SMILES: CCOP(=O)(OCC)OCCCCOC(=O)C1C(C(=O)Nc2nc(=O)c3nc(CNc4ccc(C(=O)N[C@@H](CCC(=O)O)C(=O)O)cc4)cnc3[nH]2)[C@H]2C=CC(=O)[C@@H]1N2Cc1ccccc1 ; CCOP(=O)(OCC)OCCCCNC(=O)C1C(C(=O)Nc2nc(=O)c3nc(CNc4ccc(C(=O)N[C@@H](CCC(=O)O)C(=O)O)cc4)cnc3[nH]2)[C@H]2C=CC(=O)[C@@H]1N2Cc1ccccc1 ; CCOP(=O)(OCC)OCCCCOC(=O)C1C(C(=O)Nc2nc(=O)c3nc(CNc4ccc(C(=O)N[C@@H](CCC(=O)O)C(=O)O)cc4)cnc3[nH]2)[C@H]2C(=O)C=C[C@@H]1N2Cc1ccccc1]

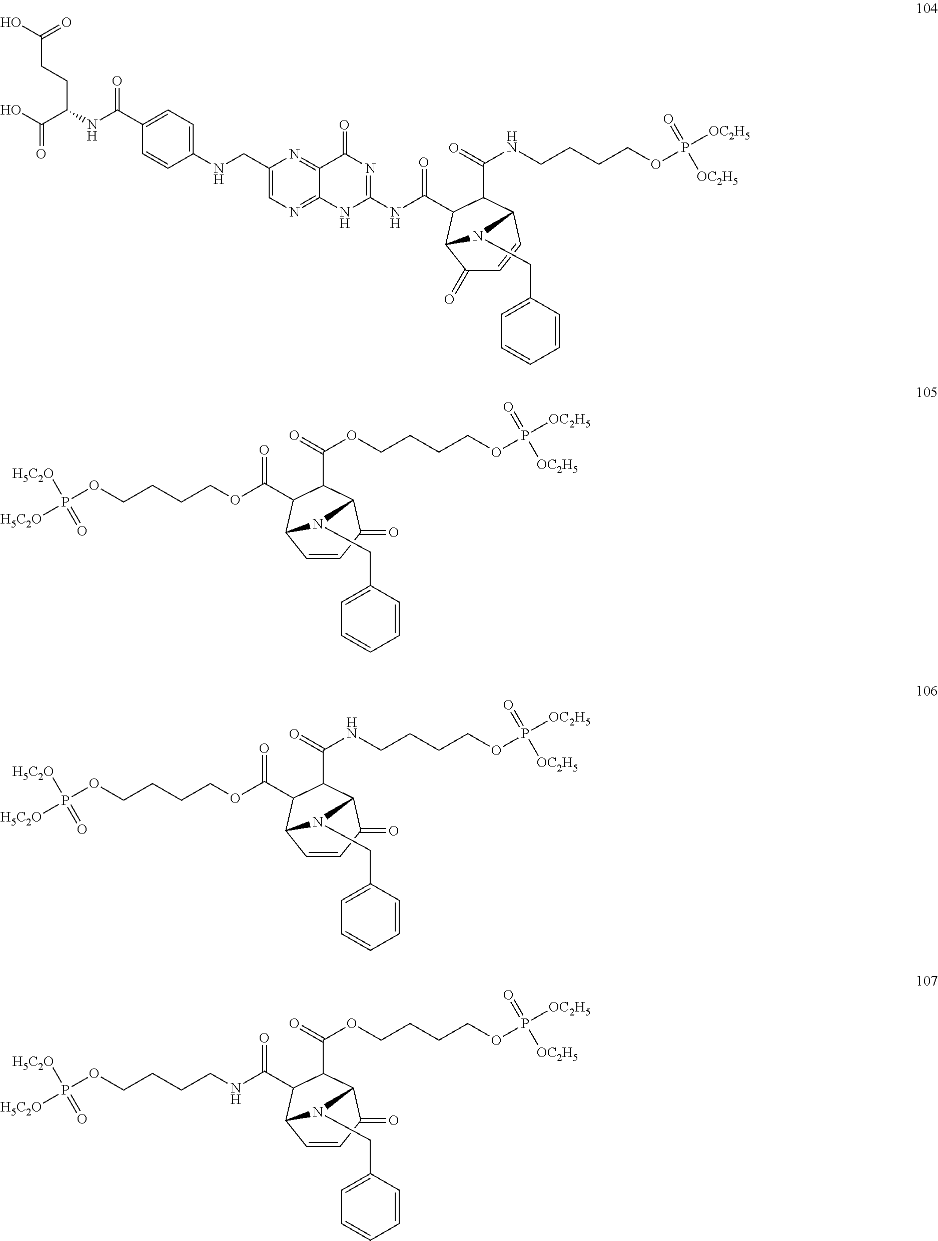

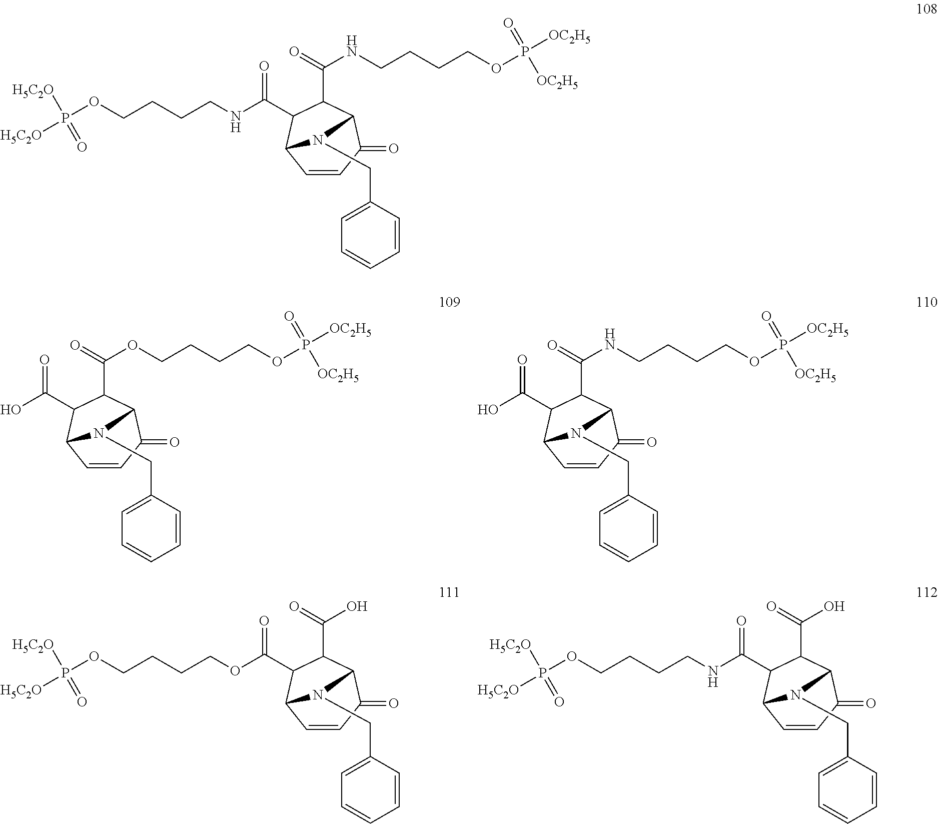

In a further embodiment of the invention, substituent A is represented by Formula A-VI. These compounds are structurally related to rigosertib (sodium (E)-2-((2-methoxy-5-(((2,4,6-trimethoxystyryl)sulfonyl)methyl)phenyl)amino)acetate). In this embodiment, the compounds of Formula I include but are not limited to the compounds 113 and 114 shown below:

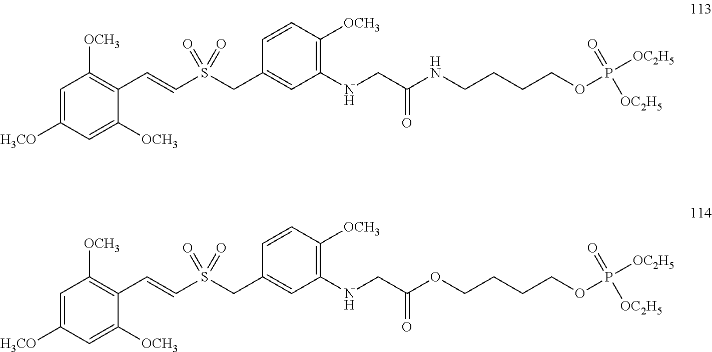 [SMILES: CCOP(=O)(OCC)OCCCCNC(=O)CNc1cc(CS(=O)(=O)/C=C/c2c(OC)cc(OC)cc2OC)ccc1OC ; CCOP(=O)(OCC)OCCCCOC(=O)CNc1cc(CS(=O)(=O)/C=C/c2c(OC)cc(OC)cc2OC)ccc1OC]

In a further embodiment of the invention, the substituent A is represented by Formula A-VII. Thus, the corresponding compounds are structurally related to valproic acid. The corresponding compounds are particularly suitable for the treatment of brain cancer and precancerous conditions of brain cancer, for instance for the treatment of glioma. In this embodiment, the compounds of Formula I include but are not limited to the compounds 115 to 118 and 511 shown below:

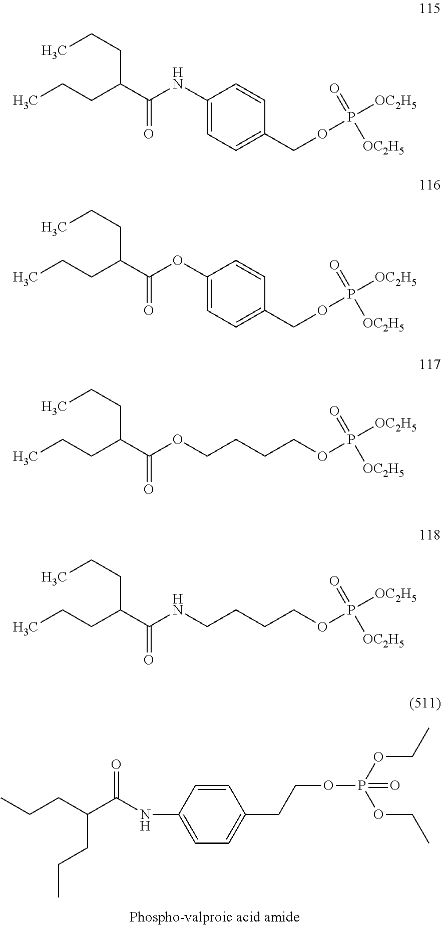 [SMILES: CCCC(CCC)C(=O)Nc1ccc(COP(=O)(OCC)OCC)cc1 ; CCCC(CCC)C(=O)Oc1ccc(COP(=O)(OCC)OCC)cc1 ; CCCC(CCC)C(=O)OCCCCOP(=O)(OCC)OCC ; CCCC(CCC)C(=O)NCCCCOP(=O)(OCC)OCC ; CCCC(CCC)C(=O)Nc1ccc(CCOP(=O)(OCC)OCC)cc1]

In a further embodiment of the invention, the substituent A is represented by Formula A-VIII. In this embodiment, the compounds of Formula I include but are not limited to the compounds 119 and 120 shown below:

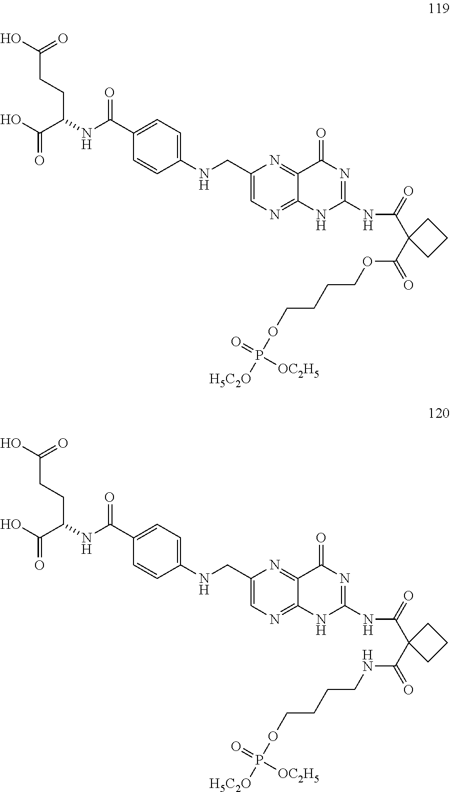

In a further embodiment of the invention, the substituent A is represented by Formula A-IX. Thus, the corresponding compounds are structurally related to naproxen. In this embodiment, the compounds of Formula I include but are not limited to the compounds such as phospho-naproxen 121, the structure of which is shown below:

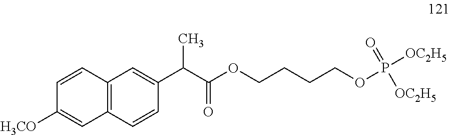 [SMILES: CCOP(=O)(OCC)OCCCCOC(=O)C(C)c1ccc2cc(OC)ccc2c1]

In a further embodiment of the invention, the substituent A is represented by Formula A-X. Thus, the corresponding compounds are structurally related to flurbiprofen. In this embodiment, the compounds of Formula I include but are not limited to the compounds such as phospho-flurbiprofen 122, the structure of which is shown below:

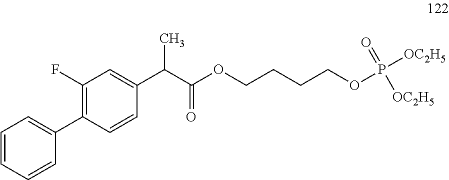 [SMILES: CCOP(=O)(OCC)OCCCCOC(=O)C(C)c1ccc(-c2ccccc2)c(F)c1]

In yet another embodiment of the invention, the substituent A is represented by Formula A-XI and thus the corresponding compounds are structurally related to salinomycin. In this embodiment, the compounds of Formula I include but are not limited to the compounds 123 and 124 shown below:

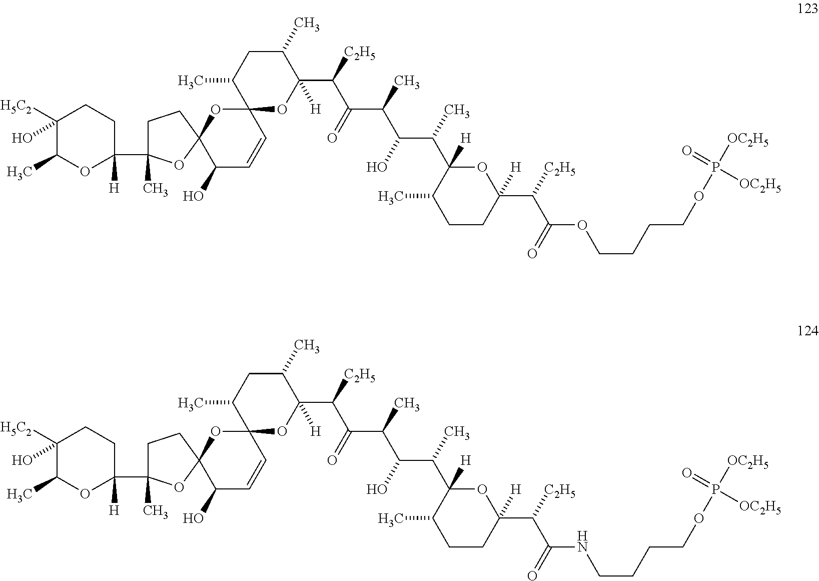

In some preferred embodiments of the present invention, the compound of Formula I is selected from the following: 2-acetoxy-benzoic acid 4-(diethoxy-phosphoryloxymethyl)-phenyl ester (27), 2-acetoxy-benzoic acid 3-(diethoxy-phosphoryloxymethyl)-phenyl ester (29), and phospho-sulindac (96), phospho-sulindac II (97), phospho-flurbiprofen (122), phospho-ibuprofen (2), phosphoaspirin I (25), phosphoraspirin II (16), and phosphovalproic acid (116).

In other preferred embodiments of the present invention, the compound of Formula I is selected from the compounds 2, 3, 7, 9, 93, 94, 96, 97 and 98. In yet other preferred embodiments of the present invention, the compound of Formula I is selected from the compounds 1, 4, 12, 15, 95 and 99.

In some embodiments, the substituent A is derived from among non-steroidal anti-inflammatory drugs (NSAIDs) ibuprofen (Formula A-I), aspirin (Formula A-II), indomethacin (Formula A-III) or sulindac (Formula A-IV):

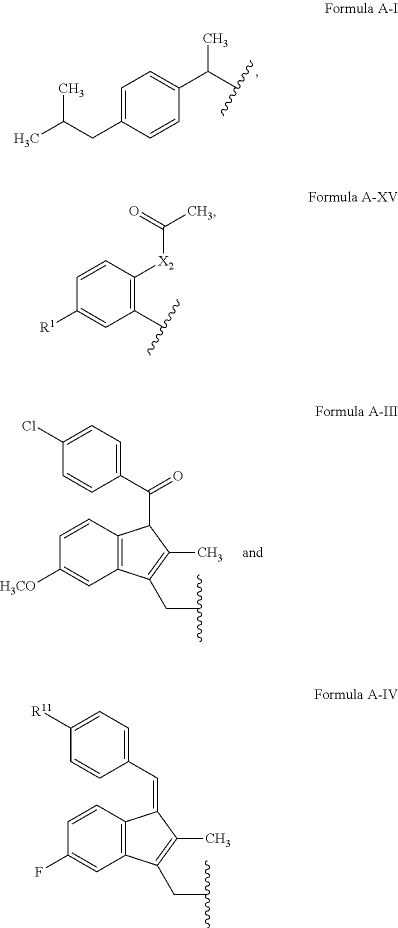

In particular embodiments of Formula A-XV, R¹ is hydrogen or trifluoromethyl and X² is selected from the group consisting of —O—, —S— and —NR⁴—, and R⁴ is hydrogen or C₁₋₆-alkyl.

In particular embodiments of Formula A-IV, R² is selected from the group consisting of —SCH₃, —S(O)CH₃, —S(O)₂CH₃. Preferably R² in Formula A-IV is S(O)CH₃.

In some other embodiments the substituent A is represented by Formulas A-V to A-IX shown below:

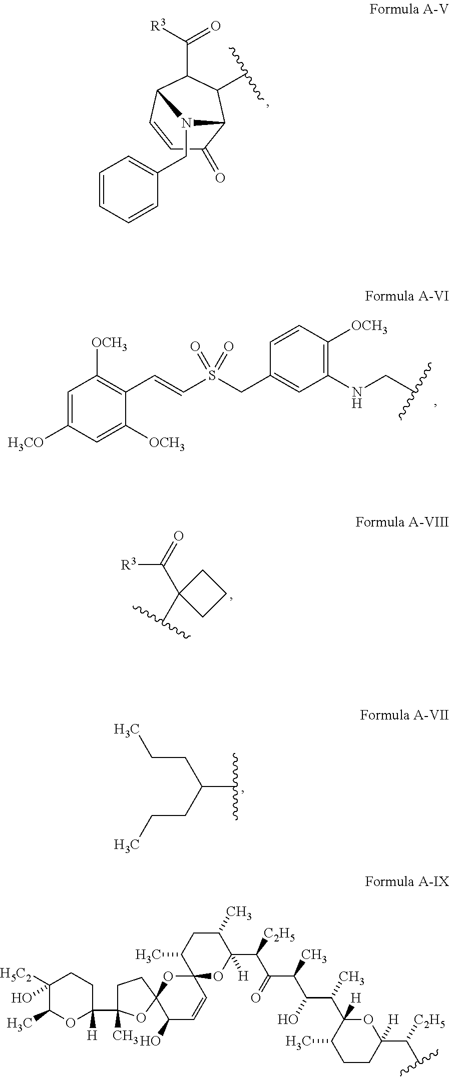

Group R³ in Formulas A-V and A-VII is generally defined above, in connection with the first appearance of Formula I. In particular embodiments where A is represented by Formula A-V or Formula A-VII, R³ is represented by hydroxy, —B—Z or

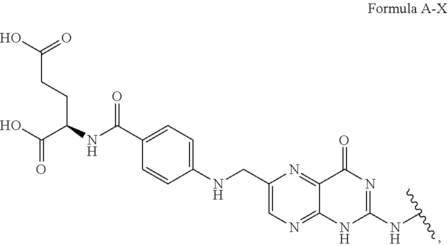 [SMILES: *Nc1nc(=O)c2nc(CNc3ccc(C(=O)N[C@H](CCC(=O)O)C(=O)O)cc3)cnc2[nH]1]

and —B— and —Z are as specified for Formula I.

The substituent represented by Formula A-X is a folic acid residue. Without wishing to be bound by any theory it is believed that compounds of the present invention having R³ represented by Formula A-X have a particularly strong anti-cancer activity. In particular, the anti-cancer activity of compounds in which R³ is represented by Formula A-X is usually higher than the anti-cancer activity of corresponding compounds in which R³ is hydroxyl group.

If A is represented by any of Formulas A-I or A-IV, and X¹ is —O—, then —B—Z is not —(CH₂)₄—O—P(O)(OC₂H₅)₂.

In addition, if A is represented by Formula A-II, X¹ is —N— or —S—; and/or —B— is an aliphatic group with 1 to 22 carbon atoms.

The substituent X¹ in Formula I can be —O—, —S— or —NR⁴—, R⁴ being hydrogen or an alkyl group having 1 to 6 carbon atoms, preferably 1 to 3 carbon atoms such as for instance methyl or ethyl, preferably methyl.

In particular embodiments of Formula I (including any of Formulas II, III, and IV), the substituent B is

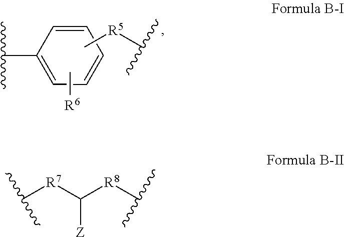

or an aliphatic group with 1 to 22 carbon atoms, preferably with 1 to 15 carbon atoms, more preferred with 1 to 10 carbon atoms and particularly preferred with 1 to 6 carbon atoms.

Substituents R⁵, R⁷, and R⁸ can be the same or different alkylene group having 1 to 3 carbon atoms. Preferably the substituent R⁵ in Formula B-I is selected from the group consisting of methylene, ethylene and trimethylene; in a more preferred embodiment R⁵ is methylene or ethylene, whereby methylene is particularly preferred.

In yet another preferred embodiment the substituent B is represented by Formula B-II and R⁷, and R⁸ are identical alkylene group having 1 to 3 carbon atoms. In a particularly preferred embodiment R⁷, and R⁸ are both methylene groups so that the substituent B in Formula I is represented by Formula B-IV:

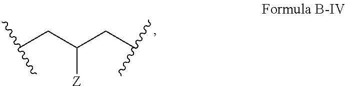

Z being defined below.

In a particularly preferred embodiment B forms a glycerol ester residue together with X¹ and Z.

R⁶ in Formula B-I can be hydrogen, an alkyl having 1 to 3 carbon atoms, halo or methoxy. Preferably, R⁶ is selected from the group consisting of hydrogen, methyl, fluoro, chloro, bromo and methoxy, preferably from the group consisting of hydrogen, methyl, chloro and fluoro. In a particularly preferred embodiment R⁶ represents hydrogen so that B is represented by Formula B-III:

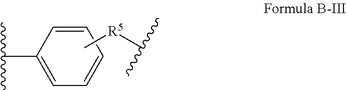

The substitution pattern of the substituent B in Formulas B-I and B-III is not particularly limited. When B is represented by Formula B-III the aromatic moiety of B can be 1,2- or 1,3- or 1,4-substituted. Preferably, the aromatic moiety of B is 1,4-substituted so that B is represented by Formula B-V:

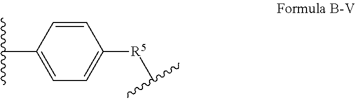

In yet another embodiment of the present invention B is an aliphatic group, preferably having 1 to 22 carbon atoms, more preferred 1 to 6 carbon atoms. In a particularly preferred embodiment B is selected from the group consisting of alkylene groups with 1 to 6 carbon atoms, alkenylene group having 2 to 6 carbon atoms and alkynylene group having 2 to 6 carbon atoms. In a more preferred embodiment B is an alkylene group with 1 to 6 carbon atoms, preferably an alkylene group with 1 to 4 carbon atoms, even more preferably a tetramethylene group.

The substituent Z in Formula I is selected from the group consisting of

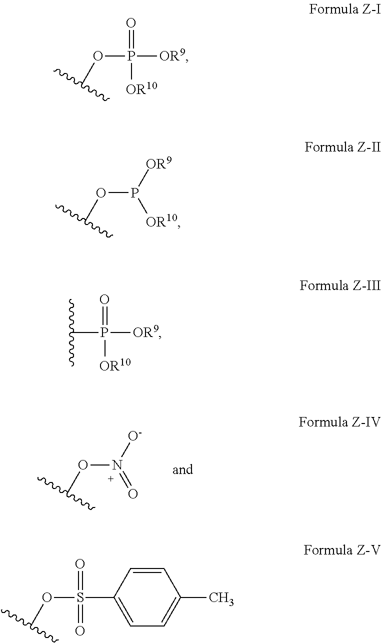

R⁹ is independently selected from hydrogen, C₁₋₆-alkyl or a polyethylene glycol group,

R¹⁰ is independently selected from C₁₋₆-alkyl or a polyethylene glycol group.

Preferably, Z is represented by Formula Z-I. In preferred embodiments R⁹ and R¹⁰ are identical. Preferably, R⁹ and R¹⁰ are alkyl groups having 1 to 3 carbon atoms, particularly preferred R⁹ and R¹⁰ are ethyl groups.

In yet another preferred embodiment R⁹ is represented by hydrogen and R¹⁰ is polyethylene glycol residue, for instance (OCH₂CH₂)ₙOCH₃, whereby n is from 40 to 50.

In a further embodiment of the invention B together with Z forms a structure

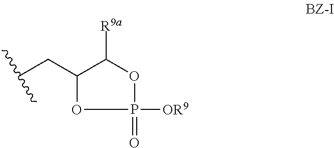

R⁹ is defined as above,

R^{9a} is independently selected from hydrogen, an aliphatic group, preferably with 1 to 22 carbon atoms, more preferably a C₁₋₆-alkyl or polyethylene glycol residue. In yet another preferred embodiment R^{9a} is hydrogen and B together with Z forms a structure

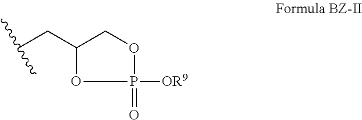

In some preferred embodiments of the present invention X¹ is N, R⁴ is hydrogen; B is selected from the group consisting of

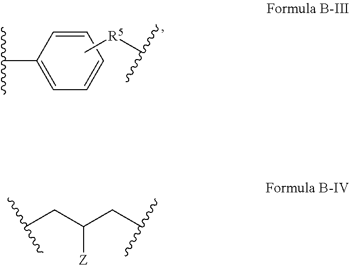

C₁₋₆-alkylene, C₂₋₆-alkenylene and C₂₋₆-alkenylene,

Z is represented by Formula Z-I and

R⁹ is independently selected from hydrogen, C₁₋₃-alkyl or (OCH₂CH₂)ₙOCH₃

R¹⁰ is independently selected from C₁₋₃-alkyl or (OCH₂CH₂)ₙOCH₃, wherein n is from 40 to 50.

In another preferred embodiment X¹ is N, R⁴ is hydrogen;

B is selected from the group consisting of C₁₋₄⁻alkenylene and

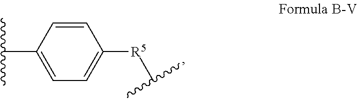

R⁵ is methylene or ethylene; and

Z is represented by Formula Z-I, and R⁹ and R¹⁰ are identical C₁₋₃-alkyl substituents.

In yet another preferred embodiment X¹ is N, R⁴ is hydrogen; B is —(CH₂)₄⁻; and Z is represented by Formula Z-I, R⁹ and R¹⁰ being identical C₁₋₃-alkyl substituents.

In yet another preferred embodiment X¹ is —NH—, —S— or —O—; B is —(CH₂)₄—; and Z is represented by Formula Z-I, R⁹ and R¹⁰ being identical C₁₋₃-alkyl substituents.

If A is represented by Formula A-IV, then R² is preferably S(O)CH₃.

In one embodiment of the invention the substituent A is presented by Formula A-I. The corresponding compounds are structurally related to ibuprofen. These compounds have a pronounced anti-cancer activity and are therefore particularly useful in the treatment of cancers such as e.g. lung cancer or colon cancer. Moreover, these compounds have a significant analgesic and anti-inflammatory effect. Accordingly, the compounds of Formula I include but are not limited to compounds 125 to 130, shown below:

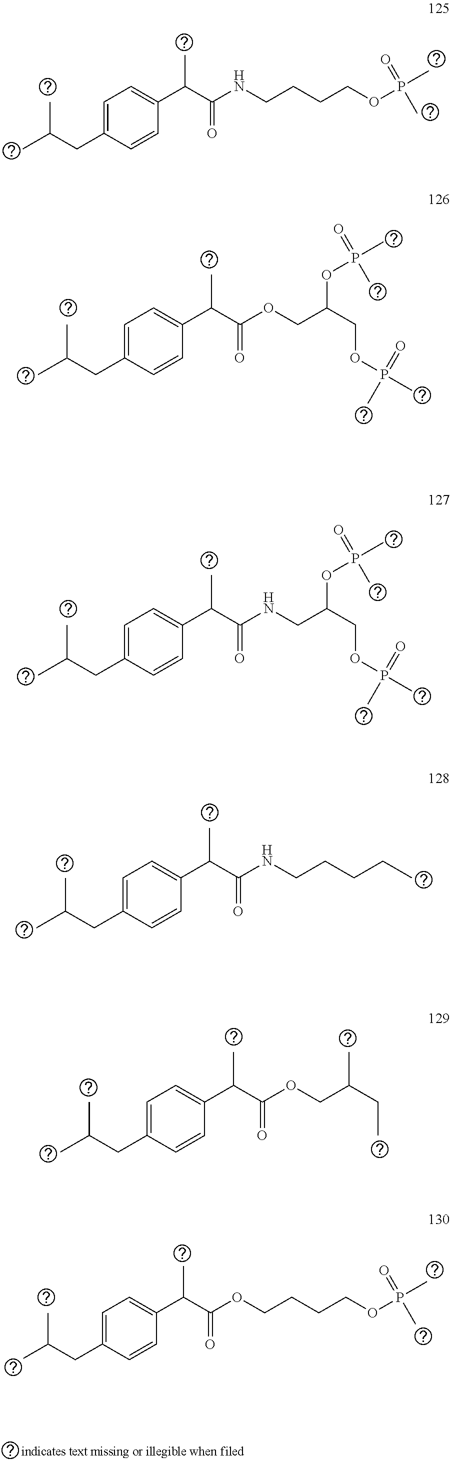

In another embodiment of the invention the substituent A is presented by Formula A-II and R¹ is hydrogen. Thus, the corresponding compounds are structurally related to aspirin. These compounds have anti-cancer activity, analgesic activity and anti-inflammatory activity and can be used in the treatment of cancer, pain and/or inflammation-related diseases. The compounds of Formula I include but are not limited to the following compounds 131 to 146:

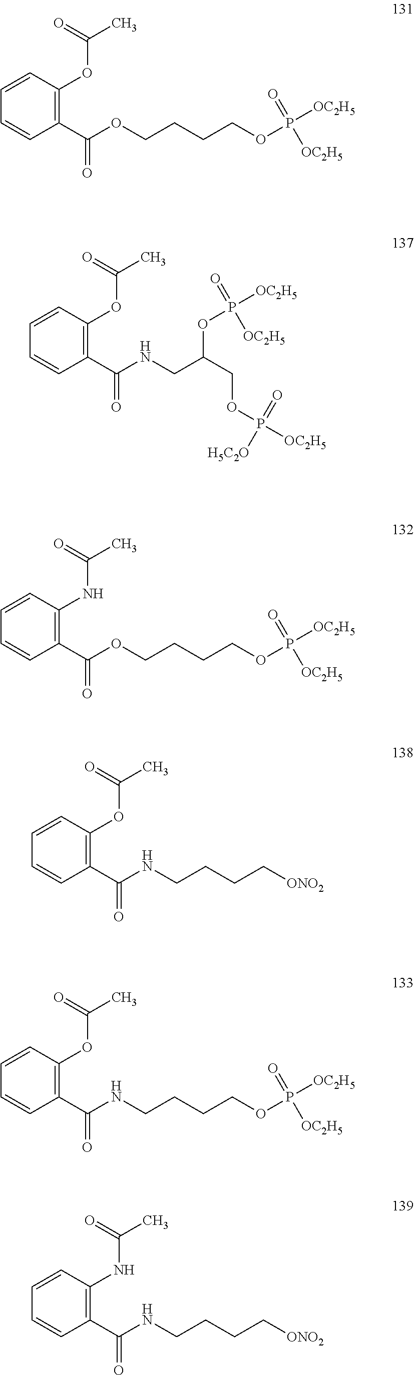

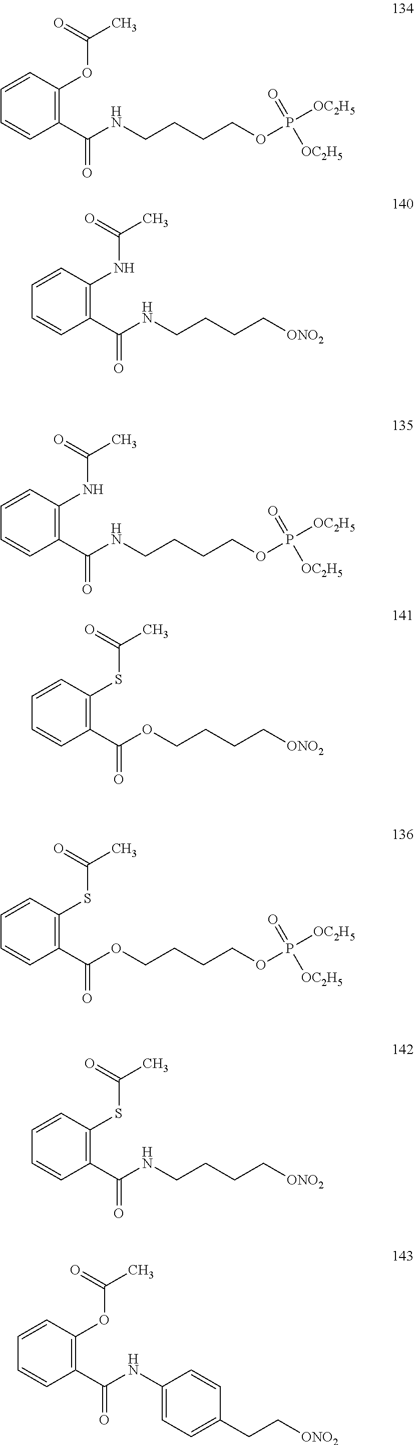

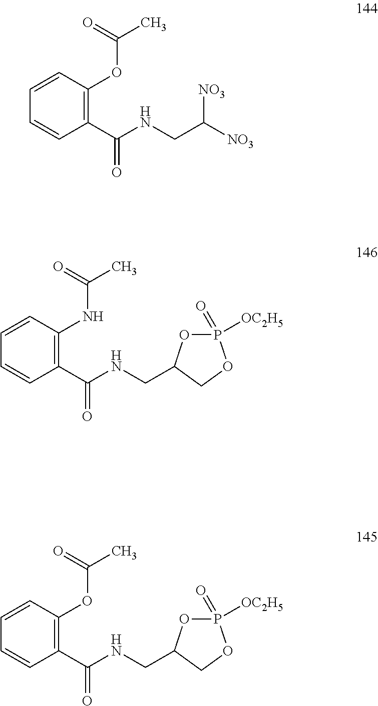

In another embodiment of the invention the substituent A is represented by Formula A-II and R¹ is trifluoromethyl. The corresponding compounds are structurally related to triflusal. These compounds have an anti-cancer and anti-inflammatory activity and can be used in the treatment of pain, inflammation-related diseases such as arthritis and cancers. According to this embodiment, the compounds of Formula I include but are not limited to the compounds 146 to 151 listed below:

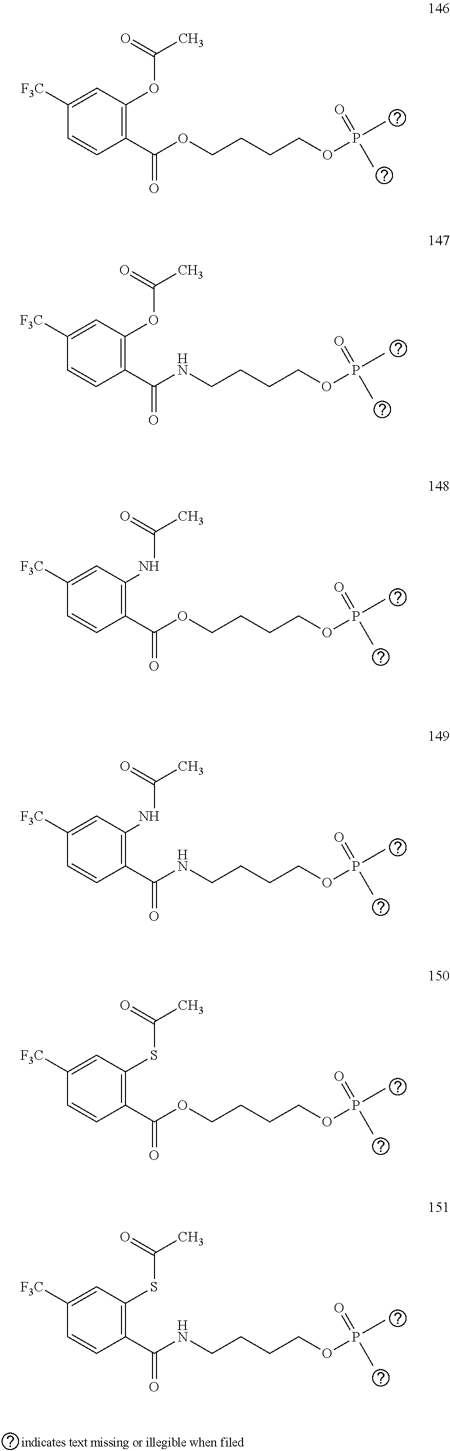 [SMILES: CC(=O)Oc1cc(C(F)(F)F)ccc1C(=O)OCCCCOP(C)(C)=O ; CC(=O)Oc1cc(C(F)(F)F)ccc1C(=O)NCCCCOP(C)(C)=O ; CC(=O)Nc1cc(C(F)(F)F)ccc1C(=O)OCCCCOP(C)(C)=O ; CC(=O)Nc1cc(C(F)(F)F)ccc1C(=O)NCCCCOP(C)(C)=O ; CC(=O)Sc1cc(C(F)(F)F)ccc1C(=O)OCCCCOP(C)(C)=O ; CC(=O)Sc1cc(C(F)(F)F)ccc1C(=O)NCCCCOP(C)(C)=O]

In a further embodiment of the invention the substituent A is represented by Formula A-III. The corresponding compounds are structurally related to indomethacin. These compounds have a significant anti-cancer activity and therefore can be used for treatment and prevention of a broad range of cancers, such as for instance colon cancer and lung cancer. They can further be used to treat inflammation and/or pain such as that related to arthritis. In this embodiment, the compounds of Formula I include but are not limited to the compounds 152 to 153 shown below:

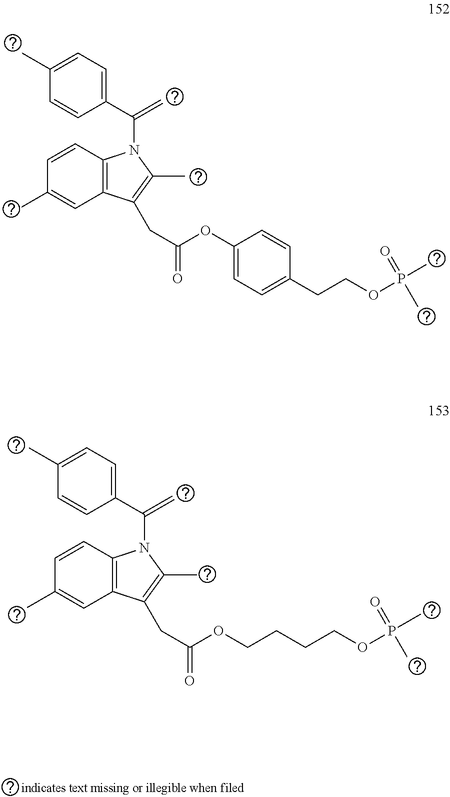

Yet another embodiment of the present invention provides compounds of Formula I in which the substituent A is represented by Formula A-IV. Preferably, R² is S(O)CH₃. The corresponding compounds are structurally related to sulindac. Such compounds have a strong anti-cancer activity and therefore can be used in the treatments of cancers, such as for instance colon cancer. These compounds are further useful in the treatment of pain and/or inflammation. These compounds include but are not limited to the compounds 154 to 156 listed below:

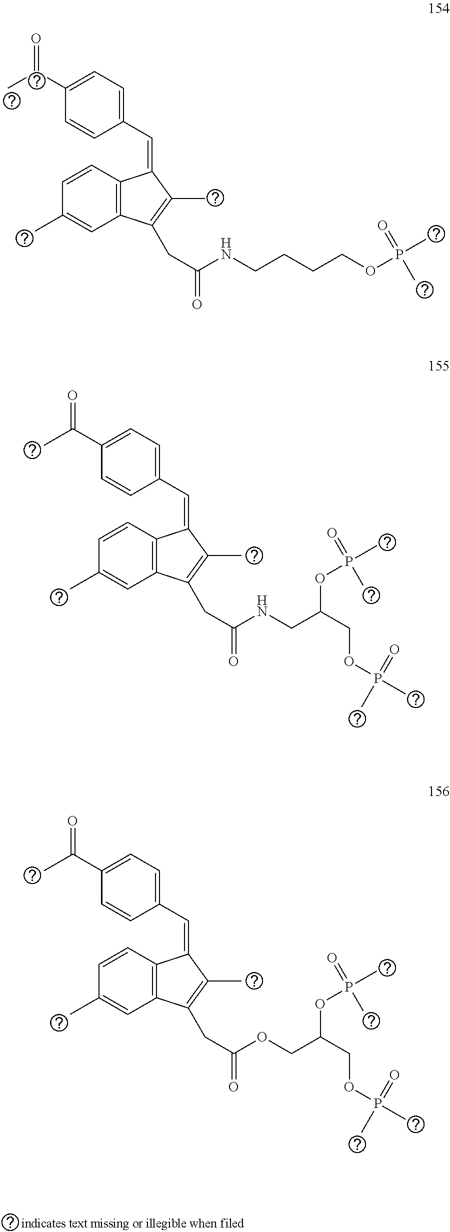

In a further embodiment of the invention the substituent A is represented by Formula A-V. In These compounds have an anti-cancer activity and can be employed in the treatment of cancers such as pancreatic cancer. They can further be used to treat pain and/or inflammation. The corresponding compounds of Formula I include but are not limited to the compounds 157 to 168 shown below:

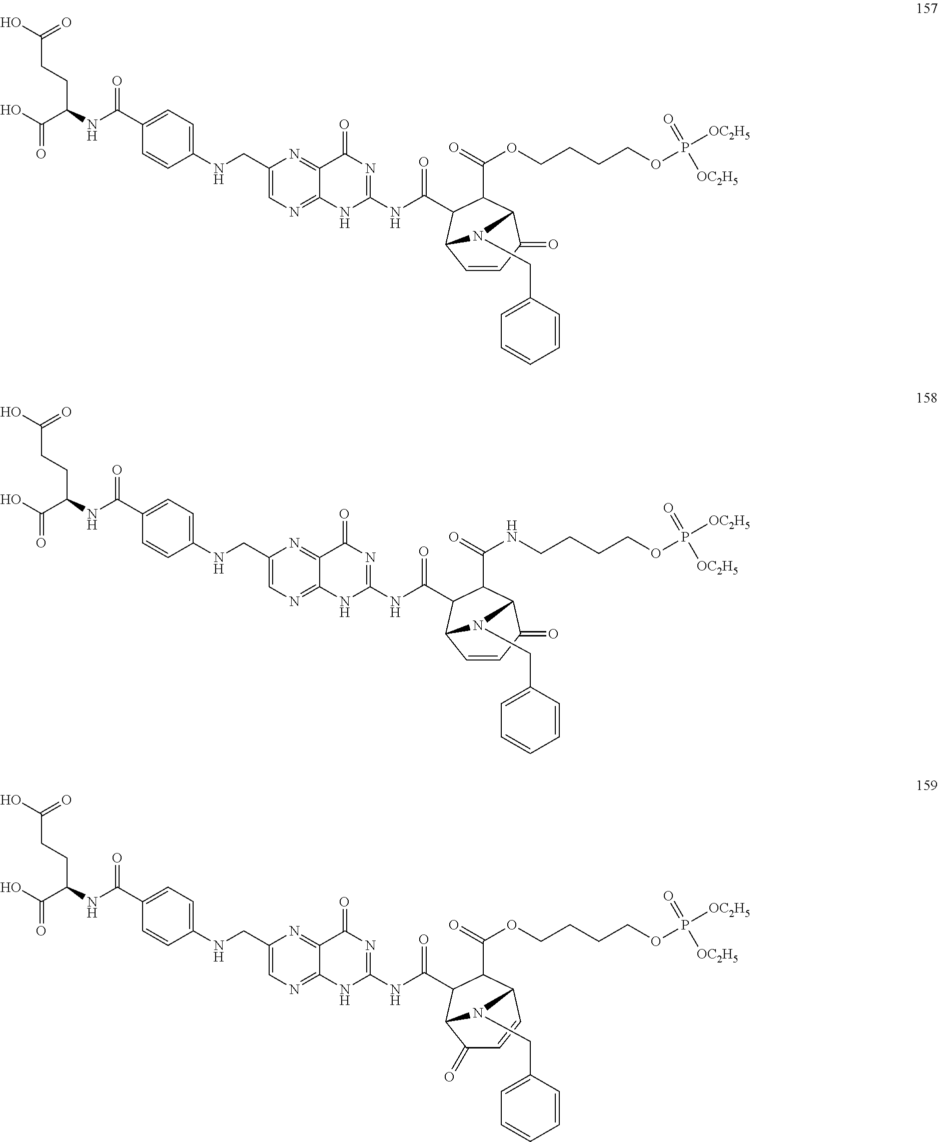 [SMILES: CCOP(=O)(OCC)OCCCCOC(=O)C1C(C(=O)Nc2nc(=O)c3nc(CNc4ccc(C(=O)N[C@H](CCC(=O)O)C(=O)O)cc4)cnc3[nH]2)[C@H]2C=CC(=O)[C@@H]1N2Cc1ccccc1 ; CCOP(=O)(OCC)OCCCCNC(=O)C1C(C(=O)Nc2nc(=O)c3nc(CNc4ccc(C(=O)N[C@H](CCC(=O)O)C(=O)O)cc4)cnc3[nH]2)[C@H]2C=CC(=O)[C@@H]1N2Cc1ccccc1 ; CCOP(=O)(OCC)OCCCCOC(=O)C1C(C(=O)Nc2nc(=O)c3nc(CNc4ccc(C(=O)N[C@H](CCC(=O)O)C(=O)O)cc4)cnc3[nH]2)[C@H]2C(=O)C=C[C@@H]1N2Cc1ccccc1]

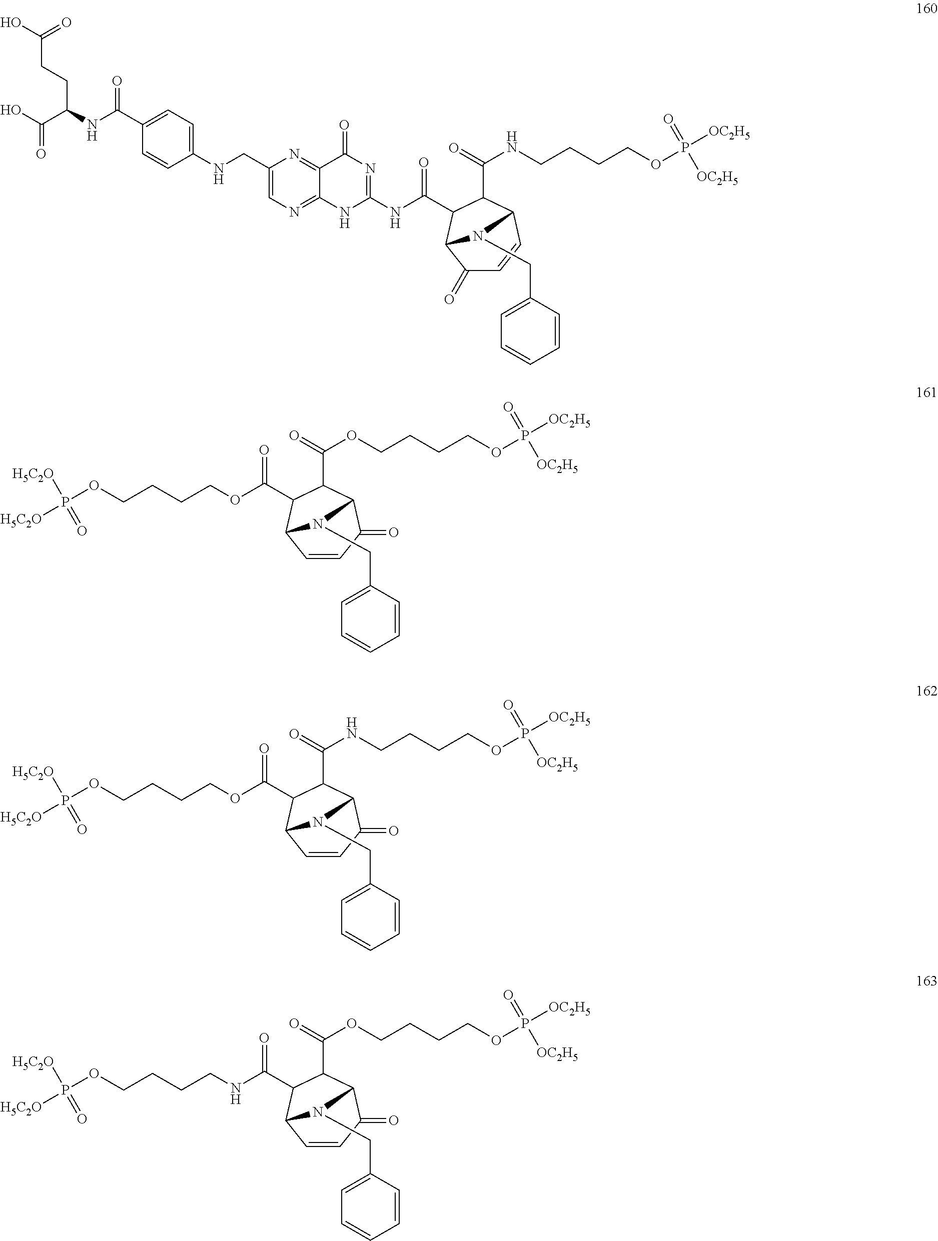

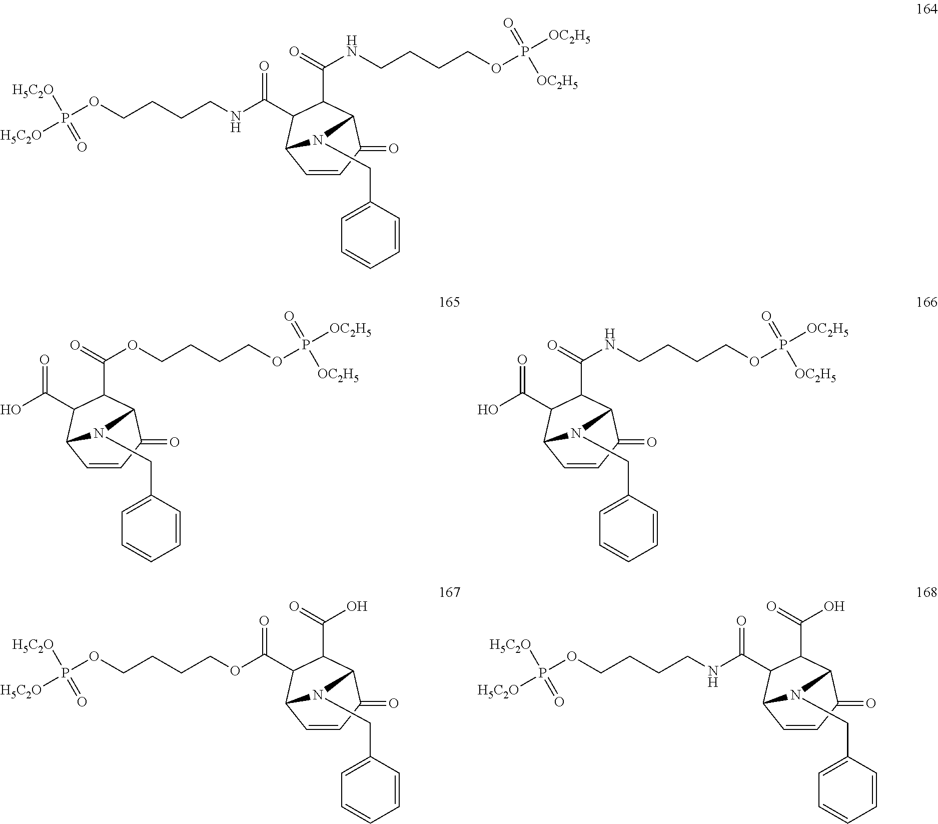

In a further embodiment of the invention the substituent A is presented by Formula A-VI. These compounds are structurally related to rigosertib. The corresponding compounds have a strong anti-cancer activity and can be used in the treatment and/or prevention of cancers such as pancreatic cancer. In this embodiment, the compounds of Formula I include but are not limited to the compounds 169 to 170 shown below:

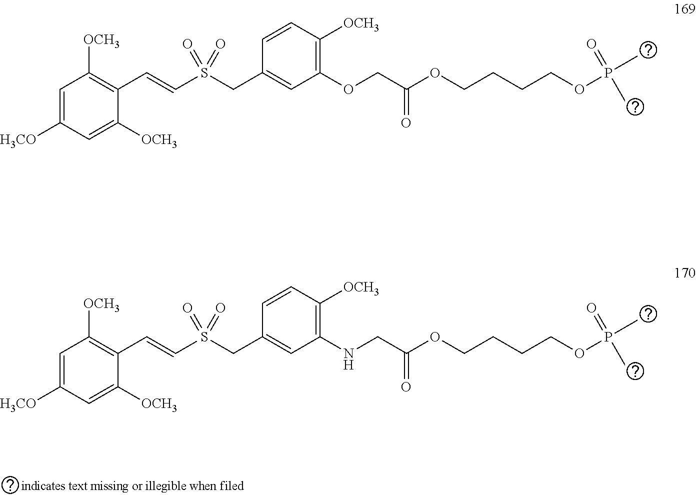 [SMILES: COc1cc(OC)c(/C=C/S(=O)(=O)Cc2ccc(OC)c(OCC(=O)OCCCCOP(C)(C)=O)c2)c(OC)c1 ; COc1cc(OC)c(/C=C/S(=O)(=O)Cc2ccc(OC)c(NCC(=O)OCCCCOP(C)(C)=O)c2)c(OC)c1]

In a further embodiment of the invention the substituent A is presented by Formula A-VII. Thus, the corresponding compounds are structurally related to valproic acid. The corresponding compounds are strong anti-cancer agents and are highly suitable in the treatment and/or prevention of cancers such as pancreatic cancer. In this embodiment, the compounds of Formula I include but are not limited to the compounds 171 to 173 shown below:

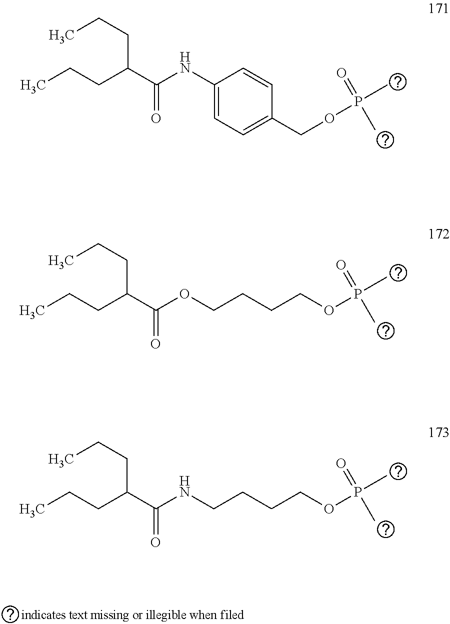

In a further embodiment of the invention the substituent A is presented by Formula A-VIII. These compounds have pronounced anti-cancer properties and are suitable for treatment and/or prevention of a broad range of cancers. In this embodiment, the compounds of Formula I include but are not limited to the compounds 174 to 175 shown below:

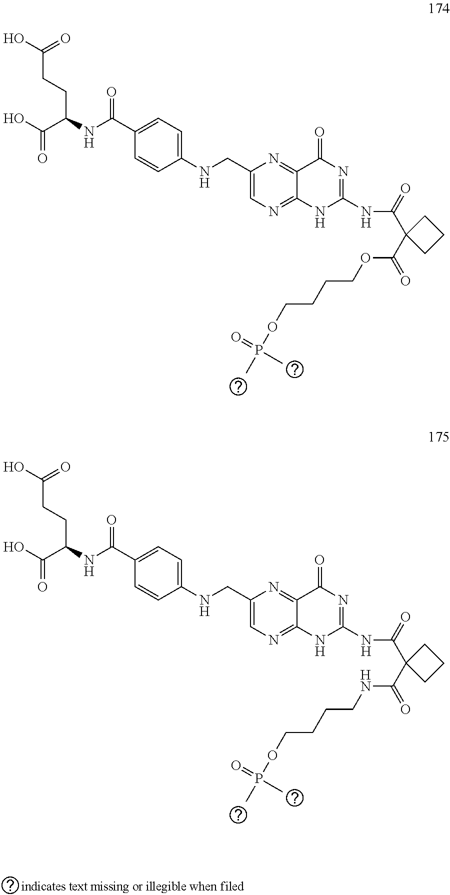 [SMILES: CP(C)(=O)OCCCCOC(=O)C1(C(=O)Nc2nc(=O)c3nc(CNc4ccc(C(=O)N[C@H](CCC(=O)O)C(=O)O)cc4)cnc3[nH]2)CCC1 ; CP(C)(=O)OCCCCNC(=O)C1(C(=O)Nc2nc(=O)c3nc(CNc4ccc(C(=O)N[C@H](CCC(=O)O)C(=O)O)cc4)cnc3[nH]2)CCC1]

In yet another embodiment of the invention the substituent A is represented by Formula A-IX and thus the corresponding compounds are structurally related to salinomycin. These compounds have a strong anti-cancer activity and can therefore be employed in the treatment of cancers such as breast cancer. In this embodiment, the compounds of Formula I include but are not limited to compounds 176 and 177.

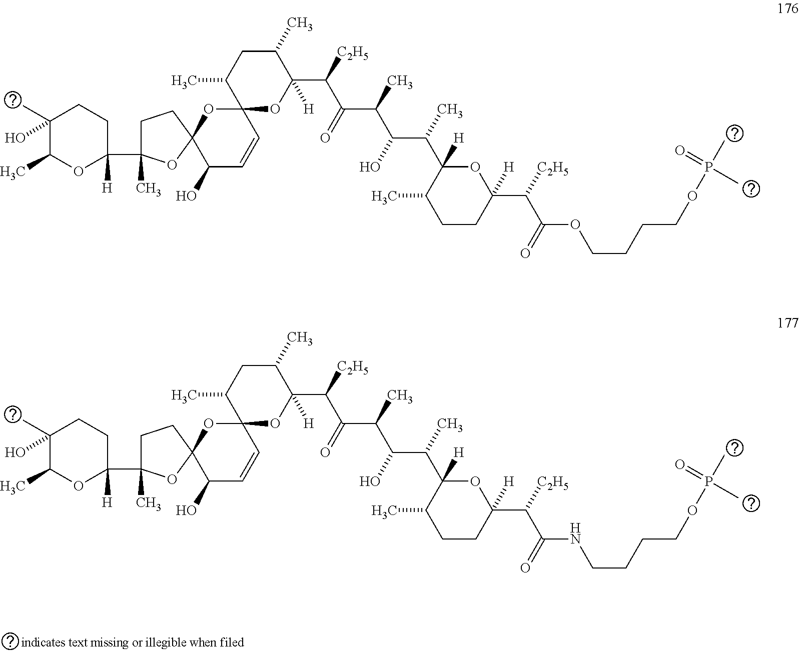

Another embodiment of the present invention provides compounds of general Formula I as shown below:

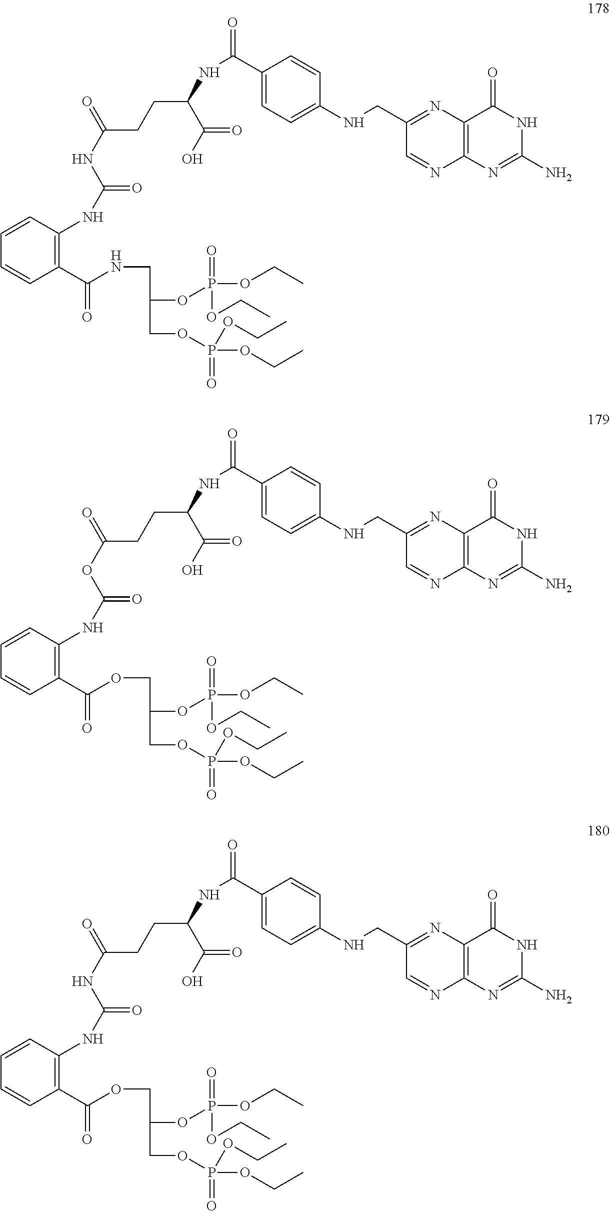

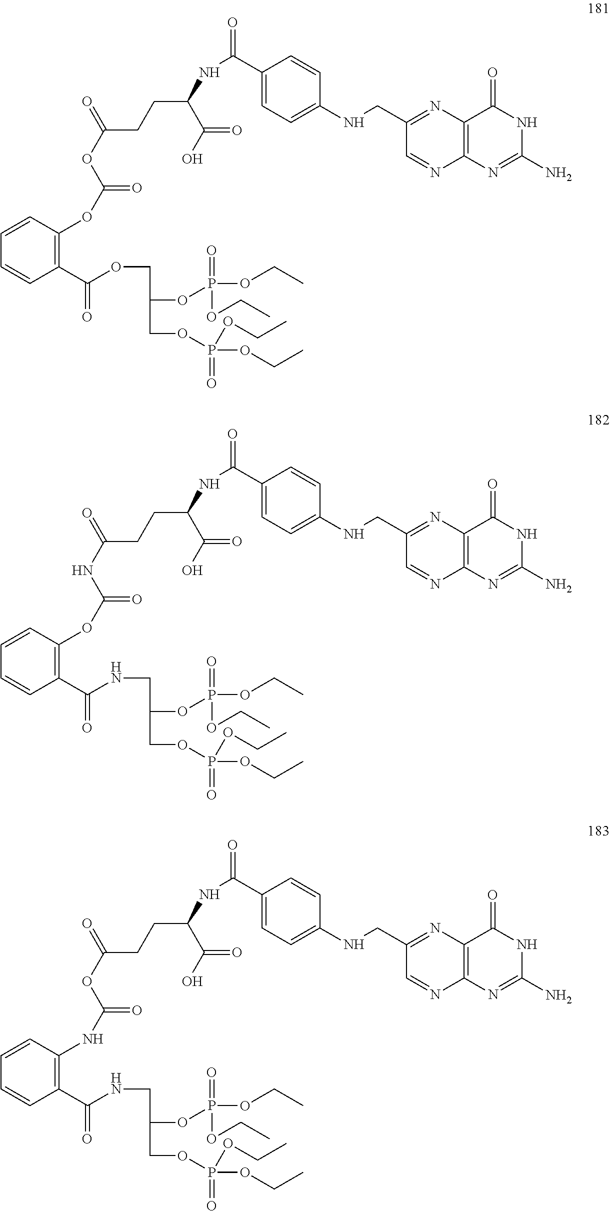

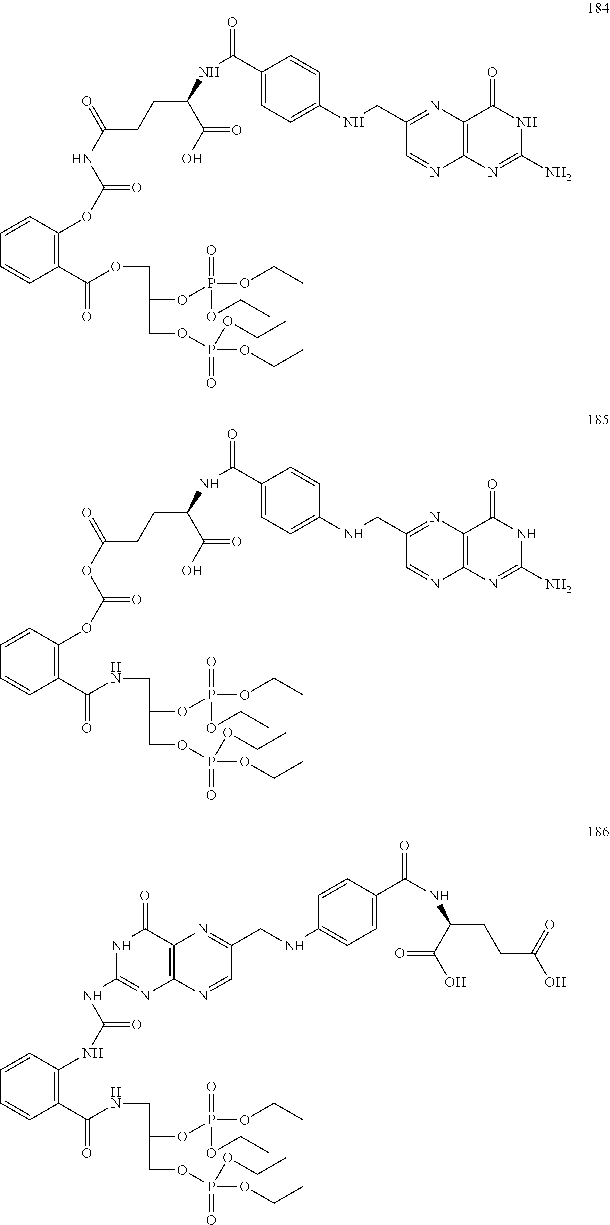

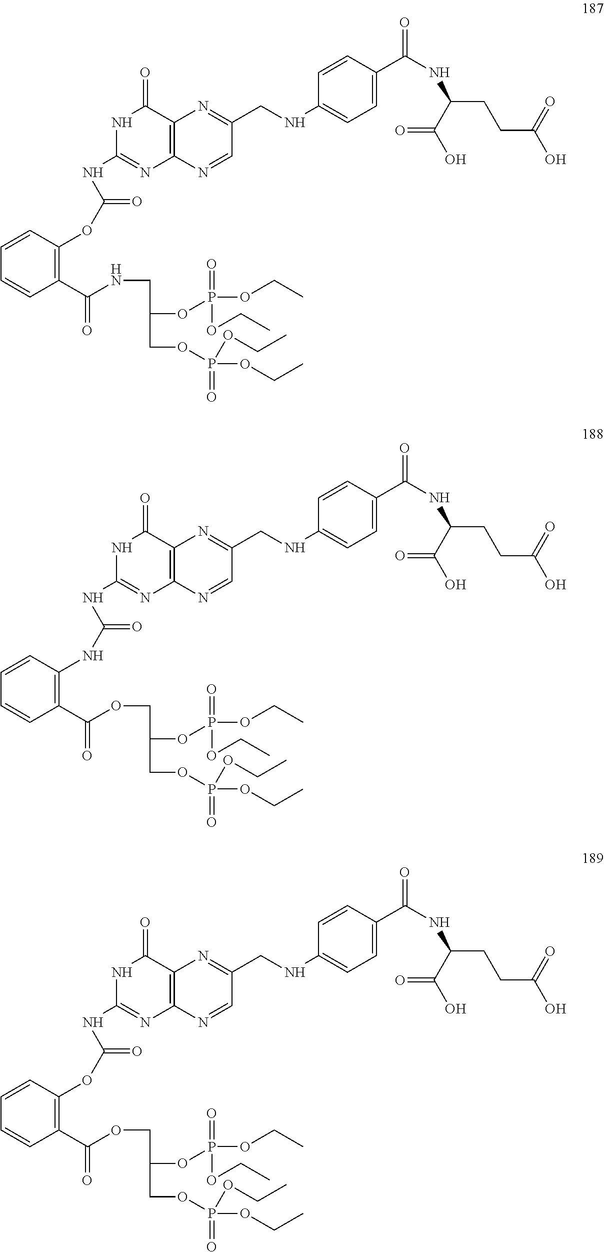

Particular embodiments of the present invention are amide derivative compounds of Formula I as shown below:

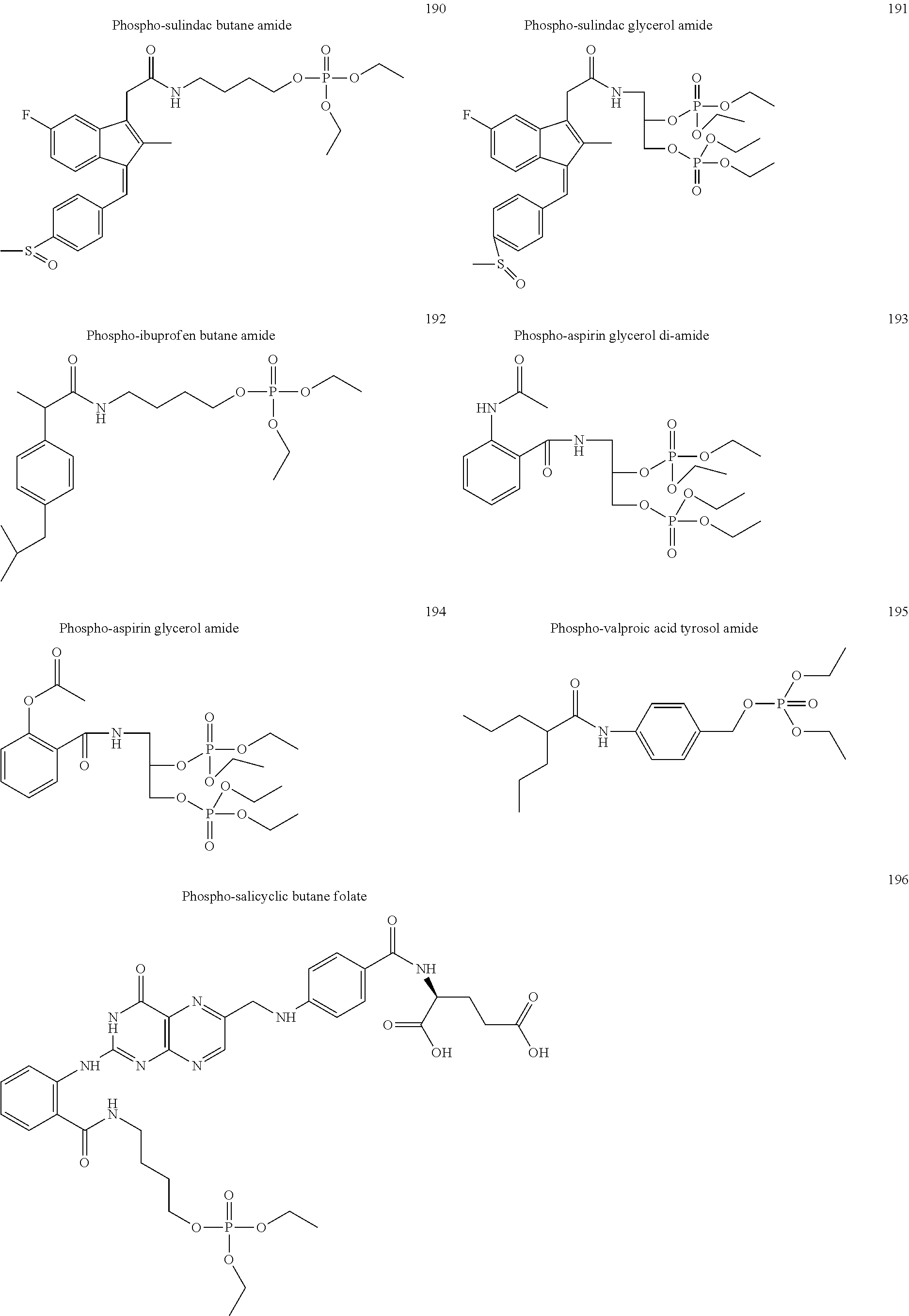

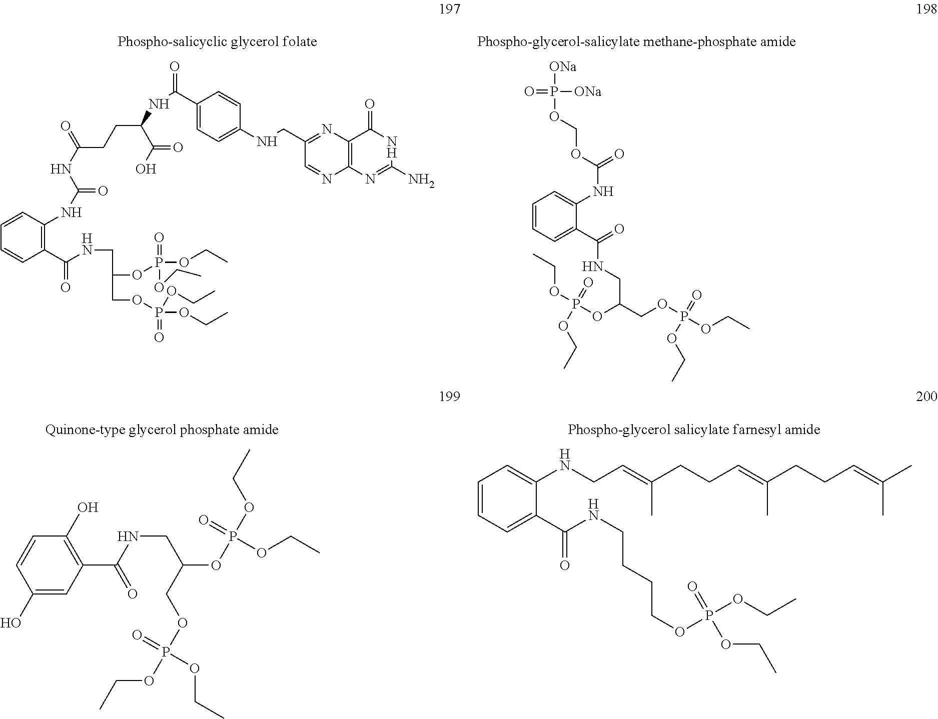

Another embodiment of the present invention provides novel therapeutics including a novel group of salicylic, 2-mercaptobenzoic and anthranilic acid derivatives of general Formula V and methods of using them in the treatment and/or prevention of disorders such as cancer and precancerous conditions and for the treatment of inflammation, pain and fever.

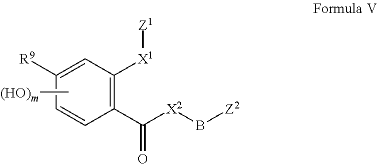

The compounds of Formula V, wherein the substituents X¹, Z¹, B, R⁹, X² and Z² are as defined below, are capable of undergoing metabolism as depicted in the scheme below. Furthermore, contrary to the compounds of Formula V, acetylsalicylic acid itself is not capable of undergoing these transformations in vivo.

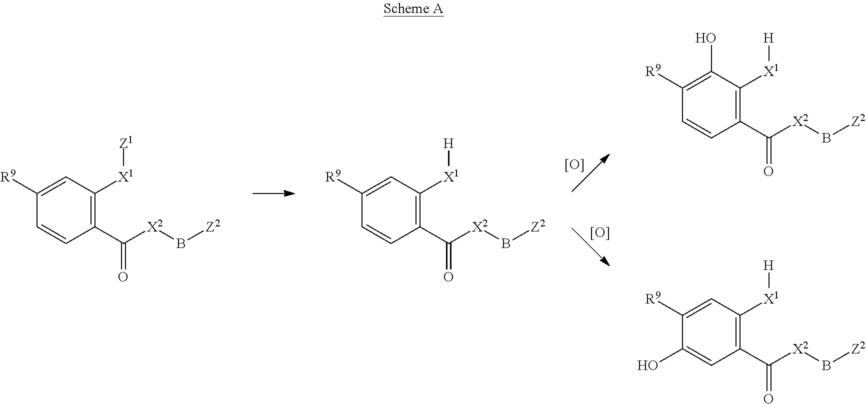

This embodiment of the invention is inter alia based at least on the surprising finding that the metabolites represented by Formula V have a pronounced anti-cancer activity and are therefore suitable for the treatment and/or prevention of cancer and precancerous conditions. Moreover, these compounds are capable of undergoing oxidation to highly reactive quinone-type derivatives, in particular to benzoquinones (see, e.g., the scheme below), the cytostatic properties of which are likely to explain the anti-cancer activity of the parent compounds of Formula IV.

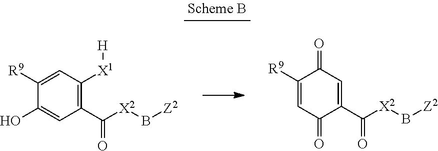

Accordingly, one aspect of the present invention relates to the compound of Formula V:

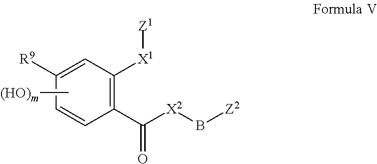

or an enantiomer, diastereomer, racemate, tautomer, salt or hydrate thereof.

The number of hydroxy substituents m may be 0 or 1. When m is 1, the position of the hydroxy group in the aromatic moiety is not particularly limited. Thus, said hydroxy group may be located in the 2, 3, 4, 5 or 6 position relative to the carbonyl group, 5 being particularly preferred.

The substituents X¹ and X² can be —O—, —S— or —NR¹—, R¹ being hydrogen or an alkyl group having 1 to 6 carbon atoms, more preferred 1 to 3 carbon atoms such as, for instance, methyl or ethyl, preferably methyl. The substituent R⁹ may be hydrogen or trifluoromethyl, hydrogen being particularly preferred.

In some preferred embodiments, Z¹ is a folic acid residue. The folic acid residue may be selected from the residues defined by the Formulae Z-III, Z-IV or Z-V which are shown below:

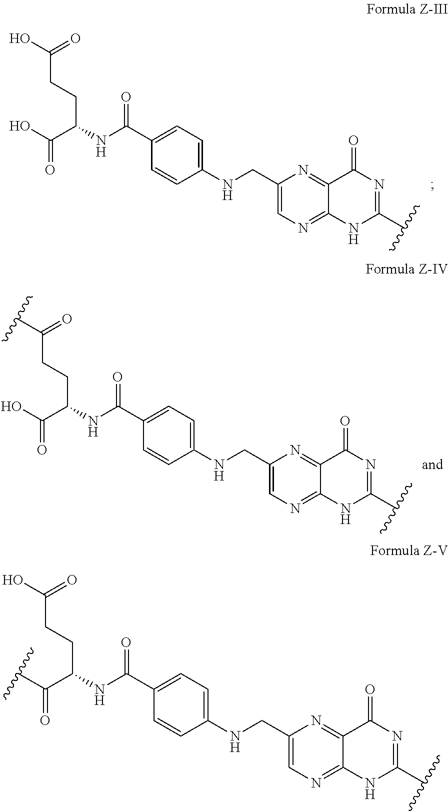 [SMILES: *c1nc(=O)c2nc(CNc3ccc(C(=O)N[C@@H](CCC(=O)O)C(=O)O)cc3)cnc2[nH]1 ; *C(=O)CC[C@H](NC(=O)c1ccc(NCc2cnc3[nH]c(*)nc(=O)c3n2)cc1)C(=O)O ; *C(=O)[C@H](CCC(=O)O)NC(=O)c1ccc(NCc2cnc3[nH]c(*)nc(=O)c3n2)cc1]

Without wishing to be bound by any theory it is believed that compounds of Formula V having Z¹ represented by Formulae Z-III to Z-V have a particularly strong anticancer activity. In particular, anti-cancer activity of these compounds is usually higher than the activity of the corresponding compounds in which Z¹ is hydrogen. In these embodiments, the substituent X¹ may, for instance, be selected from —O— and —NR¹—, whereby —O— and —NH— are particularly preferred.

In yet other preferred embodiments, Z¹ is farnesyl (IUPAC name: (2E,6E)-3,7,11-trimethyldodeca-2,6,10-trienyl) the structure of which is shown below:

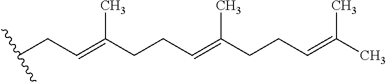 [SMILES: *C/C=C(\C)CC/C=C(\C)CCC=C(C)C]

In these embodiments, the substituent X¹ may be selected from —S—, —O— and —NH—, whereby —S— is particularly preferred.

The substituent Z² may be represented by Formulae Z-I or Z-II

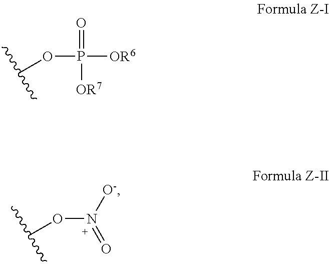

Formula Z-I being particularly preferable.

R⁶ and R⁷ are independently selected from hydrogen, C₁₋₁₀₀-alkyl and polyethylene glycol substituent. Preferably, the substituents R⁶ and R⁷ are independently selected from C₁₋₃-alkyl and (OCH₂CH₂)ₙOCH₃, wherein n is from 40 to 50, wherein it is particularly preferred that R⁶ and R⁷ are identical C₁₋₃-alkyl substituents, for instance ethyl.

The substituent B is an optionally substituted aliphatic, heteroaliphatic, aromatic, heteroaromatic or alkylaryl substituent having 1 to 40 carbon atoms. In particular, the substituent B may be selected from the group consisting of

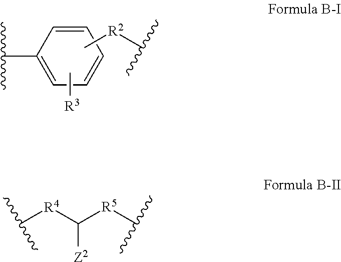

and an aliphatic substituent with 1 to 40, preferably with 1 to 22 carbon atoms.

Substituents R², R⁴, and R⁵ can be the same or different alkylene substituent having 1 to 3 carbon atoms. Preferably the substituent R² in Formula B-I is selected from the group consisting of methylene, ethylene and trimethylene; in a more preferred embodiment, R² is methylene or ethylene, whereby methylene is particularly preferred.

In yet another preferred embodiment, the substituent B is represented by Formula B-II and R⁴ and R⁵ are identical alkylene substituents having 1 to 3 carbon atoms. In a particularly preferred embodiment, R⁴ and R⁵ are both methylene substituents so that the substituent B in Formula I is represented by Formula B-IV:

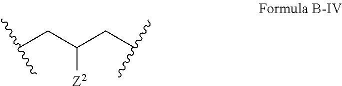

Z² is defined above.

In a particularly preferred embodiment, the substituent B together with X² and Z² forms a glycerol ester residue.

R³ in Formula B-I can be hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy substituents. In some preferred embodiments, R³ in Formula B-I is selected from hydrogen, an alkyl having 1 to 3 carbon atoms, halo and methoxy. Preferably, R³ is selected from the group consisting of hydrogen, methyl, fluoro, chloro, bromo and methoxy, preferably from the group consisting of hydrogen, methyl, chloro and fluoro. In a particularly preferred embodiment, R³ represents hydrogen so that the substituent B is represented by Formula B-III:

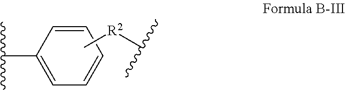

The substitution pattern of the substituent B in Formulae B-1 and B-III is not particularly limited. When the substituent B is represented by Formula B-III, the aromatic moiety of the substituent B can be 1,2- or 1,3- or 1,4-substituted.

Preferably, the aromatic moiety of B is 1,4-substituted so that B is represented by Formula B-V:

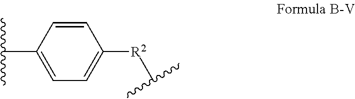

In another embodiment of the present invention, substituent B is an aliphatic substituent, preferably having 1 to 40 carbon atoms, more preferred 1 to 22 carbon atoms, further preferred 1 to 6 carbon atoms. In a particularly preferred embodiment, B is selected from the group consisting of alkylene substituents with 1 to 6 carbon atoms, alkenylene substituents having 2 to 6 carbon atoms and alkynylene substituents having 2 to 6 carbon atoms. In a more preferred embodiment, substituent B is an alkylene substituent with 1 to 6 carbon atoms, preferably an alkylene substituent with 1 to 4 carbon atoms, even more preferred a tetramethylene substituent.

In a further embodiment of the present invention, the substituent B together with the substituent Z² forms a structure

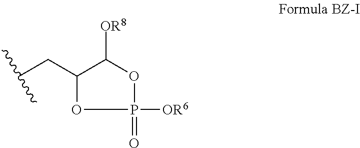

R⁶ is defined as above,

R⁸ is independently selected from hydrogen, an aliphatic substituent, preferably with 1 to 22 carbon atoms, more preferred C₁₋₆-alkyl, and a polyethylene glycol residue.

In yet another preferred embodiment, R⁸ is hydrogen and the substituent B together with the substituent Z² forms a structure

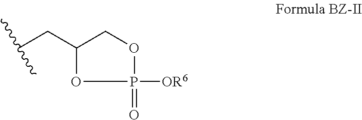

In one embodiment of the invention, m is 0, R⁹ is hydrogen and the substituent Z¹ is a folic acid residue represented by Formulae Z-III to Z-V. Accordingly, the compounds of Formula I include but are not limited to compounds 201 to 218 the structures of which are shown below:

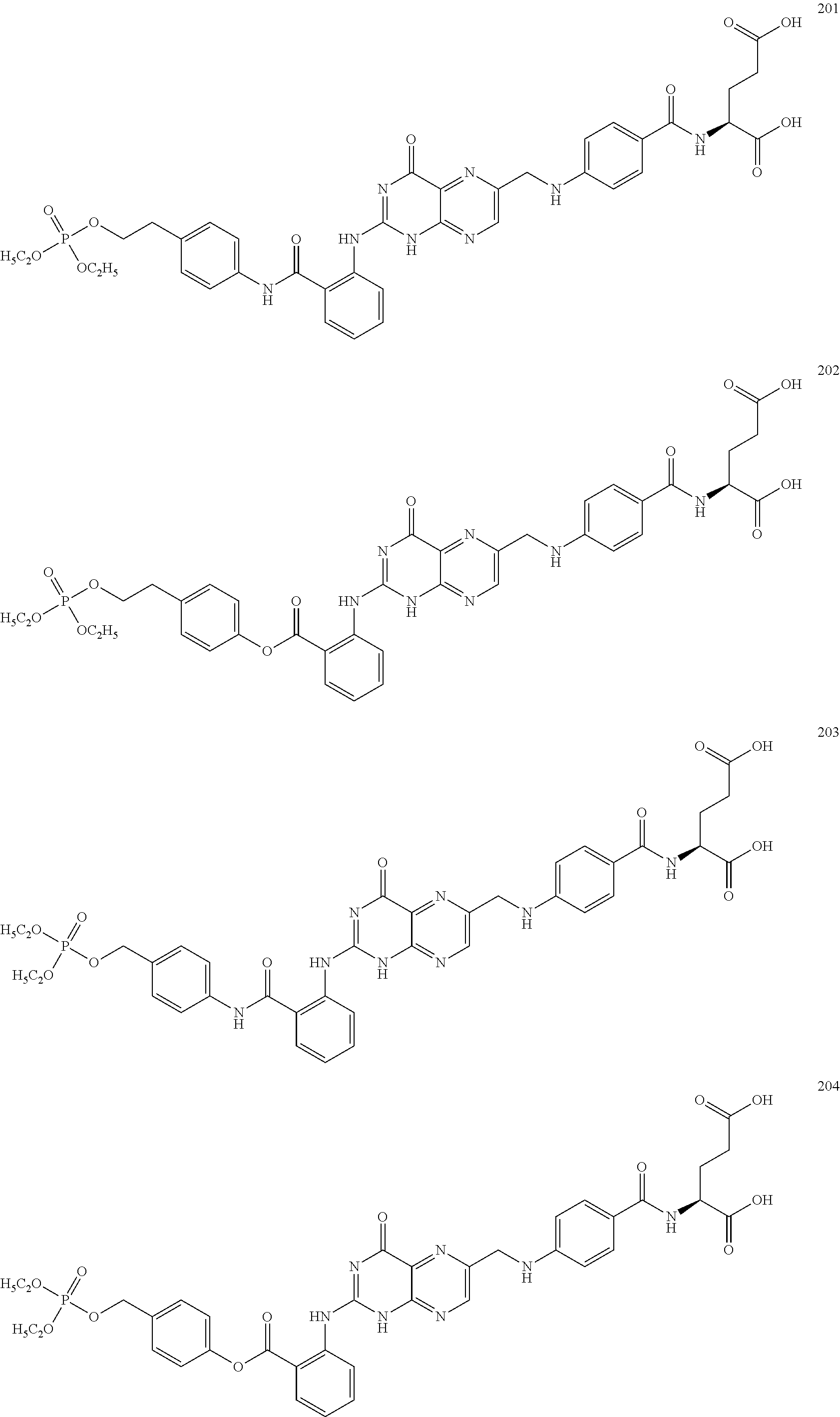

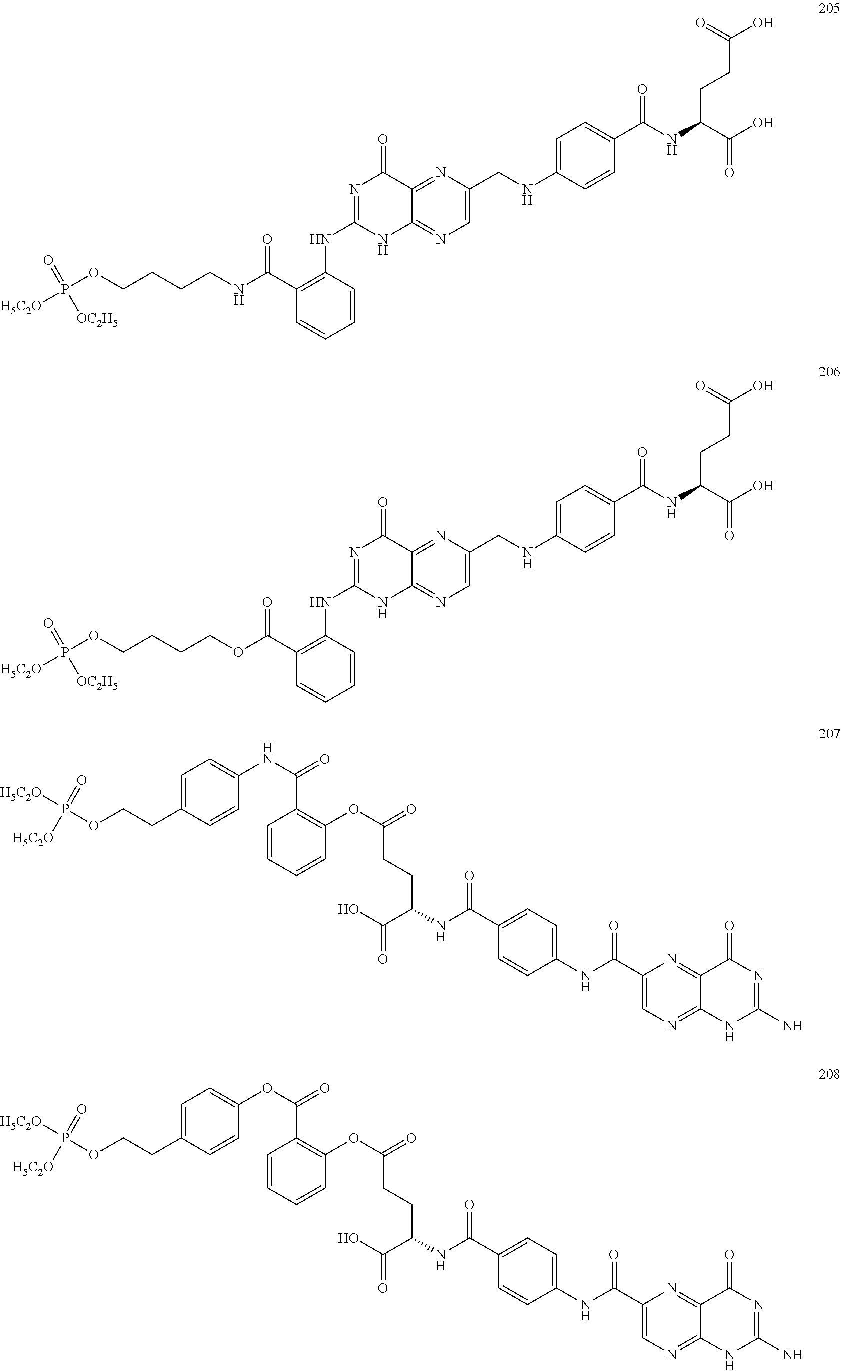

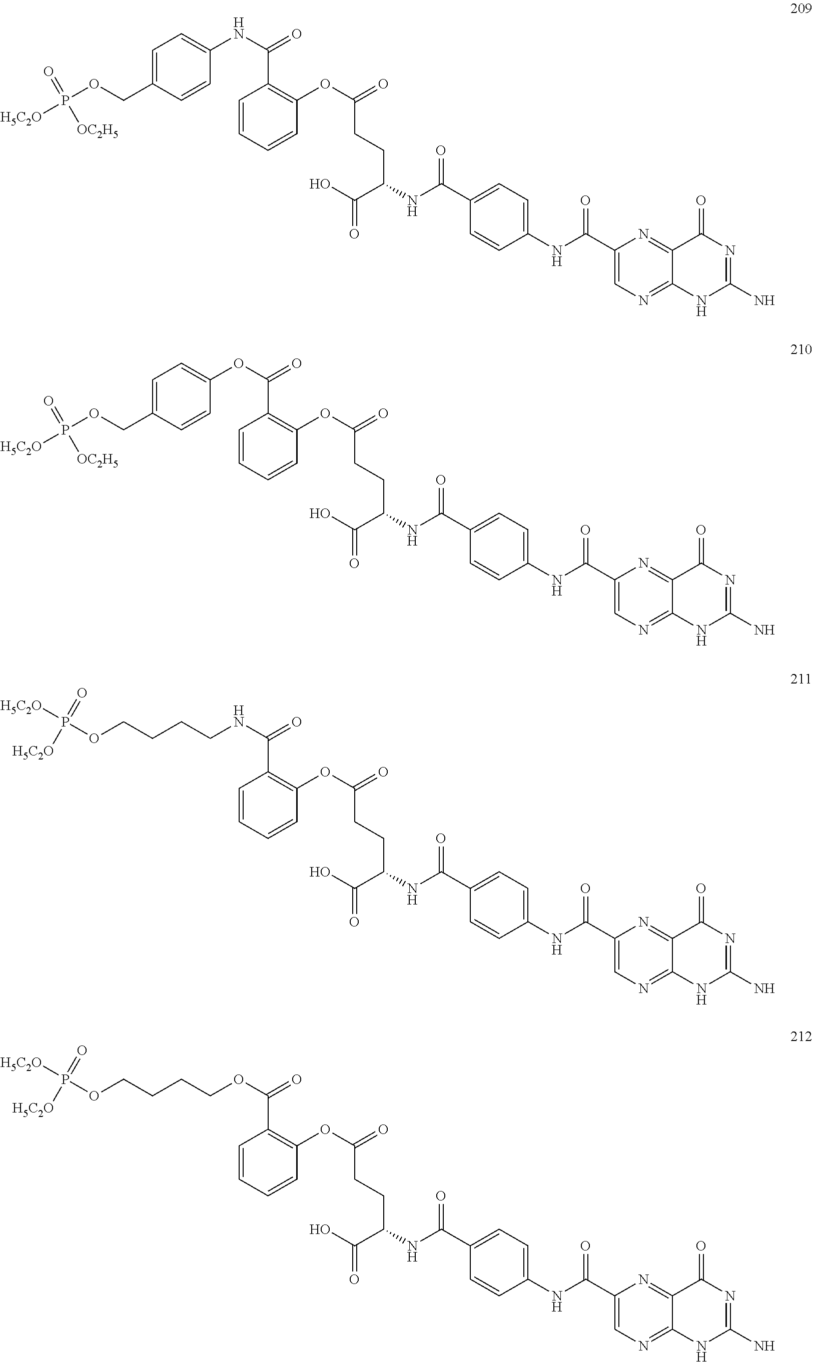

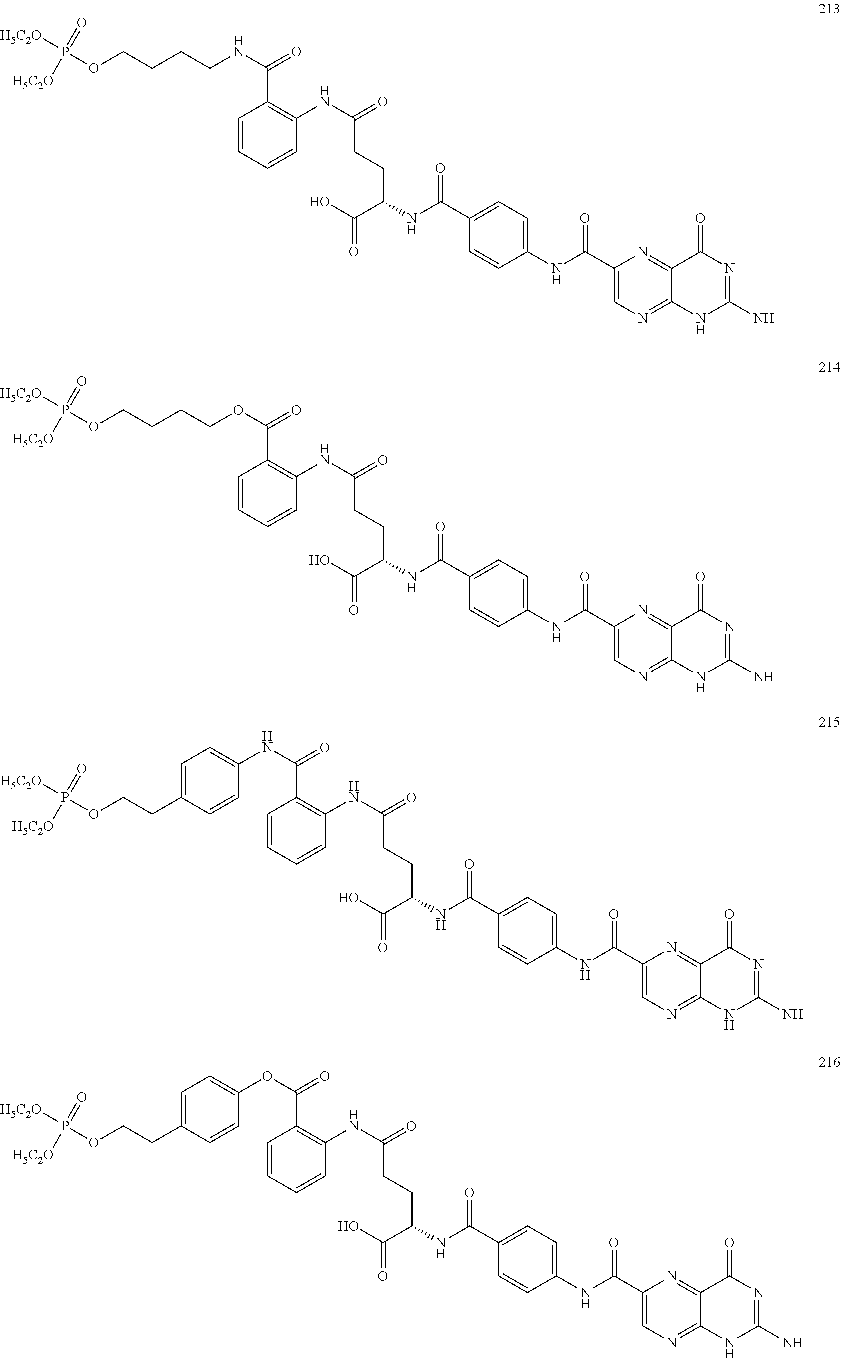

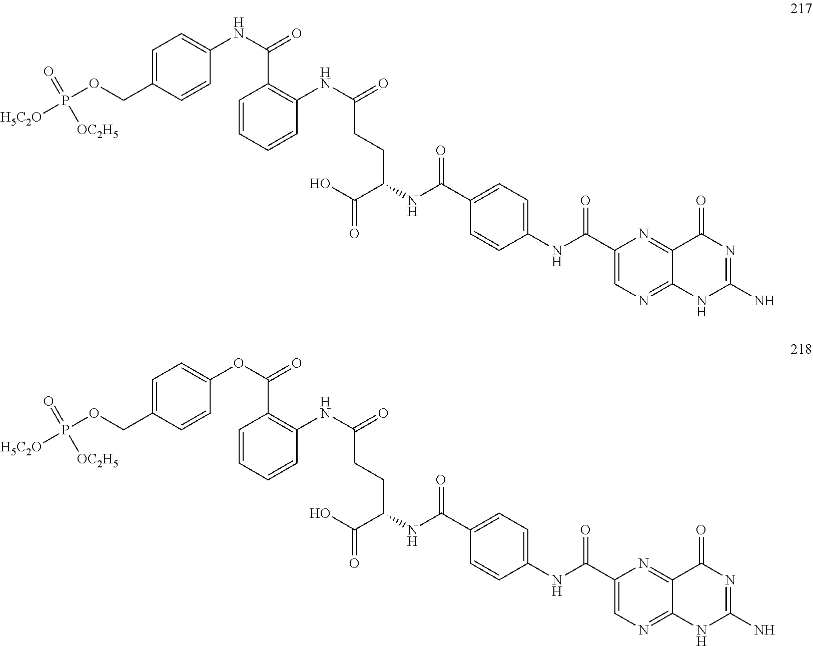

In another embodiment, m is 0, R⁹ is hydrogen and the substituent Z¹ is farnesyl. Without wishing to be bound by a theory, applicant believes that these compounds may be advantageously employed for the treatment of cancers such as pancreatic cancer. Accordingly, the compounds of Formula I include but are not limited to compounds 219 to 236 shown below:

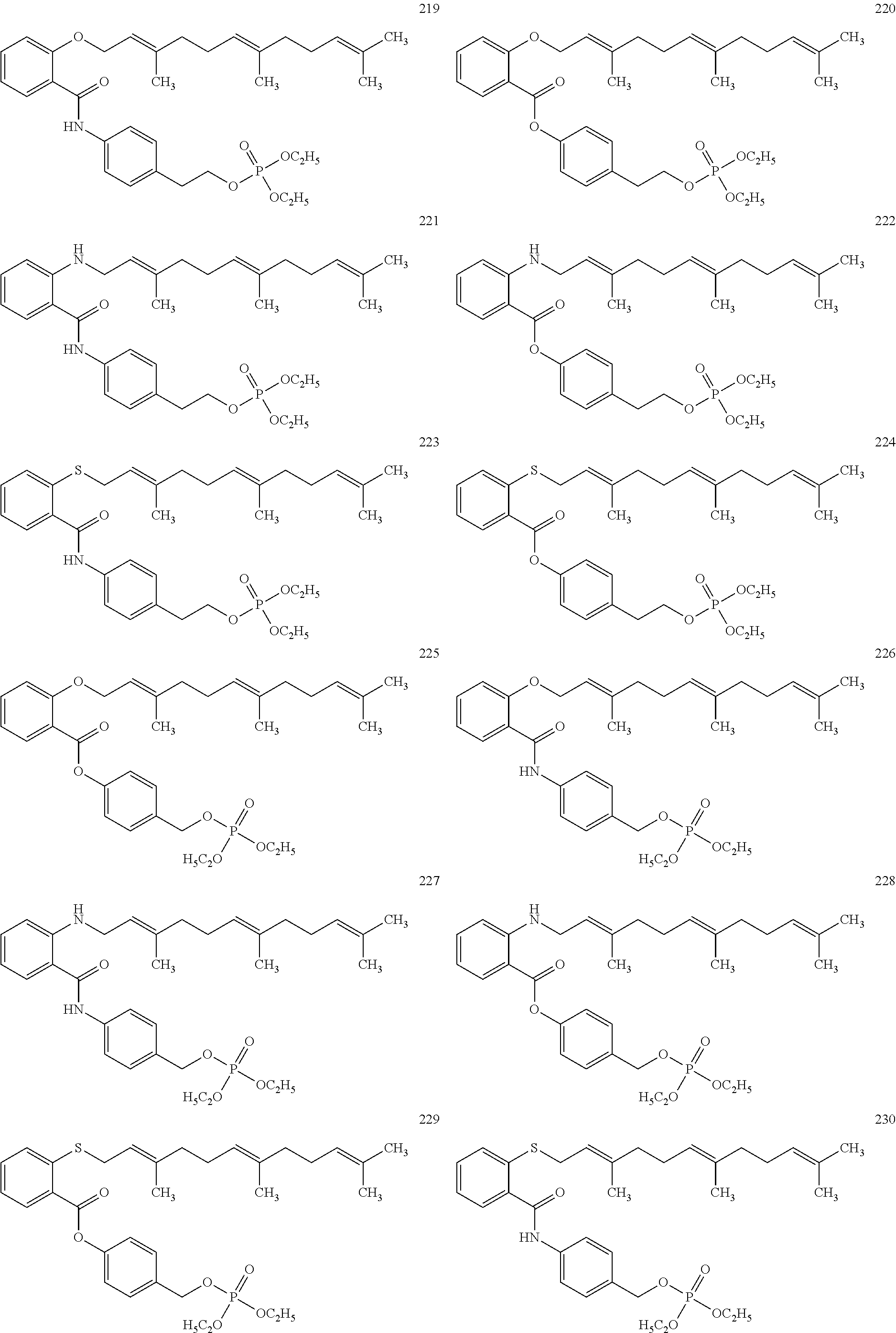

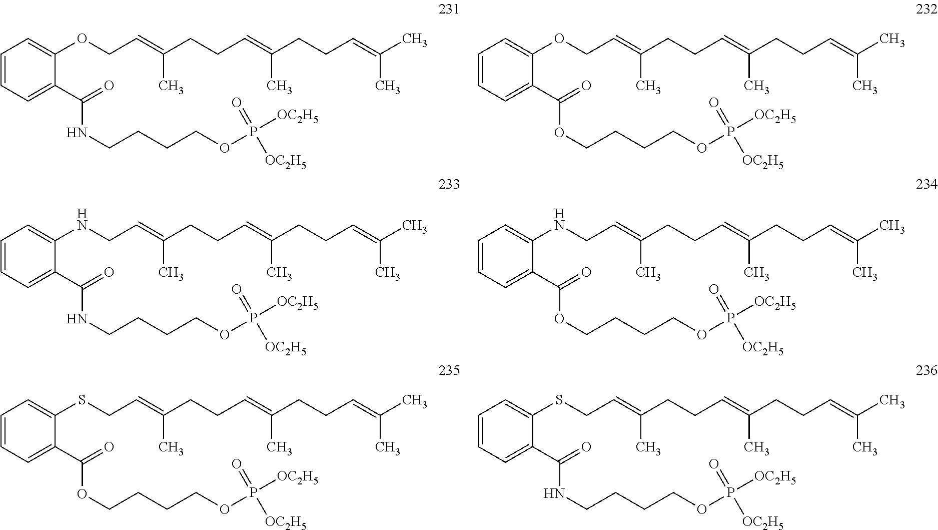 [SMILES: CCOP(=O)(OCC)OCCCCNC(=O)c1ccccc1OC/C=C(\C)CC/C=C(\C)CCC=C(C)C ; CCOP(=O)(OCC)OCCCCOC(=O)c1ccccc1OC/C=C(\C)CC/C=C(\C)CCC=C(C)C ; CCOP(=O)(OCC)OCCCCNC(=O)c1ccccc1NC/C=C(\C)CC/C=C(\C)CCC=C(C)C ; CCOP(=O)(OCC)OCCCCOC(=O)c1ccccc1NC/C=C(\C)CC/C=C(\C)CCC=C(C)C ; CCOP(=O)(OCC)OCCCCOC(=O)c1ccccc1SC/C=C(\C)CC/C=C(\C)CCC=C(C)C ; CCOP(=O)(OCC)OCCCCNC(=O)c1ccccc1SC/C=C(\C)CC/C=C(\C)CCC=C(C)C]

In a further embodiment of the invention, m is 0, the substituent X¹ is —O— and the substituents Z¹ and R⁹ are hydrogens. Thus, the corresponding compounds represented by Formula I are salicylic acid derivatives which include but are not limited to the following compounds 237 to 240.

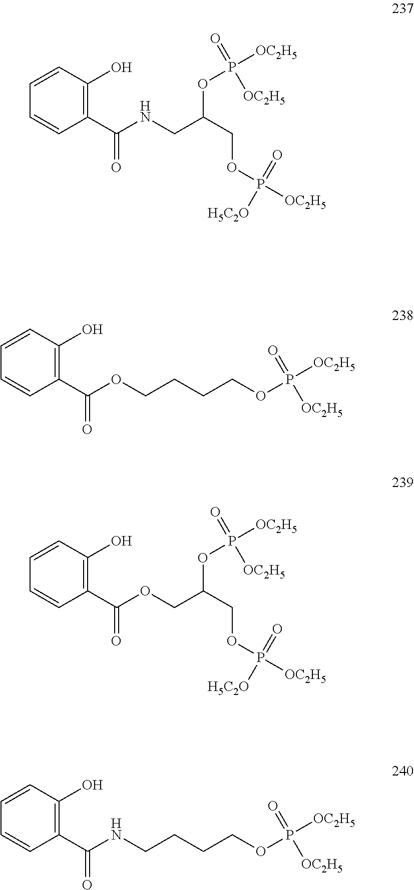

In still a further preferred embodiment, m is 1, the substituent X¹ is —O— and the substituents Z¹ and R⁹ are hydrogens. Thus, the compounds represented by Formula I are derivatives of dihydroxybenzoic acids including but not being limited to the following compounds 241 to 252.

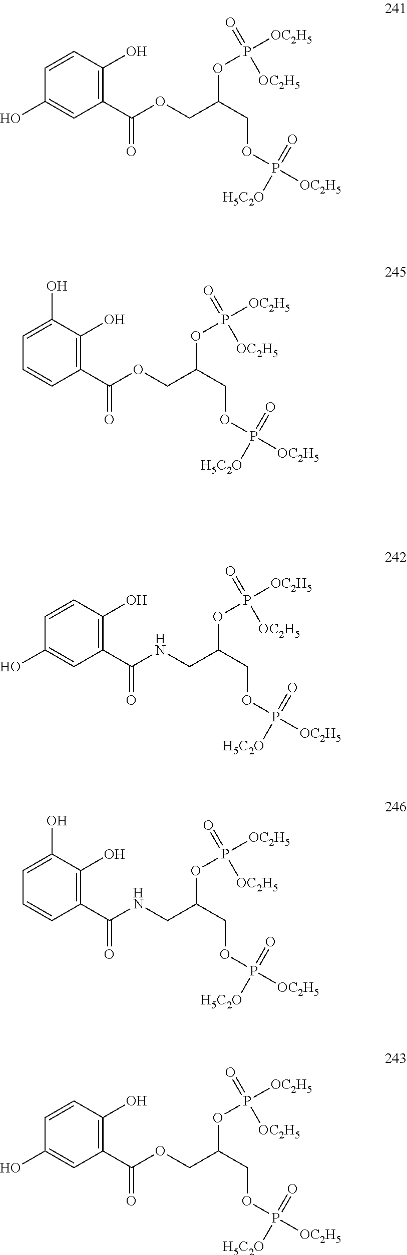

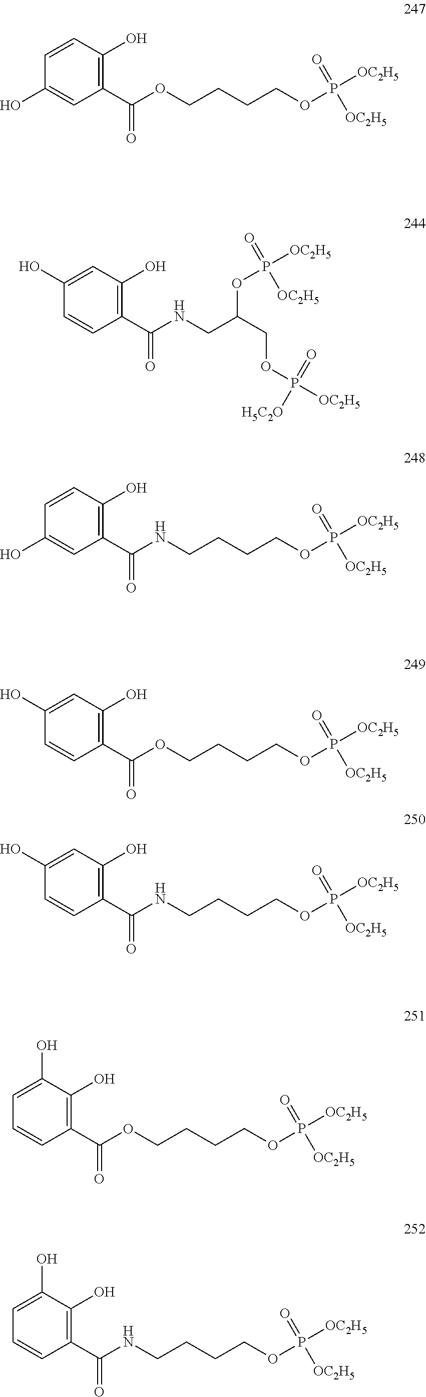

A further aspect of the invention relates to a compound of general Formula VI

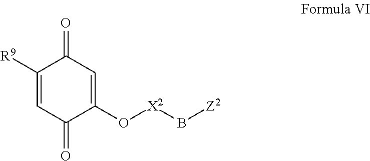

or an enantiomer, diastereomer, racemate, tautomer, salt or hydrate thereof. The substituents X², B and Z² are as defined above.

In a further preferred embodiment of the invention, the compound is represented by Formula VI and the substituent R⁹ is hydrogen. Thus, the corresponding compounds are p-benzoquinone derivatives such as compounds 253-256.

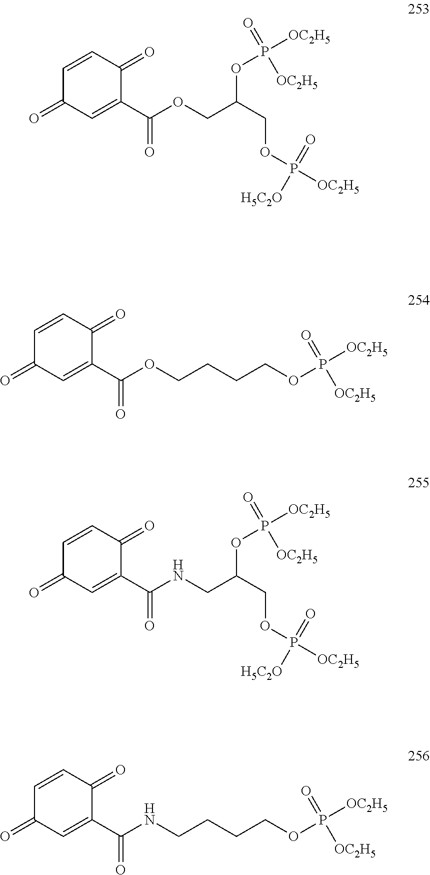

In further embodiments, preferred com pounds of the present invention may be described by the general Formula VII: A-D-Y. The compounds of Formula VII include but are not limited to the following:

| Com- | | Com- | | Com- | |
|---|---|---|---|---|---|
| pound | Compound | pound | Compound | pound | Compound |
| No. | structure | No. | structure | No. | structure |
| 238 | A¹-D¹-Y¹ | 257 | A¹-D¹-Y² | 258 | A¹-D¹-Y³ |
| 259 | A¹-D²-Y¹ | 260 | A¹-D²-Y² | 261 | A¹-D²-Y³ |
| 262 | A¹-D³-Y¹ | 263 | A¹-D³-Y² | 264 | A¹-D³-Y³ |
| 265 | A²-D¹-Y¹ | 266 | A²-D¹-Y² | 267 | A²-D¹-Y³ |
| 268 | A²-D²-Y¹ | 269 | A²-D²-Y² | 270 | A²-D²-Y³ |
| 271 | A²-D³-Y¹ | 272 | A²-D³-Y² | 273 | A²-D³-Y³ |
| 274 | A³-D¹-Y¹ | 275 | A³-D¹-Y² | 276 | A³-D¹-Y³ |
| 277 | A³-D²-Y¹ | 278 | A³-D²-Y² | 279 | A³-D²-Y³ |
| 280 | A³-D³-Y¹ | 281 | A³-D³-Y² | 282 | A³-D³-Y³ |
| 283 | A⁴-D¹-Y¹ | 284 | A⁴-D¹-Y² | 285 | A⁴-D¹-Y³ |
| 286 | A⁴-D²-Y¹ | 287 | A⁴-D²-Y² | 288 | A⁴-D²-Y³ |
| 289 | A⁴-D³-Y¹ | 290 | A⁴-D³-Y² | 291 | A⁴-D³-Y³ |
| 292 | A⁵-D¹-Y¹ | 293 | A⁵-D¹-Y² | 294 | A⁵-D¹-Y³ |
| 295 | A⁵-D²-Y¹ | 296 | A⁵-D²-Y² | 297 | A⁵-D²-Y³ |
| 298 | A⁵-D³-Y¹ | 299 | A⁵-D³-Y² | 300 | A⁵-D³-Y³ |
| 301 | A⁶-D¹-Y¹ | 302 | A⁶-D¹-Y² | 303 | A⁶-D¹-Y³ |
| 304 | A⁶-D²-Y¹ | 305 | A⁶-D²-Y² | 306 | A⁶-D²-Y³ |
| 307 | A⁶-D³-Y¹ | 308 | A⁶-D³-Y² | 309 | A⁶-D³-Y³ |
| 232 | A⁷-D¹-Y¹ | 310 | A⁷-D¹-Y² | 311 | A⁷-D¹-Y³ |
| 225 | A⁷-D²-Y¹ | 312 | A⁷-D²-Y² | 313 | A⁷-D²-Y³ |
| 220 | A⁷-D³-Y¹ | 314 | A⁷-D³-Y² | 315 | A⁷-D³-Y³ |
| 234 | A⁸-D¹-Y¹ | 316 | A⁸-D¹-Y² | 317 | A⁸-D¹-Y³ |
| 228 | A⁸-D²-Y¹ | 318 | A⁸-D²-Y² | 319 | A⁸-D²-Y³ |
| 222 | A⁸-D³-Y¹ | 320 | A⁸-D³-Y² | 321 | A⁸-D³-Y³ |
| 322 | A⁹-D1-Y¹ | 323 | A⁹-D¹-Y² | 324 | A⁹-D¹-Y³ |
| 325 | A⁹-D2-Y¹ | 326 | A⁹-D²-Y² | 327 | A⁹-D²-Y³ |
| 328 | A⁹-D³-Y¹ | 329 | A⁹-D³-Y² | 330 | A⁹-D³-Y³ |
| 331 | A¹⁰-D¹-Y¹ | 332 | A¹⁰-D¹-Y² | 333 | A¹⁰-D¹-Y³ |
| 334 | A¹⁰-D²-Y¹ | 335 | A¹⁰-D²-Y² | 336 | A¹⁰-D²-Y³ |
| 337 | A¹⁰-D³-Y¹ | 338 | A¹⁰-D³-Y² | 339 | A¹⁰-D³-Y³ |
| 212 | A¹¹-D¹-Y¹ | 340 | A¹¹-D¹-Y² | 341 | A¹¹-D¹-Y³ |
| 210 | A¹¹-D²-Y¹ | 342 | A¹¹-D²-Y² | 343 | A¹¹-D²-Y³ |
| 208 | A¹¹-D³-Y¹ | 344 | A¹¹-D³-Y² | 345 | A¹¹-D³-Y³ |
| 206 | A¹²-D¹-Y¹ | 346 | A¹²-D¹-Y² | 347 | A¹²-D¹-Y³ |
| 204 | A¹²D²-Y¹ | 348 | A¹²D²-Y² | 349 | A¹²D²-Y³ |
| 202 | A¹²D³-Y¹ | 350 | A¹²D³-Y² | 351 | A¹²-D³-Y³ |
| 352 | A¹³-D¹-Y¹ | 353 | A¹³-D¹-Y² | 354 | A¹³-D¹-Y³ |
| 355 | A¹³-D²-Y¹ | 356 | A¹³-D²-Y² | 357 | A¹³-D²-Y³ |
| 358 | A¹³-D³-Y¹ | 359 | A¹³-D³-Y² | 360 | A¹³-D³-Y³ |
| 361 | A¹⁴-D¹-Y¹ | 362 | A¹⁴-D¹-Y² | 363 | A¹⁴-D¹-Y³ |
| 364 | A¹⁴-D²-Y¹ | 365 | A¹⁴-D²-Y² | 366 | A¹⁴-D²-Y³ |
| 367 | A¹⁴-D³-Y¹ | 368 | A¹⁴-D³-Y² | 369 | A¹⁴-D³-Y³ |

wherein each Group A, D, Y is represented by the corresponding structure below.

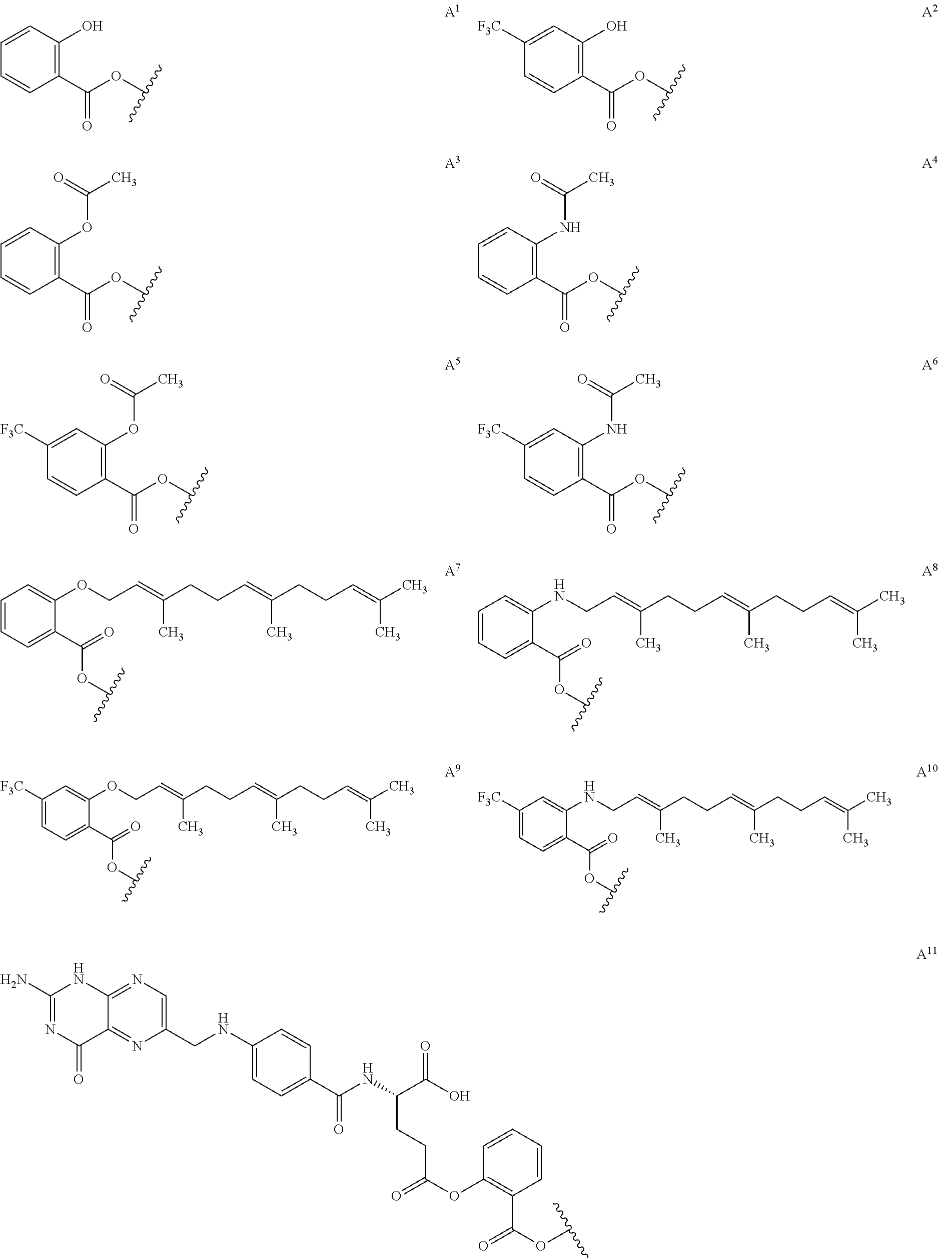

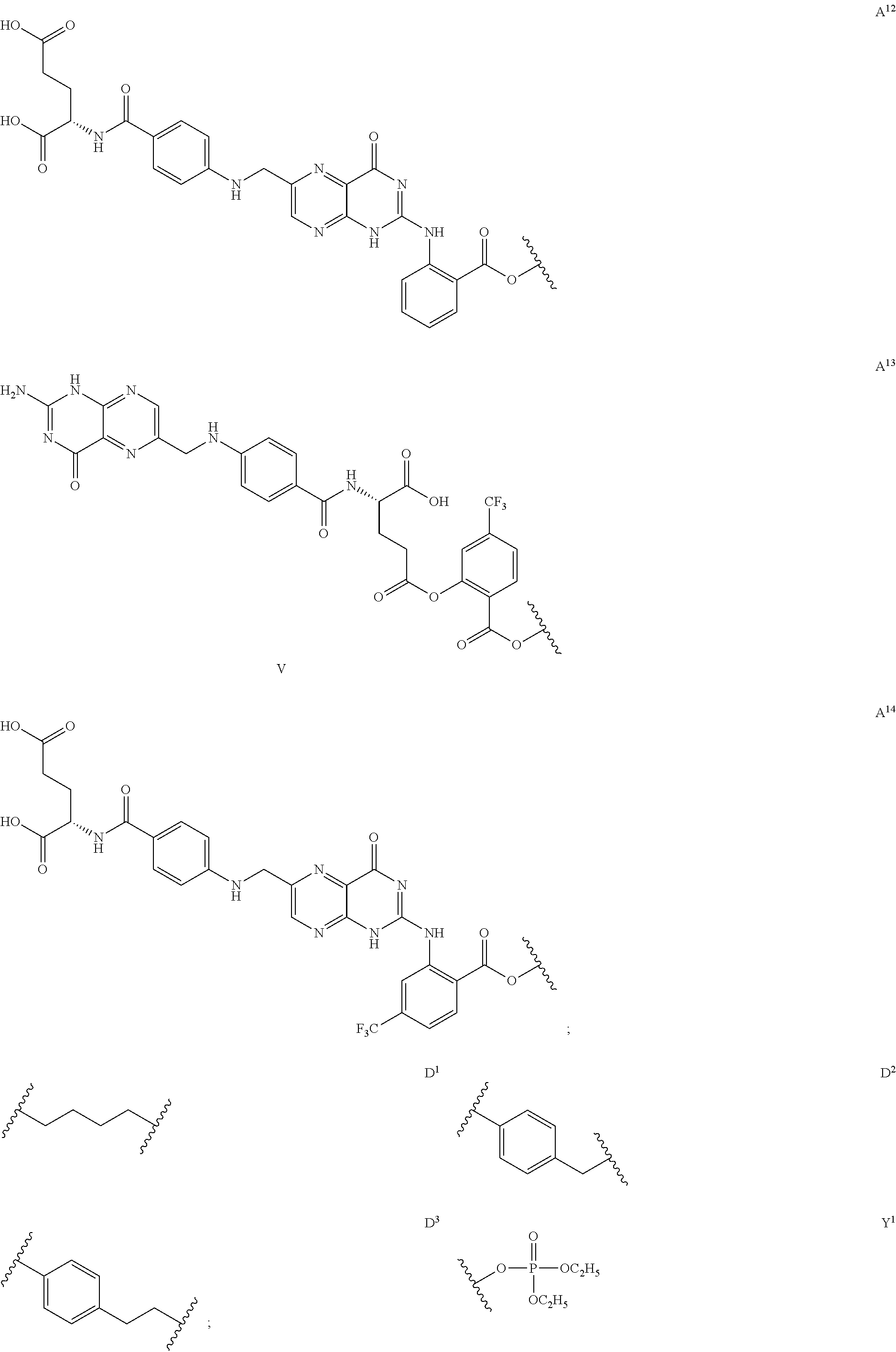

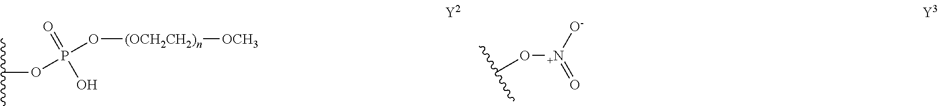

In Group D, n is preferably between 40 and 50.

Another embodiment of the invention features a compound of general Formula VIII:

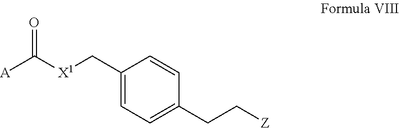

or a pharmaceutically acceptable salt thereof.

In Formula VIII: A is an optionally substituted aliphatic, heteroaliphatic, aromatic, heteroaromatic substituent or alkylaryl substituent having 1 to 100 carbon atoms or is selected from:

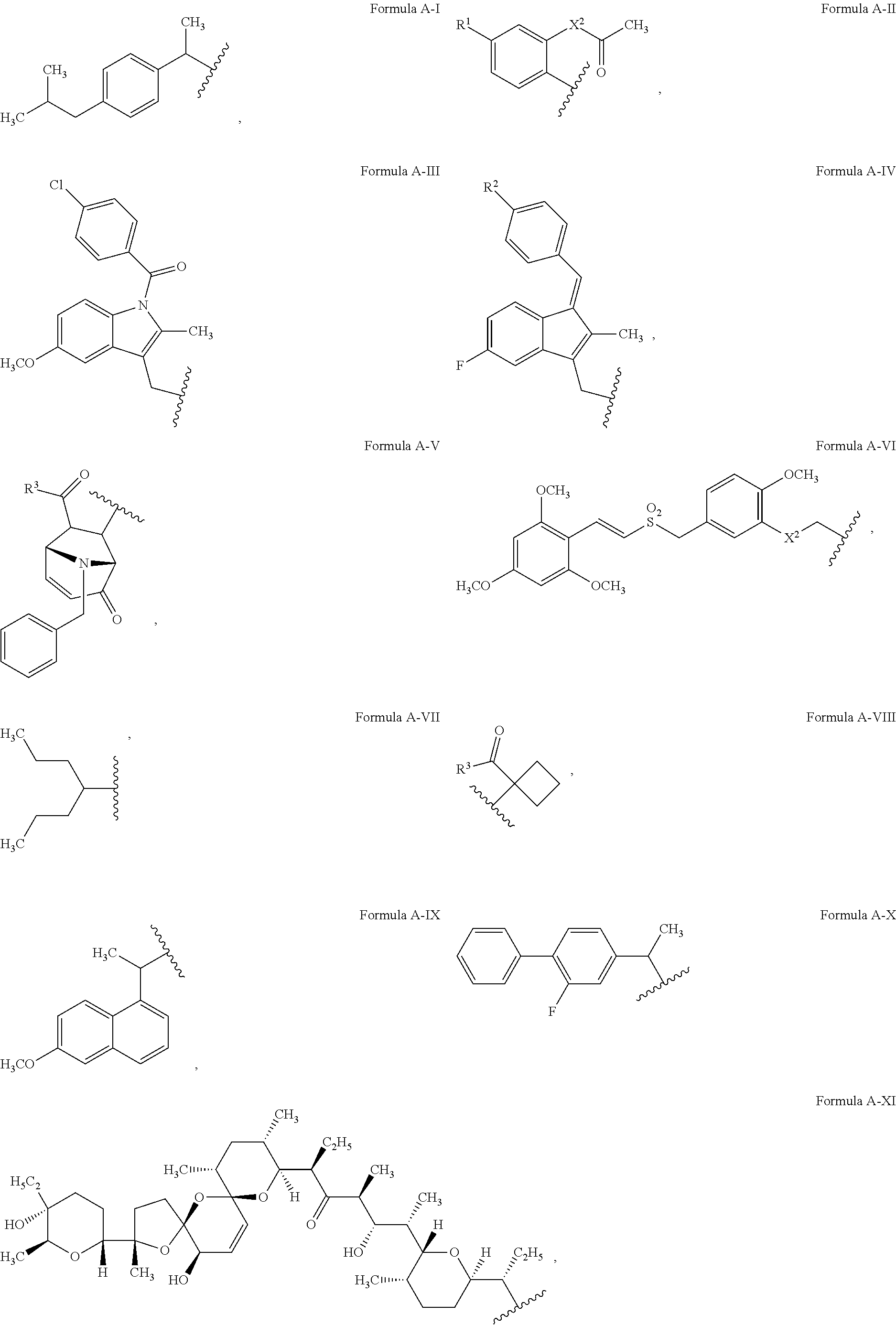

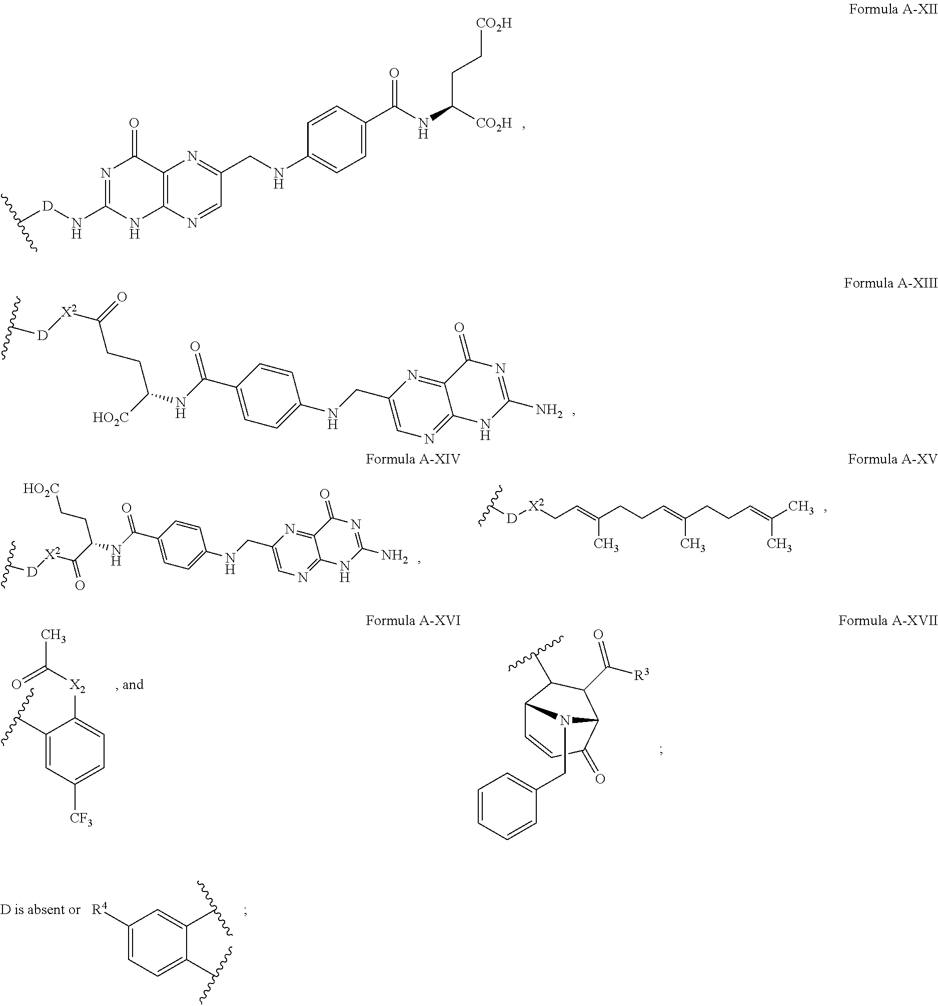

X¹ and X² are independently selected from —O—, —NR—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, —X¹—(CH₂)₄—Z, and

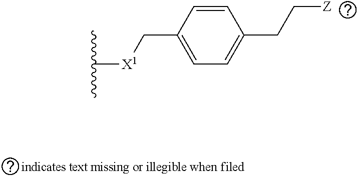

R⁵ is selected from hydrogen and C₁₋₆ alkyl;

Z is selected from;

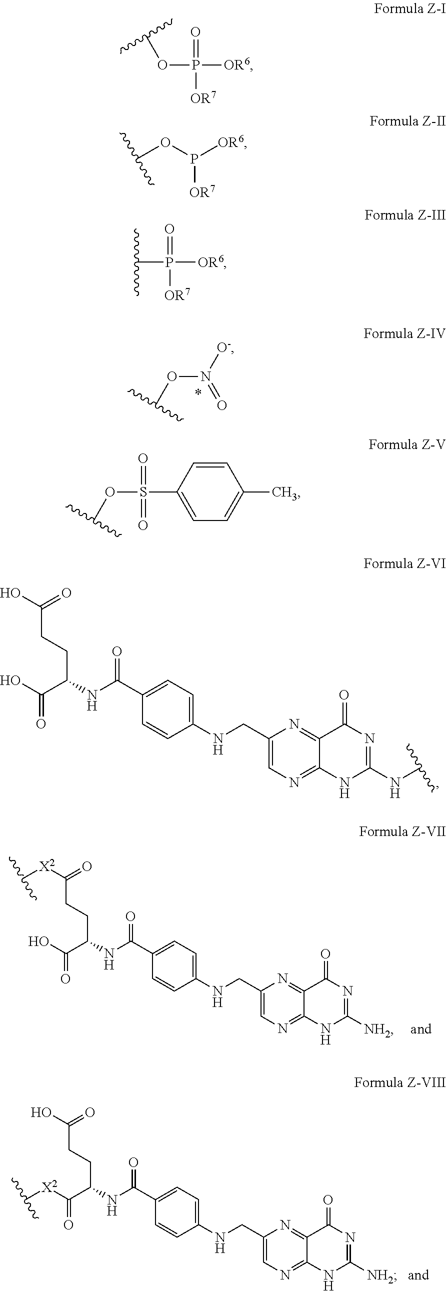

R⁶ and Ware independently selected from hydrogen, C₁₋₆ alkyl, and polyethylene glycol residue.

In some embodiments, X¹ is —NR⁵—, and R⁵ is selected from hydrogen, methyl, and ethyl. In other embodiments, X¹ is —O—.

In certain embodiments, Z is Z-III and R⁶ is selected from ethyl and a polyethylene glycol residue, and R⁷ is selected from hydrogen and ethyl.

In still other embodiments, A is selected from:

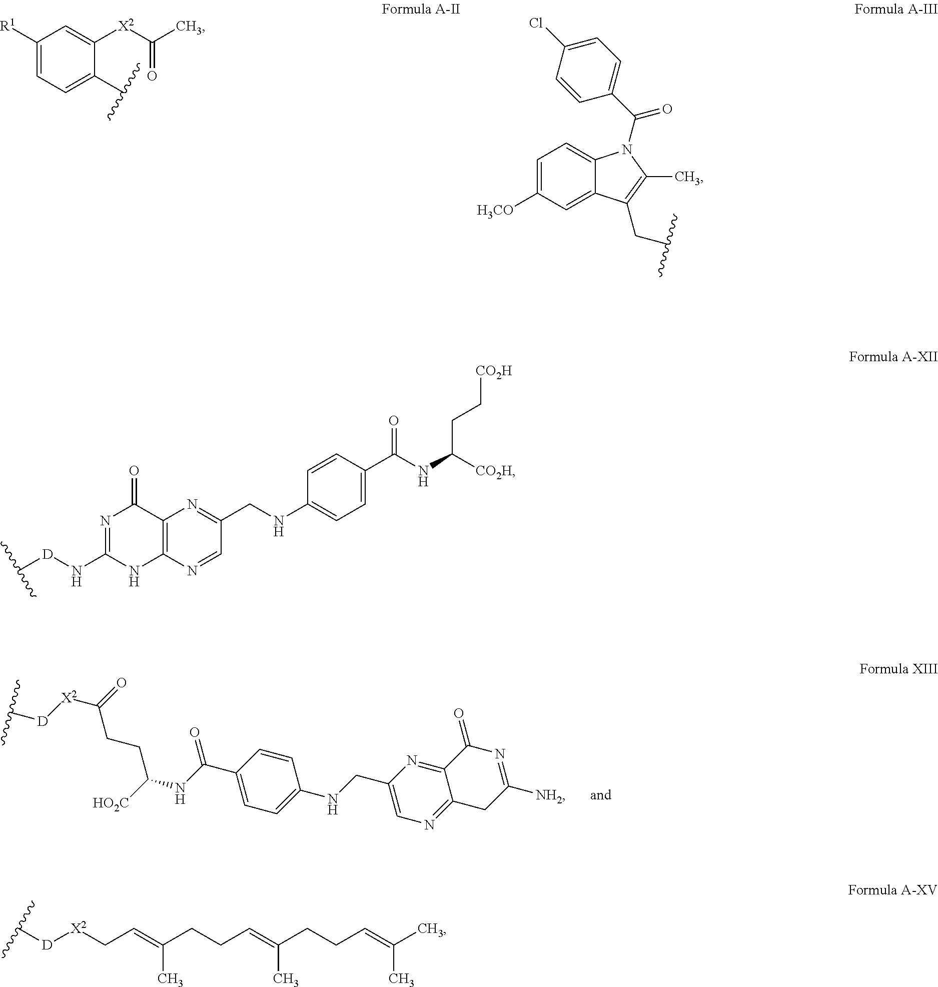

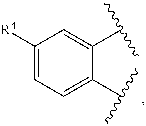

wherein D is

R¹ and R⁴ are independently selected from hydrogen and triflouoromethyl, and X² is selected from —O—, —S—, and —NH—.

In some embodiments, X¹ is —O—, Z is —O—P(O)(CH₂CH₃)₂, and A is:

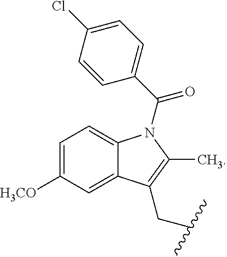 [SMILES: *Cc1c(C)n(C(=O)c2ccc(Cl)cc2)c2ccc(OC)cc12]

In certain embodiments, X¹ is selected from —O— and —NH—, Z is —O—P(O)(CH₂CH₃)₂, A is:

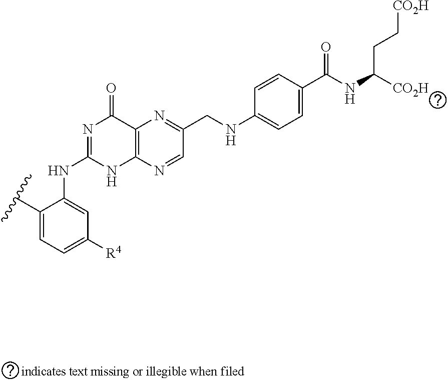

and R⁴ is selected from hydrogen and trifluoromethyl.

In other embodiments, X¹ and X² are independently selected from —O— and —NH—, Z is —O—P(O)(CH₂CH₃)₂, A is:

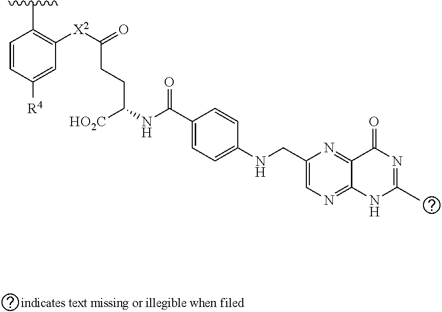

and R⁴ is selected from hydrogen and trifluoromethyl.

In other embodiments, X¹ and X² are independently selected from —O— and —NH—, Z is —O—P(O)(CH₂CH₃)₂, A is:

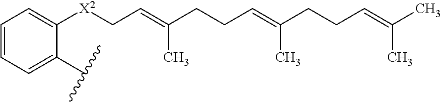

In some embodiments, X¹ is selected from —O—, —S—, and —NH—, Z is selected from O—P(O)(CH₂CH₃)₂ and —ONO₂, A is:

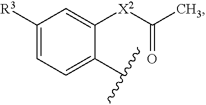

and R¹ is selected from hydrogen and trifluoromethyl, and X² is selected from —O—, —S— and —NH—.

In certain embodiments, X¹ is selected from —O— and —NH—, Z is —ONO₂, and A is:

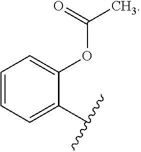 [SMILES: *c1ccccc1OC(C)=O]

Accordingly, compounds of Formula VII include but are not limited to:

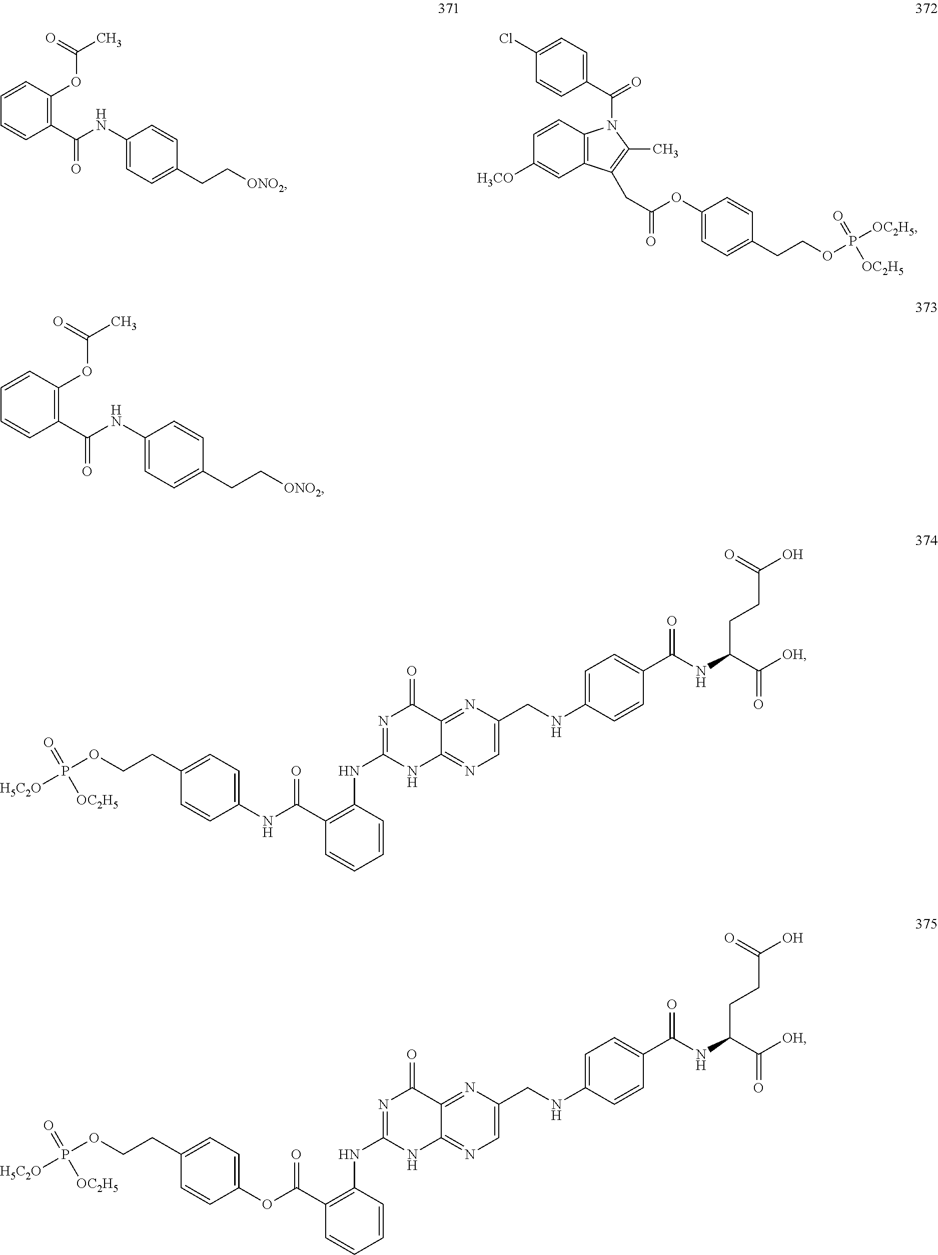

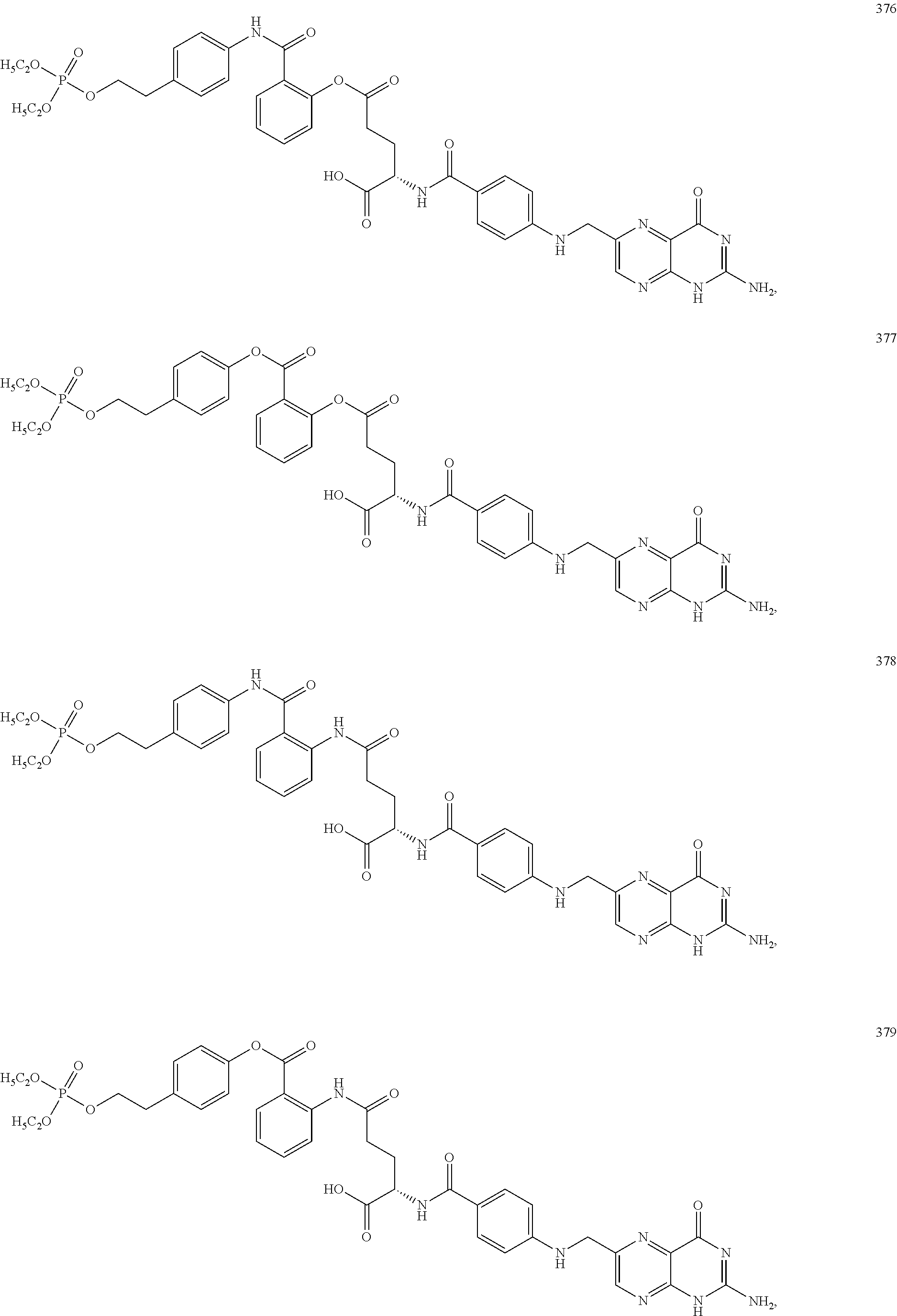

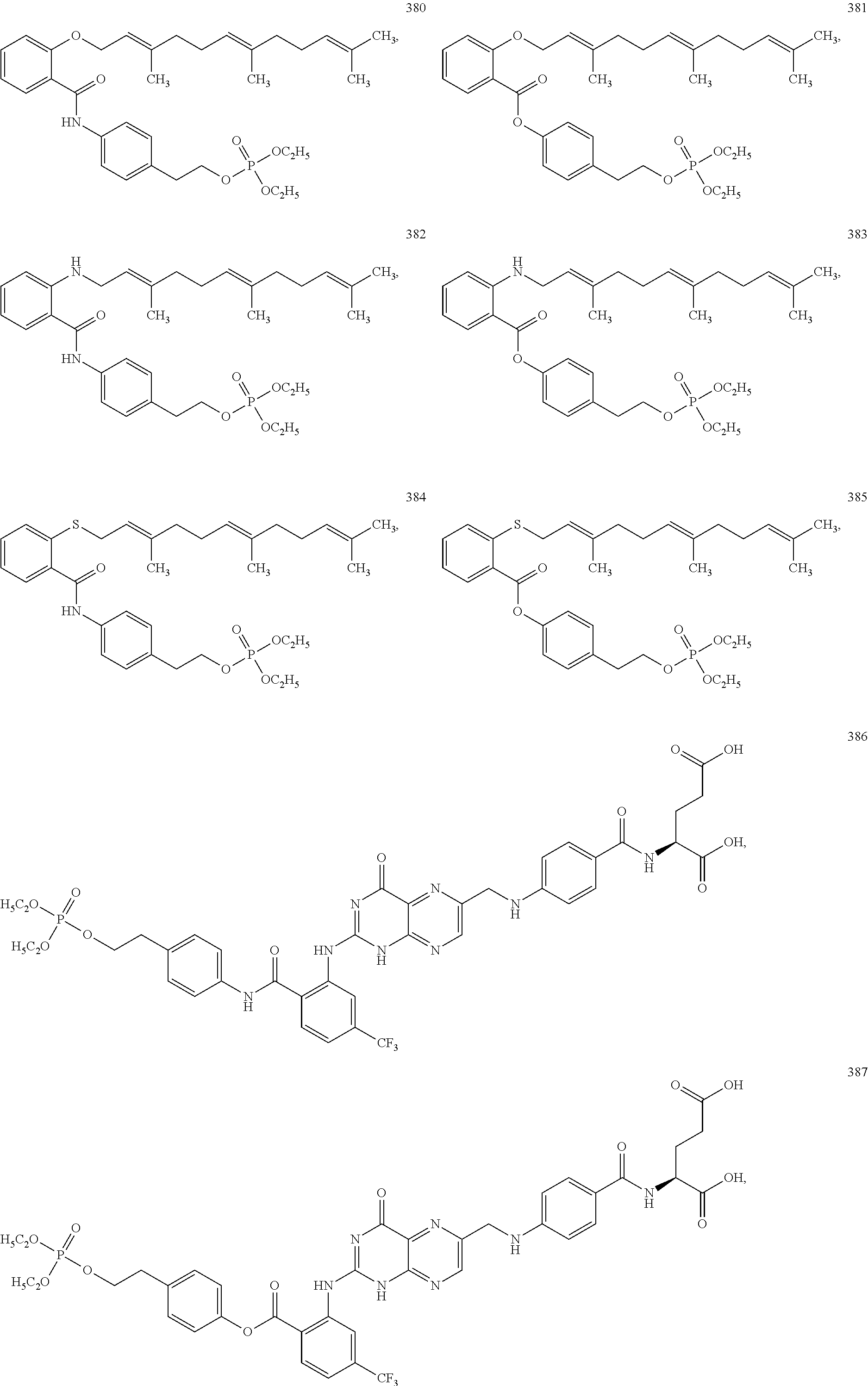

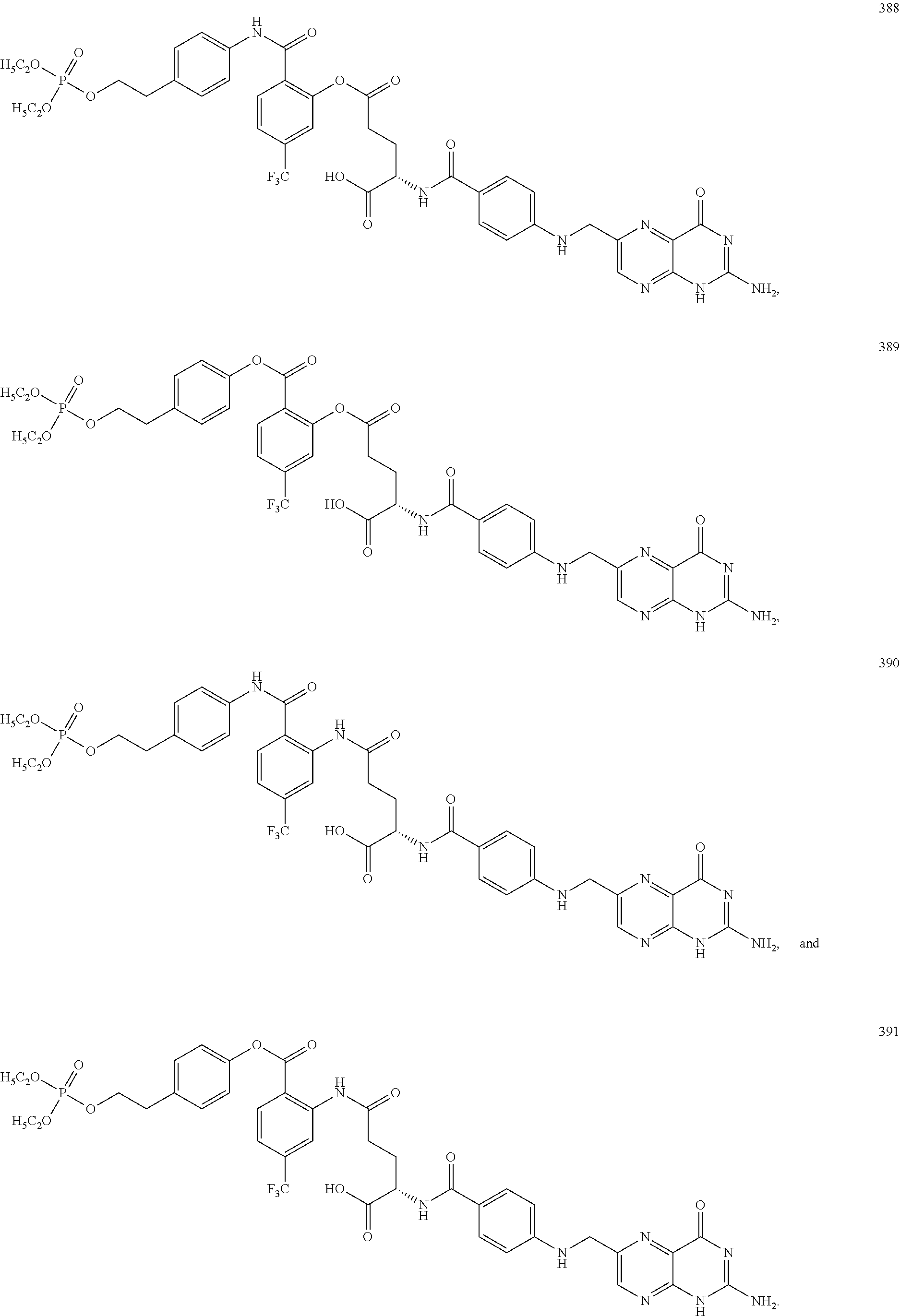

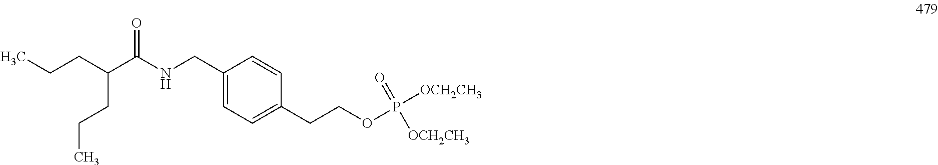 [SMILES: CCCC(CCC)C(=O)NCc1ccc(CCOP(=O)(OCC)OCC)cc1]

Another embodiment features a compound of general Formula IX:

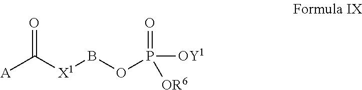

or a pharmaceutically acceptable salt thereof.

In Formula IX: Y¹ is a polyethylene glycol residue;

R⁶ is selected from hydrogen, C₁₋₆-alkyl, and polyethylene glycol residue;

A is an optionally substituted aliphatic, heteroaliphatic, aromatic, heteroaromatic substituent or alkylaryl substituent having 1 to 100 carbon atoms or selected from:

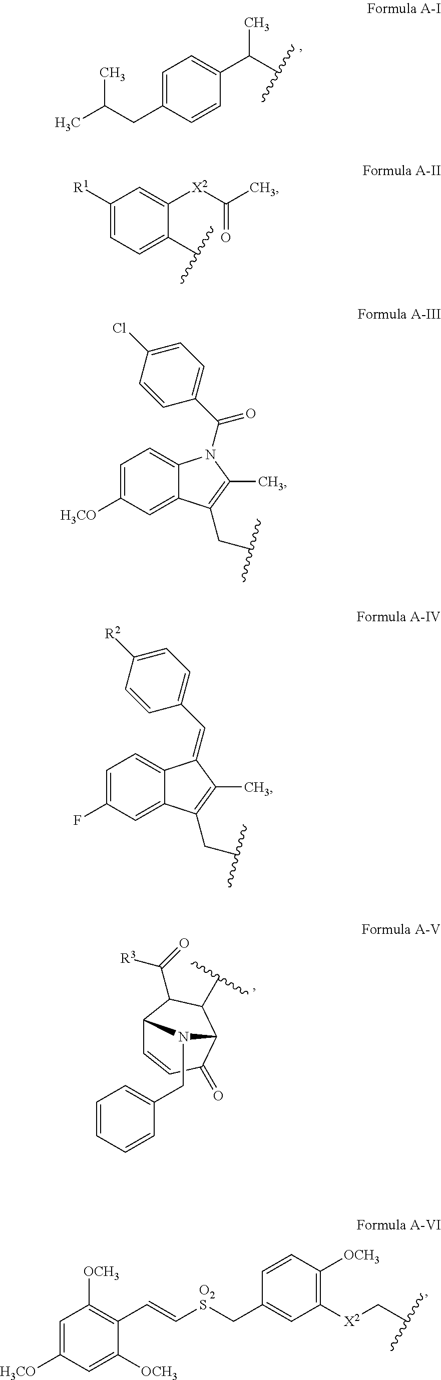

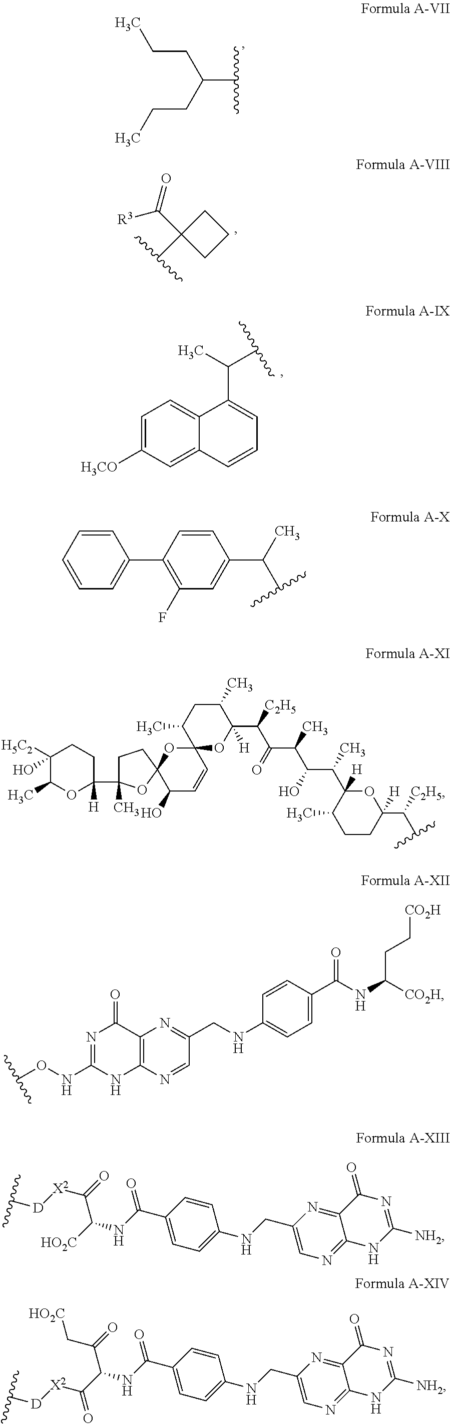

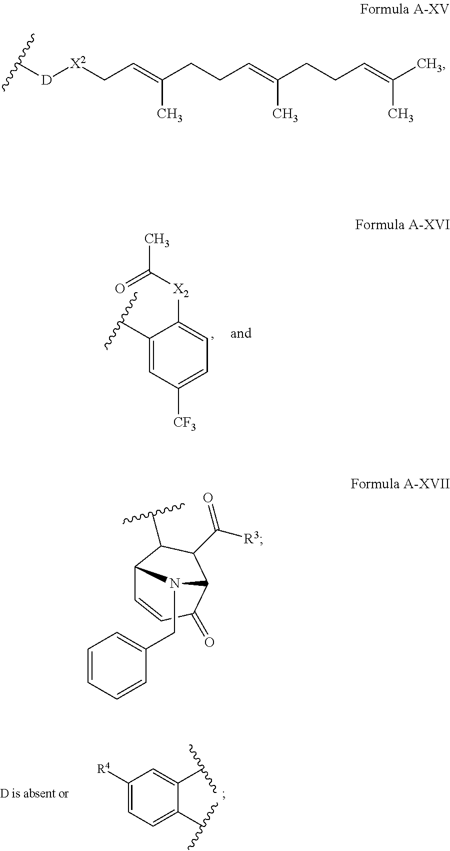

X¹ and X² are independently selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R₃ is selected from hydroxyl, Z, and —X¹—B—Z;

R₅ is selected from hydrogen and C₁₋₆ alkyl;

B is selected from:

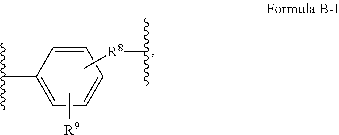

a single bond, and an aliphatic group with 1 to 22 carbon atoms;

R⁸ is a C₁₋₄ alkylene; and

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O-C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxyl.

In further embodiments, Y¹ is a polyethylene glycol residue described by —O(CH₂CH₂O)ₘR¹⁰, wherein m is 1 to 100 (e.g. 20 to 100, 20 to 50, 40 to 50), and R¹⁰ is selected from hydrogen, alkyl and alkoxy, and R⁶ is hydrogen.

In still other embodiments, Y¹ is —O(CH₂CH₂O)me wherein m is 45, R¹⁰ is —OCH3, and R⁶ is hydrogen.

In some embodiments, X¹ is —O—.

In other embodiments, X¹ is —NR⁵— and R⁵ is selected from hydrogen, methyl and ethyl.

In certain embodiments, B is —(CH₂)₄—.

In some embodiments, A is:

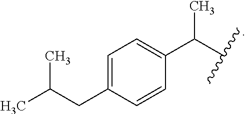 [SMILES: *C(C)c1ccc(CC(C)C)cc1]

In other embodiments, the compound is:

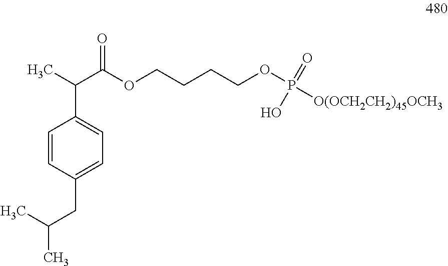

In another embodiment, the invention features a compound of general Formula I as first described, or a pharmaceutically acceptable salt thereof.

In this embodiment of Formula I, A may be selected from:

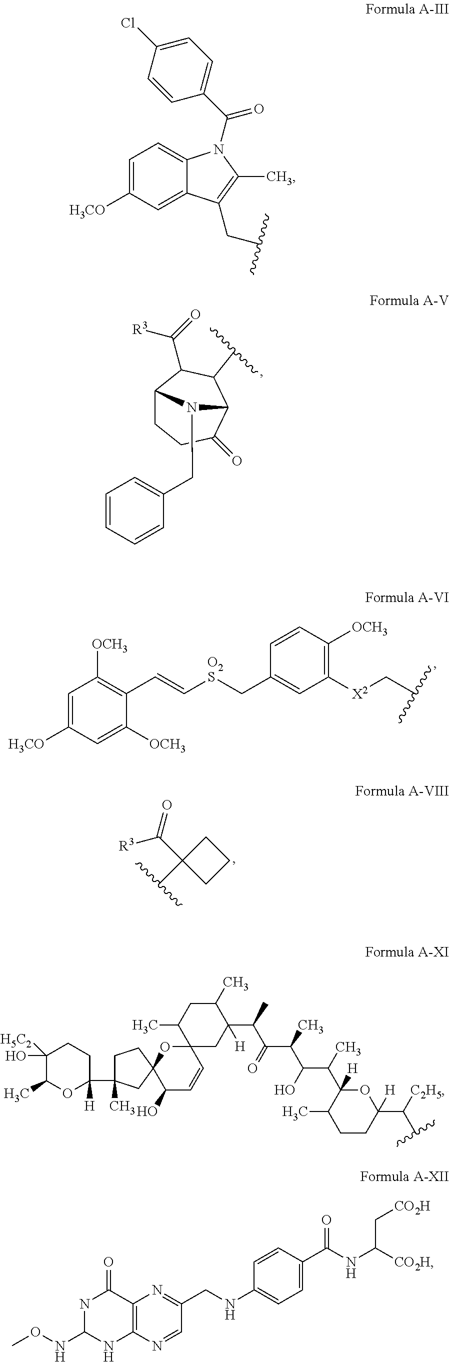

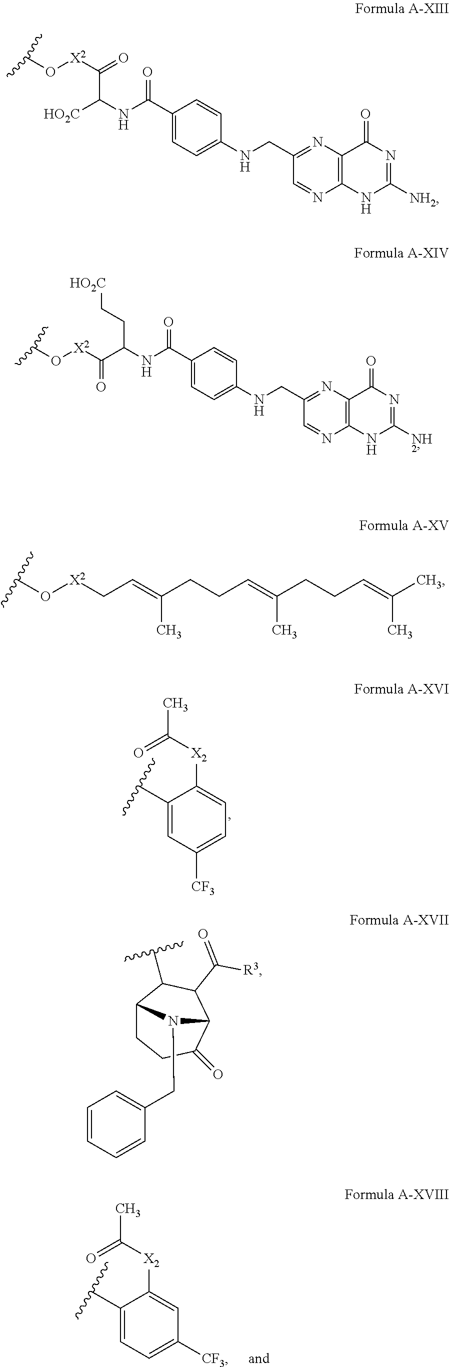

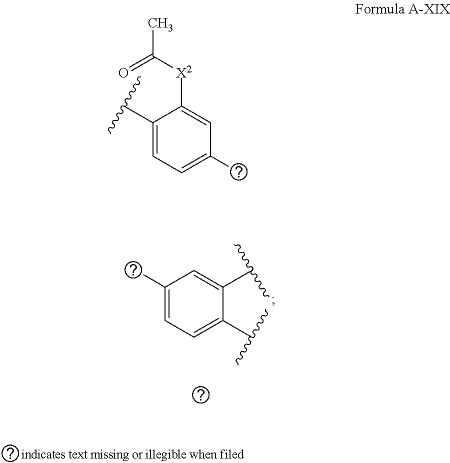

X¹ and X² are independently selected from —O—, —NR^{S}—, and —S—;

R¹ and R⁴ are independently selected from hydrogen and trifluoromethyl;

X³ is selected from —S— and —NH—;

R³ is selected from hydroxyl, Z, and —X¹—B—Z;

R⁵ is selected from hydrogen and C₁₋₆ alkyl;

B is selected from

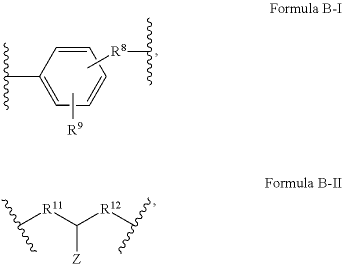

a single bond, and an aliphatic group with 1 to 22 carbon atoms;

R⁸, R¹¹, and R¹² are the same or different C₁₋₄-alkylene;

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O-C₁₋₆-alkyl, —OC(O)—C₁₋₆-alkyl, —C(O)NH₂, —C(O)NH—C₁₋₆-alkyl, —S(O)—C-₁₋₆-alkyl, —S(O)₂—C-₁₋₆-alkyl, —S(O)₂—NHC₁₋₆-alkyl, cyano, halo or hydroxyl;

Z is selected from:

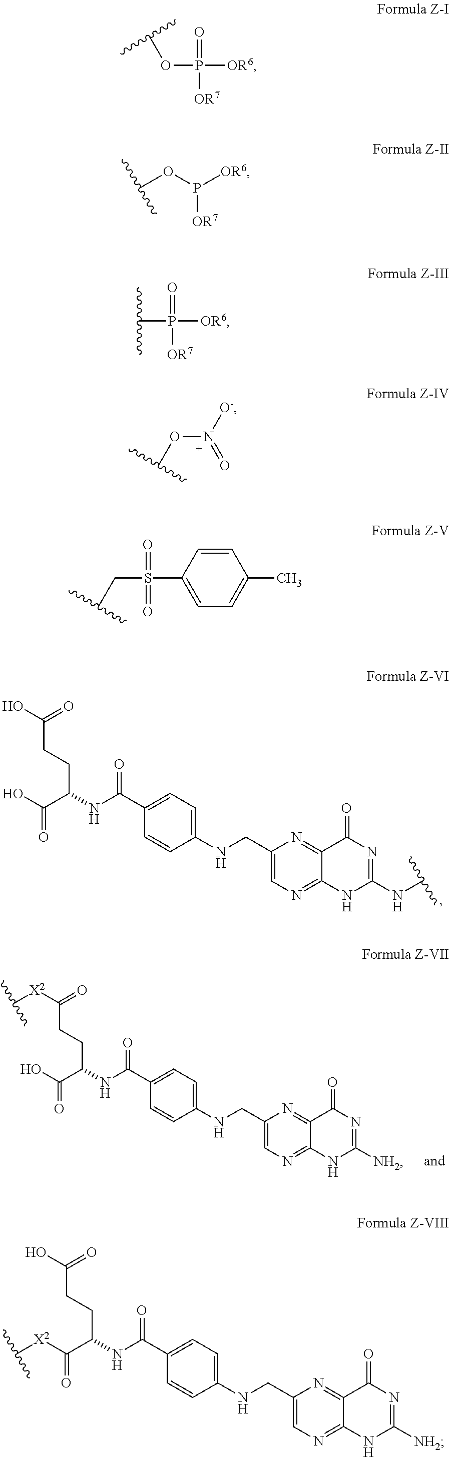

or B together with Z forms a structure:

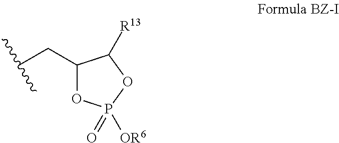

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆₋alkyl), and polyethylene glycol residue.

In still other embodiments, X¹ is —O—.

In certain embodiments, X¹ is —NR⁵— and R⁵ is selected from hydrogen, methyl, and ethyl.

In some embodiments, B is selected from:

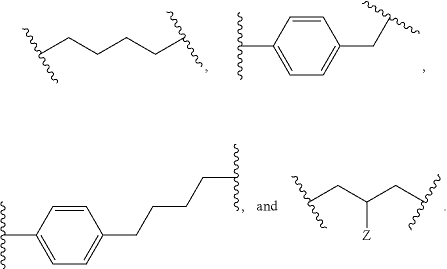

In other embodiments, Z is selected from —OP(O)(OCH²CH³)² and —ONO².

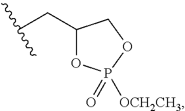 [SMILES: *CC1COP(=O)(OCC)O1]

In further embodiments, B—Z is

In certain embodiments, X¹ is selected from —O— and —NH—, B is selected from

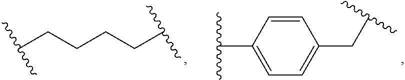 [SMILES: *CCCC* ; *Cc1ccc(*)cc1]

### Z is —OP(O)(OCH₂CH₃)₂, and A is

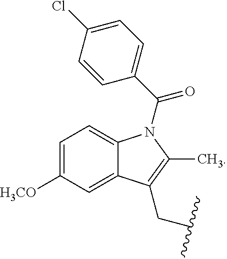 [SMILES: *Cc1c(C)n(C(=O)c2ccc(Cl)cc2)c2ccc(OC)cc12]

In certain embodiments, X¹ is selected from —O— and —NH—, B is selected from

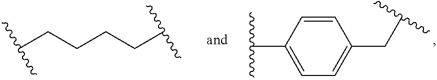 [SMILES: *CCCC* ; *Cc1ccc(*)cc1]

### Z is —OP(O)(OCH₂CH₃)₂, and A is

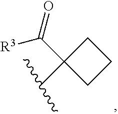

### and R³ is:

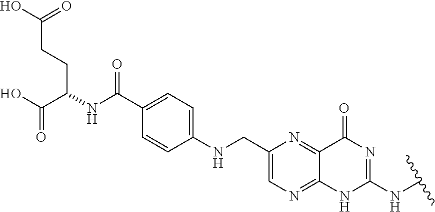 [SMILES: *Nc1nc(=O)c2nc(CNc3ccc(C(=O)N[C@@H](CCC(=O)O)C(=O)O)cc3)cnc2[nH]1]

In some embodiments, wherein X¹ is selected from —O— and —NH—, B is selected from

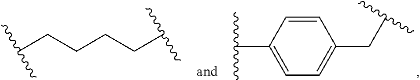 [SMILES: *CCCC* ; *Cc1ccc(*)cc1]

### Z is —OP(O)(OCH₂CH₃)₂, A is:

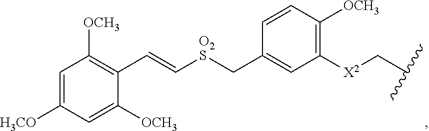

and X² is selected from —O— and —NH—,

In other embodiments, X¹ is selected from —O— and —NH—, B is selected from

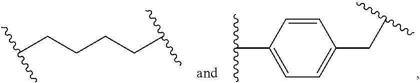 [SMILES: *CCCC* ; *Cc1ccc(*)cc1]

### Z is —OP(O)(OCH₂CH₂CH₃)₂, and A is:

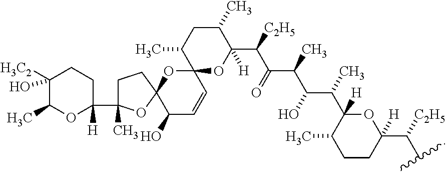

In further embodiments, X¹ is selected from —O— and —NH—, B is selected from

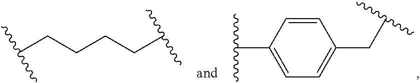 [SMILES: *CCCC* ; *Cc1ccc(*)cc1]

### Z is —OP(O)(OCH₂CH₃)₂, A is:

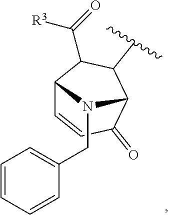

and R³ is hydroxyl or selected from:

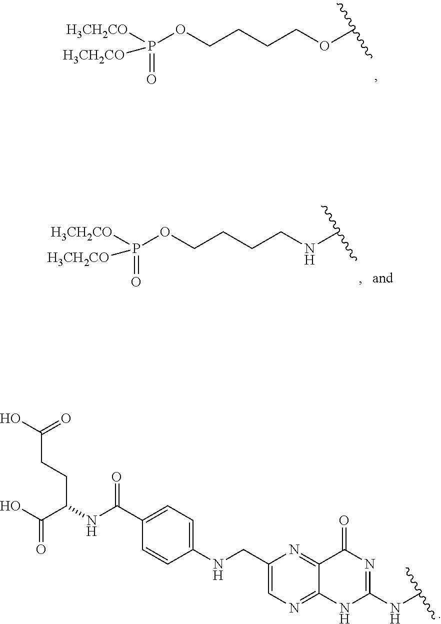

In certain embodiments, X¹ is selected from —O— and —NH—, B is selected from

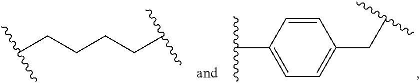 [SMILES: *CCCC* ; *Cc1ccc(*)cc1]

### Z is —OP(O)(OCH₂CH₃)₂, A is:

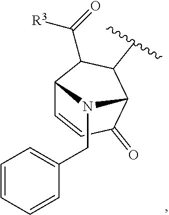

R³ is hydroxyl or selected from:

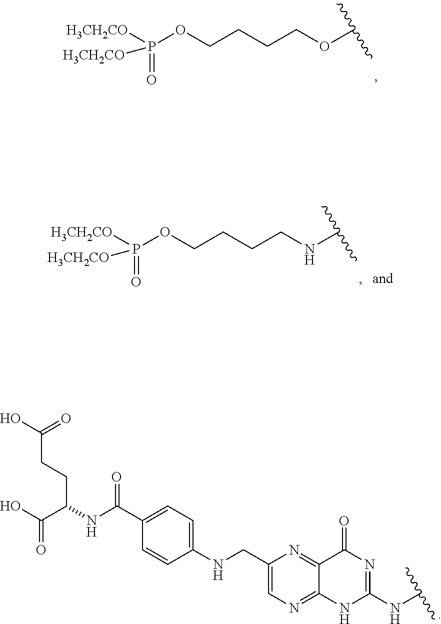

In some embodiments, X¹ is selected from —O— and —NH—, B is selected from

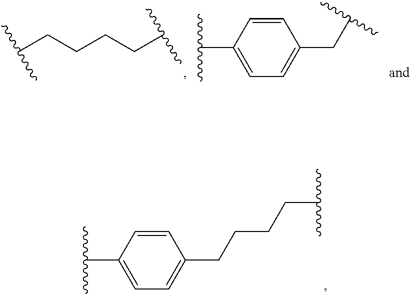

Z —OP(O)(OCH₂CH₃)₂, A is:

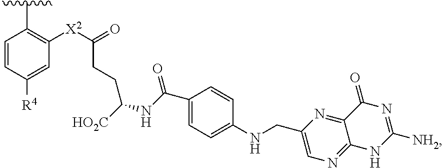

and R⁴ is selected from hydrogen and trifluoromethyl.

In some embodiments, X¹ is selected from —O— and —NH—, B is selected from

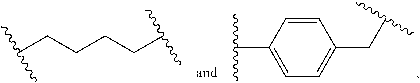 [SMILES: *CCCC* ; *Cc1ccc(*)cc1]

### Z is —OP(O)(OCH₂CH₃)₂, A is:

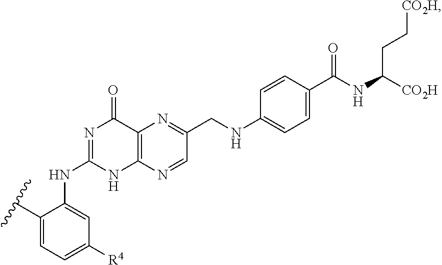

and R⁴ is selected from hydrogen and trifluoromethyl.

In other embodiments, X¹ is selected from —O— and —NH—, B is selected from

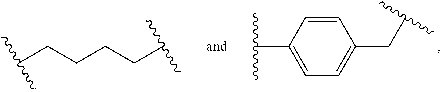 [SMILES: *CCCC* ; *Cc1ccc(*)cc1]

### Z is —OP(O)(OCH₂CH₃)₂, A is:

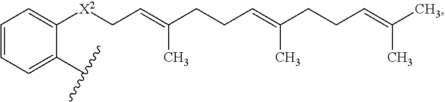

and X² is selected from —O—, —S—, and —NH—.

In other embodiments, X¹ is selected from —O— and —NH—, B is selected from

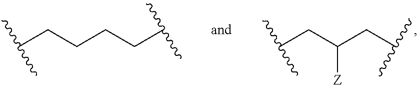

Z is selected from —OP(O)(OCH₂CH₃)₂, and —ONO₂, A is:

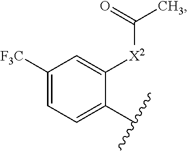

and X² is selected from —O—, —S—, and —NH—.

In some embodiments, X¹ is selected from —O— and —NH—, B is —(CH₂)₄—, Z is —ONO₂, A is:

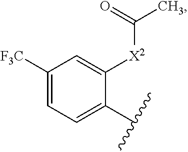

R¹ is selected from hydrogen and trifluoromethyl, and X³ is selected from —S—, and —NH—.

In other embodiments, X¹ is —NH—, A is

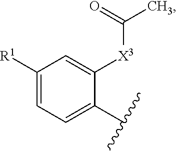

R¹ is selected from hydrogen and trifluoromethyl, and X³ is selected from —S—, and —NH—.

Accordingly, the compounds of Formula X include but are not limited to compounds of which the structures are shown below (Compounds 392-462 and 481-486):

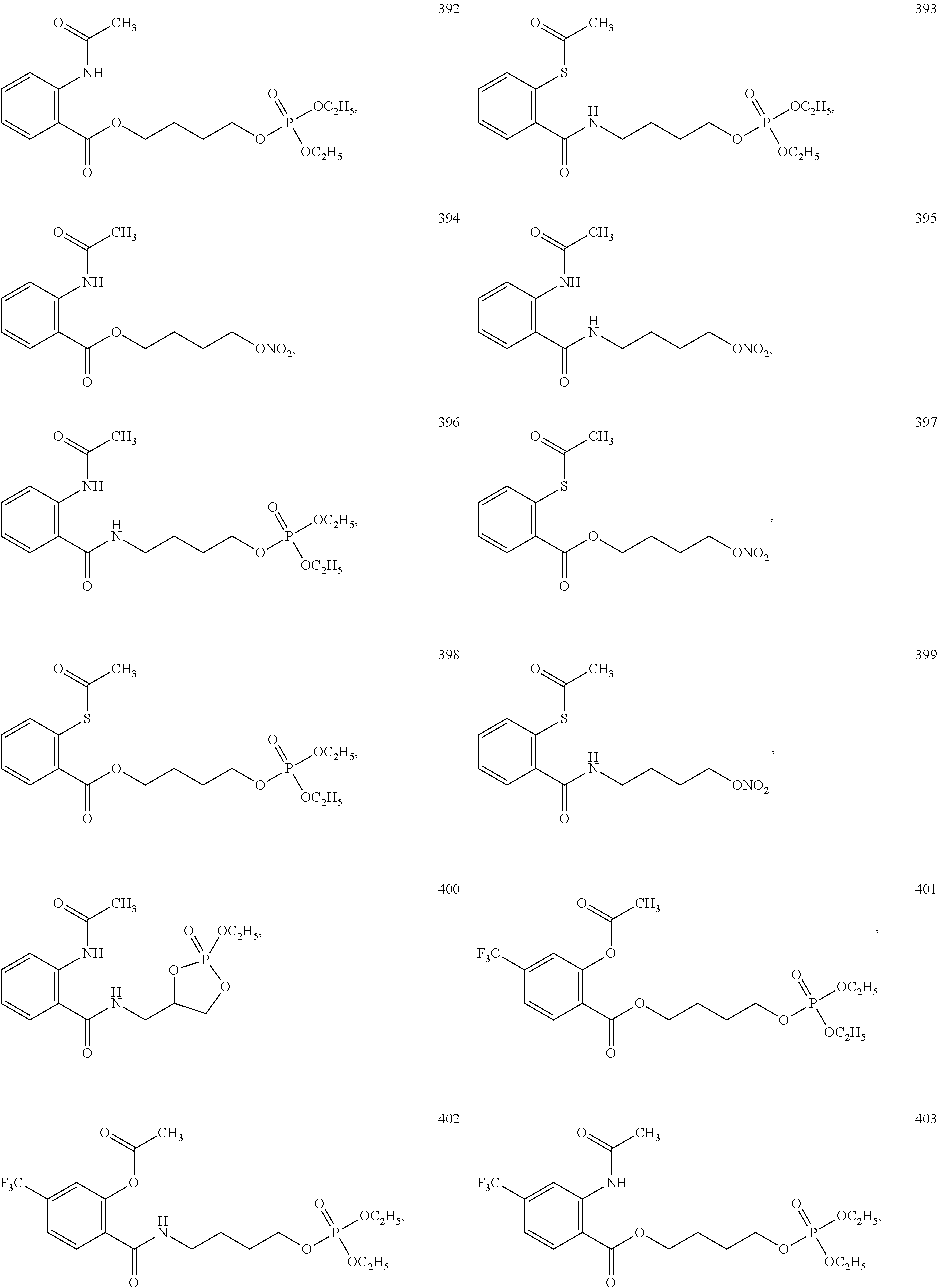

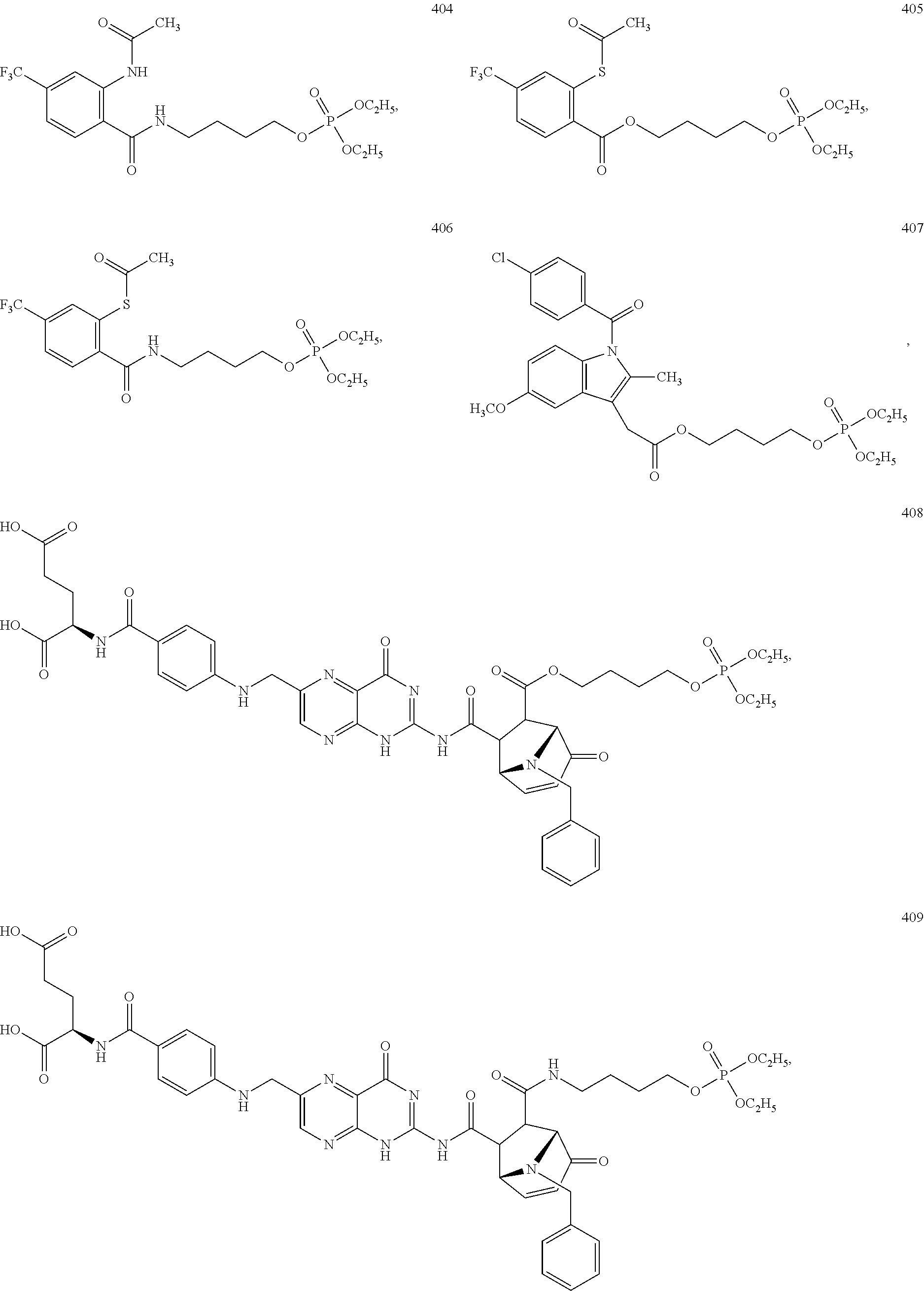

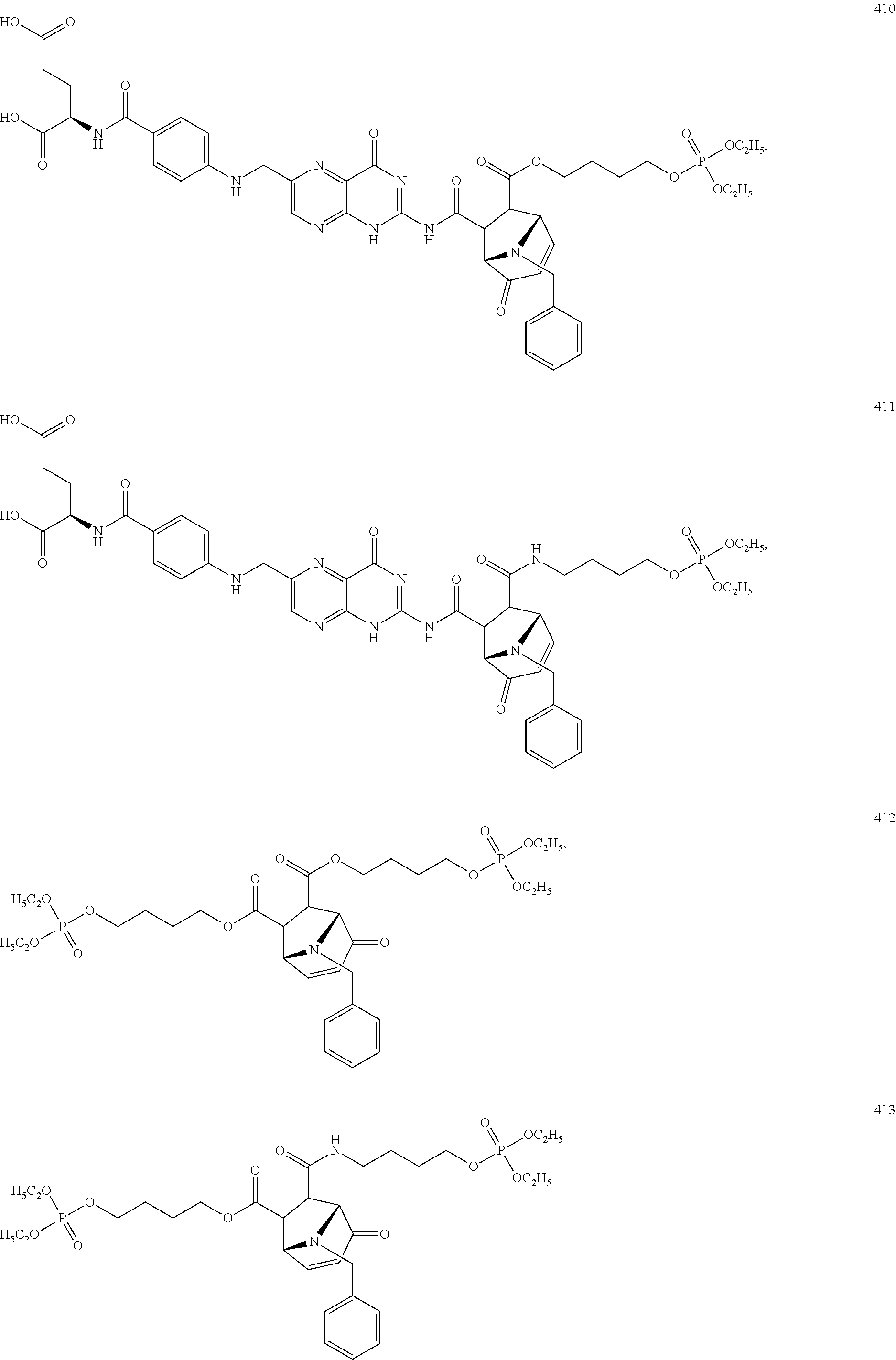

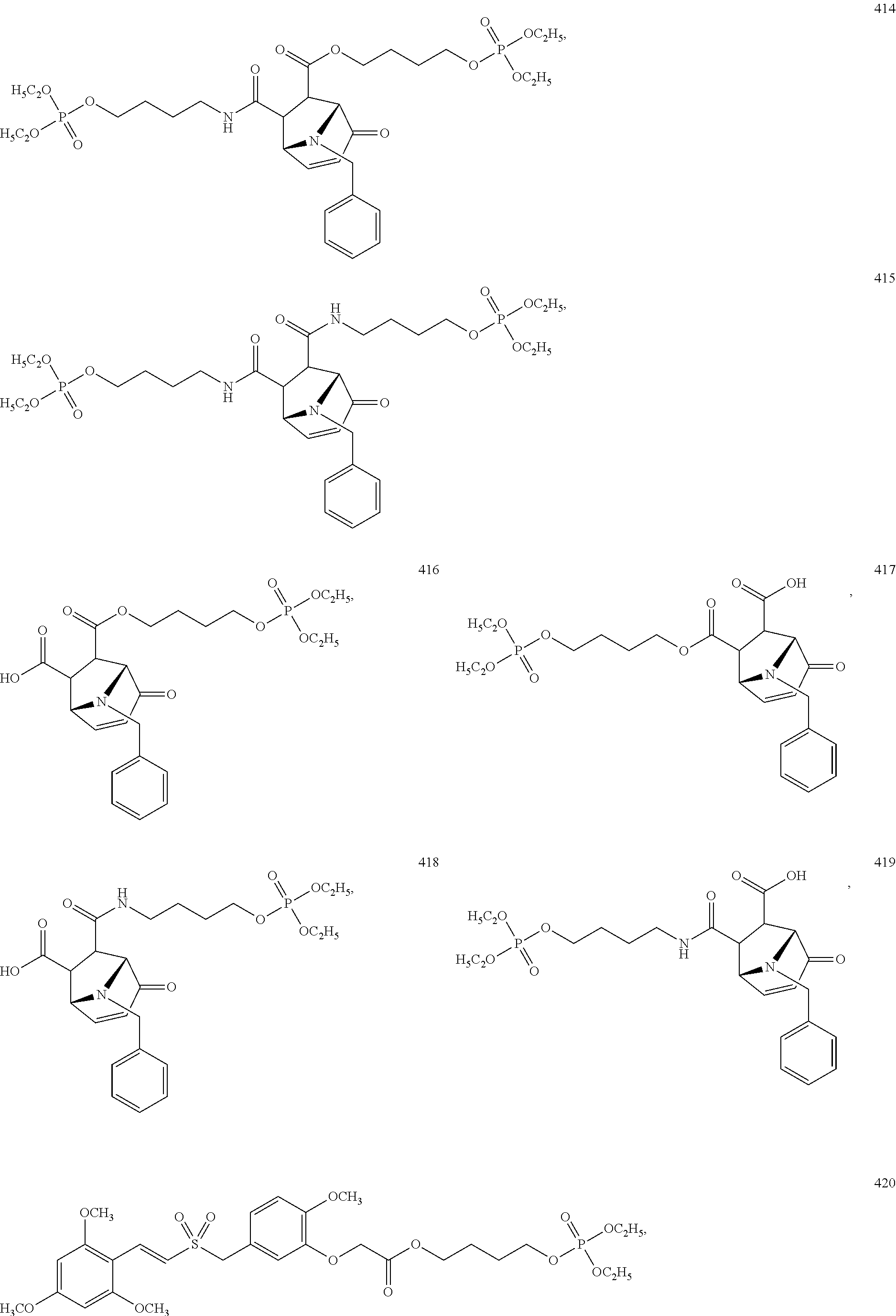

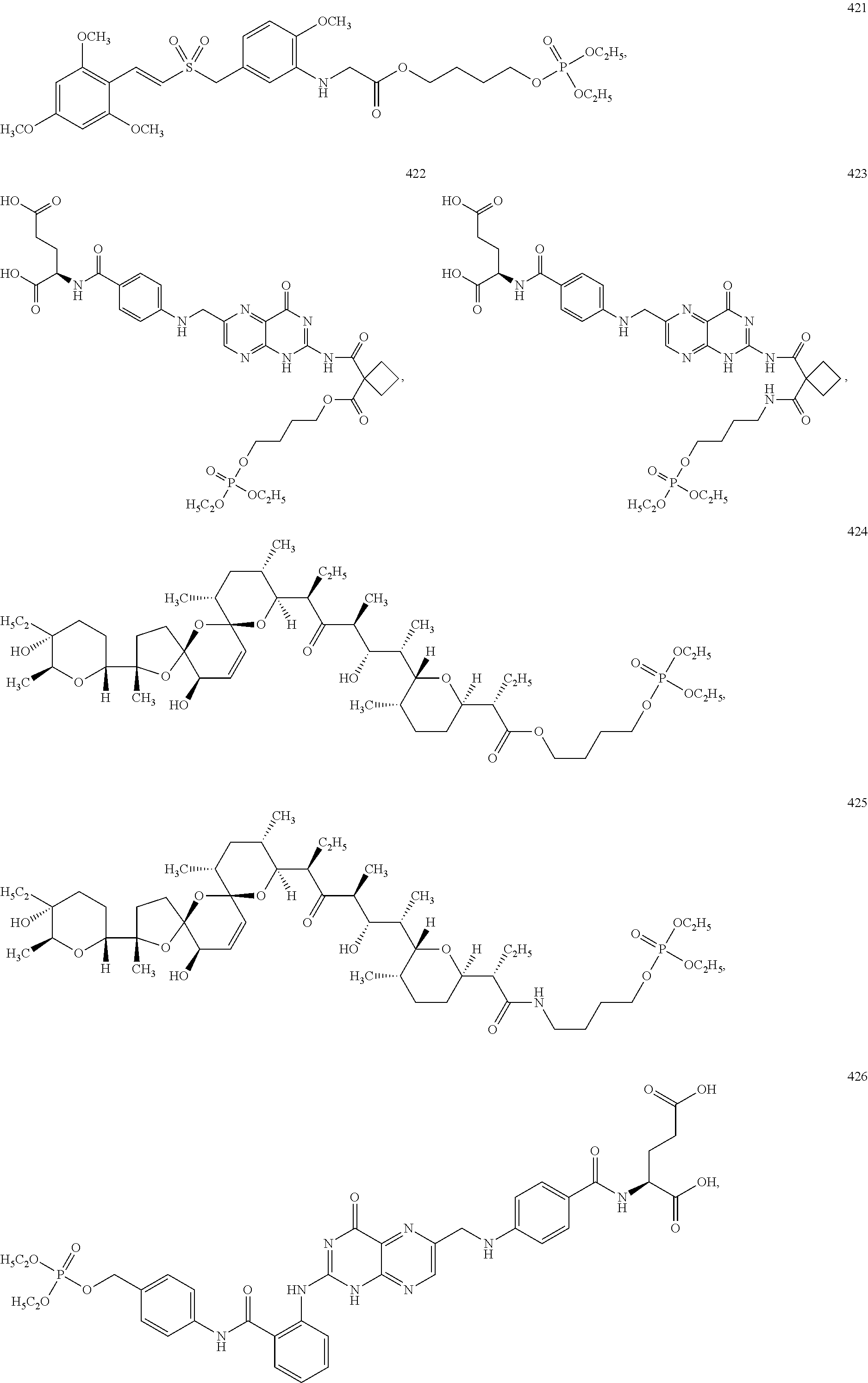

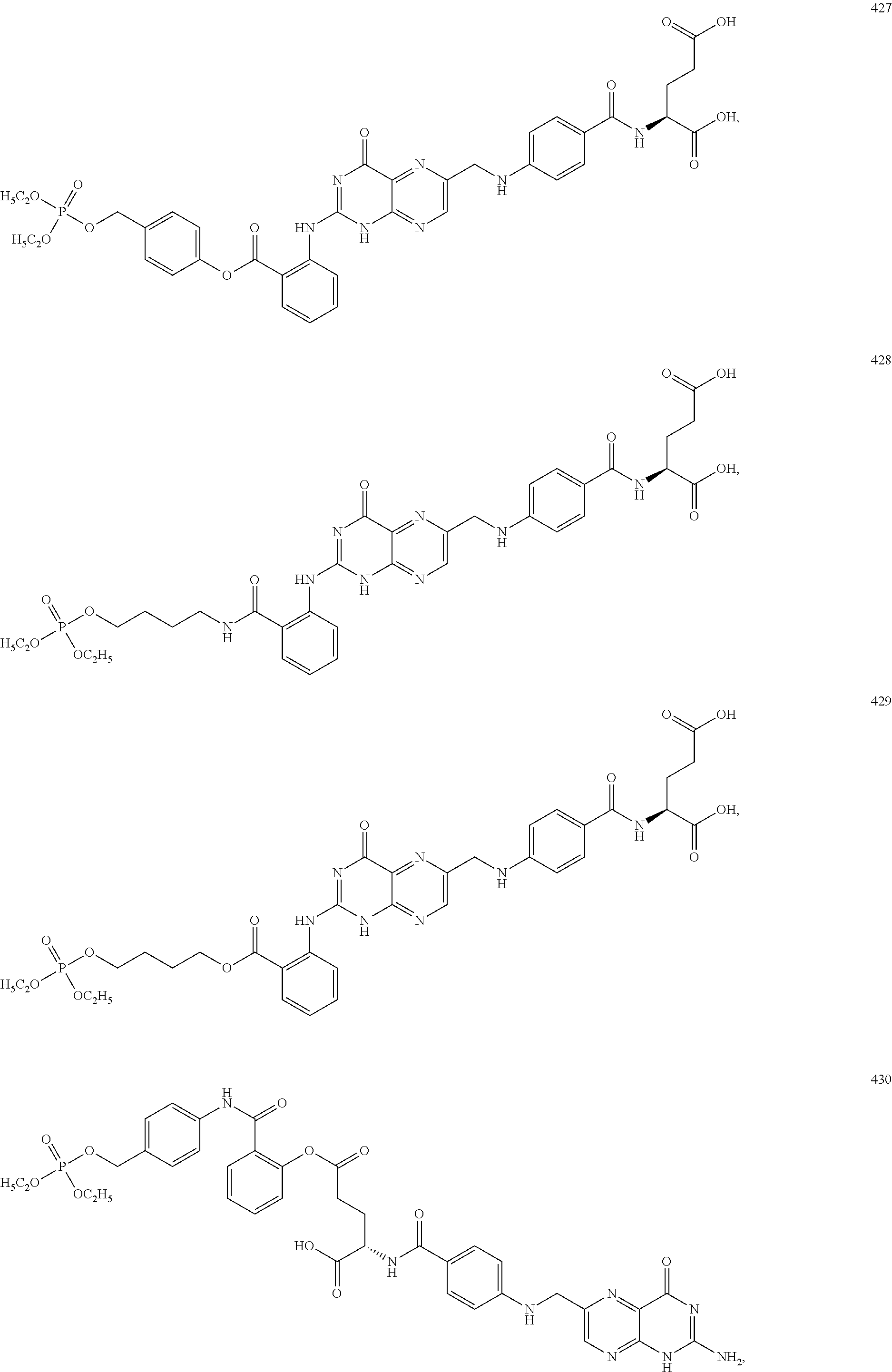

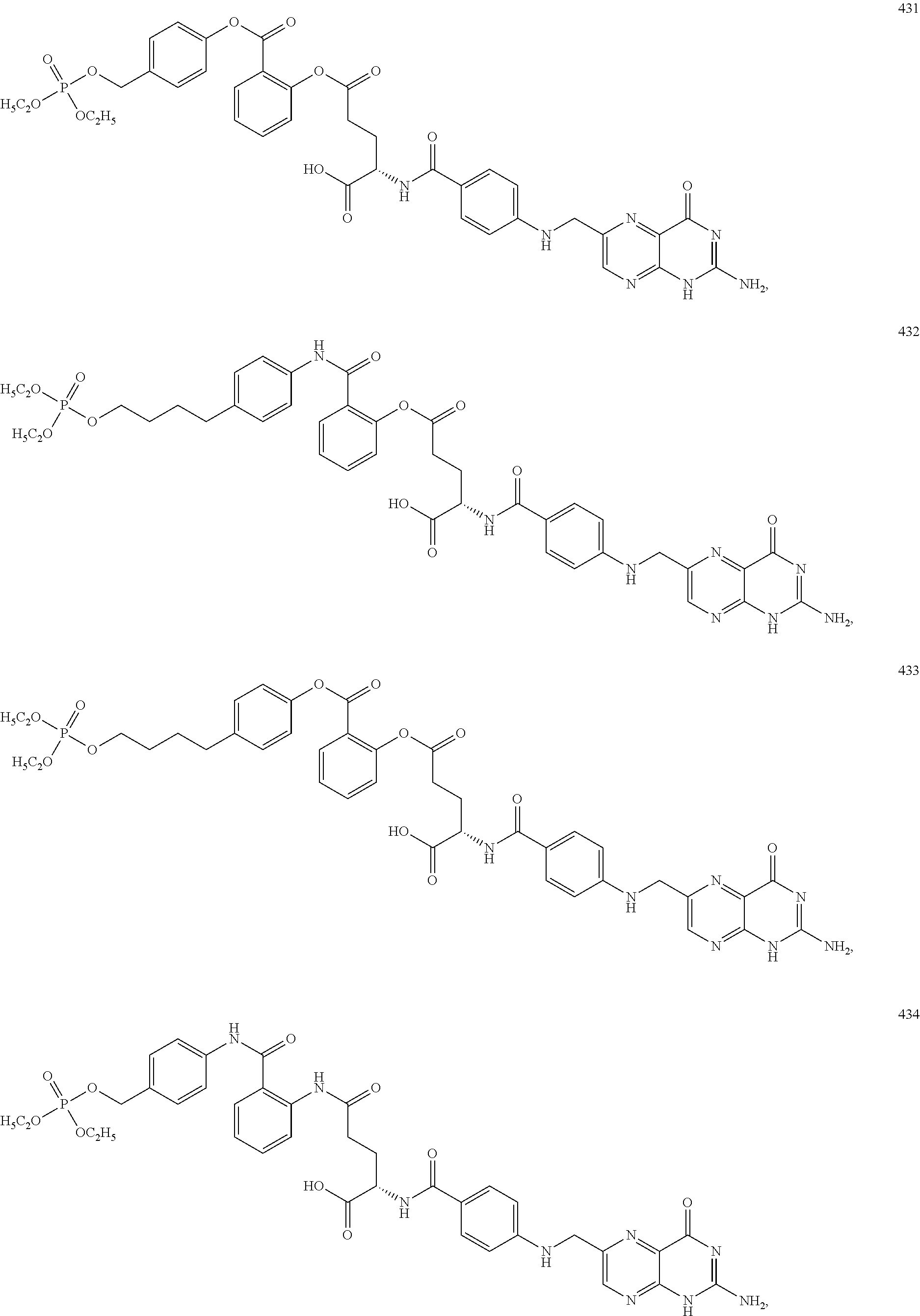

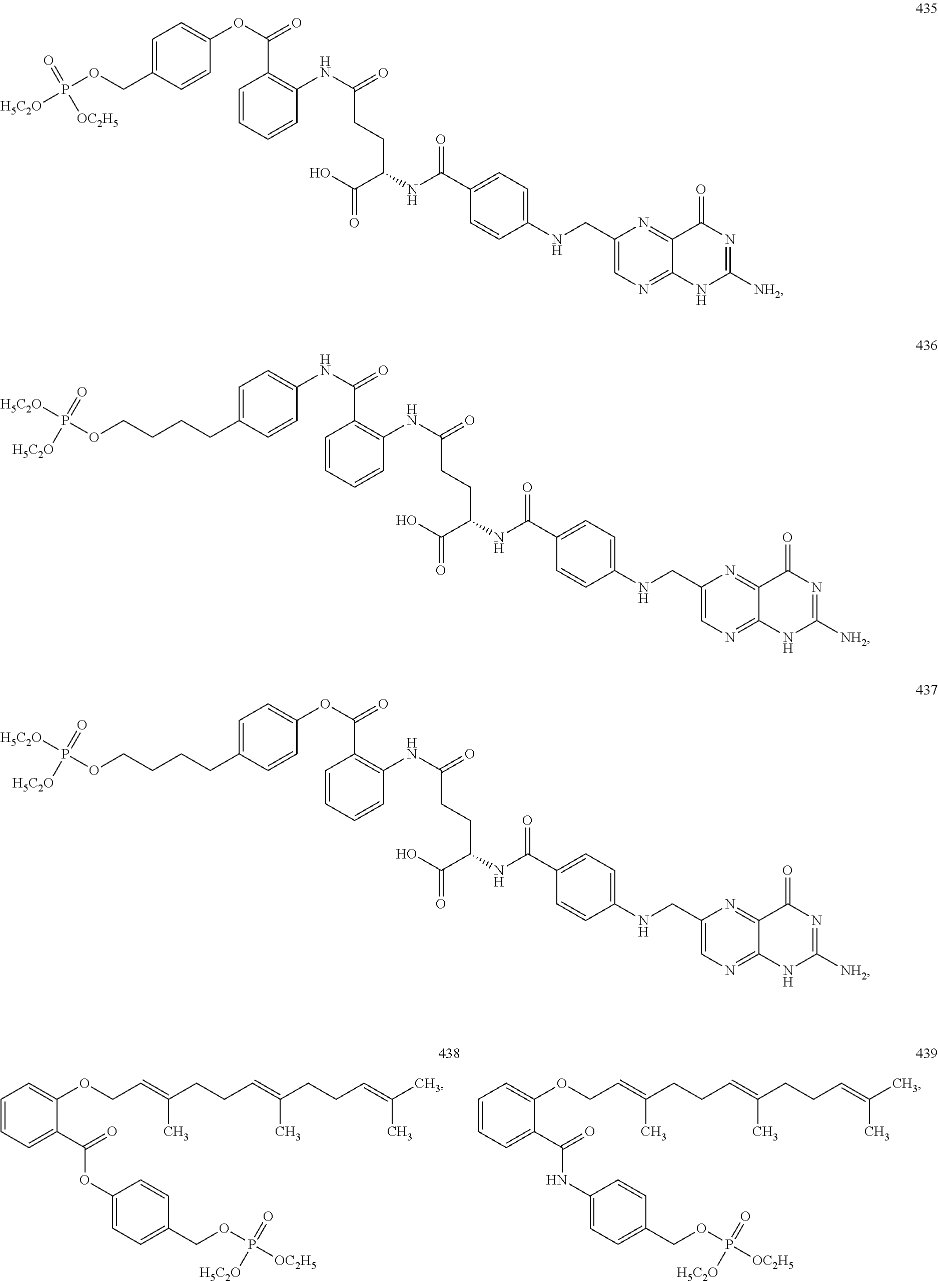

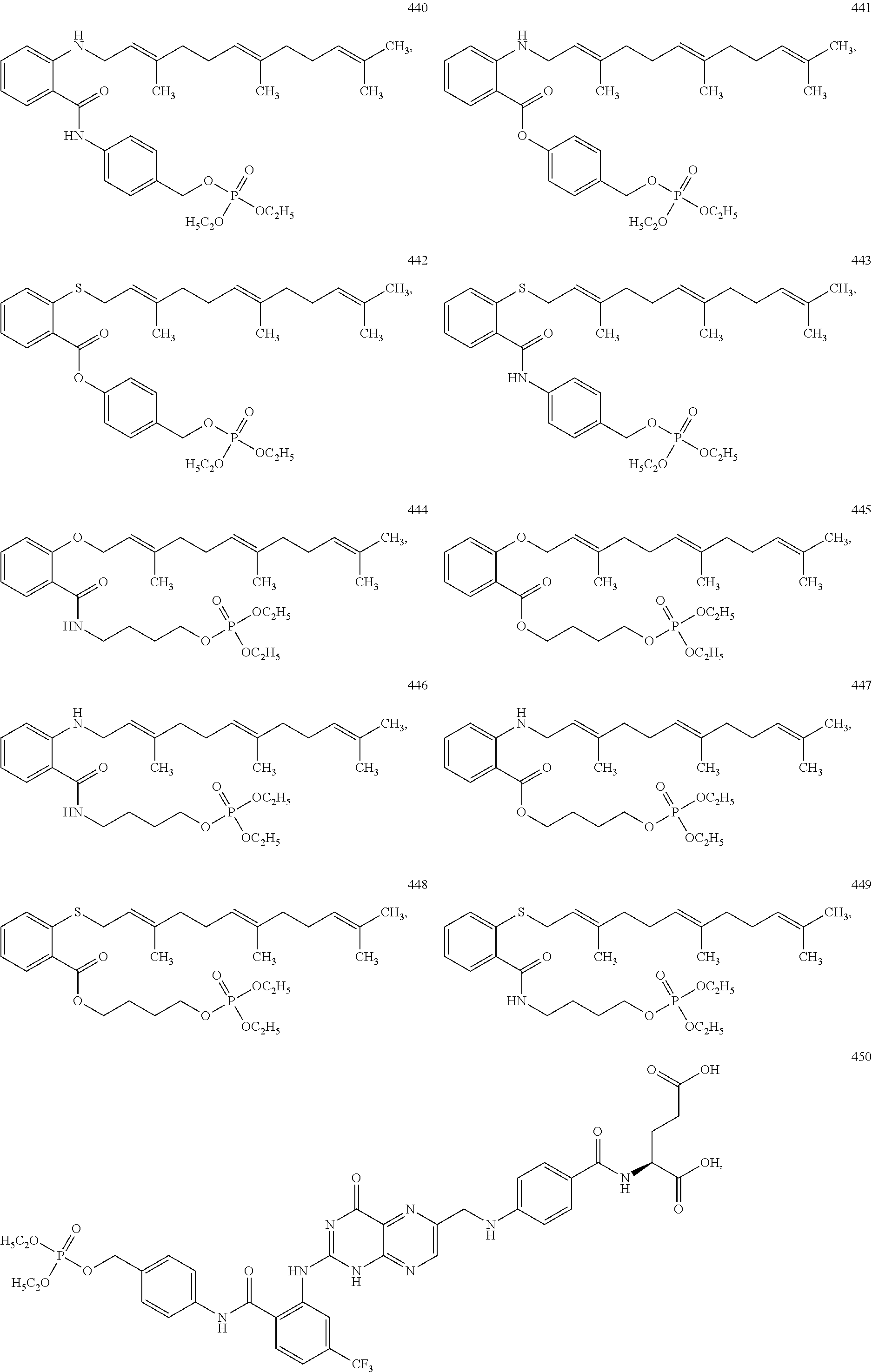

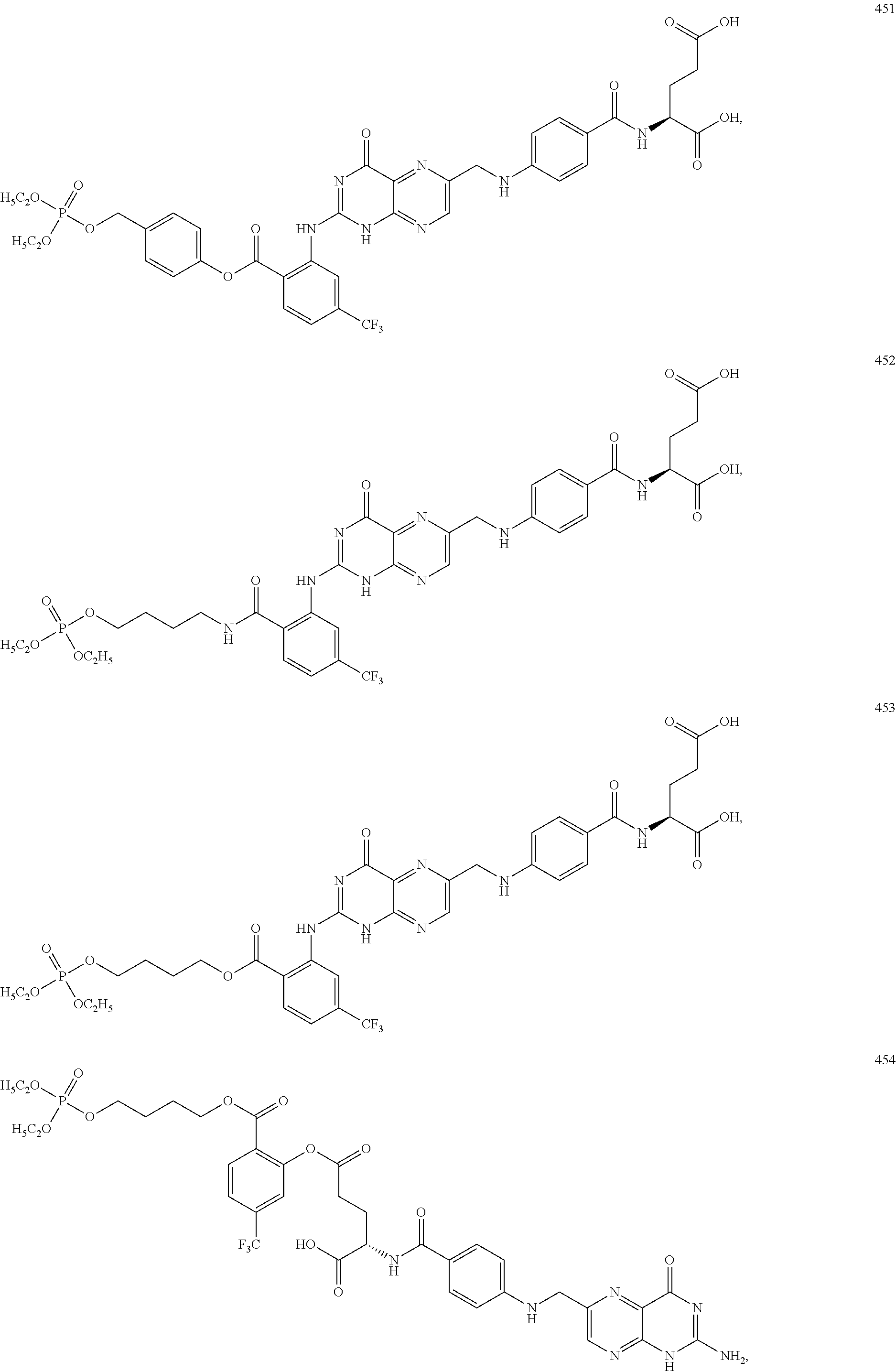

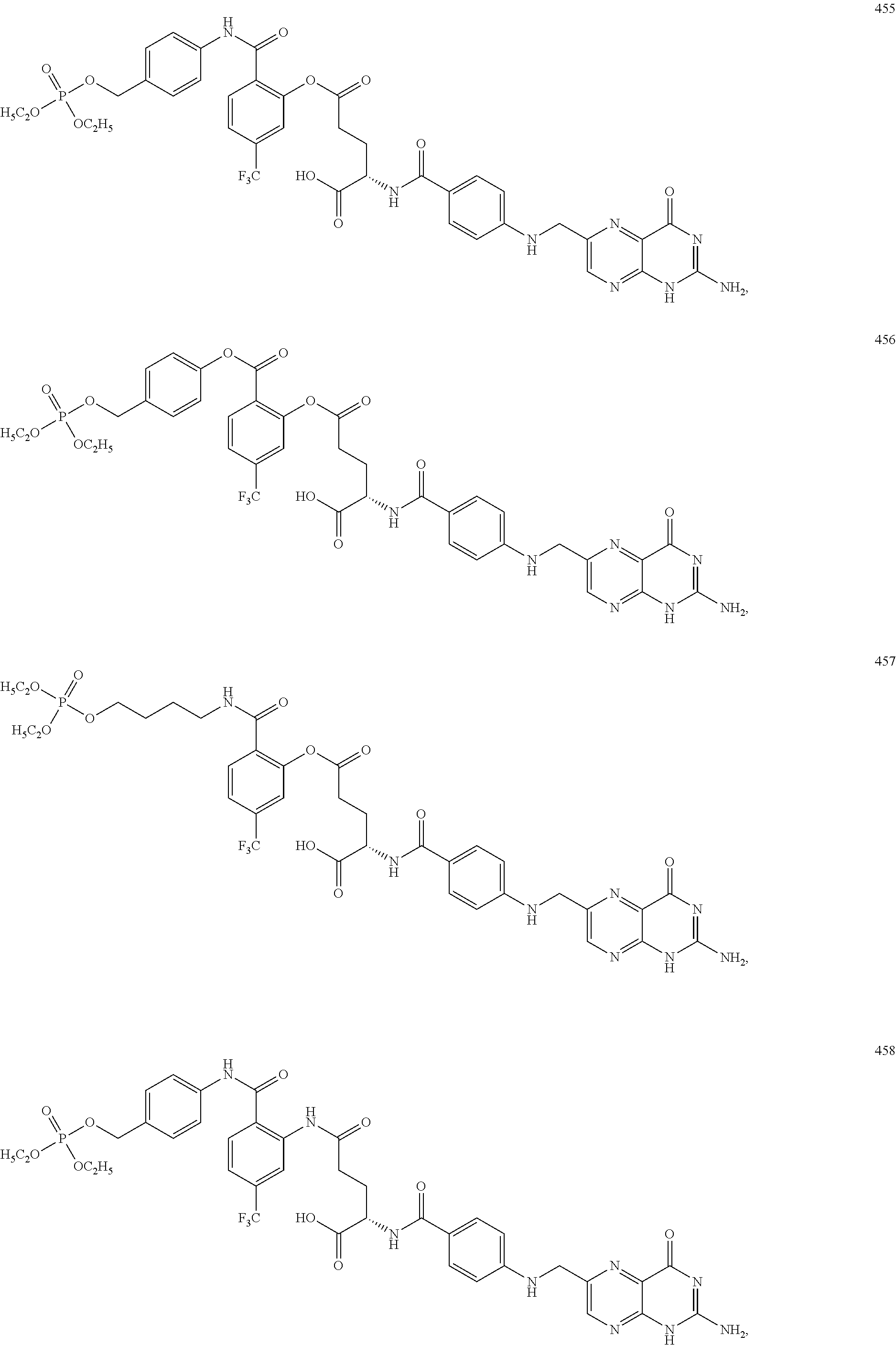

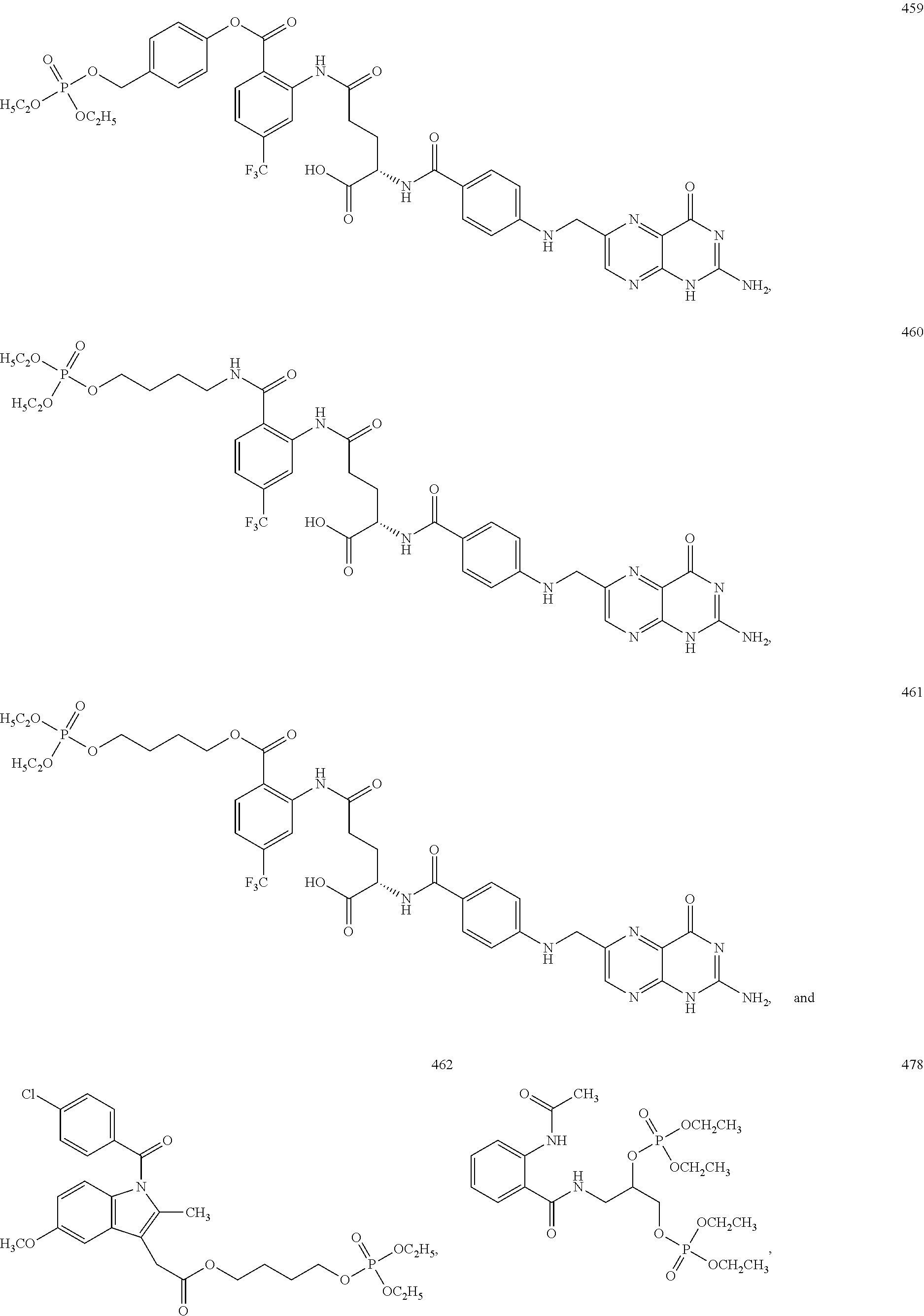

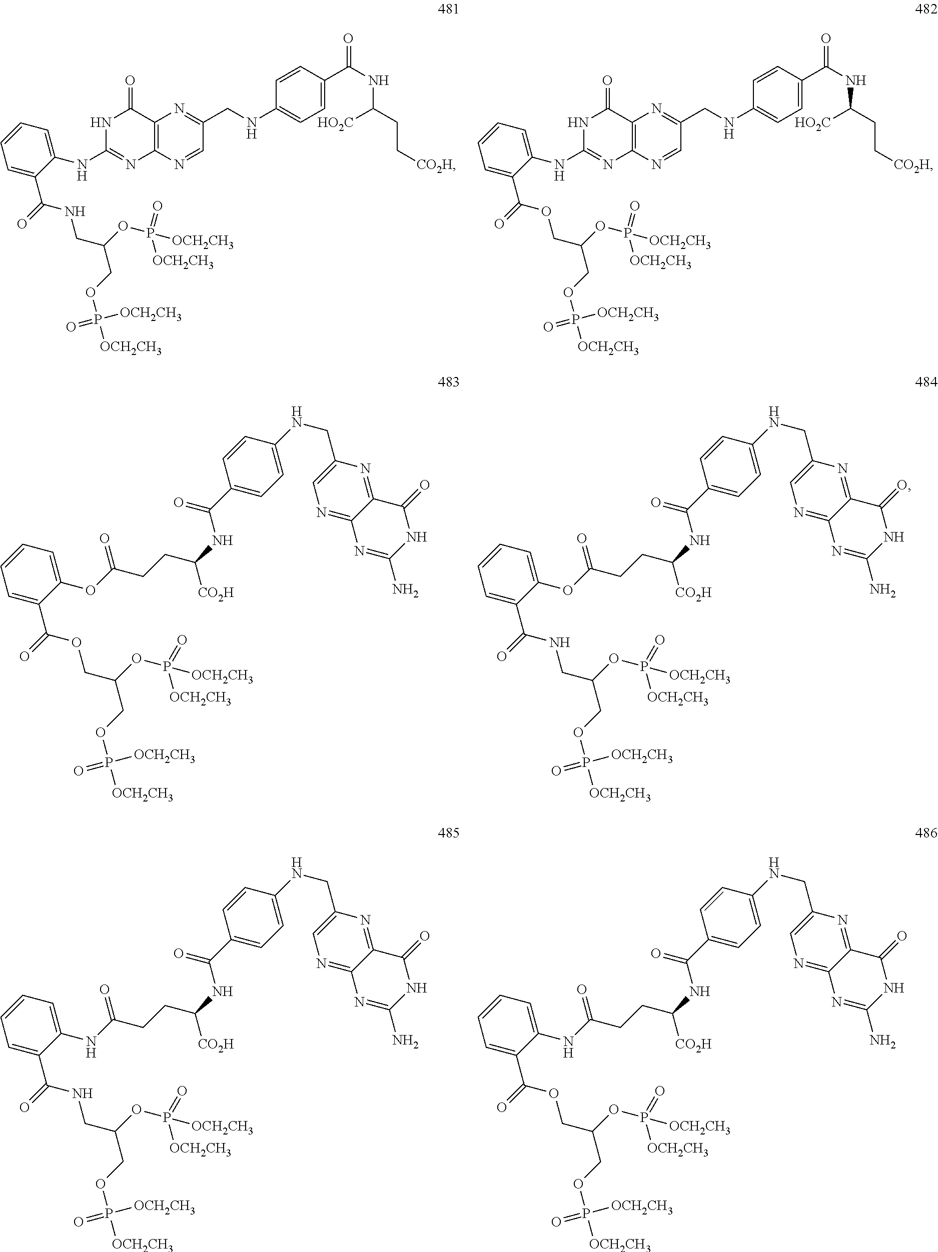

In another aspect the invention features a compound of general Formula X:

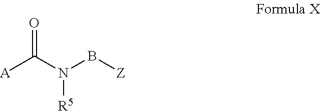

or a pharmaceutically acceptable salt thereof.

In Formula X, A is an optionally substituted aliphatic, heteroaliphatic, aromatic, heteroaromatic substituent or alkylaryl substituent having 1 to 100 carbon atoms or is selected from:

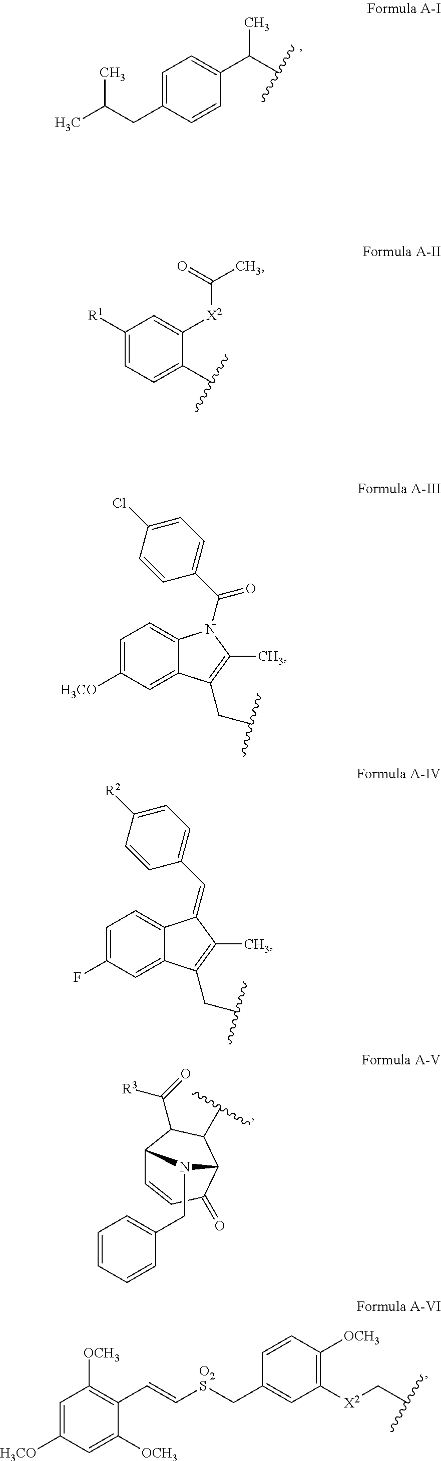

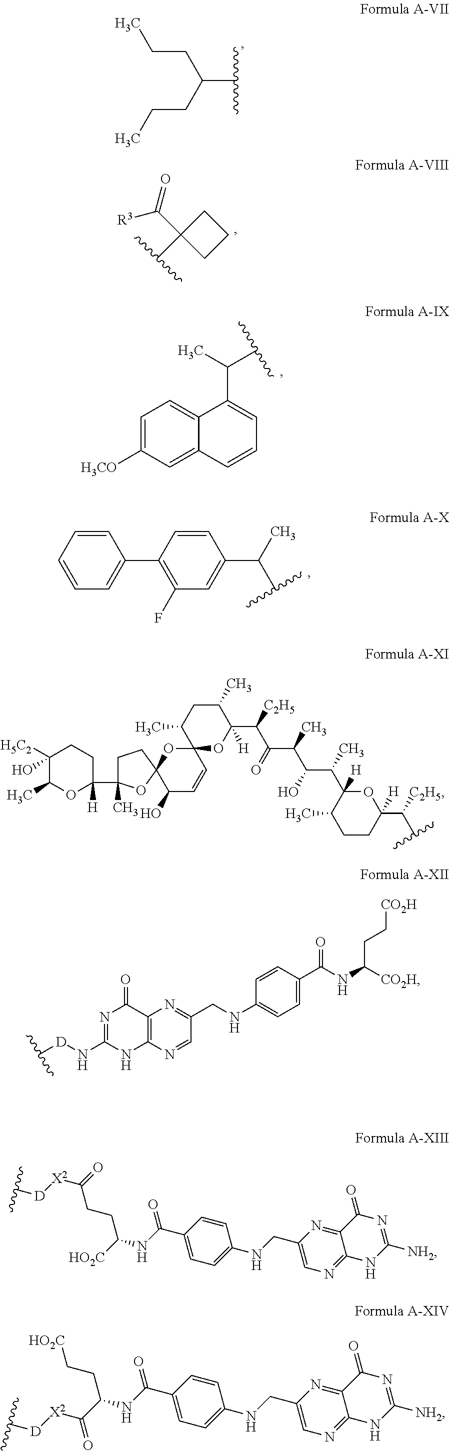

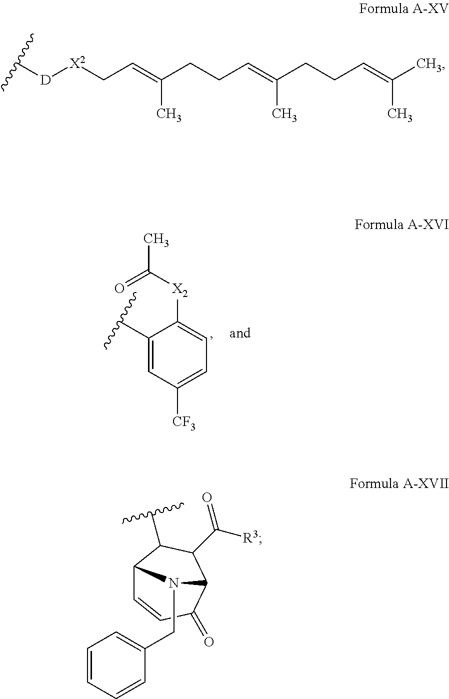

D is absent or

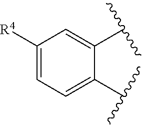

X² is selected from —O—, —NR⁵—, and —S—;

R¹ and R⁴ are independently selected from hydrogen and trifluoromethyl;

R² is selected from —SCH₃, —S(O)CH₃, and —S(O)₂CH₃;

R³ is selected from hydroxyl, Z, and —X¹—B—Z;

R⁵ is selected from methyl and ethyl;

B is selected from:

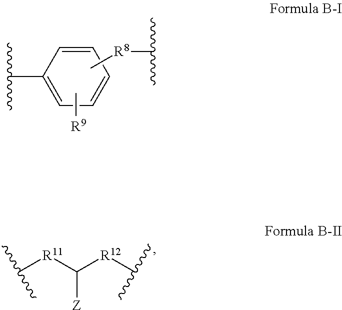

a single bond, and an aliphatic group with 1 to 22 carbon atoms;

R⁸, R¹¹, and R¹² are the same or different C₁₋₄ alkylene;

R⁹ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O-C₁₋₆-alkyl, —OC(O)—C₁₋₆-alkyl, —C(O)NH₂, —C(O)NH—C₁₋₆-alkyl, —S(O)₂—C₁₋₆-alkyl, —S(O)₂NH—C₁₋₆-alkyl, cyano, halo or hydroxy;

Z is selected from:

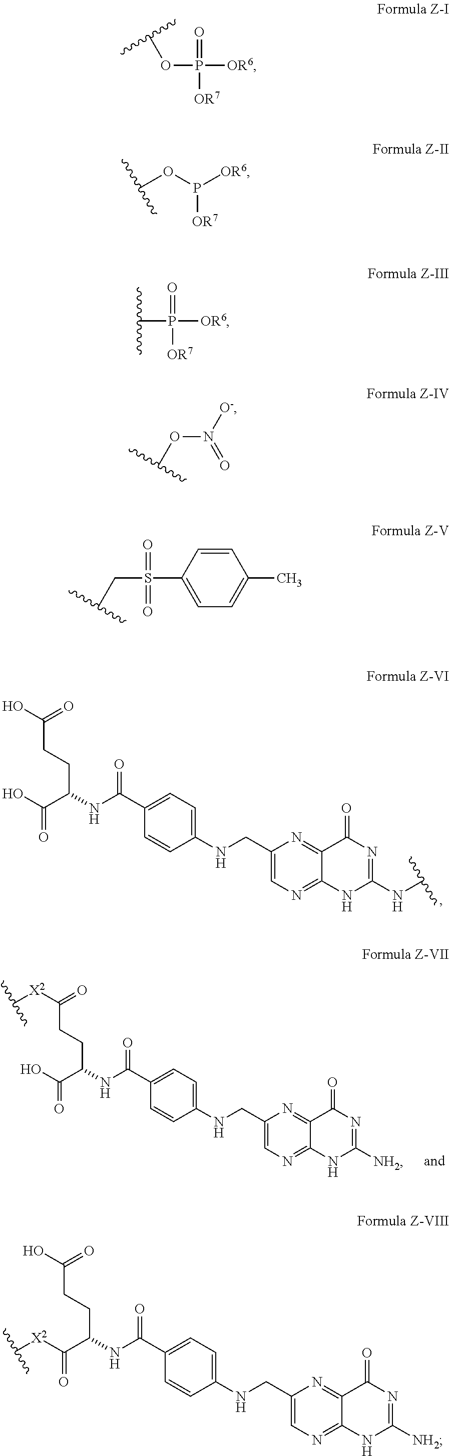

or B together with Z forms a structure:

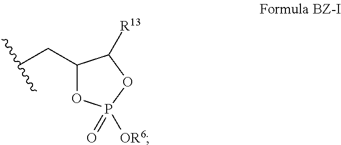

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue.

In another embodiment, the invention features a compound having a structure of compounds 463-487:

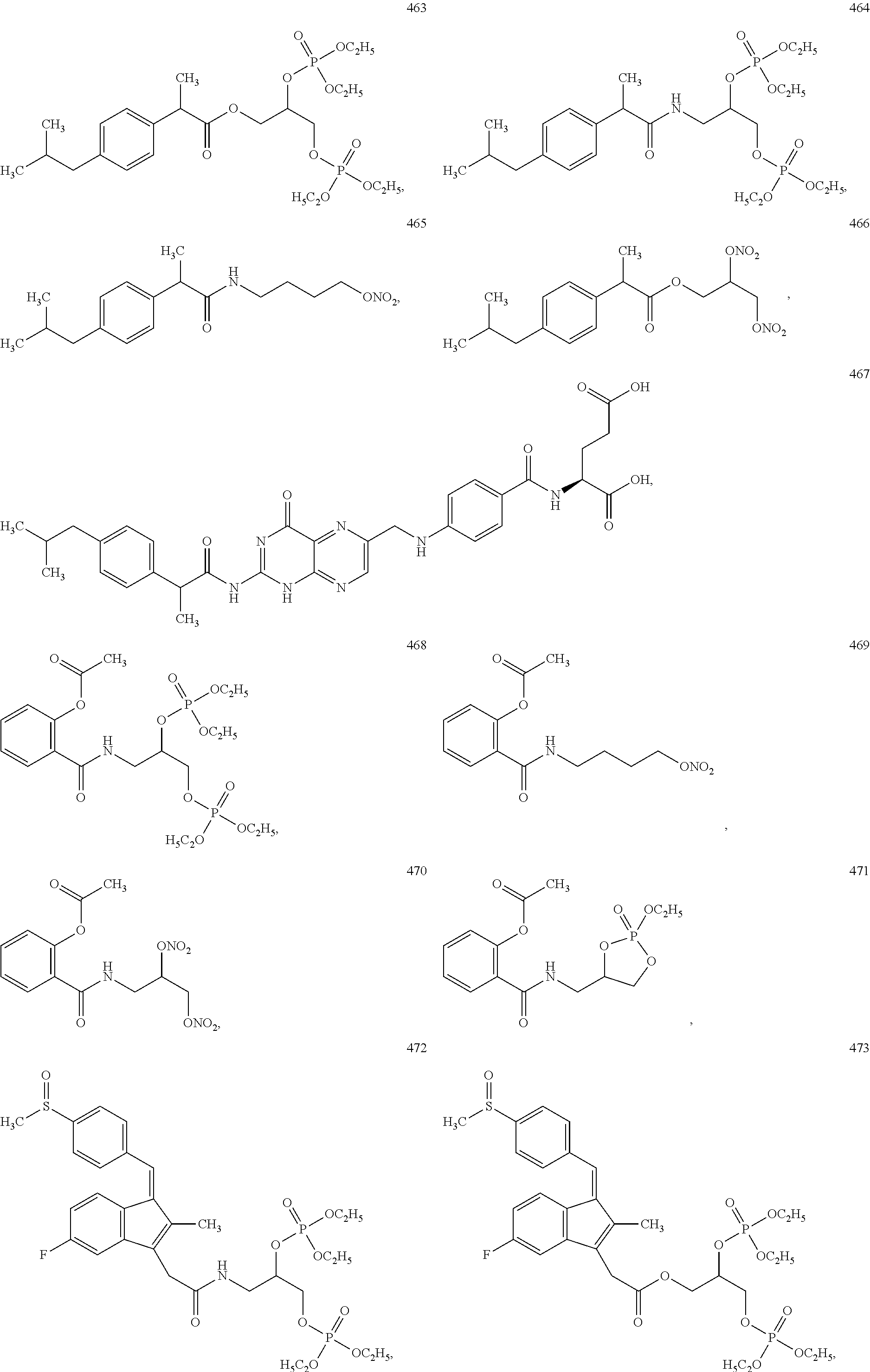

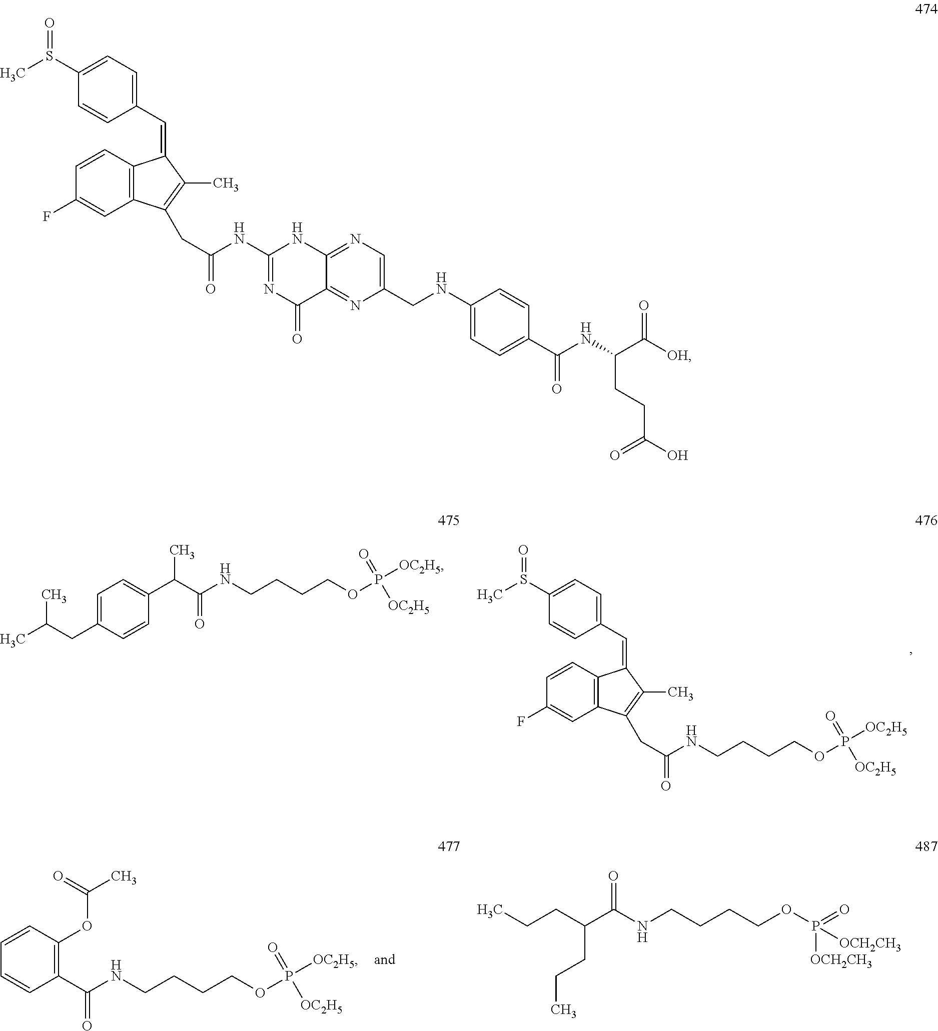

In another embodiment, the present invention features methods for the treatment of non-cancerous conditions of the skin or mucous membranes with an effective amount of compounds of Formula I as first described above.

In this embodiment of Formula I, A is an optionally substituted aliphatic, heteroaliphatic, aromatic, heteroaromatic substituent or alkylaryl substituent having 1 to 100 carbon atoms;

X¹ is selected from —O—, —S—, and —NR⁵—;

R⁵ is selected from hydrogen and a C₁₋₆ alkyl;

B is an aliphatic, alicyclic, heteroaliphatic, heterocyclic, aryl, aralkyl, or heteroaromatic group optionally substituted with one or more R¹⁵ moieties,

each R¹⁴ is independently, selected from hydrogen, halogen, hydroxyl, alkoxyl, —CN; an optionally substituted aliphatic, alicyclic, heteroaliphatic, heterocyclic, aryl, aralkyl, heteroaromatic moiety; —OR^{R}, —S(═O)ₙR^{d}, —NR^{b}R^{c}, —C(═O)R^{a} and —C(═O)OR^{a}; n is 0-2; R^{a}, for each occurrence, is independently selected from hydrogen and an optionally substituted aliphatic, alicyclic, heteroaliphatic, heterocyclic, aryl, aralkyl, or a heteroaromatic moiety; each of R^{b} and R^{c}, for each occurrence, is independently selected from hydrogen; hydroxyl, SO₂R^{d}, and aliphatic, alicyclic, heteroaliphatic, heterocyclic, aryl, aralkyl, heteroaromatic or an acyl moiety; R^{d}, for each occurrence, is independently selected from hydrogen, —N(R^{C})₂, aliphatic, aryl and heteroaryl, R^{c}, for each occurrence, is independently hydrogen or an aliphatic moiety; and R^{R} is an optionally substituted aliphatic, alicyclic, heteroaliphatic, heterocyclic, aryl, aralkyl, heteroaromatic or acyl moiety;

Z is selected from:

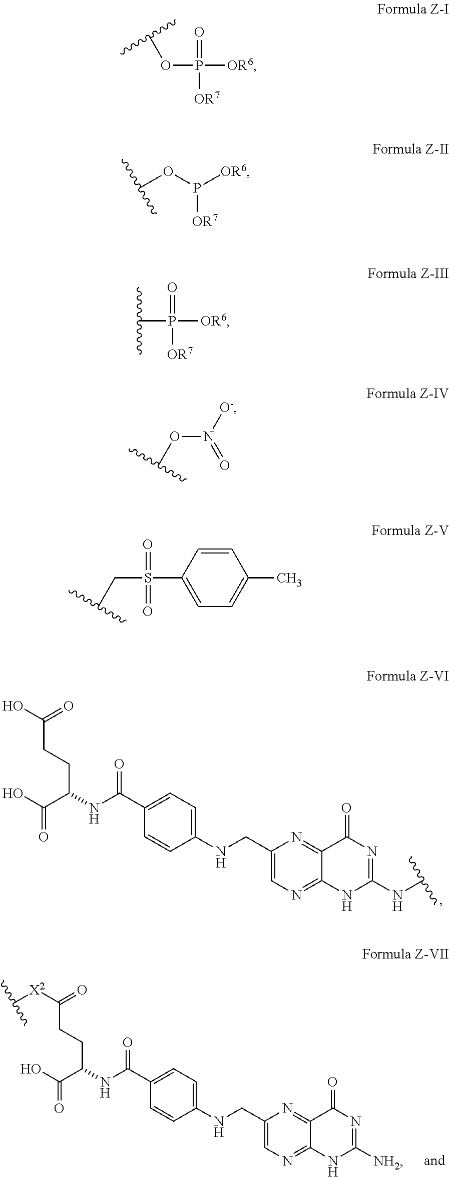

or B together with Z forms a structure:

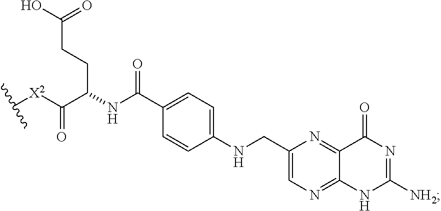

R⁶ and R⁷ are independently selected from hydrogen, C₁₋₆-alkyl, and polyethylene glycol residue; and

R¹³ is selected from hydrogen, an aliphatic group with 1 to 22 carbon atoms (e.g. C₁₋₆-alkyl), and polyethylene glycol residue;

or a pharmaceutically acceptable salt thereof.

In another embodiment, the invention features a compound having a structure exemplified by compounds 488-504. See, U.S. Pat. No. 8,236,820, incorporated by reference. For example, the compound of Formula X can be selected from:

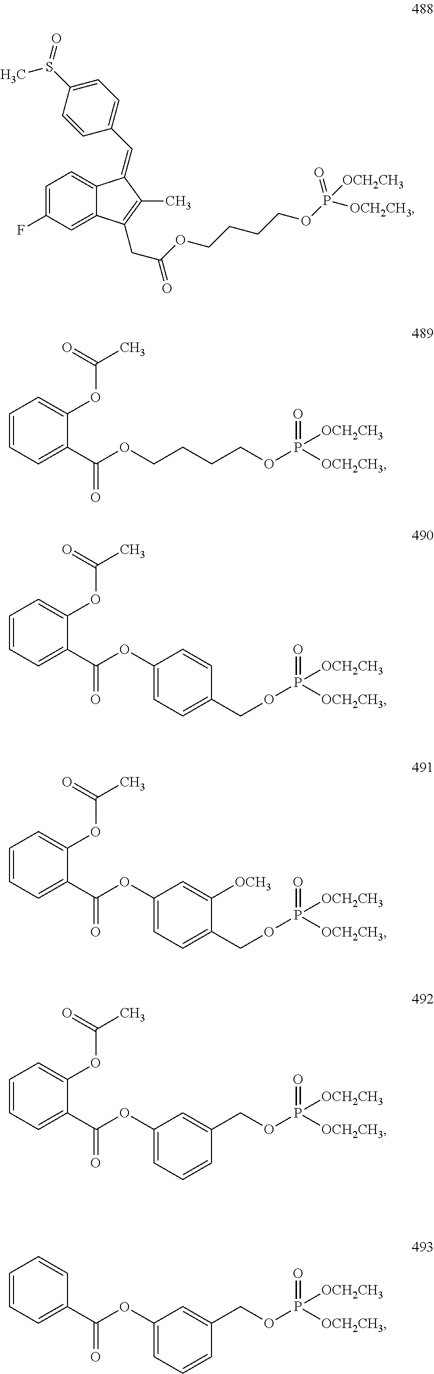 [SMILES: CCOP(=O)(OCC)OCCCCOC(=O)CC1=C(C)/C(=C/c2ccc(S(C)=O)cc2)c2ccc(F)cc21 ; CCOP(=O)(OCC)OCCCCOC(=O)c1ccccc1OC(C)=O ; CCOP(=O)(OCC)OCc1ccc(OC(=O)c2ccccc2OC(C)=O)cc1 ; CCOP(=O)(OCC)OCc1ccc(OC(=O)c2ccccc2OC(C)=O)cc1OC ; CCOP(=O)(OCC)OCc1cccc(OC(=O)c2ccccc2OC(C)=O)c1 ; CCOP(=O)(OCC)OCc1cccc(OC(=O)c2ccccc2)c1]

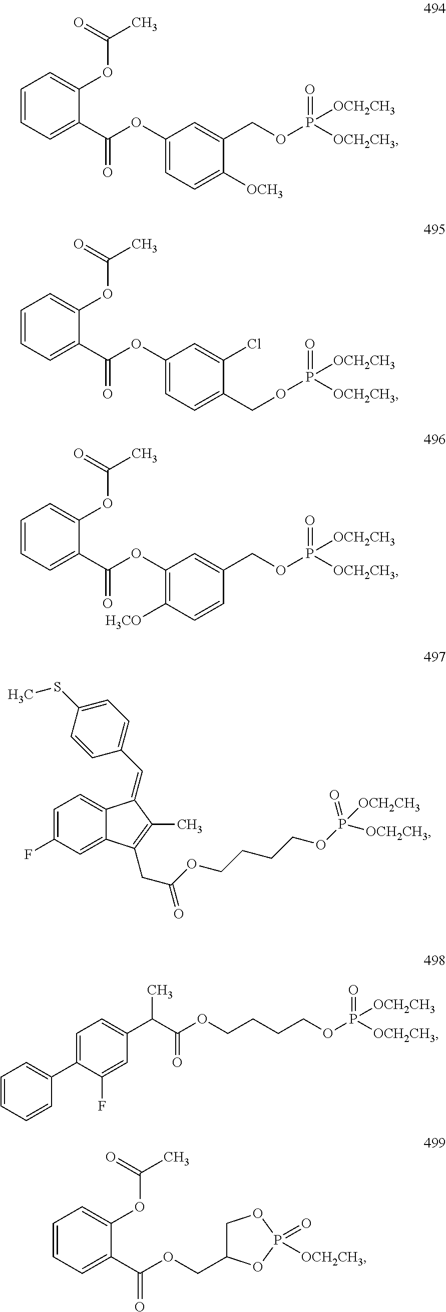 [SMILES: CCOP(=O)(OCC)OCc1cc(OC(=O)c2ccccc2OC(C)=O)ccc1OC ; CCOP(=O)(OCC)OCc1ccc(OC(=O)c2ccccc2OC(C)=O)cc1Cl ; CCOP(=O)(OCC)OCc1ccc(OC)c(OC(=O)c2ccccc2OC(C)=O)c1 ; CCOP(=O)(OCC)OCCCCOC(=O)CC1=C(C)/C(=C/c2ccc(SC)cc2)c2ccc(F)cc21 ; CCOP(=O)(OCC)OCCCCOC(=O)C(C)c1ccc(-c2ccccc2)c(F)c1 ; CCOP1(=O)OCC(COC(=O)c2ccccc2OC(C)=O)O1]

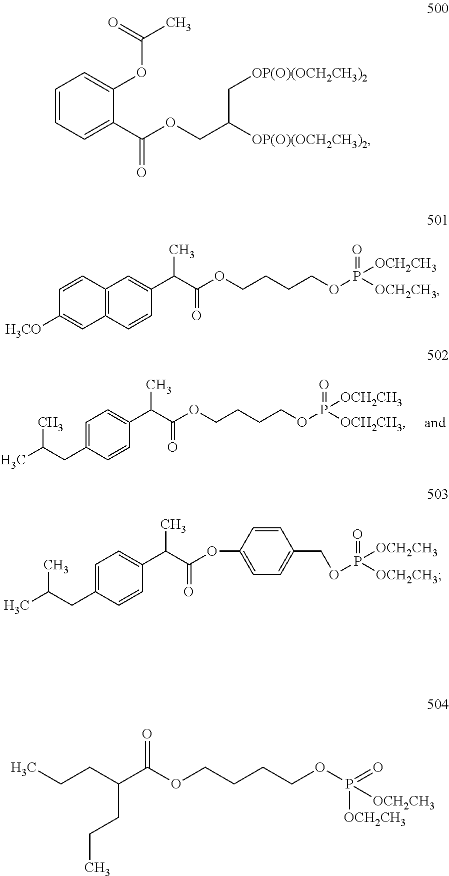

Some of the compounds of the present invention can comprise one or more stereogenic centers, and thus can exist in various isomeric forms, e.g., stereoisomers and/or diastereomers. Thus, the compounds of Formula I, and the compounds of any of its derivative formulas, including Formulas II, III, IV, V, VI, VII, VIII, IX and X (hereinafter “Formulas I through X”) and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided. Moreover, when compounds of the invention exist in tautomeric forms, each tautomer is embraced herein.

Certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Certain exemplary pharmaceutical compositions and pharmaceutically acceptable derivatives will be discussed in more detail herein below.

In one embodiment, the present invention relates to the compounds of any of Formulas I through X, for use in the treatment and/or prevention of cancer and precancerous conditions. In a further embodiment, the present invention relates to the compounds of any of Formulas I through X for use in the treatment and/or prevention of pain. In yet another embodiment, the present invention relates to the compounds of any of Formulas I through X for use in the treatment and/or prevention of inflammation-related diseases. In yet another embodiment, the present invention relates to the compounds of any of Formulas I through X for use as an antipyretic agent.

In a further aspect, the present invention is directed to a pharmaceutical composition comprising the compounds of any of Formulas I through X, as described generally herein, and a pharmaceutically acceptable excipient. In a specific embodiment, the composition is useful for prevention and/or treatment or cancer or precancerous conditions, including but not limited to precancerous conditions such as benign prostatic hypertrophy, colon adenomas, actinic keratosis and various premalignant conditions of the lung, breast, oral cavity, cervix, and pancreas, and also cancer of the mouth, stomach, colon, rectum, lung, prostate, liver, breast, pancreas, skin, brain, head and neck, bones, ovaries, testicles, uterus, small bowel, lymphoma and leukemia.

According to the invention, the compounds of any of Formulas I through X are active against cancers, particularly lung and/or brain cancer and therefore can be used in the treatment and/or prevention of lung and/or brain cancer and precancerous conditions thereof, wherein said compound is administered to a human or animal by the respiratory route. As used herein, “preventing”, “prevention” or “prevent” describes reducing or eliminating the onset of lung or brain cancer or the precancerous conditions thereof or the symptoms or complications of lung and/or brain cancer and precancerous conditions thereof.

Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), non-squamous non-small cell lung cancer, squamous non-small cell lung cancer, squamous cell carcinoma, non-squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructual heterogeneity (e.g. mixed cell types).

The term “brain cancer” as used herein refers to both primary brain tumors and metastatic brain tumors that originate from non-brain cancer cells such as lung cancer cells. Preferably, the term “brain cancer” refers to primary brain tumors. Primary brain tumors are categorized by the type of tissue in which they first develop. The most common brain tumors are called glioma; they originate in the glial tissue. There are a number of different types of gliomas: for instance, astrocytomas, brain stem gliomas, ependymomas, and oligodendrogliomas. Other types of primary brain tumors which do not originate from the glial tissue are, for instance, meningiomas, craniopharyngiomas and germinomas.

Treating lung and/or brain cancer can result in a reduction in size or volume of a tumor. A reduction in size or volume of a tumor may also be referred to as “tumor regression.”Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor or by any reproducible means of measurement.

Treating lung and/or brain cancer may further result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating lung and/or brain cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. A metastasis is a region of cancer cells, distinct from the primary tumor location resulting from the dissemination of cancer cells from the primary tumor to other parts of the body. The number of metastatic lesions may be measured by any reproducible means of measurement. The number of metastatic lesions may be measured by counting metastatic lesions visible to the naked eye or at a specified magnification. Preferably, the specified magnification is 2×, 10×, or 50×.

Treating lung and/or brain cancer can result in an increase in average survival time of a population of subjects treated according to the present invention in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with the compounds of any of Formulas I through X. An increase in average survival time of a population may also be measured, for example, by calculating for a population the average length of survival following completion of a first round of treatment with the compounds of any of Formulas I through X.

Treating lung and/or brain cancer can also result in a decrease in the mortality rate of a population of treated subjects in comparison to an untreated population. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means, for example, by calculating for a population the average number of disease-related deaths per unit time following initiation of treatment with the compounds of any of Formulas I through X. A decrease in the mortality rate of a population may also be measured, for example, by calculating for a population the average number of disease-related deaths per unit time following completion of a first round of treatment with the compounds of any of Formulas I through X.

Another embodiment of the present invention relates to a method for preventing cancer by means of administering the compounds of any of Formulas I through X or a pharmaceutical composition thereof. Accordingly, treatment of an individual with the compounds of any of Formulas I through X or a pharmaceutical composition thereof reduces the risk of the individual to develop cancer. Preferably, after the treatment, the risk of the individual to develop cancer is reduced by 5% or greater; more preferably, the risk to develop cancer is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. As used herein, reducing risk of developing cancer includes decreasing the probability or incidence of developing cancer for an individual compared to a relevant, e.g. untreated, control population, or in the same individual prior to treatment according to the invention. Reduced risk of developing cancer may include delaying or preventing the onset of a cancer. Risk of developing cancer can also be reduced if the severity of a cancer or a precancerous condition is reduced to such a level such that it is not of clinical relevance. That is, the cancer or a precancerous condition may be present but at a level that does not endanger the life, activities, and/or well-being of the individual. For example, a small tumor may regress and disappear, or remain static. Preferably, tumor formation does not occur. In some circumstances the occurrence of the cancer or the precancerous condition is reduced to the extent that the individual does not present any signs of the cancer or the precancerous condition during and/or after the treatment period.

Cell proliferative disorders of the lung include all forms of cell proliferative disorders affecting lung cells. Cell proliferative disorders of the lung can include lung cancer and precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, precancerous lung lesion and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, emphysema, and Hodgkin's disease.

The compounds of any of Formulas I through X and pharmaceutical compositions thereof are further directed at individuals at risk of developing lung cancer. Such risk may be based on the medical or social history of an individual, such as inhalation of tobacco products as it occurs for example in smokers or exposure to asbestos or in non-smokers who breathe in secondhand smoke. Another category of individuals at risk for lung cancer are those harboring genetic mutations predisposing them to lung cancer. Yet another category is individuals who have been exposed to ionizing radiation or chemotherapeutic agents. Yet, another category is individuals with a known cancer at a location other than the lungs that have a propensity to metastasize to the lungs.

The method for preventing cancer according to the present invention is beneficial both for individuals having a precancerous condition and individuals who are healthy. Individuals with lifestyle habits that could lead to cancer, particularly smokers, and individuals affected by diseases for which the probability of cancer incidence is high have a particularly high order of priority as individuals for the preventive method of the present invention. Furthermore, individuals who are likely to acquire familial cancers, and such individuals as those who are diagnosed with a risk of cancer by means of gene diagnoses based on single-nucleotide polymorphism or the like may also be targeted.

Finally, another category is individuals with prior lung cancer that has already been treated. Accordingly, the corresponding embodiment of the present invention relates to a method for preventing cancer recurrence by means of administering the compound of Formula I or a pharmaceutical composition thereof. Cancer recurrence is a re-development of the cancer in an individual, who had previously undergone a cancer treatment, after a period of time in which no cancer could be detected. The probability of a cancer recurring depend on many factors, including the type of cancer and its extent within the body at the time of the treatment.

Yet another embodiment of the present invention provides pharmaceutical compositions for the treatment of human and animal inflammation-related diseases, including, but not limited to neoplasms, rheumatologic diseases such as rheumatoid arthritis and Sjogren's syndrome; cardiovascular diseases, such as coronary artery disease, peripheral vascular disease and hypertension; neurodegenerative diseases, such as Alzheimer's disease and its variants or cerebrovascular diseases; and autoimmune diseases for example lupus erythematosus.

In addition to the compounds of any of Formulas I through X, the pharmaceutical compositions of the present invention can comprise one or more further pharmaceutical agents, for instance, compounds having anti-cancer activity. The compounds any of Formulas I through X can be administered alone or in combination with other active agents.

In yet another aspect, the present invention provides methods for treating any disorder related to undesirable inflammation comprising administering to a subject (e.g. human patient or animal) in need thereof a therapeutically effective amount of any compound of any of Formulas I through X or a pharmaceutical composition comprising a compound of the invention. In a preferred embodiment, the disorder includes, but is not limited to rheumatologic diseases such as for example rheumatoid arthritis and Sjogren's syndrome; cardiovascular diseases, such as, for example, coronary artery disease, peripheral vascular disease and hypertension; neurodegenerative diseases, such as, for example, Alzheimer's disease and its variants or cerebrovascular diseases; autoimmune diseases such as for example lupus erythematosus; and other conditions characterized by chronic inflammation of organs such as for example the lung, such as chronic bronchitis or the sinuses, such as chronic sinusitis, and the skin, including eczema or atopic dermatitis, dryness of the skin and recurring skin rashes, contact dermatitis and seborrhoeic dermatitis, neurodermatitis and discoid and venous eczema.

In a further aspect, the invention is directed to a method for inhibiting inflammation, in particular, chronic inflammation in a subject in need thereof by administering to the subject an amount of the compound or composition of the present invention effective to inhibit inflammation. The subject may be a human patient or animal, for instance a mammal. Furthermore, the present invention is directed to a method for the treatment and/or prevention of cancer in a subject in need thereof by administering to the subject an amount of the compound or composition of the present invention.

In another aspect, the present invention provides methods for treating pain and/or fever. The invention further pertains to a method for alleviating pain, comprising administering to a subject in need thereof a pharmaceutically effective amount of any compound of any of Formulas I through X of the present invention, or of a pharmaceutical composition of the present invention. The invention further pertains to a method for treating fever, comprising administering to a subject in need thereof a pharmaceutically effective amount of a compound of the present invention or of a pharmaceutical composition of the present invention.

Cardiovascular disease, the leading cause of death in Americans, encompasses an array of symptoms and cardiac events that eventually lead to heart attack and/or stroke. Atherosclerosis contributes to these cardiac events by decreasing the blood flow in the affected arteries, eventually leading to oxygen deprivation of the organs targeted by the damaged arteries (Badimon, L. & Vilahur, G. Coronary atherothrombotic disease: progress in antiplatelet therapy. Revista espanola de cardiologia 61, 501-513 (2008)). The characteristic plaques that form within the arteries of atherosclerotic patients can rupture, yielding a site of injury that platelets can recognize and adhere to. Thrombus generation is then initiated, and thrombi can enter the circulation and cause ischemic events.

Non-steroidal anti-inflammatory drugs (NSAIDs) are a class of drugs with analgesic, antipyretic, and anti-inflammatory properties. Aspirin, a common NSAID, reduces inflammation by inhibiting thromboxane production through the irreversible inhibition of cyclooxygenase-1 (COX-1) (McAdam, B. F. et al. Systemic biosynthesis of prostacyclin by cyclooxygenase (COX)-2: the human pharmacology of a selective inhibitor of COX-2. Proceedings of the National Academy of Sciences of the United States of America 96, 272-277 (1999)). Aspirin is currently used as an antiplatelet therapy for patients at risk for adverse cardiovascular events.

Aspirin is effective in reducing 30% of adverse cardiac events. However, there are side effects associated with the prophylactic aspirin treatment, including increased hemorrhagic events especially in the gastrointestinal tract (Serebruany, V. L. et al. Analysis of risk of bleeding complications after different doses of aspirin in 192,036 patients enrolled in 31 randomized controlled trials. The American journal of cardiology 95, 1218-1222, (2005); Eikelboom, J. W. et al. Aspirin-resistant thromboxane biosynthesis and the risk of myocardial infarction, stroke, or cardiovascular death in patients at high risk for cardiovascular events. Circulation 105, 1650-1655 (2002)). Therefore, development of new therapies to prevent thrombus formation is needed. An unexpected finding with the compounds claimed herein is that they are free of bleeding side effects. Two sets of data support this conclusion: 1) No animal treated with any of our compounds showed any increase in gastrointestinal damage or bleeding. In fact in all cases either they had no gastrointestinal toxicity or when compared to their parent NSIAs had significantly reduced gastrointestinal mucosal damage and no bleeding (Mackenzie G G et al; Gastroenterology, 2010; 139(4): 1320-32; and Huang L et al; Br J. Pharmacol. 2011; 162(7):1521-33). And 2) There is no prolongation of the bleeding time, a direct measure of a compound's ability to cause bleeding side effects, in mice treated with phospho-sulindac 96 or phospho-sulindac amide 95.

In another embodiment, any compound of any of Formulas I through X of the present invention, or of a pharmaceutical composition of the present invention, is directed to the treatment of cardiovascular disease. These compounds are characterized by complete absence of hemorrhagic events, since they do not inhibit cyclooxygenase. In a further embodiment, any compound of any of Formulas I through X of the present invention, or of a pharmaceutical composition of the present invention, may be administered to a human or animal patient as an anti-thrombotic and/or anti-therapeutic.

In another embodiment, the compounds of any of Formulas I through X of the present invention may be used for the manufacture of pharmaceutical compositions for treatment of any diseases and disorders listed above.

The compounds of the present invention have high in vivo stability. Preferably, the concentration of the compounds of any of Formulas I through X in blood plasma of an animal after 3 hours of administration is at least 30% of its initial concentration, more preferred at least 40% of its initial concentration, and particularly preferred at least 50% of its initial concentration. The corresponding tests can be carried out with animals such as mice according to the method described by Xie et al. (Xie G, Nie T, Mackenzie G, Sun Y, Fluang L, Ouyang N, et al. Br. J. Pharmacol. 2011).

In addition, the compounds of the present invention have cellular uptake values, which can be determined by using cancer cells, for instance human non-small cell lung cancer cells A549 and subsequently assaying their intracellular levels by HPLC. The tests can be performed according to the method outlined in Example 2. Preferably, the cellular uptake values of the compounds are higher than 0.1 nmol/mg protein, more preferred higher than 1.0 nmol/mg protein, even more preferred higher than 10.0 nmol/mg protein and particularly preferred higher than 50.0 nmol/mg protein.

In one embodiment, the compounds of any of Formulas I through X have an n-octanol-water partition coefficient (log P) value higher than 2, more preferred higher than 3 and particularly preferred higher than 4. Log P is defined as the ratio of concentrations (mol/volume) of the compounds of Formula I in n-octanol and in water. Suitable methods for the measurement of n-octanol-water coefficients are, for instance described in Octanol-Water Partition Coefficients: Fundamentals and Physical Chemistry, John Wiley and Sons Ltd., 1997, ISBN: 0-417-97397 1. Both solvents are mutually saturated before the measurement. At equilibrium the n-octanol phase contains 2.3 mol/l of water and the aqueous phase contains 4.5×10⁻³ mol/l of n-octanol. The measurement is carried out at the isoelectric point of the compound of Formula I at temperature of 25° C. The log P of the compounds of Formula I is preferably determined by the shake-flask method, which is, for example, described in the review of J. Sangster (J. Phys. Chem. Ref. Data 18, 1989; 3:1111-1227). The measurement is carried out under the conditions described by T. Fujita et al. (J. Am. Chem. Soc. 1964; 86:5175-5180) and the concentration of the compound of Formula I in each of the two phases is determined by high performance liquid chromatography (HPLC).

As discussed above, this invention provides novel compounds for use in the treatment and prevention of lung and/or brain cancer and precancerous conditions thereof, wherein said compounds are administered to a human or animal by the respiratory route. The term “respiratory route” as used herein refers to both nasal and pulmonary respiratory routes. Administration by the nasal respiratory route includes nasal administration, and nose to brain delivery whereby the composition of the present invention is sprayed into the nasal cavity and delivered to the brain via the olfactory and trigeminal neural pathways. Nasal drug delivery is known to a person skilled in the art and is, for instance, described in L. Ilium (J. Control. Release 87 (2003), pp. 187-198). Administration by nasal respiratory route and nose to brain delivery is particularly suitable for the treatment of brain cancer and the corresponding precancerous conditions.

Preferably, the permeability of the nasal mucosa to the compounds of any of Formulas I through X is high, and subsequently, their bioavailability upon nasal administration is more than 60%, preferably more than 70% and even more preferred more than 80%. When the composition of the present invention is administered by the nasal respiratory route, more than 50 wt.-%, preferably more than 60 wt.-% and particularly preferred more than 70 wt.-% of the compounds of any of Formulas I through X is absorbed through the nasal mucosa and enters the systemic circulation of the patient. Thus, this embodiment of the present invention allows a rapid and effective administration of the compound of any of Formulas I through X. Furthermore, if the aerosol particles have mass median aerodynamic diameter of less than 10 ppm, up to 40 wt.-%, preferably up to 50 wt.-% and more preferred up to 60 wt.-% of the compounds of any of Formulas I through X is delivered to the lungs of the patient. Accordingly, the compound of any of Formulas I through X is delivered to the lung cancer of the patient both locally and systemically.

The composition for nasal administration may be an aqueous solution designed to be administered to the nasal passages in form of drops or sprays. Preferably, this composition is isotonic to nasal secretions and slightly buffered to maintain a pH of 5.5 to 6.5. Antimicrobial agents and/or preservatives may be also present in this composition. In another embodiment of the invention, the composition is administered by the oral respiratory route.

For administration by the respiratory route, the compounds can be delivered in the form of an aerosol spray from a pressurized container or dispenser, which contains a suitable propellant, e.g. hydrofluoroalkanes, chlorofluorocarbons, carbon dioxide, or a nebulizer. In the case of a pressurized aerosol, the dosage unit may be determined by providing a valve to deliver a metered amount. Capsules and cartridges of e.g. gelatine for use in an inhaler or insufflator may be formulated containing a powder mix of the compound and a suitable pharmaceutically acceptable carrier.

Administration by the respiratory route usually requires the use of pharmaceutical compositions suitable for the dispensing of the compounds of any of Formulas I through X. Typically, each pharmaceutical composition is specific to the type of device employed and may involve the use of an appropriate propellant material, in addition to the usual diluents, adjuvants and/or carriers. Also, the use of liposomes, microcapsules or microspheres, inclusion complexes, or other types of carriers is contemplated. The compounds of any of Formulas I through X may be prepared in different pharmaceutical compositions depending on their physical and chemical properties or the type of device employed.

Pharmaceutical compositions suitable for use with a nebulizer, either jet or ultrasonic, will typically comprise the compounds of any of Formulas I through X dissolved in a solvent at a concentration of about 0.1 to 25 mg of the compounds of any of Formulas I through X per 1 ml of solution. The pharmaceutical composition may also include a buffer, for instance, an aminoacid, and a simple sugar (e.g. for stabilization and regulation of osmotic pressure). The solvent in the pharmaceutical composition may be selected from the group consisting of water, ethanol, 1,3-propylene glycol, glycerol or a mixture of any of those. Nebulized pharmaceutical compositions may also contain a surfactant, to reduce or prevent surface induced aggregation of the compound of Formula I caused by atomization of the solution in forming the aerosol.

Pharmaceutical compositions for use with a metered-dose inhaler device generally comprise a finely divided powder containing the compounds of any of Formulas I through X (or a pharmaceutically acceptable derivative thereof) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Pharmaceutical compositions for dispensing from a powder inhaler device will comprise a finely divided dry powder containing the compounds of any of Formulas I through X and may also include a bulking agent, such as lactose, sorbitol, sucrose, or mannitol in amounts, which facilitate dispersal of the powder from the device, e.g. 50 to 90% by weight of the formulation. The compounds of any of Formulas I through X should most advantageously be prepared in a particulate form with an average particle size of less than 10 μm, preferably less than 5 μm and more preferred less than 1 μm, for effective delivery to the distal lung.

In another aspect of the present invention, pharmaceutical compositions are provided, which comprise the compounds of any of Formulas I through X (or a pharmaceutically acceptable salt, cocrystal or other pharmaceutically acceptable derivative thereof), and optionally comprise a pharmaceutically acceptable carrier. In certain embodiments, these compositions optionally further comprise one or more additional therapeutic agents. In a specific embodiment, the composition further comprises difluoromethylornithine or cimetidine.

Alternatively, the compounds of this invention may be administered to a patient in need thereof in combination with the administration of one or more other therapeutic agents. For example, additional co-administered therapeutic agents included in a pharmaceutical composition with a compound of this invention may be an approved anti-inflammation or analgesic agent, or it may be any one of a number of agents undergoing approval in the Food and Drug Administration that ultimately obtain approval for the treatment of any disorder related to inflammation and pain. Such additional therapeutic agents may also be provided to promote the targeting of the compounds of the invention to the desired site of treatment, or may increase their stability, increase their plasma half-life, and further improve their biodistribution and pharmacokinetics. It will also be appreciated that certain of the compounds of present invention can exist in a free form for treatment, or where appropriate, as a pharmaceutically acceptable derivative thereof. According to the present invention, a pharmaceutically acceptable derivative includes, but is not limited to, pharmaceutically acceptable salts, esters, salts or cocrystals of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt or cocrystals” refers to those salts or cocrystals which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts or cocrystals of amines, carboxylic acids, and other types of compounds, are well known in the art. For example, S. M. Berge et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts.

Examples of pharmaceutically acceptable, nontoxic acid addition salts are salts of an amino substituent formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts and coformer molecules for cocrystal formation, include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester substituents include, for example, those derived from pharmaceutically acceptable aliphatic carboxylic acids, particularly alkenoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals without undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are transformed in vivo to yield the parent compound of the above formula, for example, by hydrolysis in blood. A thorough discussion is provided in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention.

Some examples of materials which can serve as pharmaceutically acceptable carriers include, but are not limited to volatile solid materials, such as menthol, sugars such as lactose, glucose and sucrose; excipients such as cocoa butter; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal® 53 MCT, Phosal® 50 PG, Phosal® 75 SA, Phospholipon® 90H, Phospholipon® 90G and Phospholipon® 90 NG; soy-phosphatidylcholine (SoyPC) and DSPE-PEG2000 are particularly preferred; buffering agents such as amino acids; pyrogen-free water; isotonic saline; Ringer's solution; ethyl alcohol, and phosphate buffer solutions, as well as other non-toxic compatible lubricants such as sodium lauryl sulfate as well as releasing agents, coating agents, sweetening, flavoring and perfuming agents, preservatives and antioxidants can also be present in the pharmaceutical composition, according to the judgment of the formulator.

The compounds of any of Formulas I through X are also suitable for incorporation into nanoparticulate systems such as liposomes, polymeric nanoparticles, polymeric micelles, lipid nanoparticles, micro- and nano-emulsions, nanogels, liposomes being particularly preferred. The corresponding nanoparticulate systems are known in the prior art and are, for instance, described in the review by Wu and Mansour (X. Wu and H. M Mansour, Invited Paper. International Journal of Nanotechnology: Special Issue-Nanopharmaceuticals, 2011, 8, 1/2, 115-145).

Nanoparticulate systems typically have an average particle size ranging from 1 to 1000 nm, preferably from 50 to 500 nm. The term “liposomes” as used herein refers to phospholipid vesicles with average particle size ranging from 50 to 1000 nm, which are formed by one or several lipid bilayers with an aqueous phase both inside and between the bilayers. The term “polymeric nanoparticles” refers to solid colloidal particles comprising polymeric materials. The average particle size of polymeric nanoparticles ranges from 30 to 300 nm. Polymeric micelles are particles formed through the self-assembly of amphiphilic block copolymers containing hydrophobic and hydrophilic blocks.

Lipid nanoparticles may be in the form of solid lipid nanoparticles, nanostructured lipid carriers or lipid drug conjugates. Microemulsions are typically characterized by the average internal globule size of less than 150 nm. Microemulsions require a surfactant concentration of at least 10 wt.-%, preferably of at least 50 wt.-% and more preferred of at least 20 wt.-%, based on the weight of the composition.

The term “nanogel” refers to aqueous dispersions of hydrogel particles formed by physically or chemically cross-linked polymer networks of nanoscale size. Nanogels can be prepared by a variety of methods such as self-assembly of polymers, polymerization of monomers, cross-linking of preformed polymers or template-assisted nanofabrication.

Use of nanoparticulate systems according to the present invention provides sustained-release of the compounds of any of Formulas I through X in the lung tissue, resulting in a reduction of dosing frequency and improved patient compliance and further enabling uniformity of drug dose distribution among the alveoli. Moreover, by formulating the compounds of any of Formulas I through X as in nanoparticulate systems, one can achieve a dose that is higher than that of other pharmaceutical compositions, which are limited by the solubility volatility of the compounds of any of Formulas I through X. Nanoparticles can be internalized by a variety of cell types. Besides macrophages, other cells like cancer cells and epithelial cells are also able to take up nanoparticles. Therefore, usage of nanoparticulate systems for delivering the compounds of any of Formulas I through X is highly advantageous for the treatment and prevention of lung cancer.

Nanoparticulate formulations can further be advantageously used for the nasal delivery of the compounds of any of Formulas I through X. In this embodiment, multiple-unit mucoadhesive nanoparticles are preferably used in order to prolong the contact of the compound of Formula I with the nasal mucosa.

The resulting compositions can be advantageously employed for administration by the respiratory route. Preferred liposome compositions are those which in addition to other phospholipids, incorporate pegylated phospholipids, such as DSPE-PEG2000, and exhibit long circulation times by avoiding uptake and clearance by the reticuloendothelial system (RES) and thus, are able to reach and treat lung cancer tumors.

In some embodiments, the pharmaceutical composition may further comprise an additional compound having anticancer activity. The additional compound having anticancer activity can be selected from the group of compounds such as chemotherapeutic and cytotoxic agents, differentiation-inducing agents (e.g. retinoic acid, vitamin D, cytokines), hormonal agents, immunological agents and anti-angiogenic agents. Chemotherapeutic and cytotoxic agents include, but are not limited to, alkylating agents, cytotoxic antibiotics, antimetabolites, vinca alkaloids, etoposides, and others (e.g., paclitaxel, taxol, docetaxel, taxotere, cis-platinum). A list of additional compounds having anticancer activity can be found in L. Brunton, B. Chabner and B. Knollman (eds). Goodman and Gilman's The Pharmacological Basis of Therapeutics, Twelfth Edition, 2011, McGraw Hill Companies, New York, N.Y.

In a preferred embodiment, the additional compound having anticancer activity is a tyrosine kinase inhibitor (TKI). A TKI inhibits the tyrosine kinase activity of at least one tyrosine kinase. The inhibition may be reversible or irreversible. TKIs include, but are not limited to, agents such as imatinib, dasatinib, nilotinib, gefitinib, erlotinib, lapatinib, sunitinib, sorafenib and pazopanib. Various TKIs are, for instance, described in Hartmann et al. (J. Th. Hartman et al. Cur. Drug Metab, 2009, 10, pp. 470-481).

In another embodiment, the additional compound having anticancer activity is a compound with oxidative stress-inducing ability. These compounds increase the oxidative stress of cancer cells by inhibiting the mechanisms that cancer cells utilize to compensate for reactive oxygen species (ROS) and/or activating cellular signaling pathways that lead to immunocytotoxicity. Examples of the anticancer drug include platinum formulation such as cis-platin, carboplatin, and oxaliplatin, thiostrepton, cyclophosphamide, fluorouracil, etoposide, doxorubicin, bleomycin, and mitomycin. The term “reactive oxygen species” relates to highly reactive metabolites of molecular oxygen, which are generated in a tissue environment. ROS can be free radicals, ions or molecules. Examples of ROS include, but are not limited to, superoxide ion radical (O₂), hydroxyl radical (OH), peroxide (ROO), alkoxyl radicals (RO), hydrogen peroxide (H₂O₂), organic peroxide (ROOR′), ozone (O₃), singlet oxygen (¹O₂). Additional compounds having anticancer activity are preferably difluoromethylornithine, erlotinide and thiostrepton.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with an anti-inflammation or anticancer agent), or they may achieve different effects (e.g. control of any adverse effects).

In certain embodiments, the pharmaceutical compositions of the present invention further comprise one or more additional therapeutically active ingredients (e.g. anti-inflammatory and/or palliative). For purposes of the invention, the term “palliative” refers to treatment that is focused on the relief of symptoms of a disease and/or side effects of a therapeutic regimen, but is not curative. For example, palliative treatment encompasses painkillers, antinausea medications and anti-sickness drugs.

### EXAMPLES

The following examples are representative of the invention. Compounds of the examples may be accompanied by a number, which refers to the Compound Number given in the structures and tables throughout this disclosure.

### Materials and Methods

All reagents and solvents were ACS grade. All experiments involving moisture- or air-sensitive compounds were conducted under dry nitrogen. The starting materials and reagents, unless otherwise specified, were of the best grade commercially available (Aldrich, Fluke) and used without further purification. All new products, after purification, showed a single spot on TLC analysis in two different solvent systems. All experiments were performed under atmospheric pressure of 100.3±5 kPa and room temperature unless stated otherwise. The term “room temperature” refers to a temperature of 20±2° C.

### Example 1

### Phosphoric acid diethyl ester 4-[2-(4-isobutyl-phenyl)-propionylamino]-butyl ester (phospho-ibuprofen amide, 1)

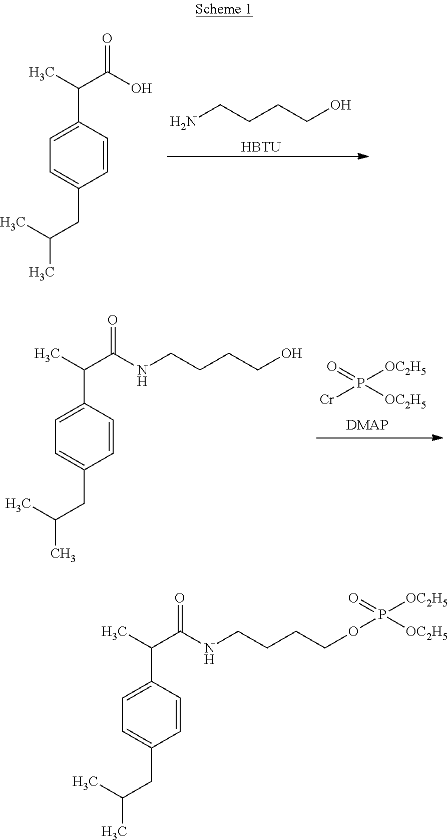

### Step. 1.1 Synthesis of N-4-Hydroxy-butyl)-2-(4-isobutyl-phenyl)-propionamide

Ibuprofen (0.228 g, 1 mmol), 4-amino-1-butanol (0.138 ml, 1.5 mmol) and O-(Benzotriazol-1-yl)-N,N,N′,N′-tetramethyluronium hexafluorophosphate (HBTU) (0.57 g, 1.5 mmol) were dissolved in 5 ml of N,N-dimethylformamide (DMF) containing N,N-diisopropylethylamine (DIPEA) (0.17 ml, 1 mmol). The reaction mixture was stirred at room temperature for 4 hours. The reaction was monitored by thin layer chromatography (TLC). The resulting reaction mixture was dissolved in ethyl acetate, and then washed with 1M HCl, saturated aqueous NaHCO₃ solution, distilled water, brine and dried over sodium sulfate (Na₂SO₄). After the solvent was removed, the crude product was purified by flash column chromatography to give as a white solid in 95% yield.

### Step 1.2 Synthesis of phosphoric acid diethyl ester 4-[2-(4-isobutyl-phenyl)-propionylamino]-butyl ester

Under nitrogen, diethyl chlorophosphate (0.43 g, 1.25 mmol) was added drop-wise to a solution of alcohol (0.299 g, 1 mmol) in dichloromethane (10 ml) containing DIPEA (0.17 ml, 1 mmol), and 4-(dimethylamino)pyridine (DMAP) (6 mg, 0.05 mmol). The reaction mixture was stirred overnight and monitored by TLC. The obtained reaction solution was washed with water (2×25 ml), dried over anhydrous Na₂SO₄, filtered and concentrated. The crude residue was purified by column chromatography using n-hexane:ethyl acetate (60:40) as eluent. The pure fractions were combined and evaporated to give a slightly yellow liquid in 85% yield.

### Biodistribution of Phospho-Ibuprofen Amide (Compound 1)

### Methods

Phospho-ibuprofen amide 1 was formulated in liposomes following the standard protocols described by Mattheolabakis et al. (G. Mattheolabakis, T. Nie, P. P. Constantinides, B. Rigas, Pharm. Res. 2012; 29:1435-43) and administered intravenously to mice as a single 200 mg/kg i.v. dose. After 1 hour, blood and all major organs were collected and drug concentration in the organs was determined following the methods described in T. Nie et al. Br J. Pharmacol. 2012; 166(3):991-1001.

### Results

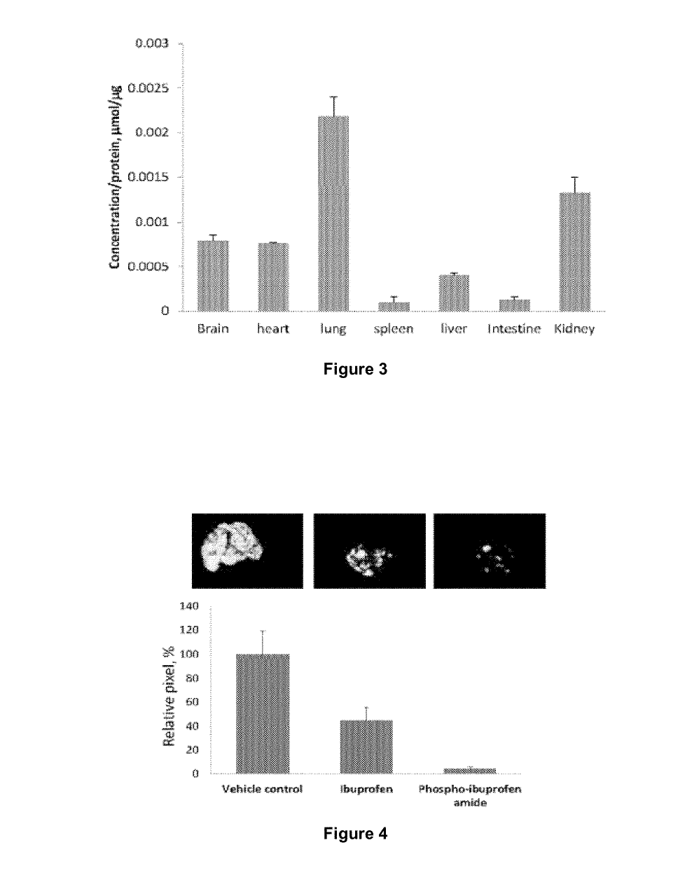

As shown in FIG. 3, liposomal phospho-ibuprofen amide 1 preferentially accumulated in lungs.

### Efficacy of Phospho-Ibuprofen Amide (Compound 1): Inhibition of Lung Cancer

### Methods

Female Ncr nude mice (6-7 weeks old) were injected i.v. (via their tail vein) with 6×10⁶ A549 human non-small lung cancer cells engineered to stably express green fluorescence protein (GFP). These iv injected cells resided preferentially and exclusively in the lungs, i.e. they became lung tumor implants (orthotopic lung tumor model). Three groups (n=6) of such mice were treated with a) liposomal phospho-ibuprofen amide 1 200 mg/kg, or b) ibuprofen 200 mg/kg or c) vehicle (empty liposomes), once a week for 8 weeks. Mouse fluorescence of GFP was monitored using an in vivo imaging system (Maestro, Woburn, Mass.). Relative green fluorescence intensity units (from 7.5×10⁴ to 3.0×10⁵) were used as a marker for tumor initiation in the lungs. Day 0 was designated as initial detection of disease and the day before start of treatment. At the end of the study, animals were sacrificed and their tumors were removed, weighed and imaged.

### Results

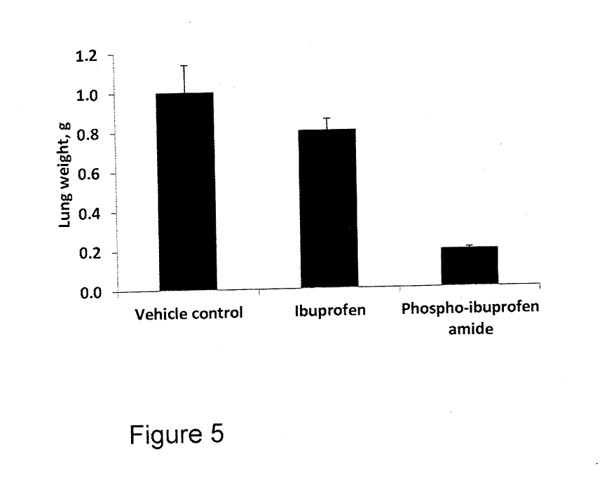

FIG. 4 shows, in addition to representative fluorescence images of lungs from control (left), ibuprofen (center) and phospho-ibuprofen amide 1 (right) treated mice, the amount of lung tumor per group (based on fluorescence intensity). FIG. 5 depicts the lung weight of the same groups of animals. Values (% control) are mean±SEM.

Phospho-ibuprofen amide 1 essentially eliminated lung cancer, reducing it by 95% based on fluorescence and by 80% based on lung tumor weight. In contrast, ibuprofen reduced tumor fluorescence by 57% and lung weight by 19%. The differences between phospho-ibuprofen amide 1 and ibuprofen were statistically significant (p<0.01). These findings underscore the efficacy of the compounds of the invention.

### Example 2

### [1-(4-Chloro-benzoyl)-5-methoxy-2-methyl-1H-indol-3-yl]acetic acid 4-2-(diethoxy-phosphoryloxy)-ethyl]-phenyl ester (phospho-tyrosol-indomethacin) (Compound 93)

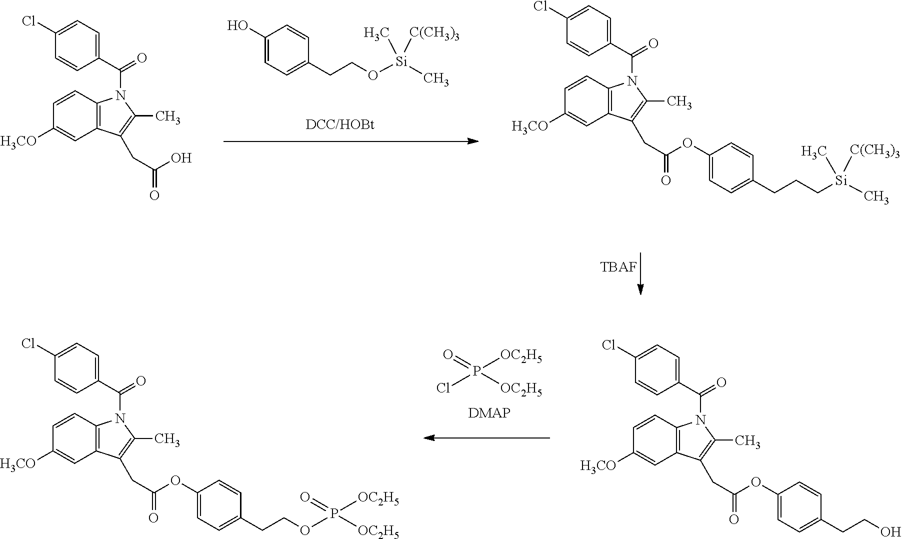

### Step 2.1 Synthesis of 1[1-(4-chloro-benzoyl)-5-methoxy-2-methyl-1H-indol-3-yl]-acetic acid 4-(2-hydroxy-ethyl)-phenyl ester

Under nitrogen atmosphere, indomethacin (1.0 g, 3 mmol), N,N′-dicyclohexylcarbodiimide (DCC) (0.9 g, 3.2 mmol), 1-hydroxybenzotriazole (HOBt) (0.6 g, 3 mmol) and dichloromethane (20 ml) were added to a flask and stirred at room temperature for 1 hour. Then, a solution of the phenol (0.9 g, 3.2 mmol) and DMAP (60 mg) in dichloromethane (10 ml) were added. The resulting solution was stirred at room temperature overnight. The reaction was monitored by TLC. The insoluble solids were removed by filtration and the solvent was evaporated. The remnant was dissolved in ethyl acetate, and then washed with 2% NaHCO₃ solution, distilled water, brine, and dried over Na₂SO₄. After the solvent was removed under reduced pressure, the crude product was purified by flash column chromatography to yield as a pale yellow oil in 90% yield.

### Step 2.2 Synthesis of [1-(4-chloro-benzoyl)-5-methoxy-2-methyl-1H-indol-3-yl]-acetic acid 4-(2-hydroxy-ethyl)-phenyl ester

Compound obtained in step 2.1 above was dissolved in tetrahydrofuran (THF) (40 ml) and reacted with 1 M solution of tetrabutylammonium fluoride (TBAF) in THF (7.2 mmol) and acetic acid (7 ml) at room temperature for 3 hours. Alcohol was obtained as a pale yellow solid in 88% yield. MS: 477 (M+).

### Step 2.3 Synthesis of [1-(4-Chloro-benzoyl)-5-methoxy-2-methyl-1H-indol-3-yl]-acetic acid 4-[2-(diethoxy-phosphoryloxy)-ethyl]-phenyl ester

Diethylchlorophosphate (2.5 ml, 17.26 mmol) was added drop-wise to a solution of alcohol (6.64 mmol) in dichloromethane (10 ml) containing DIPEA (2.2 ml, 13.28 mmol), followed by DMAP (25 mg) as a solid. The reaction mixture was heated under reflux overnight. The reaction solution was washed with water (2×25 ml), dried over anhydrous Na₂SO₄, filtered and concentrated. The crude residue was purified by column chromatography using n-hexane:ethyl acetate (40:60) as eluant. The pure fractions were combined and evaporated to give a viscous yellowish oil in 82% yield. MS: 613.16 (M+).

### Toxicity Assessment of Phospho-Tvrosol-Indomethacin (PTI) (Compound 93)

### Gastrointestinal Toxicity

Gastrointestinal toxicity of phospho-tyrosol-indomethacin (PTI) 93 was determined in rats following a standard protocol (see e.g. Whiteley, P. E. and S. A. Dalrymple, Models of inflammation: measuring gastrointestinal ulceration in the rat. Curr. Protoc. Pharmacol, 2001. Chapter 10: p. Unit 10.2). Six-week-old Sprague-Dawley rats (n=5/group) were administered by gavage vehicle or indomethacin (4.75 mg/kg/day, positive control) or PTI (10 mg/kg/day) for 4 days. On day 5, animals were sacrificed and the stomach was collected. Gastrointestinal toxicity was evaluated by H&E staining of the stomach section. On day 5, 100% of the rats treated with indomethacin developed ulcers compared to 40% of the PTI-treated rats, representing a 60% reduction (p<0.01).

### Cardiotoxicity

Heart tissue sections from mice treated with PTI for about 1.5 months were examined histologically. There were no differences between PTI-treated and healthy control mice.

### Genotoxicity

Genotoxicity of phospho-tyrosol-indomethacin (PTI) was evaluated by measuring the ability of PTI to induce reverse mutations of two strains of Salmonella Typhimurium (TA98 and TA100) in the presence and absence of rat liver S9 activation. These studies showed no genotoxicity.

### Pharmacokinetics

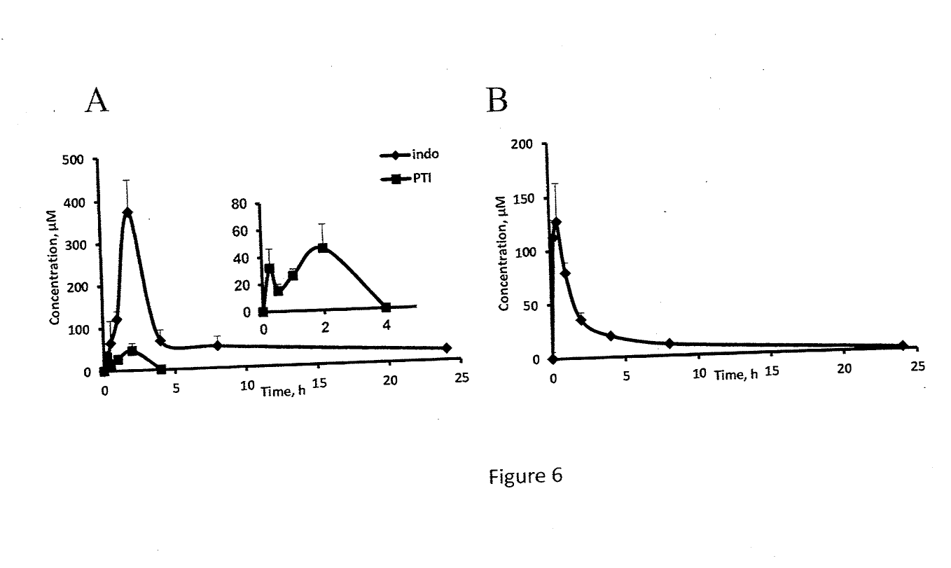

FIG. 6 illustrates a pharmacokinetics study of PTI in mice. Following a single i.p. dose of 100 mg/kg PTI (left) or 58 mg/kg indomethacin (equimolar to PTI) (Indo; right) the plasma levels of intact PTI and indomethacin (hydrolysis product of PTI) were determined at the indicated time points.

Plasma levels of PTI reached the maximum concentration (Cₘₐₓ=46 μM) at 2 hours and became undetectable 4 hours post administration. Its metabolite, indomethacin, reached its Cₘₐₓ=378 μM at 2.5 hours and was minimal at 24 hours. Conventional indomethacin (given alone as above) Cₘₐₓ=127 μM at 1 hour, with its levels becoming negligible at 24 hours. PTI generated a cumulative AUC0.24 h (PTI plus indomethacin) of 1,700 μM×h, while that of indomethacin was 500 μM×h. That PTI delivers to the blood far greater amounts (3.4 fold) of drug than indomethacin (given at the equimolar dose) indicates its superior performance as a drug and explains in part its higher efficacy. This result was totally unexpected.

### Efficacy of PTI (Compound 93) Against Cancer

### Methods

A549 human non-small cell lung cancer cells (1.5×10⁵) were injected subcutaneously in the left and right flanks of 5-6-week-old female NOD SCID mice (Taconic Farms, Germantown, N.Y.). When the average tumor volume reached 100 mm³, mice were treated by oral gavage of 10 or 15 mg/kg/day PTI or vehicle (corn oil) for 2 weeks, when they were sacrificed and the tumors were harvested.

### Results

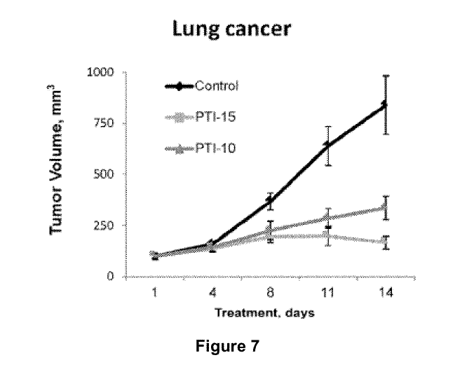

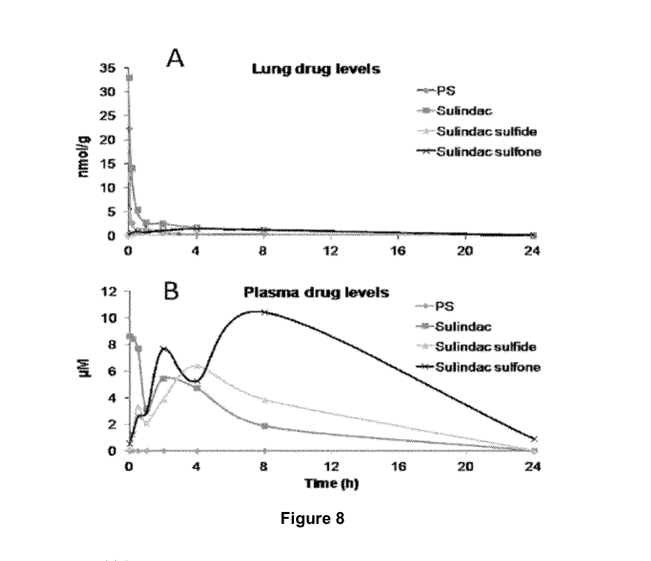

As shown in FIG. 7 (values are Mean±SEM), at the end of the study at 10 mg/kg/day PTI 93 reduced tumor volume by 85%, and at 15 mg/kg/day by 96% (factoring in the 0 time value). These changes are significant (p<0.001, compared to vehicle treated controls).

### Prevention of Colon Cancer:

Six-week-old female athymic nude mice, i.e. lacking a thymus gland and having no T-cells (Taconic Farms, Germantown, N.Y.; in =6/group) were treated by oral gavage of PTI 10 mg/kg/day, or vehicle (corn oil) for a total of 43 days. Five days later, 1.2×10⁶ SW480 colon cancer cells suspended in 100 μl of phosphate buffered saline (PBS) were inoculated subcutaneously to each flank of the mice. Tumor size was monitored by measuring the length (L) and width (W) with a digital caliper and the volume was calculated according to the formula, L×W×(L+W/2)×0.56.

### Results

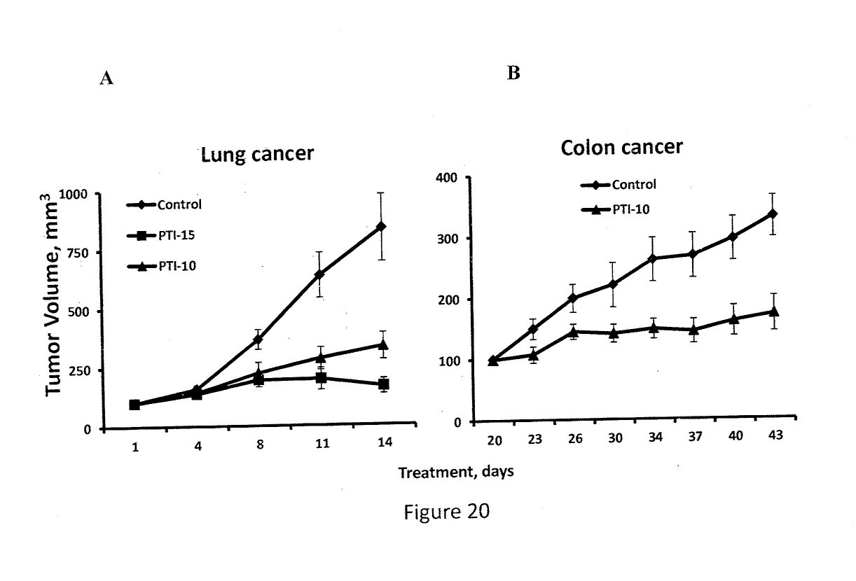

As shown in FIG. 20, Compared to control at the end of the study PTI 10 mg/kg/day reduced tumor growth by 85%.

### Example 3

### Aerosol Delivery of Phospho-Sulindac (PS) (Compound 96) Prevents Non-Small Cell Lung Cancer

Inhalation Exposure System:

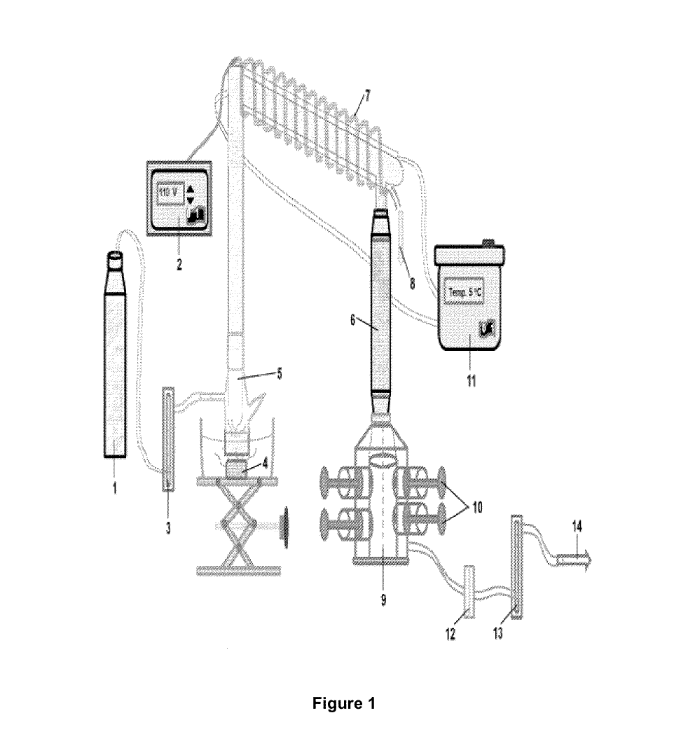

Since PS 96 is a solid powder, if it is to be delivered directly to the lung by inhalation it must undergo aerosolization. The term aerosolization, commonly used in medicine, refers to the process of generating airborne substances suitable for inhalational delivery to the lungs. To accomplish this, the device shown in FIG. 1 was used.

PS dissolved in ethanol was placed in the baffle and aerosolized with the ultrasonic atomizer. The aerosol passed through an ascending stainless steel column, followed by a reflux column which is maintained at a temperature gradient by a heating tape (82° C.) and a chiller (5° C.) to condense and remove ethanol. PS aerosol exiting the reflux column then passed through a charcoal column which served to remove residual traces of ethanol from aerosol before it entered the animal-holding chamber. Experimental animals were held in nose-only air-tight tubes for designated time intervals. The air flow in the system used to deliver by inhalation the test drug to mice was controlled by an inlet air regulator and a vacuum pump which draws air from the system.

Orthotopic Lung Cancer Model:

BALB/c nude mice (7 weeks old) were divided into control and treatment groups (15 mice/group) and treated with aerosol generated from ethanol (control group) or generated from the PS solution (treatment group). Mice received 50 mg/mL PS for 8 minutes. After one week of treatment, 1 million A549 human lung cancer cells stably expressing green fluorescence protein (GFP-A549) were injected into the left lung through a 5 mm incision) was made to the left side of the chest. GFP allows the detection and quantification of these cells. (See Doki et al. Br. J. Cancer, 79, 7-8, pages 1121-1126, 1999). Inhalation treatment, suspended for 2 days post-surgery, continued for 6 weeks. At the end of the treatment period, mice were euthanized, and blood and lung tissues were collected. Luminosity of the GFP-A549 tumors was measured and the lungs were weighed.

Results

Animal survival and tumor size were used to gauge the efficacy, of PS.

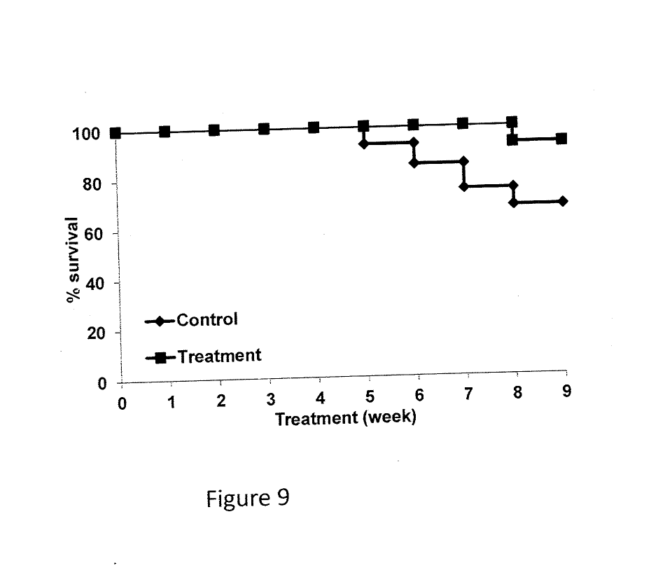

a) Survival: By the end of the study, 40% of the mice in the control group died from the disease while the death rate in the treatment group was less than 10% (p<0.03). The results are illustrated by FIG. 9.

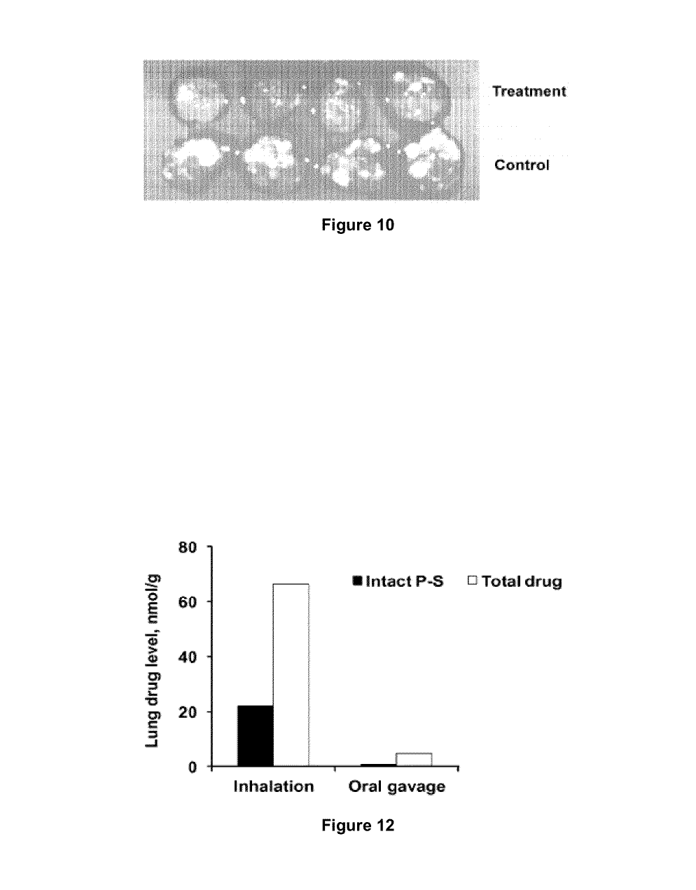

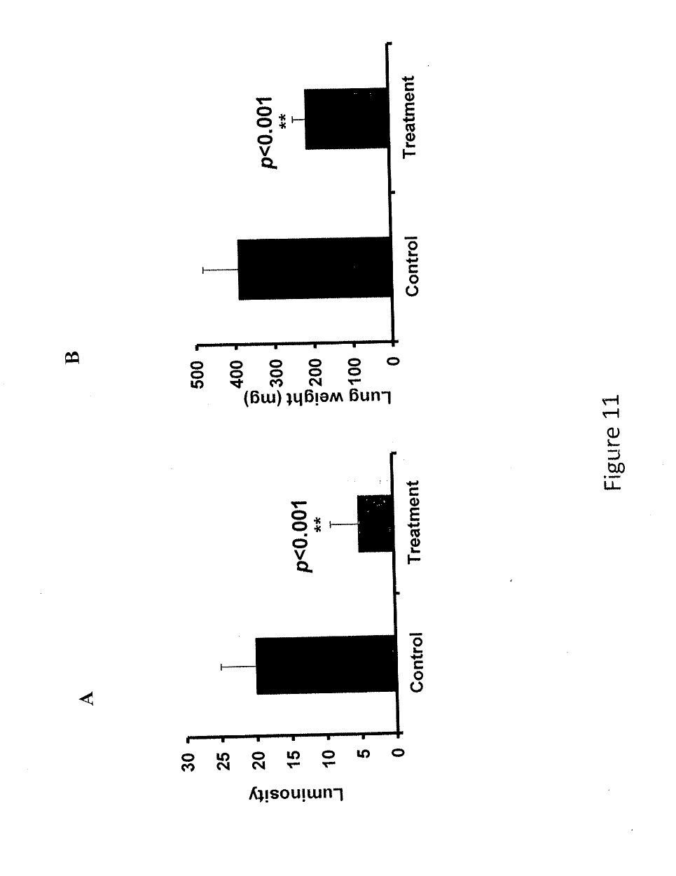

b) Tumor size: At sacrifice, the tumor size was (all values, Mean±SEM) determined a) by luminosity: control=19.85±4.33, treatment=5.05±2.97 (p<0.001). The results are shown in FIG. 10 (upper photograph: after treatment; lower photograph: control group) and FIG. 11 (left), and b) by lung weight: control=385.7±85.2 mg, treatment=204.4±39.4 mg (p<0.001). The results are shown in FIG. 11 (right).

These findings establish the strong chemopreventive efficacy of PS 96 against lung cancer.

### Example 4

### The Pharmacokinetic Parameters of PS (Compound 96) after Inhalational Administration

PS 96 was administered to BALB/c nude mice by inhalation as above for 8 minutes. Mice were euthanized at various time points and drug levels were analyzed by HPLC in plasma and lung tissues. The results are summarized in the two tables below and are graphically illustrated in FIG. 8.

TABLE 1

| | Pharmacokinetic parameters in lung | | |
|---|---|---|---|
| | AUC | C [symbol] nmol/g | Tₘₐₓ, h |
| PS | 7.7 | 22.2 | 0 |
| Sulindac | 30.1 | 32.9 | 0 |
| Sulindac sulfide | 18.9 | 1.4 | 4 |
| Sulindac sulfone | 57.5 | 4.8 | 8 |

[symbol]

indicates data missing or illegible when filed

TABLE 2

| | Pharmacokinetic parameters in plasma | | |
|---|---|---|---|
| | AUC | Cₘₐₓ, μM | Tₘₐₓ, h |
| PS | 0 | 0 | — |
| Sulindac | 49.5 | 6.6 | 0 |
| Sulindac sulfide | 66.9 | 6.4 | 4 |
| Sulindac sulfone | 142.4 | 10.4 | 8 |

These findings indicate the following: a) inhalation provides intact PS 96 to the lungs, which is more cytotoxic to human cancer cells in the lung than either of its three metabolites, sulindac, sulindac sulfide and sulindac sulfone; and b) there are sufficient anti-cancer concentrations of sulindac, sulindac sulfide and sulindac sulfone in the circulation, as metabolites of inhaled PS, and for prolonged periods of time (little or no PS reaches plasma circulation). Sulindac, sulindac sulfide and sulindac sulfone are established cancer chemopreventive agents and thus, when derived from inhaled PS, they can prevent smoking/nicotine-related cancers at the lung and at sites other than the lung, e.g., in the colon, lung and others (J Natl Cancer Inst (2002); 94 (4): 252-266.)

### Example 5

### Inhalation Delivery of Aerosolized PS (Compound 96) to the Lungs of Mice Leads to Higher Drug Levels than Oral Administration

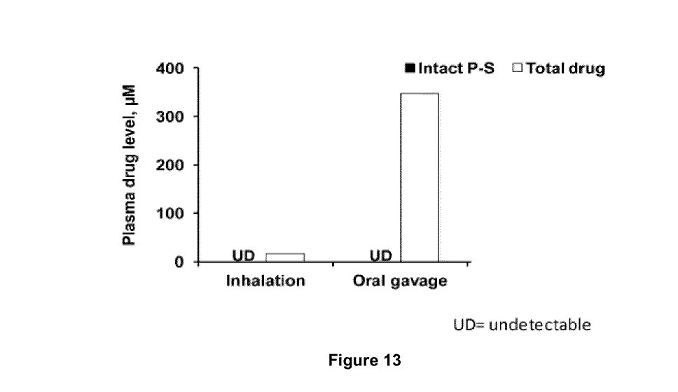

The delivery of aerosolized phospho-sulindac (PS) 96 to the lungs of mice was compared to that following its oral delivery using the same inhalation device as in Example 3. The PS doses were: inhalational=6.5 mg/kg body weight; oral=150 mg/kg body weight. The level of PS in the lungs and plasma after inhalation vs. after oral gavage are shown in FIGS. 12 and 13, respectively.

Lungs: PS Levels:

The aerosol-exposure system delivered a high level of intact PS to the lungs of mice (>20 nmol/g); while there were only trace levels of intact PS (<2 nmol/g) by oral administration.

Total Drug Levels:

It represents the total level of PS plus its metabolites. The main metabolites of PS are sulindac, sulindac sulfide and sulindac sulfone; at least the first two can cause gastrointestinal and renal side effects. The levels achieved by inhalation were significantly higher compared to those by oral administration.

Plasma: PS Levels: Undetectable.

Total drug level after inhalation treatment (17 μM) was lower than that after oral (348 μM) administration. Thus, inhalation delivery leads to blood levels of sulindac that can be chemopreventive for various non-lung cancers, but which are not so high as to have significant potential toxicity. Of the three main metabolites of PS, at least sulindac and sulindac sulfide can cause gastrointestinal and renal side effects. Thus, PS can be effectively delivered to lung cells by inhalation. It is also clear that aerosolized PS can be delivered to the lungs concurrently with tobacco smoke the main lung carcinogen.

### Example 6

### Inhibition of the Growth of Glioblastoma Cell Lines

The 24-hour growth inhibitory concentration (24-h IC₅₀) was determined for sulindac, ibuprofen phospho-sulindac 96, phospho-ibuprofen 2, Phospho-ibuprofen glycerol 3 and phospho-ibuprofen glycerol amide 4 in the U87 glioblastoma cell line (formerly U-87 MG), as specified by Huang et al. (Huang L, Mackenzie G G, Sun Y, Ouyang N, Xie G, Vrankova K, et al. Cancer Res. 2011; 71: pp. 7617-27).

The results, summarized in Table 3, indicate that the compounds of the present invention inhibited the growth of the U87 glioblastoma cells much more potently than the conventional NSAIDs sulindac and ibuprofen.

TABLE 3

| | 24-h 1c₅₀, μM |
|---|---|
| Sulindac | ≧1000 |
| Ibuprofen | ≧1000 |
| Phospho-sulindac, 96 | 114 |
| Phospho-ibuprofen 2 | 98 |
| Phospho-ibuprofen glycerol 3 | 105 |
| Phospho-ibuprofen glycerol amide 4 | 87 |

### Example 7

### Phosphovalproic Acid (PV) (Compound 116) and Phospho-Ibuprofen Gylcerol Amide (PGIA) (Compound 4) Synergize Strongly to Inhibit the Growth of Glioblastoma and Lung Cancer

### Methods

The potential synergy between PV and PGIA in inhibiting the growth of U87 glioblastoma cells was assessed by isobolographic analysis. After treatment with PV 116 or PGIA 4 alone or in combination for 24 hours, the following as determined: a) cell growth using the MTT assay (Promega, Madison, Wis.) and b) apoptosis by staining with Annexin V-FITC (Invitrogen) and propidium iodide 0.5 μg/ml and analyzing the fluorescence intensity by FACS caliber.

### Results

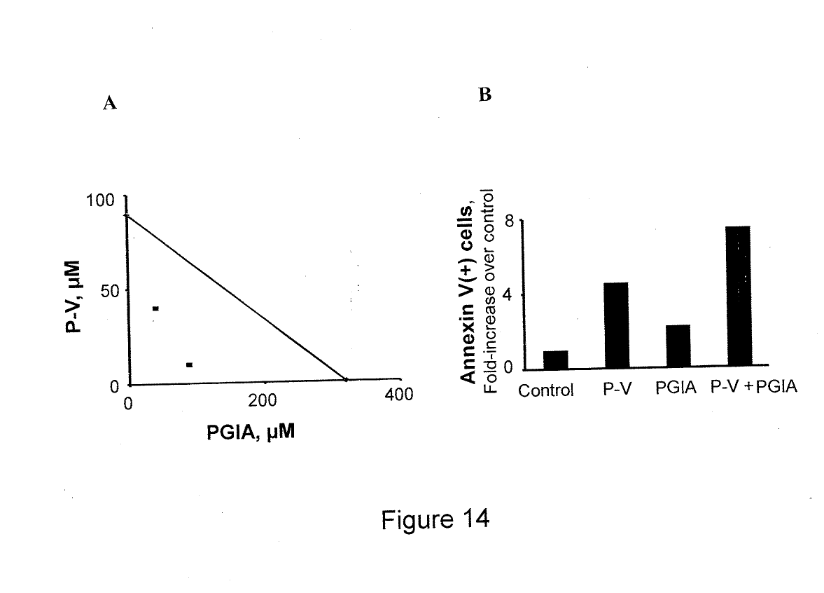

There is a clear-cut pharmacological synergy between PV 116 and PGIA 4 in inhibiting the growth of U87 glioblastoma cells (FIG. 14, left panel), and in the induction of their apoptosis (right panel). For example, after 24 hours of incubation with PGIA 4 200 μM and PV 116 40 μM, the fold-increase of annexinV (+) cells was 8.0, compared to 3.0 for PV 116 40 μM alone and 1.8 for PGIA 4 200 μM (FIG. 14, right panel).

Synergy between compounds 116 and 4 regarding cell growth inhibition and induction of apoptosis was obtained in the glioblastoma cell lines U118, LN-18 and LN-229; and in the A549 lung cancer and MIA PaCa-2 pancreatic cancer cell lines.

### Example 8

### The Cellular Uptake of Ibuprofen, Phospho-Ibuprofen 2 and Phospho-Ibuprofen Phosphate Test Compounds

Ibuprofen, phospho-ibuprofen 2, and phospho-ibuprofen phosphate 505. Their structures are shown below.

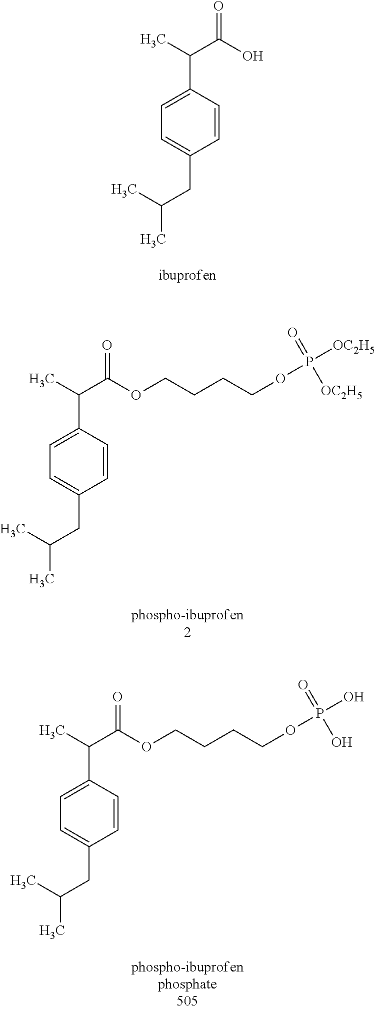 [SMILES: CC(C)Cc1ccc(C(C)C(=O)O)cc1 ; CCOP(=O)(OCC)OCCCCOC(=O)C(C)c1ccc(CC(C)C)cc1 ; CC(C)Cc1ccc(C(C)C(=O)OCCCCOP(=O)(O)O)cc1]

### Methods

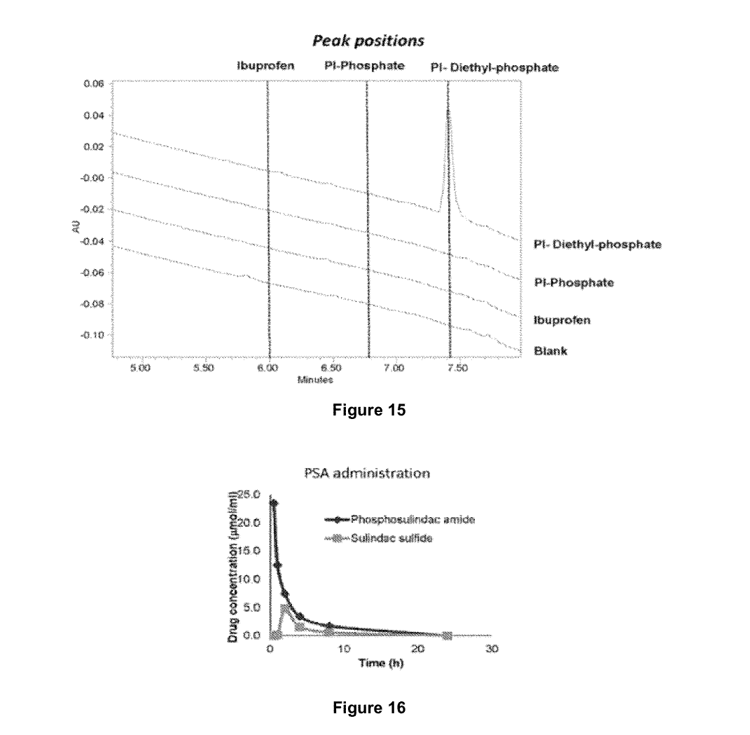

A431 skin cancer cells were seeded into 6-well culture plates (5×10⁵ per well). After overnight incubation, the cells were incubated with 100 pM of each compound alone for 1 hour. After the media were removed and the monolayers were washed three times with PBS (1% BSA), the cells were collected in PBS. Intracellular drugs were extracted with acetonitrile and their levels were determined by HPLC analysis (FIG. 15). The compounds evaluated have equivalent molar absorptivity.

### Results

As shown in FIG. 15, there was no significant accumulation in A431 cells of either ibuprofen or phospho-ibuprofen phosphate 505 after 1 hour incubation (limit of detection: 2.5 pmol). In contrast, phospho-ibuprofen 2 (it has diethylphosphate, not phosphate in its structure) was present at 750 pM inside the cells, representing at least a 300-fold increase over the other two compounds.

The first HPLC chromatogram illustrates that after 1 hour of incubation a significant amount of phospho-ibuprofen 2 (retention time: 7.43 minutes) was accumulated in the cells. Importantly, neither ibuprofen (retention time: 6.00 minutes) nor phospho-ibuprofen-phosphate 505 (retention time: 6.78 minutes), which could potentially result from the intracellular hydrolysis of phospho-ibuprofen-diethyl phosphate, were detected in the cellular extract.

Thus, phosphor-ibuprofen 2 is taken up by human cells A431 to a significantly higher extent compared to phospho-ibuprofen-phosphate 505 or ibuprofen.

### Example 9

### Phosphoric acid diethyl ester 4-(2-[6-fluoro-3-(4-methanesulfinyl-benzylidene)-2-methyl-3H-inden-1-yl]-acetylamino}-butyl ester (phosphosulindac amide, Compound 95)

Phosphosulindac amide 95 was synthesized according to procedure shown in the scheme

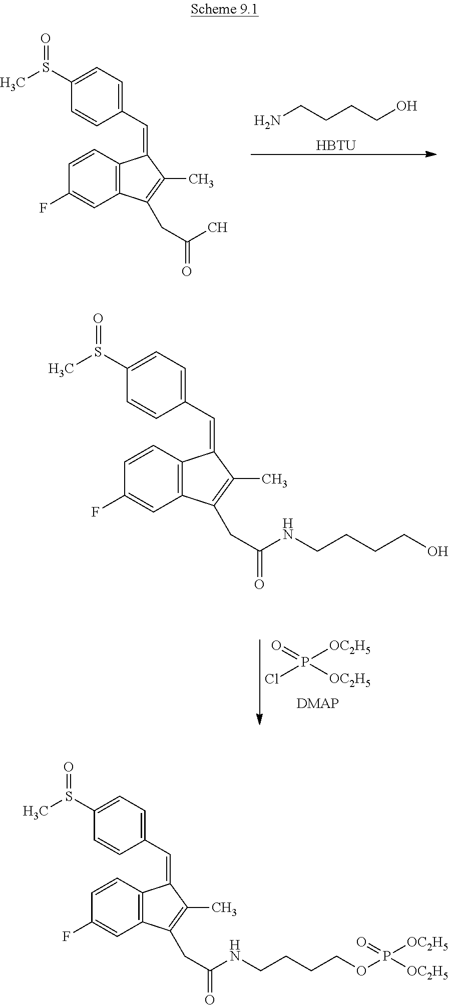

### Step 9.1 Synthesis of 246-fluoro-3-(4-methanesulfinyl-benzylidene)-2-methyl-3H-inden-1-yl]-N-(4-hydroxy-butyl)-acetamide

Sulindac (0.356 g, 1 mmol), 4-amino-1-butanol (0.138 ml, 1.5 mmol) and HBTU (0.57 g, 1.5 mmol) were dissolved in 5 ml of DMF further containing DIPEA (0.17 ml, 1 mmol). The reaction mixture was stirred at room temperature for 4 hours. The reaction was monitored by TLC. The remnant was dissolved in ethyl acetate, and then washed with 1 M HCl, saturated NaHCO₃ solution, distilled water, brine, and dried over Na₂SO₄. After the solvent was removed under reduced pressure, the crude product was purified by flash column chromatography to give as a white solid in 95% yield.

### Step 9.2 Synthesis of phosphoric acid diethyl ester 4-[2-{6-fluoro-3-(4-methanesulfinyl-benzylidene)-2-methyl-3H-inden-1-yl]-acetylamino]-butyl ester

Under nitrogen, diethyl chlorophosphate (0.43 g, 1.25 mmol) was added drop-wise to a solution of alcohol (0.427 g, 1 mmol) in dichloromethane (10 ml) containing DIPEA (0.17 ml, 1 mmol), and DMAP (6 mg, 0.05 mmol). The reaction mixture was stirred overnight and monitored by TLC. The reaction solution was washed with water (2×25 ml), dried over anhydrous Na₂SO₄, filtered and concentrated. The crude residue was purified by column chromatography using ethanol:ethyl acetate (10:90) as eluant. The pure fractions were combined and evaporated to give a slightly yellow liquid in 85% yield.

### Pharmacokinetic Studies of Phosphosulindac Amide (Compound 95) in Mice

### Methods

Mice were administered a single oral dose of 100 mg/kg of phosphosulindac amide 95 (PSA). Mice (2-3 per time point) were sacrificed at designated time points when blood was collected, centrifuged immediately and the resulting plasma was deproteinized by immediately mixing it with a 2-fold volume of acetonitrile. PSA 95 and its metabolites were analyzed by HPLC as described by Xie et al. (Xie G, Nie T, Mackenzie G, Sun Y, Huang L, Ouyang N, et al. The metabolism and pharmacokinetics of phosphosulindac (OXT-328) and the effect of difluoromethylornithine. Br. J. Pharmacol. 2011).

### Results

As shown in FIG. 16, PSA 95 was detected in serum for several hours; with the following parameters Cₘₐₓ=24 μM; and Tₘₐₓ=15 minutes; and AUC₀₋₂₄ₕ=54.04 μM×h. A single metabolite of 95, sulindac sulfide was detected with AUC₀₋₂₄ₕ=17.86 μM×h. Surprisingly, PSA 95 generated no detectable sulindac or sulindac sulfone.

### Compound Efficacy×Inhibition of Colon Cancer

### Methods

### Efficacy in Xenografts

Female Ncr nude mice (5-6 weeks old; Harlan, Taconic Farms, Germantown, N.Y.) were inoculated subcutaneously in their right and left flanks, each with 2.0×10⁶ SW-480 colon cancer cells suspended in 100 pl of PBS. When the average tumor size reached 100 mm³, the animals were divided into two groups of 6 and treated orally for 3 weeks either with vehicle (PBS) or PSA (Compound 95), 100 mg/kg/d in PBS. Tumors were measured twice a week with a digital microcaliper, and tumor volumes were calculated (tumor volume=[length×width×(length+width/2)×0.56]). At the end of the study, animals were sacrificed and their tumors were removed and weighed.

Efficacy in Apc^{Min/+} mice

Eleven week old male C57BL/6J Apc^{Min/+} (n=6/group) were treated for 6 weeks with PSA 95 100 mg/kg/d or vehicle (corn oil) given by oral gavage. At the end of the study, animals were sacrificed and their small intestine and colon were removed, opened longitudinally and all tumors counted under a magnifying lens.

### Results

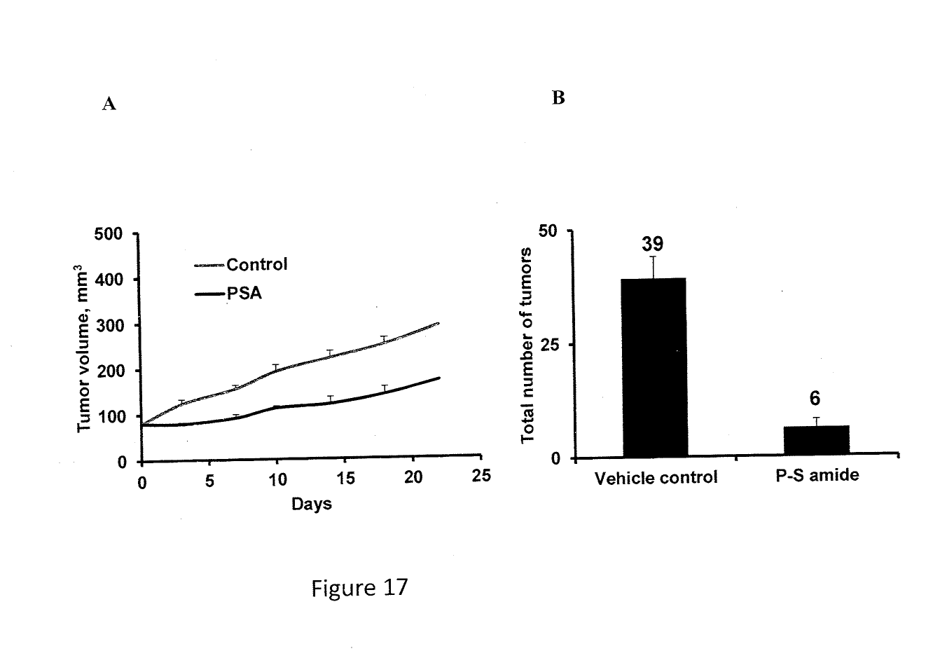

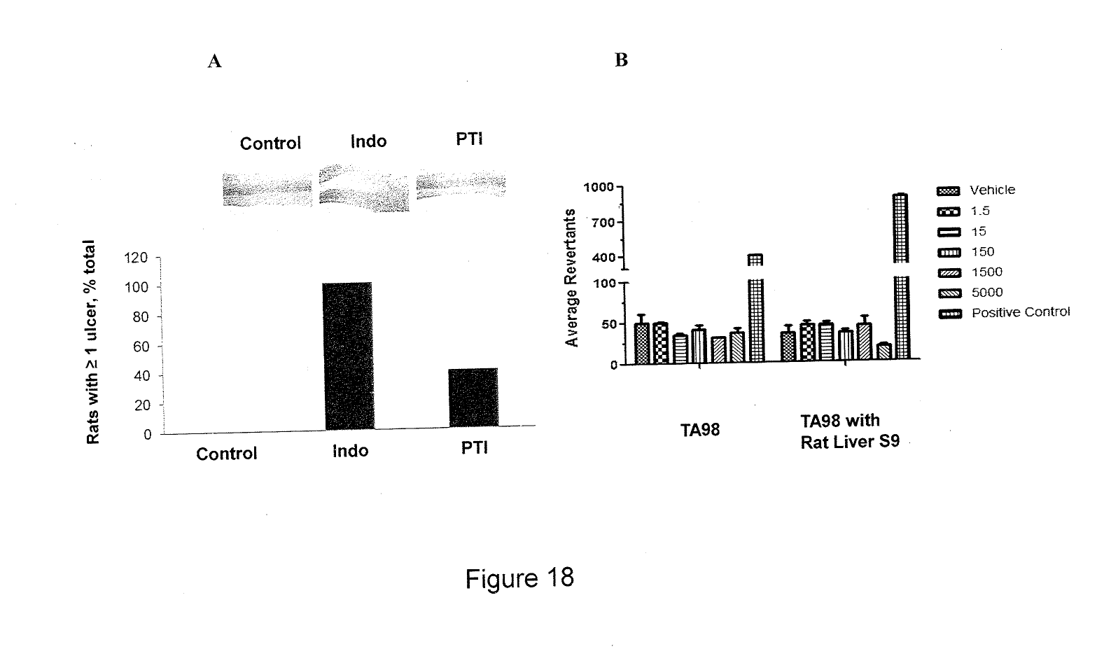

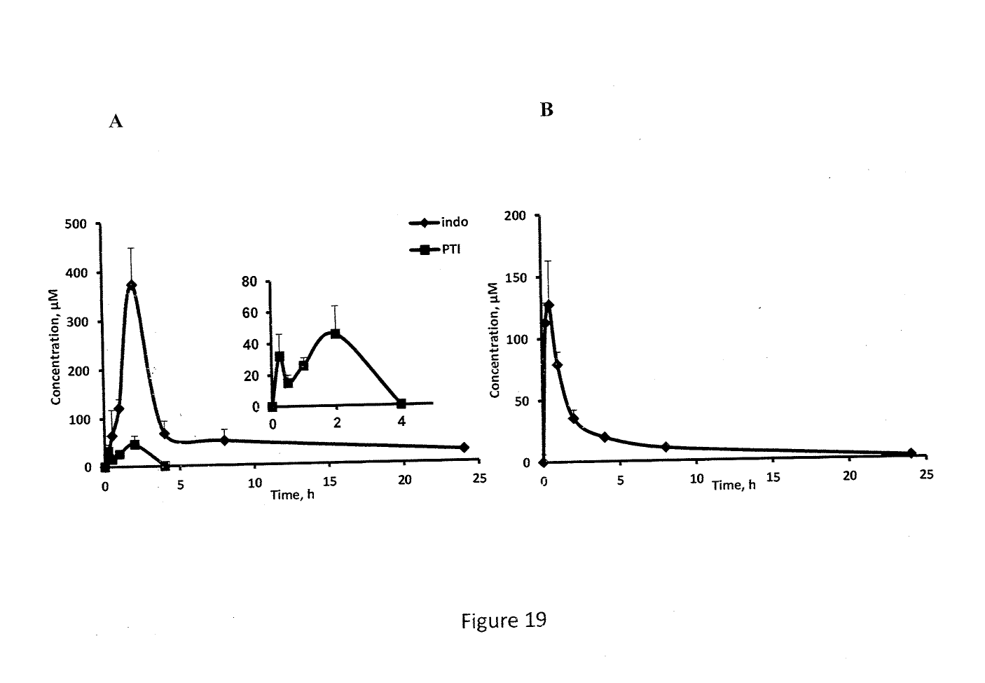

As shown in FIG. 17: (a) compared to control, at the end of the study PSA 95 100 mg/kg/day reduced the growth of colon cancer xenografts by 41% (p<0.02); and (b) in the Apc^{Min/+} mouse model, PSA 95 reduced the number of all intestinal tumors by 85%, compared to the control group (p<0.001).

### Example 10

### PEGylated Phospho-Ibuprofen (Compound 7)

PEGylated phosphor-ibuprofen 7 was prepared using a modified methodology of H-phosphonate synthesis according to Trirosh et al. (Tirosh, Kohen R, Katzhendler J, Gorodetsky R, Barenholz Y., Novel synthetic phospholipid protects lipid bilayers against oxidation damage: Role of hydration layer and bound water. J. Chem. Soc. Perkin Trans. 2. 1997:383-9). Accordingly, the title compound was synthesized as shown in the Scheme below.

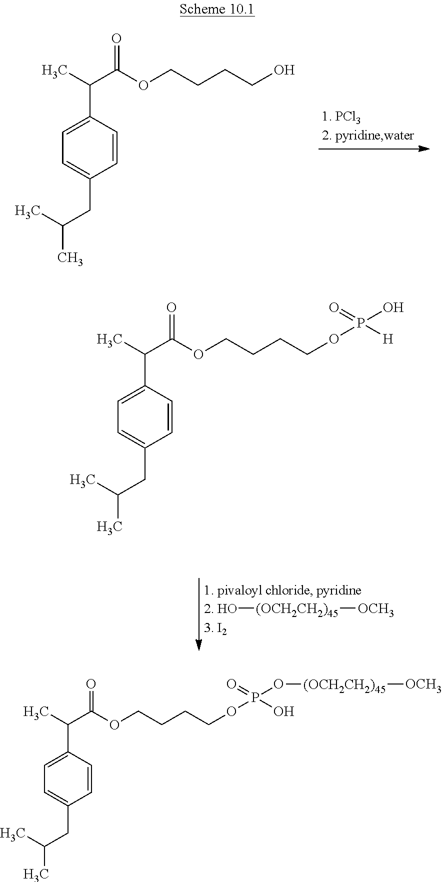

### Step 10.1 Synthesis of the H-phosphonate

A stirred solution of phosphorus trichloride in dichloromethane was prepared and a solution of ibuprofen-butanol was added in equimolar amounts. The stirring was continued for 30 minutes until the mixture was quenched by the addition of 100 ml of water-pyridine (1:4 v/v). After 15 min, the compound was extracted with chloroform from the reaction mixture, washed twice with water and dried using Na₂SO₄. The organic solvent was removed by rotary evaporation.

### Step 10.2 Synthesis of PEGylated phospho-ibuprofen (Compound 7)

The residue obtained in step 10.1 above was dissolved in 50 ml of dichloromethane. Lyophilized mPEG, pivaloyl chloride and pyridine were added to the reaction and the solution was stirred for 10 minutes followed by removal of the organic solvent by rotary evaporation. A solution consisting of water-pyridine (1:1 v/v) was added to oxidize the H-phosphonate. The oxidation was stopped by adding 100 ml of 5% aqueous sodium thiosulfate solution. The final product, PI-PEG, was extracted from the aqueous medium with chloroform, which was then washed with water and brine, dried over magnesium sulfate and finally evaporated under reduced pressure. The solid residue was purified by acetone precipitation.

The isolated PI-PEG was characterized by ¹H-NMR and its purity was confirmed by both HPLC and ¹H-NMR.

### Animal Studies

Mice were treated with various amounts of PI-PEG 7 by oral, i.p. and i.v. administration. The maximum dosage used for i.v. treatment was 1600 mg/kg and for i.p. and oral treatment 4000 mg/kg. In all cases, PI-PEG 7 was dissolved in phosphate buffered saline pH 7.4 (PBS). No signs of toxicity, discomfort or changes in the normal mouse behavior were observed.

### Pharmacokinetics in Mice

PI-PEG 7 and PI 1 were injected i.p. in mice at equimolar doses and at predetermined time points the animals were sacrificed and blood was collected through heart puncture. PI-PEG and PI were extracted by adding a 2-fold volume of acetonitrile. After centrifugation for 10 minutes at 5000×g, the supernatants were subjected to HPLC analysis. PI-PEG 7 exhibited prolonged stability and improved circulation times compared to PI as shown in FIG. 10, while PI was rapidly hydrolyzed to its metabolite, ibuprofen, whose levels are not shown in FIG. 21. This demonstrates the superiority of PI-PEG 7 over PI 1, and the potential superiority of pegylated compounds of the invention over corresponding non-pegylated compounds of the invention, particularly the carboxylic acid esters of the invention.

### Anticancer Efficacy Studies

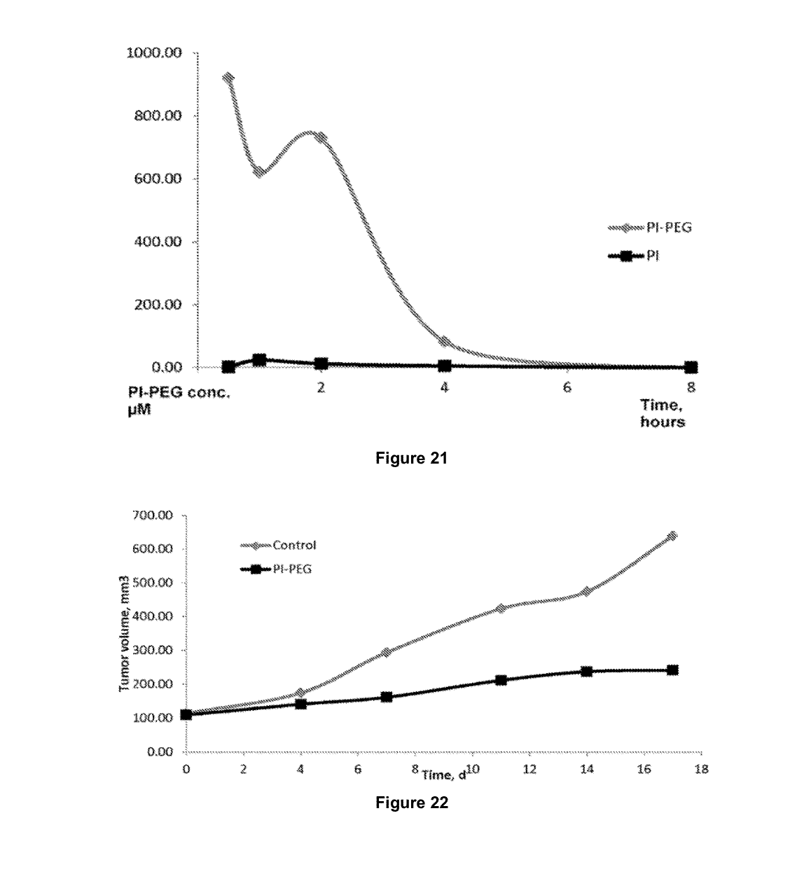

A tumor growth mouse model was used to assess the potential anticancer efficacy of PI-PEG 7. Human colon cancer SW-480 xenografts in nude mice were treated with daily ip injection of PI-PEG 7 4,000 mg/kg in PBS. FIG. 22 shows a 72% tumor growth inhibition after 18 days of treatment compared to controls (p<0.01).

Additionally; Apc^{Min} mice were used, a mouse model of colon cancer (Lipkin M, Yang K, Edelmann W, Xue L, Fan K, Risio M, et al. Preclinical mouse models for cancer chemoprevention studies. Annals of the New York Academy of Sciences. 1999; 889:14-9), to determine the efficacy of PI-PEG 7 in tumor prevention. Apc^{Min} mice were given 2400 mg/kg of PI-PEG 7 orally once a day, 5 times per week for 10 weeks. At the end of the 10th week, PI-PEG 7 reduced the number of tumors on the gastrointestinal track of these mice by 80% compared to the phosphate buffered saline (PBS) control group (n=8 mice/group). This effect was even pronounced in the tumors of the colon (93% reduction); note that Apc^{Min} mice grow tumors in both the small intestine (predominantly) and the colon. Of interest, PEG alone administered at an equimolar dose to a third group of Apc^{Min} mice (n=8) had no effect on their number of tumors.

### Example 11

### Analgesic Effects of Phosphosulindac (PS) (Compound 96), Phospho-Ibuprofen (PI) (Compound 2), PI-PEG (Compound 7) and PI Amide (Compound 1)

The analgesic effect of each of phosphosulindac 96, PI 2, PI-PEG 7 and PI amide 1 was determined in mice by measuring their antinociceptive effect to an acute thermal stimulus. A hot-plate test was employed, according to a standard protocol by Bannon A W (Bannon A W. Models of Pain: Hot-plate and formalin test in rodents. Current Protocols in Pharmacology: John Wiley & Sons, 1998).

### Methods

Tested compounds: PS 96, PI 2, PI amide 1, each at 100 mg/kg and PI-PEG 7 1,600 mg/kg.

Animals: Male CD mice (Charles River Labs), 25-30 g, divided into 5 study groups (n=5-8).

Testing: After 30 minutes of acclimation to the test room environment, baseline measurements were performed, mice were administered a single intraperitoneal dose of each test compound or vehicle (control). Thirty minutes post dosing, each animal was placed on a 55° C. hot plate and the latency to respond was recorded, i.e. the time until the animal shows a nociceptive response.

### Results

The following latency values were obtained after 30 minutes (seconds; mean±SD)

| | | |
|---|---|---|
| Control | 1.91 ± 0.46 | |
| PI amide 1 | 3.80 ± 0.46 | p < 0.05 |
| PI 2 | 8.53 ± 0.81 | p < 0.001 |
| PI-PEG 7 | 5.21 ± 0.74 | p < 0.01 |
| PS 96 | 4.55 ± 1.00 | p < 0.01 |

Note:
p values refer to the comparison to control.
It can be concluded that all compounds tested had a significant analgesic effect.

### Example 12

### Anti-Cancer Activity and Pharmacokinetics of Phospho-Aspirin III (PA-111, Compound 506) and Phospho-Aspirin IV (PA-IV, Compound 507)

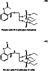

The ability of phospho-aspirin III (PA-III) 506 to prevent breast cancer (BC) was evaluated using MDA-MB-231 human BC cells xenografted into one of the mammary glands of nude mice (orthotopic xenografts). PA was administered orally, 120 mg/kg/d 1 week prior to inoculating the cells (the standard prevention protocol); acetylsalicylic acid (ASA) was given at an equimolar dose of 40 mg/kg/d. This was the highest dose of ASA that these mice could tolerate on a long-term basis. The dose of PA-III 506 represents <10% of its maximum tolerated dose (MTD>1,600 mg/kg), but was chosen so that a comparison between PA-III 506 and ASA was possible.

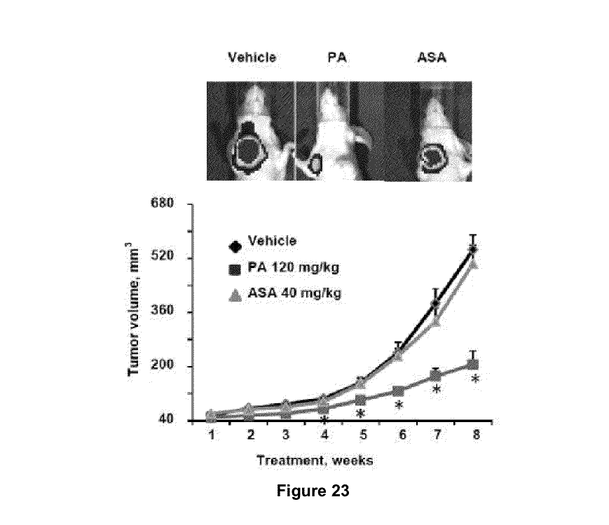

FIG. 23 illustrates the growth of orthotopic MDA-MB231 xenografts treated with PA-III 506 or ASA, starting 1 week prior to cell implantation. Cells were stably transferred with luciferase allowing imaging of the xenografts (upper images). Lower diagram: tumor volume, mm³. Volume calculations were based on luminescence and caliper measurements that agreed closely. *, p<0.001-05.

PA-III 506 displayed a strong chemoprevention effect (FIG. 1). Of the 20 treated mice: 20% had no tumors; 30% had tumors <100 mm³, while the smallest tumor in controls was 326 mm³ and the average tumor volume was reduced by 62% compared to controls. In sharp contrast, ASA showed no effect on BC xenograft growth.

The levels of PA-III 506 and some of its metabolites in blood and xenografts from these mice were determined by HPLC according to the methods described by G. Xie et al. (Br J. Pharmacol. 2012; 167(1):222-32).

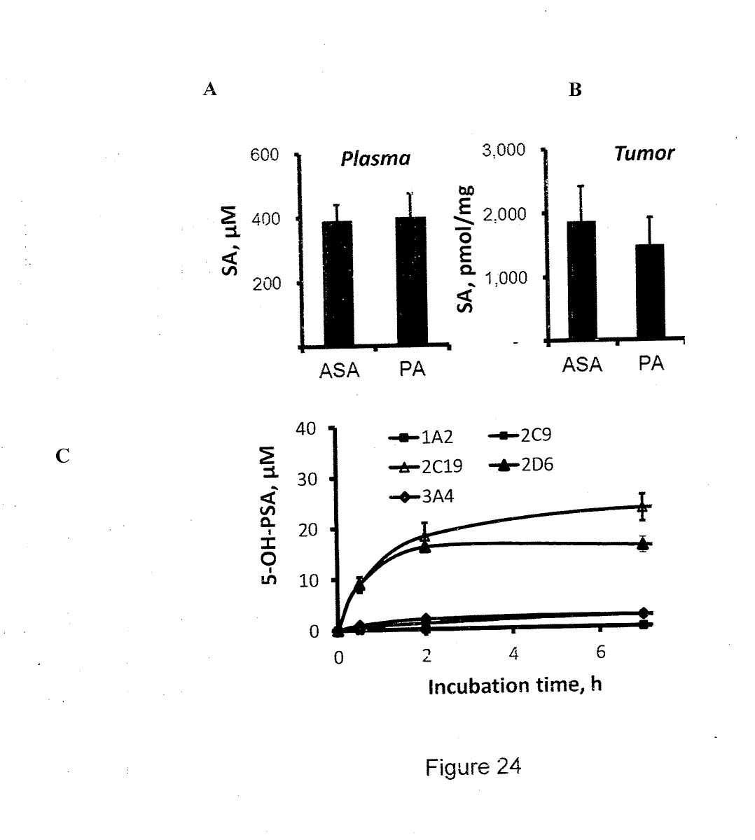

FIG. 24 shows the measured levels of PA-III 506 metabolites in plasma and tumors; and the effect of CYP isoforms. Upper panels: Only salicylic acid (SA) was detected in the plasma and xenograft tumors of mice treated with PA-III 506 or ASA (study shown in FIG. 23). Differences in SA levels were not significant. Lower panel: In vitro metabolism of PSA 95 by human CYPs. PSA 95 was incubated with each of the indicated CYP isoforms and its rate of conversion to 5-OH-PSA was monitored. Only 2C19 and 2D6 showed appreciable activity.

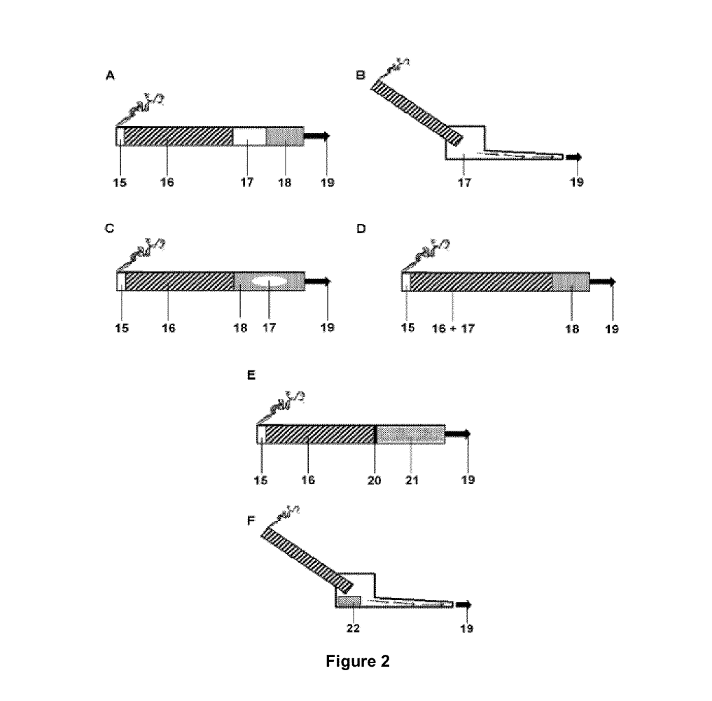

No intact PA-III 506 in either plasma or xenografts was detected; salicylic acid (SA) being the only measurable metabolite (FIG. 2). Moreover, there was no difference in SA levels between the two groups. These results were an unexpected finding based on what is conventionally understood regarding drug action. Thus additional agents may be involved in the differential action of these two compounds. Work on identifying these agents is included below, as described in Example 13 (metabolism of PA-III 506).

### Example 13

### Formation of Phospho-Aspirin III (PA-III) (Compound 506) Metabolites

Using standard approaches, included human and animal liver microsomes, cultured breast cancer cells and mice, as well as established analytical methods, the metabolism of PA-III 506 was investigated. Results are summarized in the Scheme below (Xie G, Wong C C, Cheng K W, Huang L, Constantinides P P, Rigas B. In Vitro and In Vivo Metabolic Studies of Phospho-aspirin (MDC-22). Pharm Res. 2012, in press).

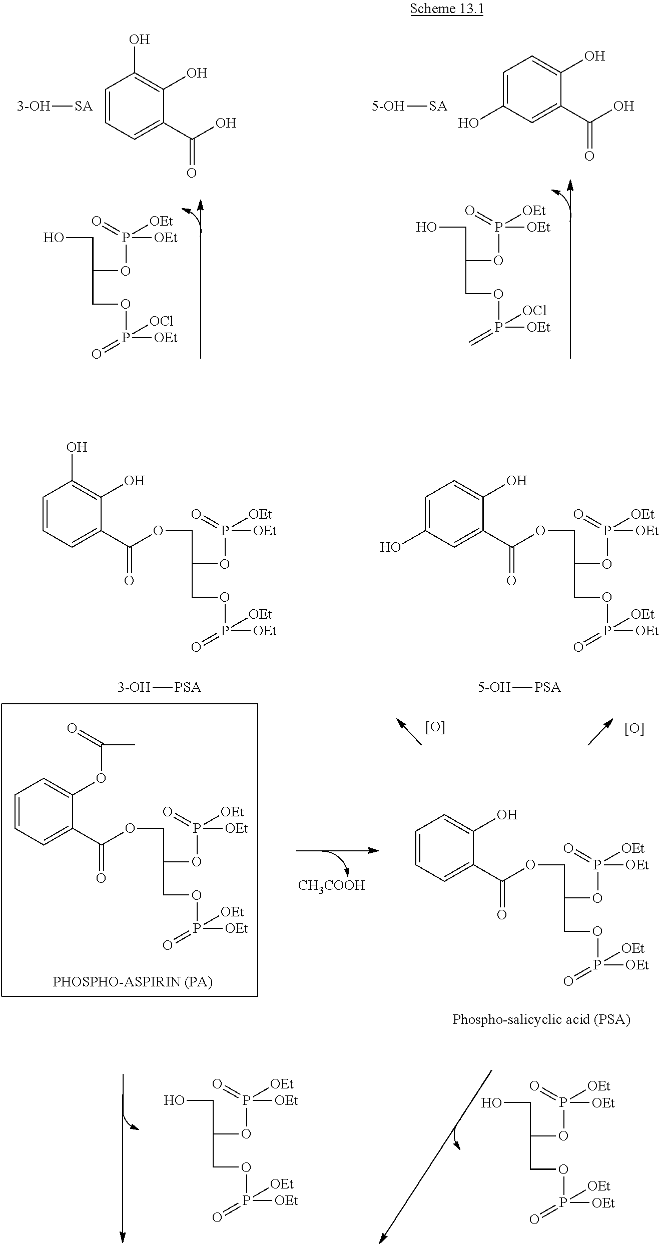

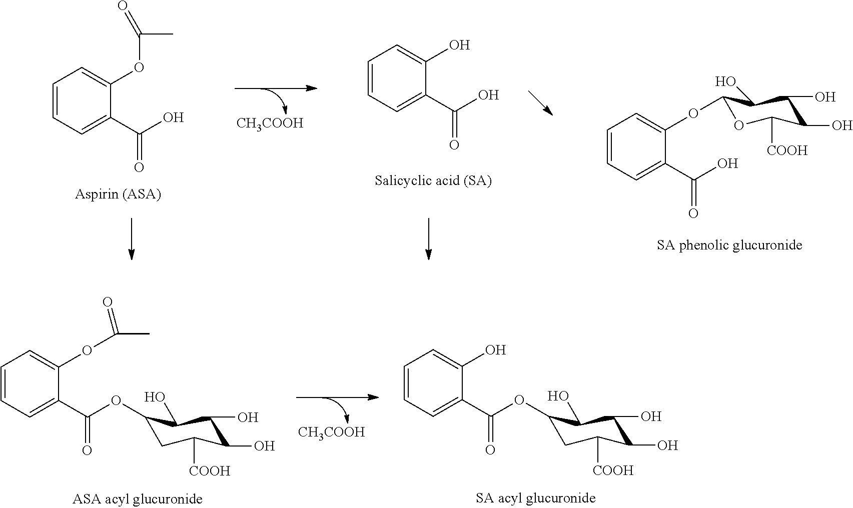

Subsequently, the metabolism of PSA 95 shown in scheme 13.1 by human CYP isoforms was further investigated.

Method:

PSA 95 was pre-incubated at 37° C. for 5 minutes with an NADPH-regenerating solution in 0.1 M potassium phosphate buffer (pH 7.4). The reaction was initiated by the addition of individual recombinant human CYP isoforms (25 μmol/ml) in a total volume of 1 ml and samples were maintained at 37° C. for various time periods. At each designated time-point, aliquots were extracted with acetonitrile, and subjected to HPLC analysis.

### Results:

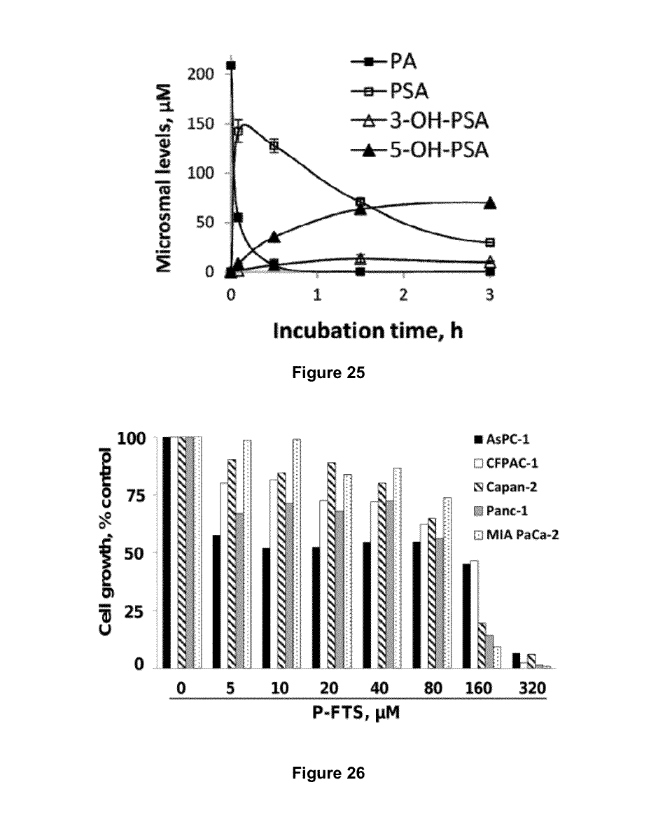

As shown in FIG. 25, three major metabolites of PA-III 506 were detected in PA-treated liver microsomes by HPLC: phospho-salicylic acid (PSA) 95, 3-OH-PSA and 5-OH—OH-PSA. This result indicates that PA-III 506 can be readily deacetylated at its ASA moiety to form PSA 95, which is oxidized to 3-OH-PSA and 5-OH-PSA. In contrast to PSA 95, conventional SA and ASA were not appreciably oxidized by liver microsomes under the same experimental conditions. Thus, a) only PSA 95 can be oxidized to generate 3-OH-PSA and 5-OH-PSA (structures shown in scheme above), which are metabolites unique to PA vis-à-vis ASA or SA. Their generation represents regioselective oxidation; b) CYPs 2C19 and 2D6 catalyze appreciably this oxidation, with 1A2, 2C9 and 3A4 being minimally active; and c) ASA cannot be oxidized by any of the five CYPs tested, not even by whole liver microsomes.

These results indicate that PA-III 506 can generate metabolites, which ASA cannot generate; and the production of these metabolites is catalyzed by a specific subset of CYP isoforms.

### Example 14

Formation of Reactive Phospho-Aspirin III (PA-III) (Compound 506) Metabolites In Vivo

As shown in the scheme below, 5-OH-PSA, having two hydroxyl groups, is dehydrogenated by CYPs to form a quinone-type highly reactive intermediate, which was trapped by GSH. The Scheme below also shows the GSH adduct of a quinone-type reactive intermediate of PA-III 506, which was subsequently identified by LC-MS/MS analysis. Likewise, 3-OH-PSA leads to the analogous reactive intermediate, which was also trapped by GSH (data not shown). Quinones are highly redox-active molecules leading, among others, to reactive oxygen species (J. L. Bolton et al. Chem Res Toxicol. 2000, 13, 135-60), which, in turn, can induce cancer cell apoptosis (B. Rigas, Y. Br J Cancer. 2008, 98, 1157-60). Indeed, as recently shown, phospho-NSAIDs including PA-III 506, act by inducing oxidative stress selectively in cancer cells (Y. Sun et al. J Pharmacol Exp Ther. 2011, 338, 775-83).

These data explain a) the efficacy of PA-III 506 for breast cancer prevention, as shown in Example 12 (PA-III 506 produces the highly reactive quinone-type intermediates) and b) the lack of efficacy of ASA in the same study (ASA is unable to generate these reactive metabolites).

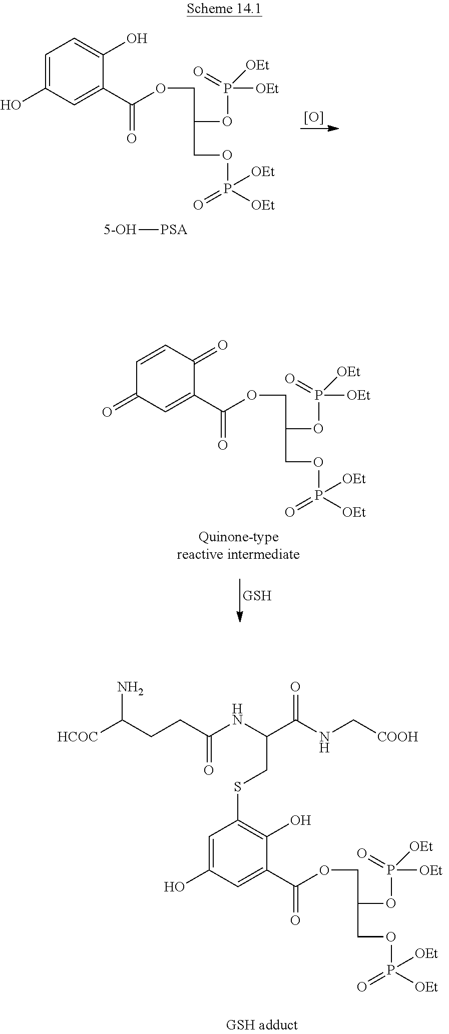

### Example 15

### Phospho-Farnesylthiosalicylic Acid (P-FTS, Compound 67) Inhibits the Growth of Human Pancreatic Cancer Cells in Culture

Cell growth was determined in the human pancreatic cancer cell lines AsPC-1, CFPAC-1, Capan-2, Panc-1 and MIA PaCa-2 after treatment with escalating concentrations of P-FTS 67 for 24 hours. Results, expressed as % control, are show that P-FTS decreases pancreatic cancer cell growth in a concentration-dependent manner in all cell lines (FIG. 26).

P-FTS 67 Inhibits the Growth of Human Pancreatic Xenografts in Nude Mice.

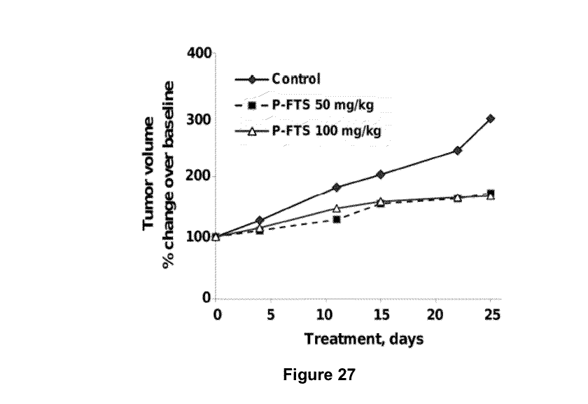

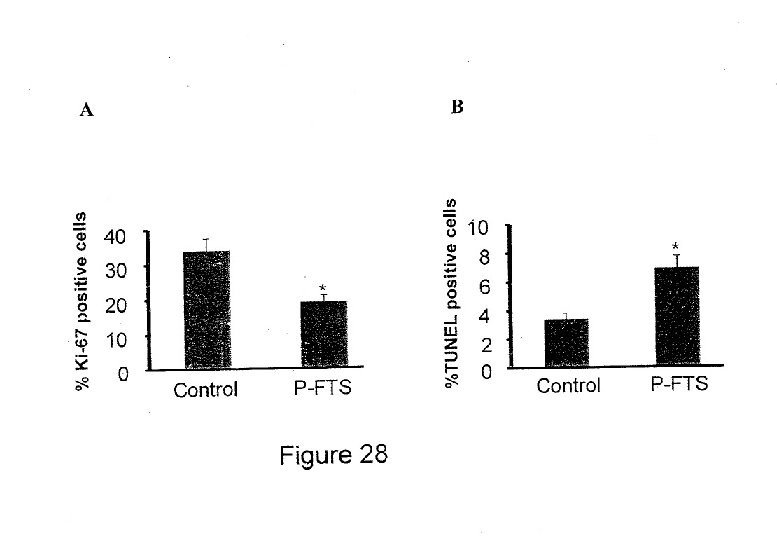

The in vivo chemotherapeutic potential of P-FTS 67 was assessed using a pancreatic cancer xenograft model. MIA PaCa-2 cells were injected subcutaneously into the flank areas of nude mice. When palpable tumors were observed, the mice received P-FTS 67, 50 or 100 mg/kg/d by oral gavage in corn oil or just corn oil (control) for 25 days. On day 25 of treatment, P-FTS 67, 50 mg/kg/d, and P-FTS 67, 100 mg/kg/d reduced the tumor volume growth by 62% and 65%, respectively (p <0.05; FIG. 27).

### Safety of P-FTS (Compound 67) in Mice

In the efficacy study described above, P-FTS 67 was well tolerated, with the mice showing no weight loss or other signs of toxicity. The toxicity of P-FTS in mice was further examined. Groups of 6 week-old female BALB/c mice (5 mice/group) were given by oral gavage once a day for 3 weeks P-FTS: 0, 75, 150, 250 and 350 mg/kg. All P-FTS-treated animals showed no weight loss or other signs of toxicity. The (sub-chronic) maximum tolerated dose of P-FTS 67 (3-week period of observation) was determined to be at least 350 mg/kg/d.

### P-FTS (Compound 67) Inhibits Ras Activation and its Downstream Effectors ERK and AKT

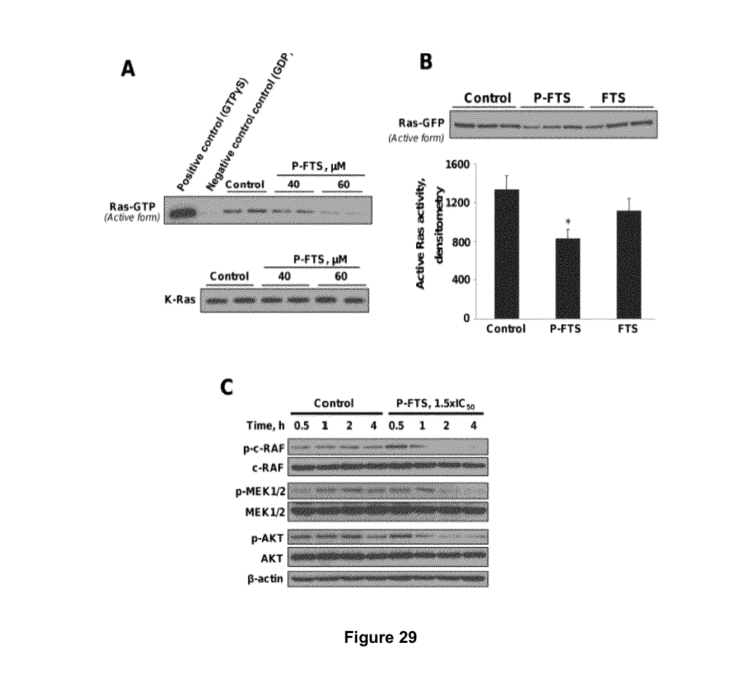

In the following experiments it was examined whether the pancreatic cancer growth inhibitory effect of P-FTS 67 is associated with down regulation of Ras signaling. For this purpose, the Ras-GTP pull down assay was used. In Panc-1 cells, P-FTS 67 significantly inhibited active Ras (Ras-GTP) in a concentration-dependent manner, and this decrease was more pronounced that the one observed with the parent compound (FIG. 29). The inhibition of Ras by P-FTS 67 led to a significant time-dependent inhibition of the RAF/MEK/ERK and PI3K/AKT pathways, two downstream effectors of Ras (FIG. 29C).

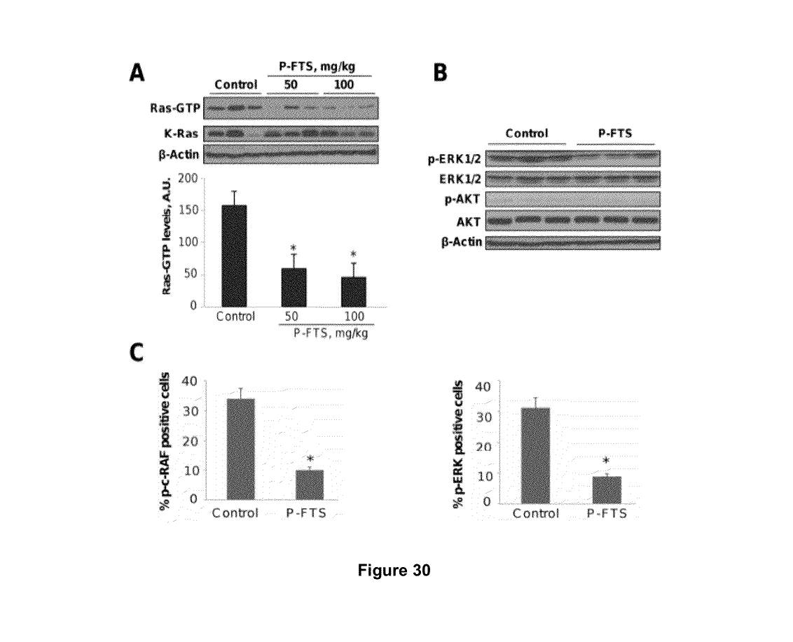

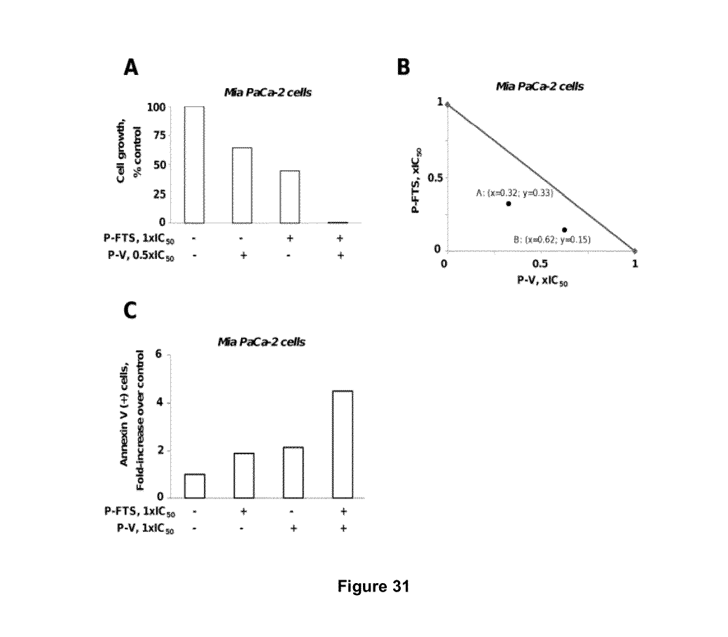

The inhibition of Ras by P-FTS 67 was further confirmed in vivo. The Ras-GTP pull down assay was used to test the capacity of P-FTS 67 to inhibit active Ras in fresh protein lysates from MIA PaCa-2 xenografts. The observed results are summarized in FIG. 30. Compared to controls, P-FTS 67, 50 and 100 mg/kg reduced RAS activation in xenografts by 62% and 70%, respectively (p<0.01, for both; FIG. 30A). The suppression of Ras was accompanied by inhibition of p-ERK and p-AKT, as determined by immunoblotting (FIG. 30B). Moreover, immunostaining of xenograft tissue sections revealed that P-FTS 67 reduced the expression of p-c-RAF and p-ERK1/2 by 73% and 71%, respectively, compared to control (p <0.01; FIG. 30C).

In summary, the obtained in vivo results suggest, in agreement with the in vitro results, that Ras is a critical molecular target of P-FTS 67, likely accounting for its pancreatic cancer growth inhibitory effect.

P-FTS (Compound 67) Synergizes with Phospho-Valproic Acid (P-V) (Compound 116) to Enhance Human Pancreatic Cancer Growth Inhibition

The potential synergy between P-FTS with phospho-valproic acid (PV) 116, a novel STAT3 inhibitor was evaluated in BxPC-3 and MIA PaCa-2 cells. The results, summarized in FIG. 31, show that P-FTS 67 and PV 116 synergize to inhibit cell growth (FIGS. 31A and B) and induce apoptosis (FIG. 31C). These results suggest that PV is a useful in combination with P-FTS in the treatment of Ras mutated cancers.

### Example 16

Efficacy of Amide Compounds

### Study Groups:

Groups of 7-15 nude mice were treated with the test drug or vehicle, the latter serving as controls. (In the case of subcutaneous xenografts they had 1-2 implants, bearing in total 10-30 xenografts/group.) The vehicle used was corn oil for ip injections, for topical application empty hydrogel and for oral administration. The effect of the test drug was compared to the respective control group.

### Results

PHOSPHO-IBUPROFEN-GLYCEROL-AMIDE (Compound 4)

| Cancer | | | Dose/duration/ | |
|---|---|---|---|---|
| Origin | Cell line | Xenograft | administration route | Reduction |
| Glioma | U87 | sc | 20 mg/kg/d alone × 12 d, ip | 52% |
| | | | 20 mg/kg/d plus phospho- | 88% |
| | | | valproic acid | |
| | | | 116 50 mg/kg/d × 12 d, ip | |

PHOSPHO-SULINDAC-AMIDE (Compound 95)

| Cancer | | | Dose/duration/ | |
|---|---|---|---|---|
| Origin | Cell line | Xenograft | administration route | Reduction |
| Lung | A549 | sc | 200 mg/kg/d × 24 d, ip | 88% |
| | | ot | 200 mg/kg/sd × 6 week, ip | 77% |
| Skin | A431 | id | Topical × 2 week | 75% |
| Gastric | AGS | sc | 200 mg/kg/d × 3 week, ip | 67% |
| Colon | HCT116 | | 250 mg/kg/d × 12 d, ip | 23% |

PHOSPHO-ASPIRIN (GLYCEROL) AMIDE (Compound 194)

| Cancer | | | Dose/duration/ | |
|---|---|---|---|---|
| Origin | Cell line | Xenograft | administration route | Reduction |
| Breast | MCF7 | sc | 120 mg/kg/d × 29 d, po, | 55% |
| | | | prevention | |
| | | | 500 mg/kg/d × 12 d, ip, | 80-100% |
| | | | treatment | |
| Colon | HCT116 | sc | 100 mg/kg/d × 12 d, ip | 18% |
| IBD* | | | | |
| Ulcerative | DSS | n/a | 300 mg/kg/d × 10 d, po | 30-48% |
| colitis | | | | |

*IBD = inflammatory bowel disease

PHOSPHO-VALPROIC ACID (Compound 116)
Cancer

| Cancer | | | Dose/duration/ | |
|---|---|---|---|---|
| Origin | Cell line | Xenograft | administration route | Reduction |
| Lung | A549 | ot | 100 mg/kg/d × 6 week, ip | 80% |
| Breast | MDA | sc | 120 mg/kg/d × 22 d, ip | 63% |
| | BT-20 | sc | 120 mg/kg/d × 28 d, ip | 37% |
| Gastric | AGS | sc | 100 mg/kg/d × 3 week, ip | 81% |
| Colon | HT-29 | sc | 120 mg/kg/d × 12 d, ip | 40% |

PHOSPHO-IBUPROFEN BUTANE AMIDE (Compound 1)

| Cancer | | | Dose/duration/ | |
|---|---|---|---|---|
| Origin | Cell line | Xenograft | administration route | Reduction |
| Lung | A549 | ot | 160 mg/kg/d × 6 week, ip | 75% |
| Skin | A431 | id | Topical, 2 week | 46% |
| Gastric | AGS | sc | 160 mg/kg/d × 3 week, ip | 34% |

These results show the strong anticancer efficacy of the various amide compounds and the significant efficacy of the compound tested on ulcerative colitis, a major subtype of inflammatory bowel disease.

Key findings of the anticancer efficacy results include the following:
a) the effect is robust, approaching in several instances tumor stasis;
b) it encompasses both prevention and treatment of cancer;
c) it is maintained with multiple routes of administration and various drug formulations;
d) it is obtained with an extensive repertoire of tumors of diverse tissue origin;
e) tumor models of varying configuration (orthotopic, subcutaneous, intradermal) respond well to these compounds indicating their anticancer properties;
f) the antitumor effect is maintained regardless of the structure or chemical class of the parent compound (e.g., NSAID subclasses or NSAIDs vs. valproic acid).
   It should be added that the efficacy of Compound 194 in colitis, a prototypical inflammation-related disease is an unexpected finding, since conventional NSAIDs such as aspirin aggravate inflammatory bowel disease rather than improving it (patients are advised to avoid these medications). Without being bound by any theory, this finding further suggests a mechanistic link between these effects, namely that the inflammatory component of cancer is different from “routine” or classical inflammation. Such anti-inflammatory action is generated by the novel chemical modifications claimed here, as conventional compounds such as valproic acid do not possess such benefits. These effects, combined with the favorable pharmacokinetics of the amide compounds, illustrate their potential for therapeutic efficacy in clinical applications.

### Example 17

### Amide Compounds are not Pro-Drugs

If the phospho-NSAIDs were prodrugs this would mean that they are initially administered to the body in an inactive (or less than fully active) form, and then are converted to their active form through the normal metabolic processes of the body. That is, the compounds of the invention, when administered, have activity independent of their metabolites, regardless of whether metabolites are formed.

Phospho-NSAIDs of the invention are not prodrugs. There is excellent evidence that the entire molecule is required for its full efficacy. These data, summarized below, are based on the pharmacological behavior of phospho-NSAIDs that are carboxylic esters. Since these compounds are easily hydrolyzed at the carboxylic ester linking the NSAID to the space moiety (in contrast to the amides), they offer an experimental system suitable to assess this question. In this example, phospho-NSAIDs refers to esters not to amides.

The hydrolysis of phospho-NSAIDs by carboxylesterase (CES) plays a key role in their inactivation in vivo (e.g., Wong C C, Cheng K W, Xie G, Zhou D, Zhu C—H, Constantinides P P, Rigas B. Carboxylesterases 1 and 2 hydrolyze phospho-NSAIDs: Relevance to their pharmacological activity. J Pharmacol Exp Ther, 2012; 340422-32). Mice express plasma carboxylesterase 1c (Ces1c), an isoform absent in humans, which enhances the inactivation of phospho-NSAIDs. In vitro, phospho-NSAIDs are extensively hydrolyzed by Ces1c over-expressed in mammalian cells. Moreover, the rate of phospho-NSAID hydrolysis in wild type mouse plasma is 6 to 530-fold higher than that in plasma of Ces1c knockout mice. The presence of plasma carboxylesterase significantly attenuates the in vitro cytotoxicity of phospho-NSAIDs in cancer cell lines, suggesting that drug integrity is critical for their anticancer activity.

In vivo, pharmacokinetic studies of phospho-sulindac (intraperitoneal and intravenous administration) using wild type and Ces1c knockout mice demonstrated approximately 2-fold less inactivation of phospho-sulindac in the latter. This is reflected in the enhanced distribution of intact phospho-sulindac in various tissues.

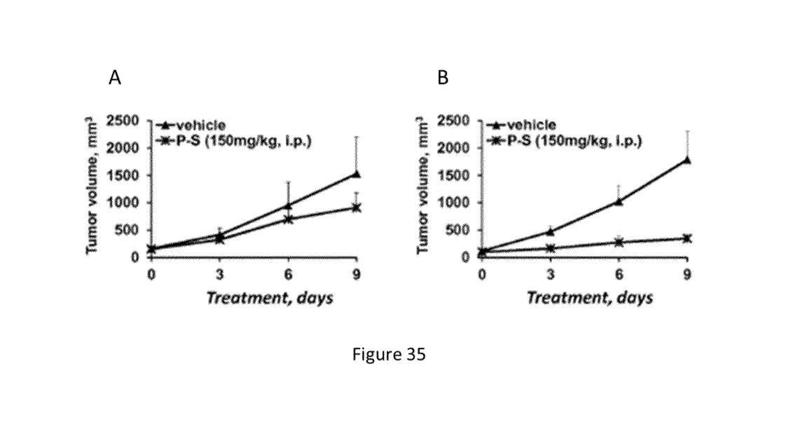

Consistent with the pharmacokinetic data, phospho-sulindac is significantly more effective (approximately 2-fold) in inhibiting the growth of lung and pancreatic carcinoma in Ces1c knockout mice, as compared to wild type mice. Results are shown in FIG. 35. Phospho-valproic acid, another phospho-modified drug, is also more efficacious towards pancreatic carcinoma in Ces1c knockout mice. These results indicate that the intact phospho-NSAID is the pharmacologically active moiety.

Administration of Esterase Inhibitors Increases the Efficacy of Hydrolysable Phospho-NSAIDs.

For example, in mice, co-administration of phospho-sulindac and bis-p-nitrophenyl phosphate, an esterase inhibitor, protected the former from esterase-mediated hydrolysis, and this combination more effectively inhibited the growth of AGS human gastric xenografts in nude mice (57%) compared with phospho-sulindac alone (28%) (p<0.037) (Wong C C, Cheng K W, Xie G, Zhou D, Zhu C F I, Constantinides P P, Rigas B. Carboxylesterases 1 and 2 hydrolyze phospho-nonsteroidal anti-inflammatory drugs: relevance to their pharmacological activity. J Pharmacol Exp Ther. 2012; 340:422-32).

Protection of phospho-NSAIDs from hydrolysis by encapsulation in nanocarriers enhances their anti-cancer efficacy. For example, PS was formulated in a nanocarrier suitable for iv administration [PLLA(10K)-PEG(2K)]. Control pharmacokinetic experiments revealed that <6% of PS in this nanocarrier is hydrolyzed to sulindac. Nude mice bearing subcutaneous A549 human lung cancer xenografts were then given intravenously PS 60 mg/kg/d ×2d/week and the equimolar dose of sulindac (40 mg/kg.) Sulindac was ineffective in inhibiting the growth of the xenografts whereas PS was (32% vs. 75%; p<0.012). These data establish that PS is not a pro-drug of sulindac. In other words, the anticancer efficacy of PS is not the result of its being metabolized to sulindac (Xie G, et al., Brit J. Pharmacol. 2012; 165:2152-66; Wong et al., J Pharmacol Exp Ther. 2012; 340:422-32).

### Safety of the Amides Compounds

The amide compounds studied here have been very safe in wild type and nude mice. In particular, at the doses used in the studies described herein, there has been no evidence of side effects. Their weight has been indistinguishable from that of controls. Their maximum tolerated doses (MTDs) were as follows:

| COMPOUND | MOUSE | ROUTE | DOSE | | DURATION | |
|---|---|---|---|---|---|---|
| Phospho-ibuprofen | wt | po | >600 | mg/kg/d | 1 | week |
| glycerol amide (4) | nude | ip | 20 | mg/kg/d | 12 | days |
| Phospho-sulindac | wt, nude | po | >500 | mg/kg/d | 1 | week |
| butane amide (95) | nude | ip | 200 | mg/kg/d | 6 | week |
| | | | 250 | mg/kg/d | 3 | week |
| Phospho-sulindac | wt, nude | po | >500 | mg/kg/d | 1 | week |
| glycerol amide (99) | | | | | | |
| Phospho-aspirin | wt | po | 240 | mg/kg/d | 1 | week |
| glycerol | nude | ip- | 150 | mg/kg/d | 1 | week |
| mono-amide (15) | >10 wks | chronic | | | | |
| | old | | | | | |
| Phospho-valproic | wt | po | 300 | mg/kg/d | | |
| acid amide | nude | ip | 100 | mg/kg/d | 6 | week |
| (116) | | | 120 | mg/kg/d | 3 | week |
| | | | 150 | mg/kg/d | 1-3 | week |
| Phospho-ibuprofen | nude | ip | 160 | mg/kg/d | 6 | week |
| butane amide (1) | | | | | | |

### Example 18

### Synthesis of Nitro Amide Compounds (e.g., Nitro-aspirin benzyl amide, Compound 508)

A general scheme for synthesizing the amide compounds of the invention is exemplified by the scheme below and by the following synthesis examples.

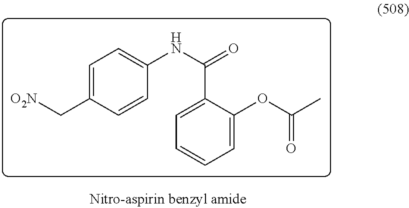 [SMILES: CC(=O)Oc1ccccc1C(=O)Nc1ccc(C[N+](=O)[O-])cc1]

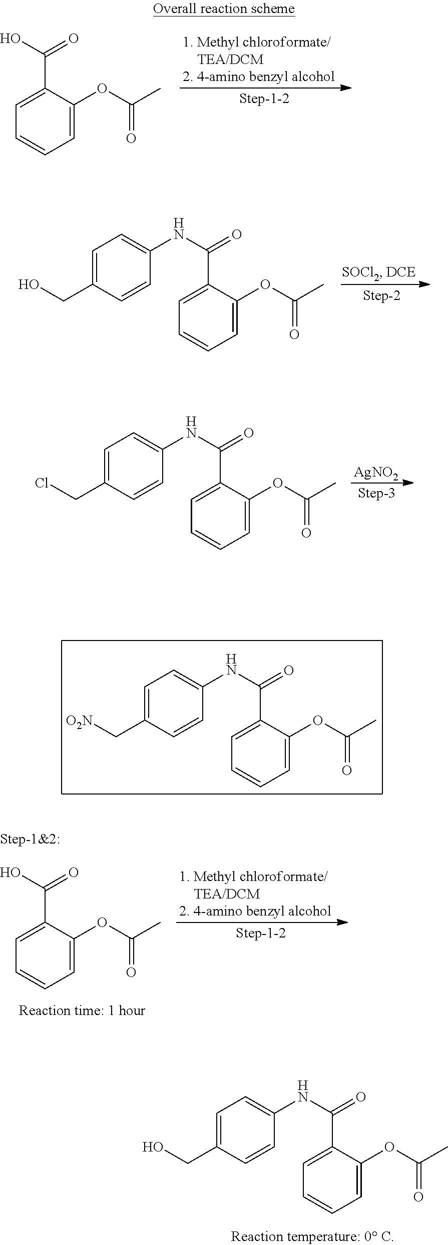

Step-1:

TEA (7.46 mL, 1.0 eq.) was slowly added to 4-amino benzyl alcohol (6.8 g, 1.0 eq.) in DCM (50 mL) at 0° C. under nitrogen.

Step-2:

Methylchloroformate (3.6 mL g, 1.0 eq.) was slowly added to stirred solution of product from Step-1 (10.0 g, 1.0 eq.) and TEA (7.46 mL, 1.0 eq.) in DCM (50 mL) at 0° C. under nitrogen. The reaction mixture was stirred for 20 minutes. The reaction mixture was filtered and filtrate was slowly added at 0° C. under nitrogen atmosphere. The resulting reaction mixture was stirred at 0° C. for 30 minutes. The reaction progress was monitored by TLC.

Work up: After completion of the reaction (TLC), the reaction mixture was diluted with water and extracted with DCM (2×150 mL), the combined organic layer was washed with water and brine solution, dried over anhydrous sodium sulphate and concentrated under vacuum to get crude compound. Purification: The crude compound was purified by silica gel (100-200 mesh) column chromatography by eluting with 50% ethyl acetate in petroleum ether.

| | |
|---|---|
| TLC system: 50% EtOAc/Pet ether | R_{f} value: 0.3 |
| Nature of the compound: Brown oil | Yield: 0.6 g (3.7%) |

### Step-3:

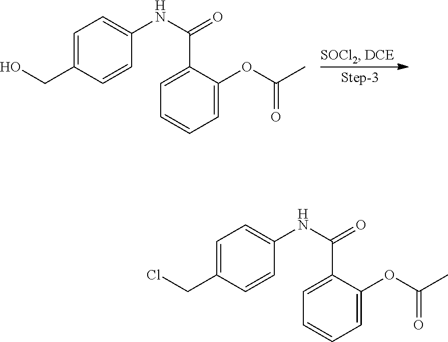 [SMILES: CC(=O)Oc1ccccc1C(=O)Nc1ccc(CO)cc1 ; CC(=O)Oc1ccccc1C(=O)Nc1ccc(CCl)cc1]

| | |
|---|---|
| Reaction time: 3 hours | Reaction temperature: 85° C. |

SOCl₂ (0.4 mL, 2 eq.) was slowly added to stirred solution of product from Step-2 (0.75 g, 1.0 eq.) in DCE (10 mL) at 0° C. The reaction mixture was stirred at 85° C. for 3 hours. The reaction progress was monitored by TLC. Work up: After completion of reaction (TLC), the reaction mixture was concentrated under vacuum completely removed (SOCl₂), to get crude compound. Purification: The crude compound was purified by silica gel (100-200 mesh) column chromatography by eluting with 20% ethyl acetate in pet ether to get the pure compound of Step-3.

| | |
|---|---|
| TLC system: 50% EtOAc/Pet ether | R_{f} value: 0.8 |
| Nature of the compound: Off-white solid | Yield: 0.07 g (8.8%) |

### Step-4:

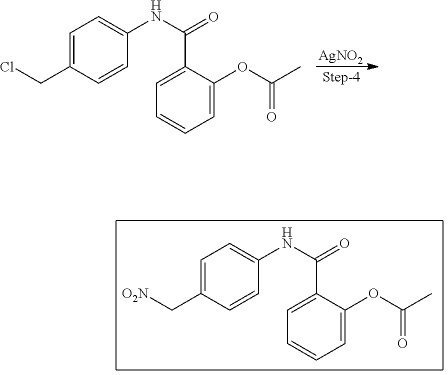

| | |
|---|---|
| Reaction time: 12 hours | Reaction temperature: RT |

To stirred solution of AgNO2 (0.16 g, 1.3 eq.) in Diethyl ether (10 mL) at RT under nitrogen and degas with nitrogen. Product from Step-3 (0.24 g, 1.0 eq) dissolved in Diethyl ether nitrogen was added to the reaction mixture. The reaction mixture was stirred for 12 hours at RT. The reaction progress was monitored by TLC.

Work up: After completion of reaction (TLC), the reaction mixture was diluted with water and extracted with Diethyl ether (2×25 mL), the combined organic layer was washed with water and brine solution dried over anhydrous sodium sulphate and concentrated under vacuum to get crude compound. Purification: The crude compound was purified by silica gel (100-200 mesh) column chromatography by eluting with 20% ethyl acetate in petroleum ether.

| | |
|---|---|
| TLC system: 50% EtOAc/Pet ether | R_{f} value: 0.7 |
| Nature of the compound: Brown solid | Yield: 0.05 g (20.1%) |

### Example 19

### Synthesis of Amides (e.g., Phospho-Aspirin Benzyl Amide Compound 509)

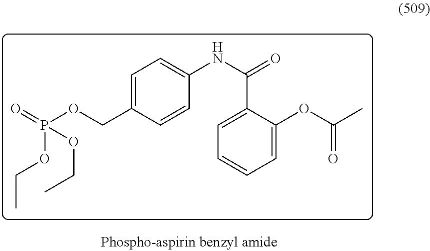 [SMILES: CCOP(=O)(OCC)OCc1ccc(NC(=O)c2ccccc2OC(C)=O)cc1]

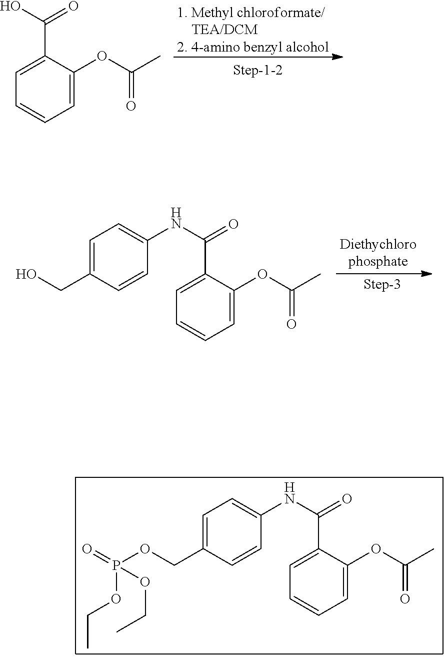 [SMILES: CC(=O)Oc1ccccc1C(=O)O ; CC(=O)Oc1ccccc1C(=O)Nc1ccc(CO)cc1 ; CCOP(=O)(OCC)OCc1ccc(NC(=O)c2ccccc2OC(C)=O)cc1]

### Step-1&2:

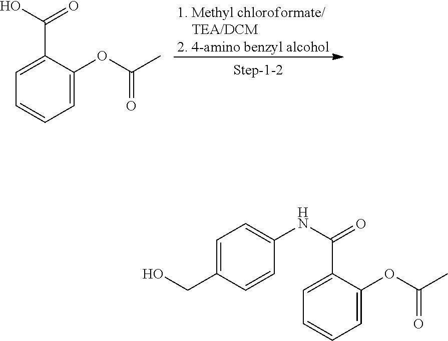 [SMILES: CC(=O)Oc1ccccc1C(=O)O ; CC(=O)Oc1ccccc1C(=O)Nc1ccc(CO)cc1]

| | |
|---|---|
| Reaction time: 1 hour | Reaction temperature: 0° C. |

### Step-1:

TEA (7.46 mlL, 1.0 eq.) was slowly added 2,4-amino benzyl alcohol (6.8 g, 1.0 eq.) in DCM (50 mL) at 0° C. under nitrogen.

### Step-2:

Methylchloroformate (3.6 mL g, 1.0 eq.) was slowly added to a stirred solution of product from Step-1 (10.0 g, 1.0 eq.) and TEA (7.46 mL, 1.0 eq.) in DCM (50 mL) at 0° C. under nitrogen. The reaction mixture was stirred for 20 minutes. The reaction mixture was filtered and filtrate was slowly added at 0° C. under nitrogen atmosphere. The resulting reaction mixture was stirred at 0° C. for 30 minutes. The reaction progress was monitored by TLC.

Work up: After completion of reaction (TLC), the reaction mixture was diluted with water and extracted with DCM (2×150 mL), the combined organic layer was washed with water and brine solution, dried over anhydrous sodium sulphate and concentrated under vacuum to get crude compound. Purification: The crude compound was purified by silica gel (100-200 mesh) column chromatography by eluting with 50% ethyl acetate in petroleum ether.

| | |
|---|---|
| TLC system: 50% EtOAc/Pet ether | R_{f} value: 0.3 |
| Nature of the compound: Brown oil | Yield: 0.6 g (3.7%) |

### Step-3:

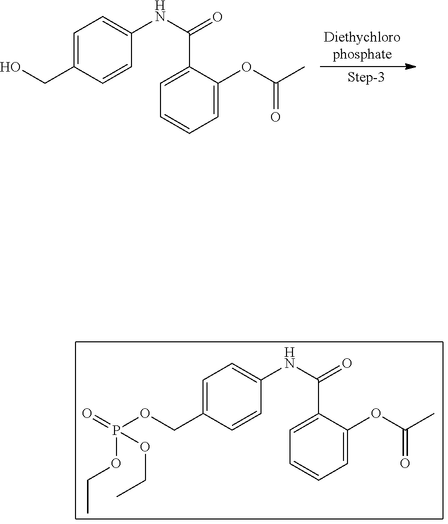 [SMILES: CC(=O)Oc1ccccc1C(=O)Nc1ccc(CO)cc1 ; CCOP(=O)(OCC)OCc1ccc(NC(=O)c2ccccc2OC(C)=O)cc1]

| | |
|---|---|
| Reaction time: 20 minutes | Reaction temperature: RT |

Diethyl choloro phosphate (2.5 g, 5.0 eq.) was slowly added to a stirred solution of the product from Step-2 (0.84 g, 1.0 eq.) and TEA (3.9 mL, 10.0 eq.) in DCM (20 mL) at RT under nitrogen. The reaction mixture was stirred for 20 minutes at RT. The reaction progress was monitored by TLC.

Work up: After completion of the reaction (TLC), the reaction mixture was diluted with water and extracted with DCM (2×25 mL), the combined organic layer was washed with water and brine solution dried over anhydrous sodium sulphate and concentrated under vacuum to get crude compound. Purification: The crude compound was purified by silica gel (100-200 mesh) column chromatography by eluting with 5% methanol in DCM.

| | |
|---|---|
| TLC system: 50% EtOAc/Petroleum ether | R_{f} value: 0.15 |
| Nature of the compound: Off-White solid | Yield: 0.2 q (16.6%) |

### Example 20

### Synthesis of Amides (e.g., Phospho-Aspirin Di-Amide, Compound 510)

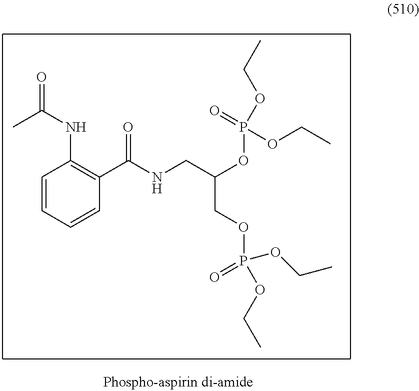 [SMILES: CCOP(=O)(OCC)OCC(CNC(=O)c1ccccc1NC(C)=O)OP(=O)(OCC)OCC]

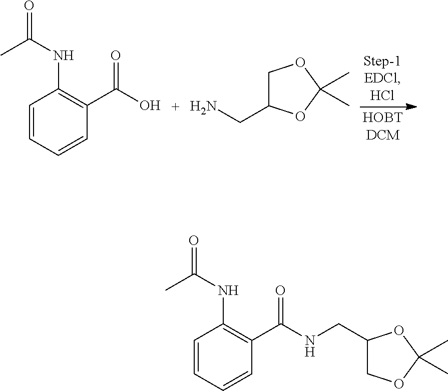

### Step-1:

To a stirred solution of first starting material (10 g, 1.0 eq) in DMF (100 ml) was added EDC.HCL (16.6 g, 1.5 eq), HOBT (7.5 g, 1.0 eq) followed by second starting material (7.31 g, 1.0 eq) at 0° C. and reaction mixture was stirred for 16 hours at room temperature. After completion of reaction the residue was dissolved in ethyl acetate and washed with water followed by brine solution. The organic layer was dried over sodium sulfate, then was concentrated under reduced pressure to get crude compound. The crude product was purified by column chromatography over a silica gel (100-200 mesh) with 30% ethyl acetate in petroleum ether to get 9.8 g (60%) of product as off white solid.

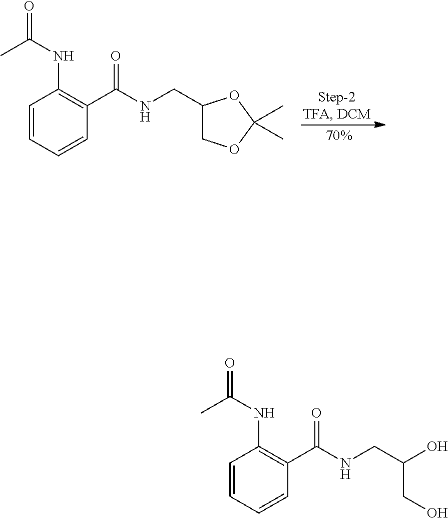

### Step-2:

To a stirred solution of product from Step-1 (10 g, 1.0 eq) in DCM (150 ml) was added TFA (15.6 g, 4 eq) at 0° C. and stirred for 3 hours at room temperature. After completion of reaction, TFA was concentrated under reduced pressure. The crude product was purified by column chromatography over a silica gel (100-200 mesh) with 40% ethyl acetate in pet ether to get 5.7 g (70%) of off white solid.

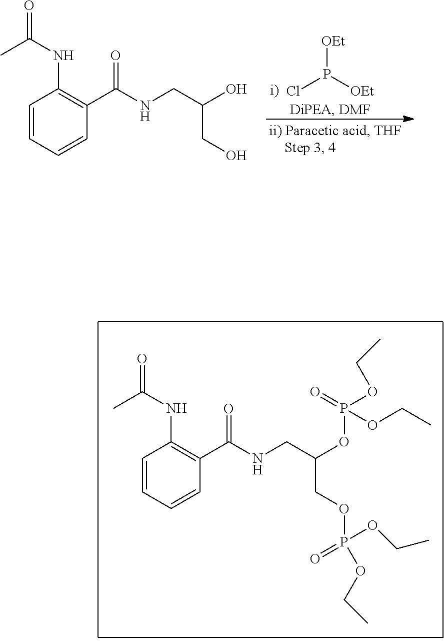 [SMILES: CC(=O)Nc1ccccc1C(=O)NCC(O)CO ; CCOP(Cl)OCC ; CCOP(=O)(OCC)OCC(CNC(=O)c1ccccc1NC(C)=O)OP(=O)(OCC)OCC]

### Step-3 & 4:

To a stirred solution of product from Step-2 (10 g, 1.0 eq) in DMF (120 ml) was added DIPEA (102.3 g, 20 eq) followed by Diethyl chlorophosphite (55.1 g, 10 eq) stirred for 16 hours at room temperature. After completion of reaction the residue was dissolved in ethyl acetate and washed with water followed by brine solution. The organic layer was dried over sodium sulfate; was then concentrated under reduced pressure to get the desired phosphite compound. The obtained phosphate compound was dissolved in THF (150 ml) was added in acetic acid (90.4 g, 30 eq) at 0° C. and stirred at room temperature (RT) for 2 hours. After completion of reaction the residue was dissolved in ethyl acetate and washed with water followed by brine solution. The organic layer was dried over sodium sulfate; and then was concentrated under reduced pressure to get the crude product. The crude product was purified by column chromatography over a silica gel (100-200 mesh) with 3% methanol in DCM to get 13.0 g (65%) of as yellow gummy liquid.

### Example 21

### Synthesis of Amides (e.g., Phospho-Aspirin IV, Compound 507)

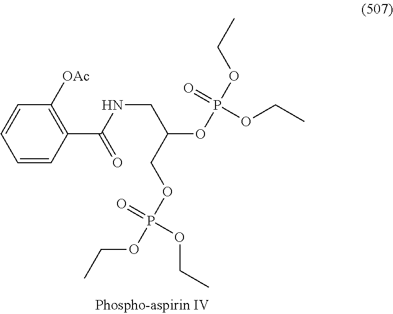 [SMILES: CCOP(=O)(OCC)OCC(CNC(=O)c1ccccc1OC(C)=O)OP(=O)(OCC)OCC]

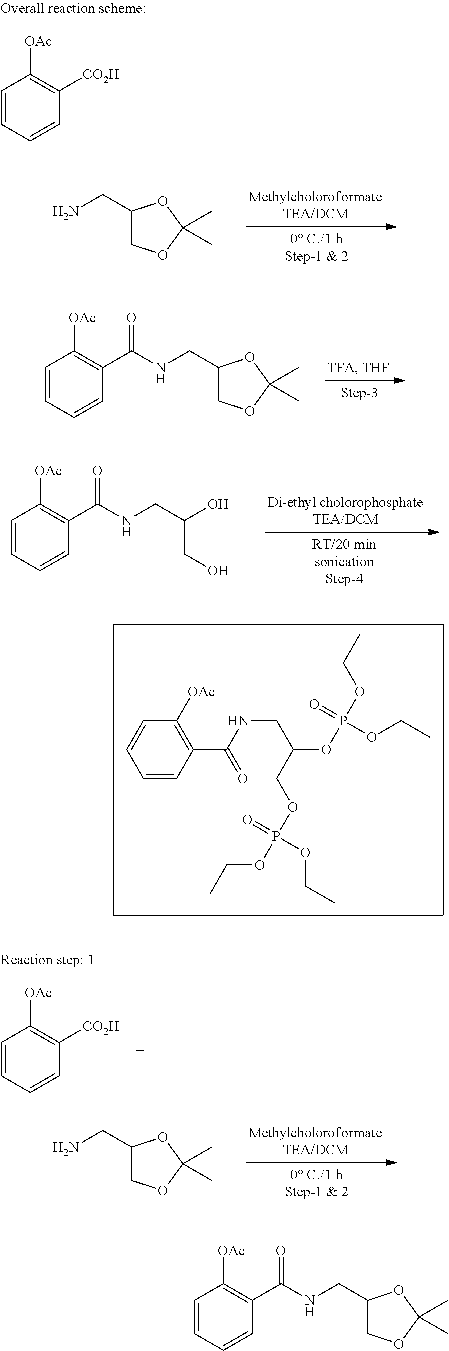

| | |
|---|---|
| Reaction time: 1 hour | Reaction temperature: 0° C. |

### Step-1:

TEA (1.12 g, 1.0 eq.) was slowly added to the second compound above (1.45 g, 1.0 eq.) in DCM (10 mL) at 0° C. under nitrogen.

### Step-2:

Methylchloroformate (1.0 g, 1.0 eq.) was slowly added to stirred solution of product from Step-1 (2.0 g, 1.0 eq.) and TEA (1.12 g, 1.0 eq.) in DCM (25 mL) at 0° C. under nitrogen. The reaction mixture was stirred for 20 minutes. The reaction mixture was filtered and filtrate was slowly added at 0° C. under nitrogen atmosphere. The resulting reaction mixture was stirred at 0° C. for 30 minutes. The reaction progress was monitored by TLC.

Work up: After completion of the reaction (TLC), the reaction mixture was diluted with water and extracted with ethyl acetate (2×150 mL), the combined organic layer was washed with water and brine solution, dried over anhydrous sodium sulphate and concentrated under vacuum to get crude compound. Purification: The crude compound was purified by silica gel (100-200 mesh) column chromatography by eluting with 30% ethyl acetate in pet ether to get pure compound of Step-3.

| | |
|---|---|
| TLC system: 30% EtOAc/Petroleum ether | R_{f} value: 0.1 |
| Nature of the compound: Yellow solid | Yield: 2.1 g (63.2%) |

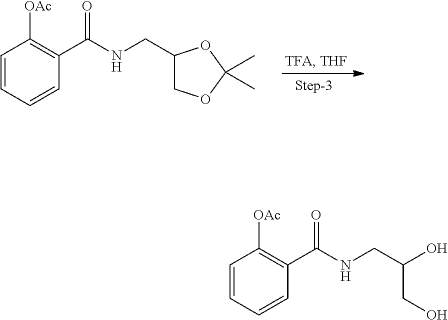

| | |
|---|---|
| Reaction time: 30 minutes | Reaction temperature: 0° C. |

TFA (1.9 g, 10 eq.) was slowly added to a stirred solution of product from Step-2 (0.5 g, 1.0 eq.) in DCM (10 mL) at 0° C. The reaction mixture was stirred at 0° C. for 30 minutes. The reaction progress was monitored by TLC.

Work up: After completion of the reaction (TLC), the reaction mixture was concentrated under vacuum completely removed (TFA), to get crude compound. Purification: The crude compound was purified by silica gel (100-200 mesh) column chromatography by eluting with 5% methanol in DCM.

| | |
|---|---|
| TLC system: 10% Methanol in DCM | R_{f} value: 0.1 |
| Nature of the compound: liquid | Yield: 0.2 g (50.0%) |

### Step-4:

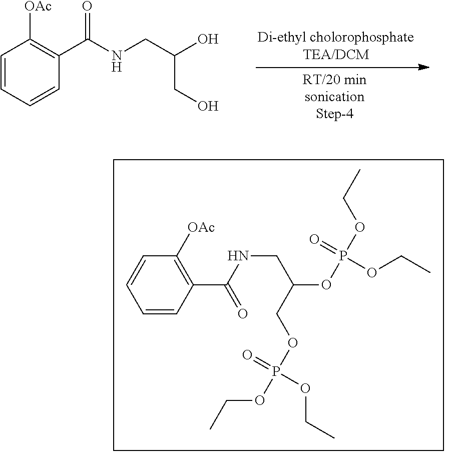 [SMILES: CC(=O)Oc1ccccc1C(=O)NCC(O)CO ; CCOP(=O)(OCC)OCC(CNC(=O)c1ccccc1OC(C)=O)OP(=O)(OCC)OCC]

| | |
|---|---|
| Reaction time: 20 minutes | Reaction temperature: RT |

Di-ethyl cholorophosphate (30.5 g, 5.0 eq.) was slowly added to a stirred solution of product from Step-3 (9.0 g, 1.0 eq.) and TEA (49.9 mL, 10.0 eq.) in DCM (100 mL) at RT under nitrogen. The reaction mixture was stirred for 20 minutes at RT. The reaction progress was monitored by TLC.

Work up: After completion of the reaction (TLC), the reaction mixture was diluted with water and extracted with DCM (2×250 mL), the combined organic layer was washed with water and brine solution dried over anhydrous sodium sulphate and concentrated under vacuum to get crude compound. Purification: The crude compound was purified by silica gel (100-200 mesh) column chromatography by eluting with 5% methanol in DCM.

| | |
|---|---|
| TLC system: 10% Methanol in DCM | R_{f} value: 0.2 |
| Nature of the compound: Thick syrup | Yield: 10.5 g (84%) |

### Example 22

### Synthesis of Amides (e.g., Phospho-valproic acid amide, Compound 511)

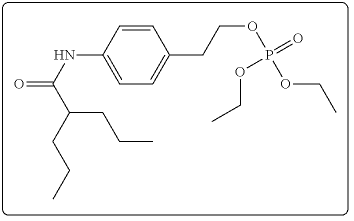 [SMILES: CCCC(CCC)C(=O)Nc1ccc(CCOP(=O)(OCC)OCC)cc1]

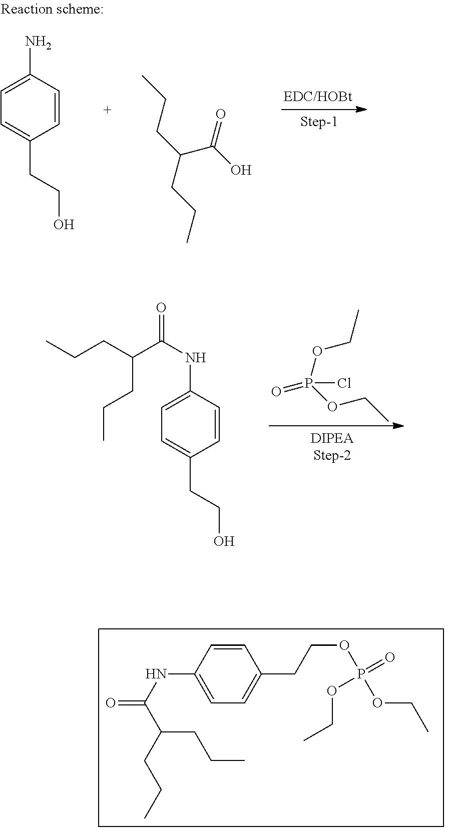

| | |
|---|---|
| Reaction time: 3 hours | Reaction temperature: 0° C. |

### Step-1:

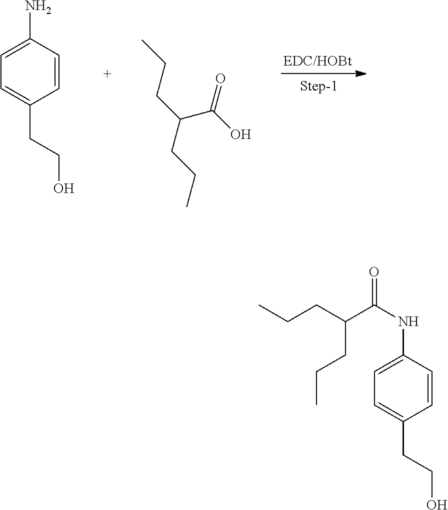

To stirred solution of first starting material (50 g, 1.0 eq) and second starting material (52.55 g, 1.0 eq) in dry DCM (100 mL) at 0° C. under nitrogen, EDC-HCl (83.95 g, 1.2 eq) and HOBt (83.7 g, 1.5 eq) was added to the reaction mixture. The reaction mixture was stirred for 3 h at 0° C. The reaction progress was monitored by TLC.

Work up: After completion of reaction (TLC), the reaction mixture was diluted with water and extracted with DCM (2×150 mL), the combined organic layer was washed with water and brine solution, dried over anhydrous sodium sulphate and concentrated under vacuum to get crude compound. Purification: The crude compound was purified by silica gel (100-200 mesh) column chromatography by eluting with 50% ethyl acetate in petroleum ether.

| | |
|---|---|
| TLC system: EtOAc | R_{f} value: 0.5 |
| Nature of the compound: Off-white solid | Yield: 30 g (31.4%) |

### Step-2:

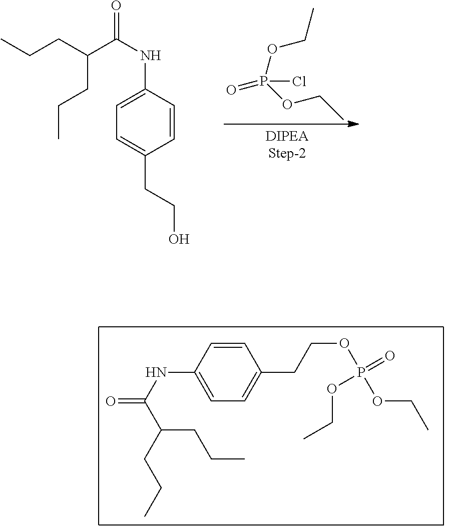

| | |
|---|---|
| Reaction time: 12 hours | Reaction temperature: RT |

Diethyl choloro phosphate (70 mL, 5.0 eq.) was slowly added to a stirred solution of product from Step-1 (25 g, 1.0 eq.) and DIPEA (175 mL, 10.0 eq.) in DCM (500 mL) at RT under nitrogen. The reaction mixture was stirred for 12 hours at RT. The reaction progress was monitored by TLC.

Work up: After completion of reaction (TLC), the reaction mixture was diluted with water and extracted with DCM (2×250 mL), the combined organic layer was washed with water and brine solution dried over anhydrous sodium sulphate and concentrated under vacuum to get crude compound. Purification: The crude compound was purified by silica gel (100-200 mesh) column chromatography by eluting with 5% methanol in DCM.

| | |
|---|---|
| TLC system: EtOAc | R_{f} value: 0.2 |
| Nature of the compound: Pale yellow solid | Yield: 10.12 q (26.6%) |

### Example 23

### Synthesis of Amides (Phospho-ibuprofen amide, compound 1)

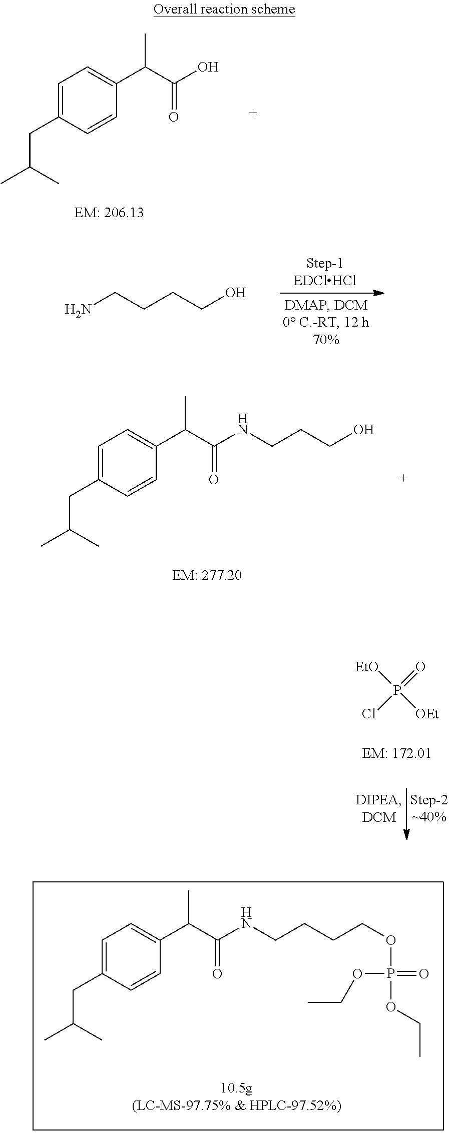 [SMILES: CC(C)Cc1ccc(C(C)C(=O)O)cc1 ; NCCCCO ; CC(C)Cc1ccc(C(C)C(=O)NCCCO)cc1 ; CCOP(=O)(Cl)OCC ; CCOP(=O)(OCC)OCCCCNC(=O)C(C)c1ccc(CC(C)C)cc1]

Step-1

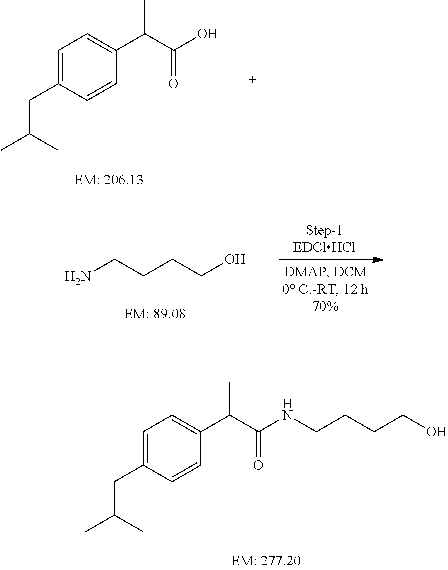 [SMILES: CC(C)Cc1ccc(C(C)C(=O)O)cc1 ; NCCCCO ; CC(C)Cc1ccc(C(C)C(=O)NCCCCO)cc1]

To a stirred solution of first starting (at the left above) material (25.0 g, 121.35 mmol) in DCM (250 mL), EDC.HCl (32.57 g, 170 mmol) was added at 0° C., followed by a catalytic amount of DMAP and second starting material (at the right above) (10.80 g, 121.35 mmol). This mixture was stirred for 10-15 minutes at 0° C. and then at room temperature for 12 hours. Reaction mass was quenched with ice water (100 mL). Separately the organic layer and the aqua's layer was extracted with DCM (2×100 mL). The combined organic layer was washed with brine (100 mL), dried over anhydrous MgSO₄, concentrated under reduced pressure and finally purified by column chromatography on silica gel (100-200 mesh) using ethyl acetate-hexane (30:70) as an eluent to afford the product as a white solid.

| | |
|---|---|
| TLC system: Product Rf: 0.6 (5% Methanol in | Yield: 23.5 (70.0%). |
| DCM). | |

Step 2:

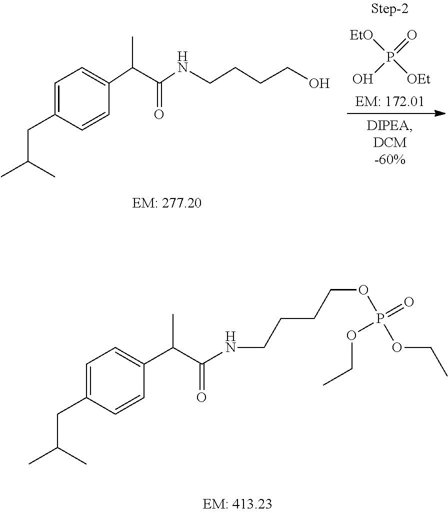 [SMILES: CC(C)Cc1ccc(C(C)C(=O)NCCCCO)cc1 ; CCOP(=O)(O)OCC ; CCOP(=O)(OCC)OCCCCNC(=O)C(C)c1ccc(CC(C)C)cc1]

To a stirred solution of product from Step-1 (10 g, 36.07 mmol) in dichloromethane (150 mL) at 0° C., diethyl phosphorochloridate (2.5 eq, 15.5 g, 90.18 mmol) was added and followed by DIPEA (4 eq, 23.92 ml, 144.38 mmol) and continued stirring for 12 hours at room temperature. Reaction mass was quenched with ice water (100 mL). The organic layer was separated and the aqua's layer was extracted with DCM (2×100 mL). The combined organic layer was washed with brine (100 mL), dried over anhydrous MgSO₄, concentrated under reduced pressure and finally purified by column chromatography on silica gel (100-200 mesh) using methanol, DCM (5:95) as an eluent to afford the product as a Syrup.

| | |
|---|---|
| TLC system: Product Rf: 0.2 (5% Methanol in | Yield: 5.96 (40.0%). |
| DCM). | |

### Example 24

### Synthesis of Amides (Phospho-Quinone Amide, Compound 199)

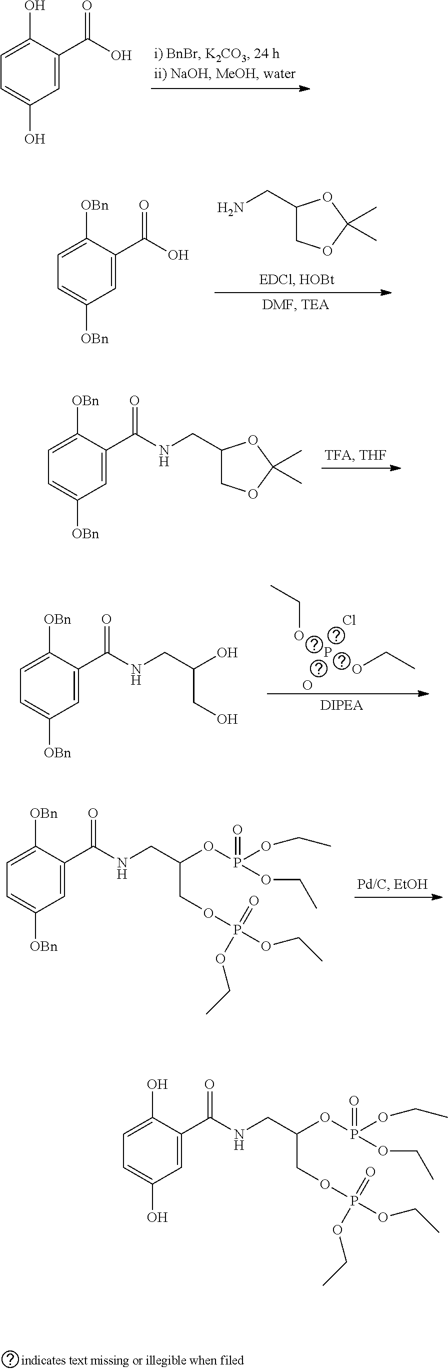

2,5-dihydroxybenzoic acid reacted with BnBr in the presence of K₂CO₃ for 24 h and the reaction was quenched with NaOH and methanol in water. The resulting compound was reacted with (2,2-dimethyl-[1,3]-dioxolan-4-yl)-methylamine, in the presence of 1-Ethyl-3-(3-dimethylaminopropyl)carbodiimide (EDCL), hydroxy-benzotriazole, triethanolamine (TEA) in dimethylformamide (DMF). The hydroxyl groups were deprotected using trifluoroacetic acid (TFA) in tetrahydrofuran (THF). After purification, the product was reacted in excess (10 eq) of diethyl chlorophosphate at room temperature overnight. The protecting groups were removed to expose the hydroxyls after treating with Pd/C in ethanol at 60PSI for 6 hours. The resulting structure was confirmed with ¹H-NMR and LC-MS.

### Example 25

### Synthesis of Amides (Phospho-Salicylate-Methane, Compound 198)

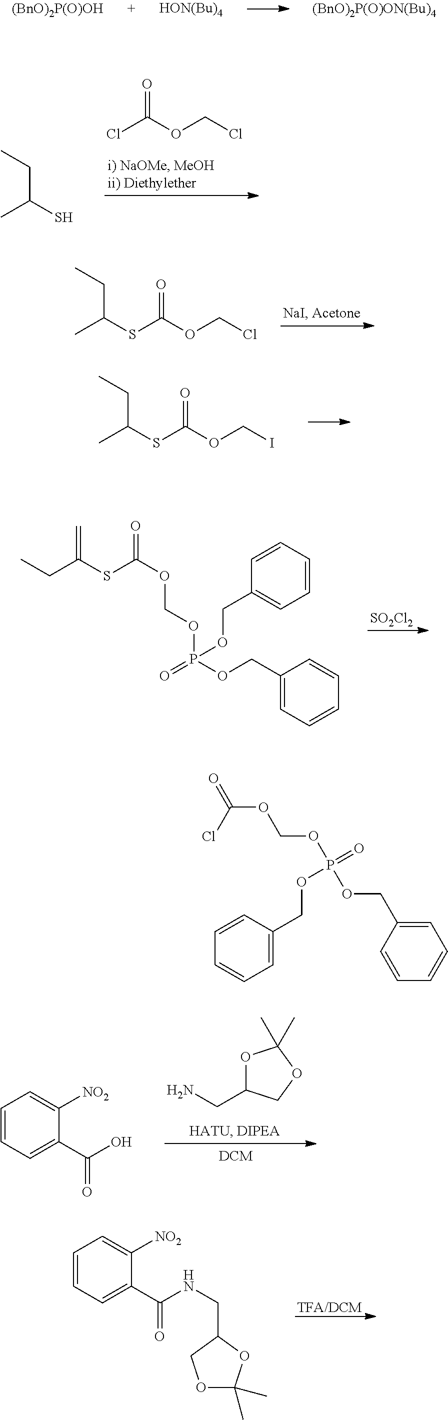

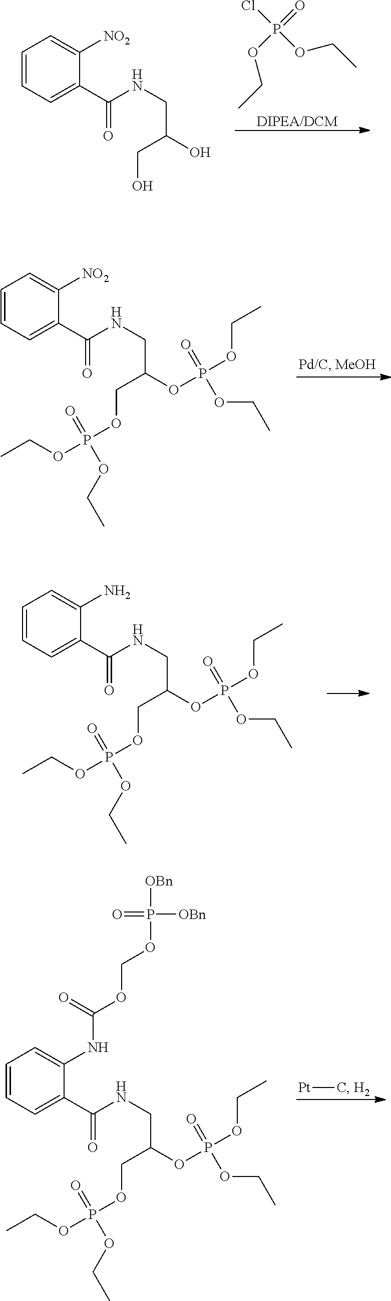

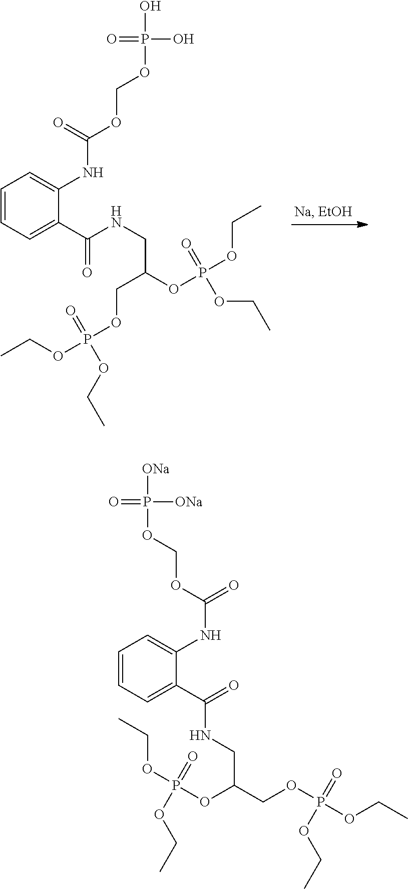

Dibenzyl phosphate was treated with tetrabutylammonium hydroxide in water and lyophilized overnight to produce the first intermediate. Butane-2-thiol was reacted with sodium methoxide in methanol followed by chloromethyl chloroformate in diethyl ether at 0° C. for 1 hour. The resulting product was treated with sodium iodide (Nal) in Acetone at RT. The resulting product was further reacted with the first intermediate (from the first reaction). Following purification, the resulting product was treated with SO₂Cl₂ to produce the second intermediate. 2-Nitrobenzoic acid reacted with (2,2-dimethyl-[1,3]-dioxolan-4-yl)-methylamine in the presence of 1-[Bis(dimethylamino)methylene]-1H-1,2,3-triazolo[4,5-b]pyridinium 3-oxid hexafluorophosphate (HATU) and N,N-Diisopropyl-ethylamine (DIPEA) in dichloromethane (DCM). The resulting product was treated with trifluoroacetic acid in DCM for 8 hours at 0° C. The resulting product was further reacted with diethyl chlorophosphate in the presence of N,N-Diisopropylethylamine (DIPEA) at 0° C. The resulting product was hydrogenated in the presence of 10% Pd/C in MeOH at RT for 4 hours. After purification, the resulting product was allowed to react with the second intermediate. The phosphate group of was debenzylated after treatment with hydrogen in the presence of platinum-on-carbon (Pt/C) and a sodium salt of the product was produced.

### Example 26

### Synthesis of Amides (e.g., Phospho-Farnesyl Salicylate Diamide, Compound 200)

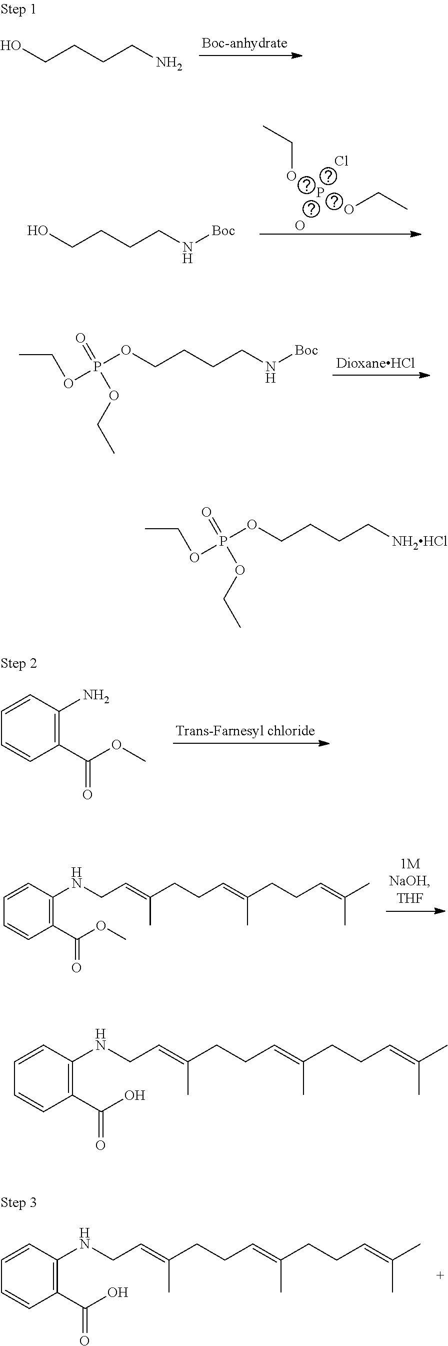

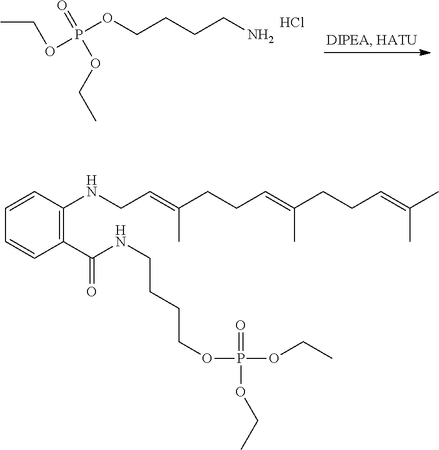 [SMILES: CCOP(=O)(OCC)OCCCCN ; Cl ; CCOP(=O)(OCC)OCCCCNC(=O)c1ccccc1NC/C=C(\C)CC/C=C(\C)CCC=C(C)C]

The amino group of 4-Amino-1-butanol, was protected by a standard reaction with Di-tert-butyl dicarbonate. The resulting product was reacted with diethyl chlorophosphate. The Boc-amino protection group was removed by treating the resulting product with Dioxane-HCl, at room temperature for 16 hours creating 4-aminobutyl diethyl phosphate hydrochloride. Methyl 2-aminobenzoate was reacted with trans-Farnesyl chloride (0.9 eq), NaI (0.3 eq) under reflux. The resulting product (40% yield) was treated with 1 M NaOH and THF at 60° C. for 6 hours. The resulting compound was slowly added inside the suspension of 4-aminobutyl diethyl phosphate hydrochloride at room temperature in the presence of 1-[Bis(dimethylamino)methylene]-1H-1,2,3-triazolo[4,5-b]pyridinium 3-oxid hexafluorophosphate (HATU) and N,NDiisopropylethylamine (DIPEA) in dimethylormamide and was left to react overnight.

### Example 27

### Antithrombotic Effect

Compounds of the present invention were assessed for their effects on a) thrombus formation in an animal model; and b) platelet aggregation.

### Inhibition of Thrombus Formation In Vivo

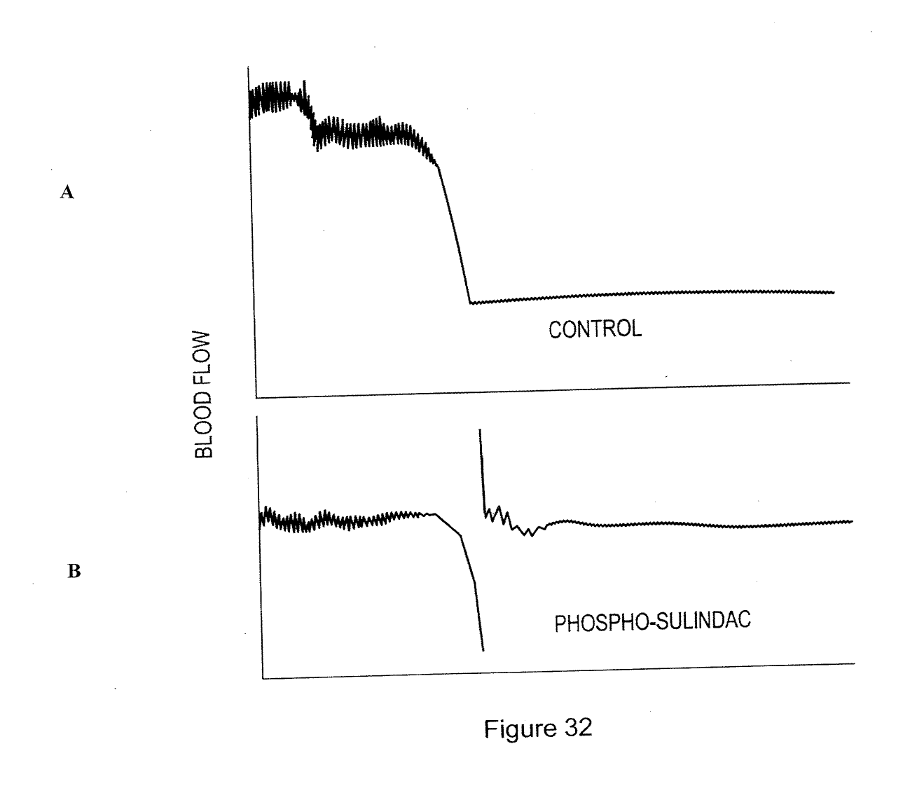

The experimental model of ferric chloride (FeCl₃)-induced thrombus (Lockyer S, Kambayashi J. Demonstration of flow and platelet dependency in a ferric chloride-induced model of thrombosis. J Cardiovasc Pharmacol 1999; 33: 718-25) was used. In this model the carotid artery is briefly exposed to FeCl₃ leading to thrombus formation at the site of the injury and occlusion of blood flow. Mice were orally administered 40% of the maximum tolerated dose (MTD) of phospho-sulindac (PS) or vehicle, and one hour later the right carotid artery was injured with 7.5% of FeCl₃ solution. Flow rate was then measured for 30 minutes (FIG. 32).

### Inhibition of Platelet Aggregation

Two methods were used to evaluate whether platelet aggregation was inhibited by compounds of the present invention: a) thromboelastography, a procedure widely used to assess whole blood or plasma hemostasis; and b) platelet aggregometry, which determines directly platelet aggregation (the clumping together of platelets in the blood) which is part of the sequence of events leading to the formation of a thrombus (clot) (Thromb Res. 2012; 129:681-7).

Thromboelastography:

A thromboelastograph (TEG 5000, Haemonetics Corp., Braintree, Mass.) was employed according to manufacturer's procedure. This method produces a signal that reflects fibrin clot formation. The signal is amplified by platelets at least several fold. The maximum signal amplitude (MA) is used to calculate the viscoelastic parameter of the clot. MA is increased by fibrin cross-linking and by platelets, reflecting platelet retraction of the clot that is mediated by fibrinogen binding to the integrin α_{11b}β₃, a fibrinogen receptor on the surface of activated platelets.

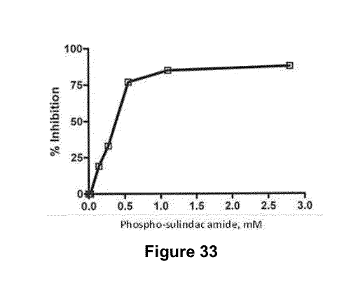

Conditions were designed to yield no fibrinogen signal so that all of MA reflected the platelet effect on the clot. This involved dilution of human platelet-rich plasma (PRP), platelet level ˜1 to 1.5×10⁶/μl, so that the platelets in clotting mixtures ranged ˜150 to 400×10³/μl. The clotting buffer, TBS, pH 7.4, contained sufficient CaCl₂ (10 mM) to neutralize the citrate in the standard ACDA anticoagulant, the excess or free CaCl₂ serving to maximize PRP clot formation. After a 10 minute incubation of reactants with phospho-silindac amide (butane spacer), clotting was induced by recombinant thrombin 1-2 U/ml. The results established a dose-dependent inhibition of MA that was platelet specific. Phospho-sulindac amide exerted no effect on clots from platelet poor plasma (PPP), n=2, or those from purified fibrinogen. PPP clots were obtained by spiking with purified fibrinogen to obtain a measurable signal. In two dose response and several additional single experiments, the MA inhibition of the PRP ranged from 0 to 85% (FIG. 33). Increasing the phospho-sulindac amide concentration beyond this point disclosed a plateau, indicating a saturable form of inhibition.

In a single set of dose response experiments (platelets 420×10³/μl) the slope increased from 0 to −0.5 mM B before reaching a plateau. Two other (single donor) batches of PRP, using one or two concentrations of phospho-silindac amide, yielded inhibition within this general range. The range of platelet concentrations, 128 to 895×10³/μl, did not significantly affect the inhibition range. This can be explained by the relatively low fibrinogen concentration, roughly one third to one fifth that of plasma, resulting from the PRP dilution used in various experiments. That is, there was a finite number of fibrinogen/fibrin receptor recognition sites (at least 2/mol with possible additional auxiliary sites) which the platelet receptors occupied, and once these links were blocked additional platelets had no effect on the clot retraction (i.e. the MA signal).

Similar results were obtained with phospho-aspirin glycerol amide 194 and phospho-sulindac 96. They inhibited platelet aggregation between 30-80% at concentrations ranging between 0.25 and 1.2 mM.

Platelet Aggregometry:

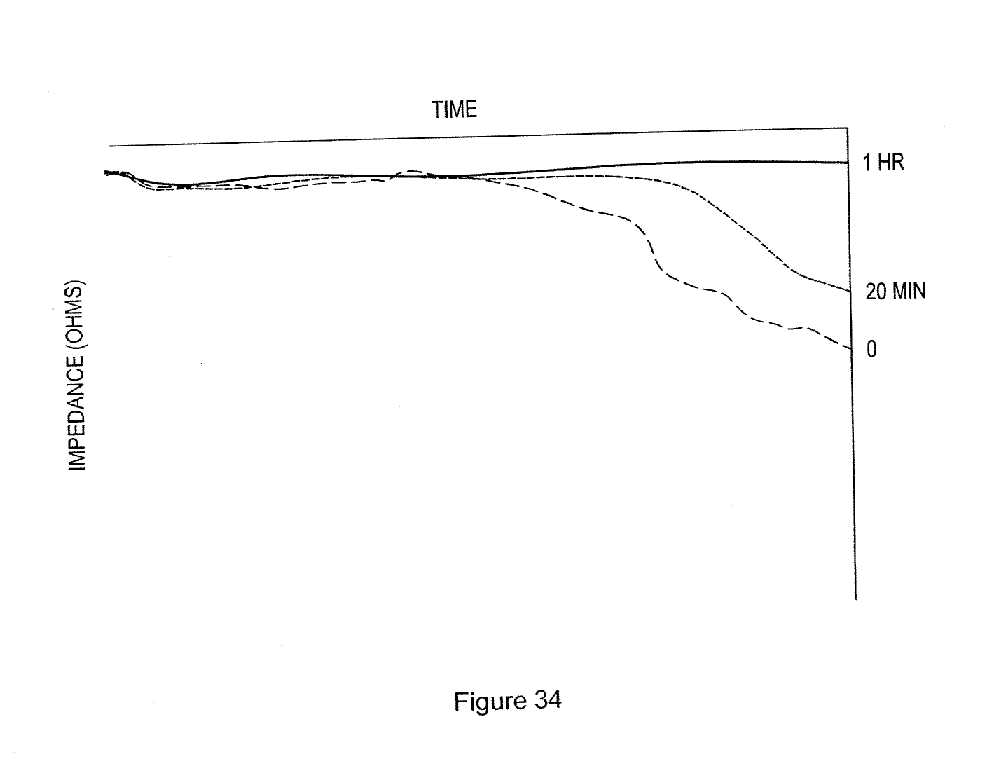

Platelet aggregation was studied using an Aggregometer (Chrono-Log, Havertown, Pa.). Mice were treated with phospho-sulindac 96, at 40% MTD and were then sacrificed at different time points for ex vivo aggregometry using collagen as the agonist at 1 μg/mL. Maximum inhibition of aggregation occurred at 1 hour post treatment (FIG. 34).

### Lack of Effect on Bleeding Time

The bleeding time is a measure of the interaction of platelets with the blood vessel wall. Thus it determines, among others, the propensity of an antiplatelet agent to cause bleeding.

Method:

The bleeding time was determined with the “tail-bleed assay”. Tail bleeding time was measured following standard protocols 1 hour after administering orally the test agent or corn oil (solvent control) to groups of 5-6 mice. The following compounds were administered: either phospho-sulindac 96 or phospho-sulindac amide 95, each at 150 mg/kg each. The incision was made 2 mm from the tip of the tail to expose the tail vein, and the bleeding time was determined.

Results:

Bleeding time: Control: 112±18 sec; phospho-sulindac 96: 88±7 sec; phospho-sulindac amide 95: 91±6 sec. Thus these two compounds inhibit platelet aggregation without prolonging the bleeding time, a clinically highly desirable combination of properties.

### Example 28

### Analgesic Effect

Analgesic effect was evaluated using the hot-plate test, following a standard protocol (Bannon A W. Models of Pain: Hot-plate and formalin test in rodents. Current Protocols in Pharmacology: John Wiley & Sons; 1998). An LE7406 hot plate (Panlab Harvard Apparatus, Spain) was maintained at 55±0.5° C. The instrument records digitally as latency the time between placing the rat on the plate and licking of the paws or jumping. Lewis rats were used. The compounds were administered ip 30 minutes prior to testing. Controls received vehicle (corn oil). The study groups (n=6) and results are shown in the Table (All differences from vehicle control are statistically significant.b

| Compound | | | Latency, sec |
|---|---|---|---|
| Number | Test compound | Dose mg/kg | Mean ± SEM |
| | Vehicle | | 12.42 ± 1.25 |
| 95 | Phospho-sulindac butane | 120 | 22.05 ± 0.55 |
| | amide | | |
| 194 | Phospho-aspirin glycerol | 150 | 19.92 ± 0.78 |
| | mono-amide | | |
| 1 | Phospho-ibuprofen butane | 120 | 23.42 ± 1.16 |
| | amide | | |

Carboxylic esters, e.g. phospho-sulindac 96 were also effective. In addition to systemic administration, they were efficacious when applied topically formulated in a hydrogel.

These exemplary amide compounds of the invention demonstrate analgesic activity, and indicate that the amide compounds of the invention have analgesic properties and are potentially effective for treating pain in animals, including mammals and humans.

### Example 29

### Synthesis of Phospho-Glycerol Ibuprofen Amide (Compound 4)

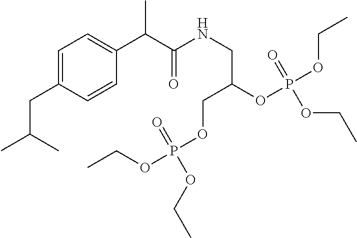 [SMILES: CCOP(=O)(OCC)OCC(CNC(=O)C(C)c1ccc(CC(C)C)cc1)OP(=O)(OCC)OCC]

Phospho Glycerol Ibuprofen Amide

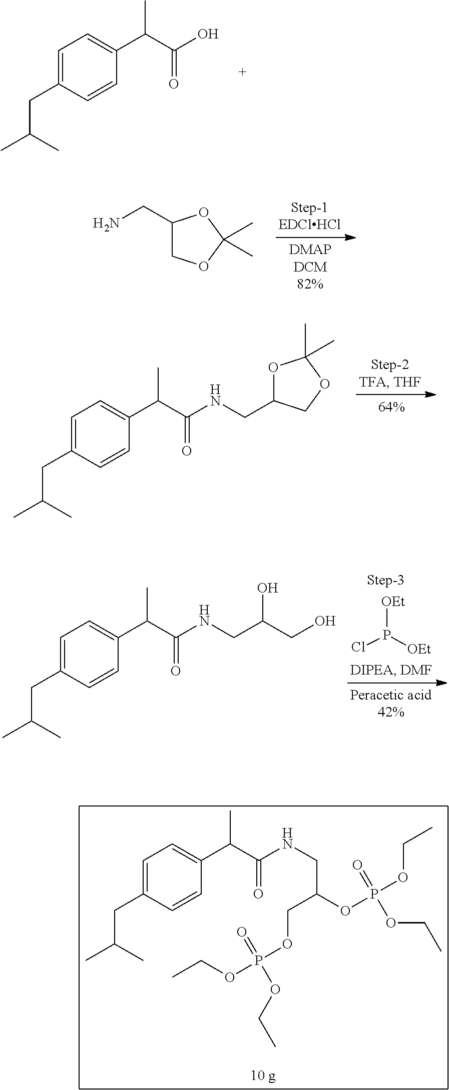

### Experimental Details

### Step 1:

To a stirred solution of Ibuprofen (25 gm, 121.35 mM) in dichloromethane (DCM) (250 ml), EDC.HCL (27.81 g, 145.6 mM) and 4-dimethylaminopyridine (DMAP) (14.80 m, 121.35 mM) were added at 0° C. After 30 minutes amine (15.91 g, 121.35 mM) was added to this suspension. The resulting reaction mixture was stirred at room temperature overnight. The reaction mixture was diluted with DCM and washed with water. The organic layer was separated and it was concentrated. The crude product was purified by flash column chromatography using 0-30% EA in hexane as eluent. 32 g (82%) of product was obtained as color less liquid.

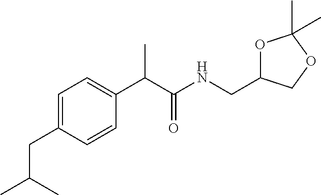 [SMILES: CC(C)Cc1ccc(C(C)C(=O)NCC2COC(C)(C)O2)cc1]

### N-((2,2-dimethyl-1,3-dioxolan-4-yl)methyl)-2-(4-isobutylphenyl)propanamide

### Step 2:

To a stirred solution of starting material (SM) (32 g, 100.313 mM) in DCM (dichloromethane), TFA (trifluoroacetate) was added slowly at 0° C. The reaction mixture was stirred at room temperature for 3 hours. The reaction mixture was concentrated and the residue was purified by flash column chromatography using 30% ethyl acetate in pet ether as eluent. 18.0 g (64%) was obtained as color less liquid.

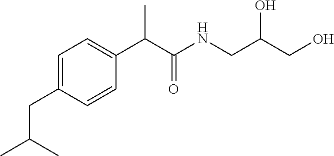 [SMILES: CC(C)Cc1ccc(C(C)C(=O)NCC(O)CO)cc1]

### N-(2,3-dihydroxypropyl)-2-(4-isobutylphenyl)propanamide

Step 3:

To a stirred solution of starting material (SM) (18 g, 64.51 mM) in DMF (100 ml), DIPEA (N,N-Diisopropylethylamine) was added at −42° c. Diethyl chlorophosphite (40.39 g, 258.06 mM) was added to this solution. The resulting reaction mixture was stirred at RT for 8 h. The reaction mixture was diluted with ethyl acetate and washed with water. The organic layer was separated and dried over Na₂SO₄. It was concentrated and the crude compound was dissolved in THF (300 ml). The solution was cooled to 0° C. To this solution, peracetic acid (300.gm, 3949.3 mM) was added and the resulting mixture was stirred at RT for 1 hour. The reaction mixture was concentrated and it was quenched with Sodium meta bisulphate solution. It was extracted with ethyl acetate. The organic layer was separated and dried over Na₂SO₄. It was concentrated. The crude compound was purified by flash silica gel column chromatography using ethylacetate and hexane as eluent. The isolated product was further purified by COMBI FLASH (reverse phase). 15.0 g (42%) of required product was obtained as greenish yellow foamy solid.

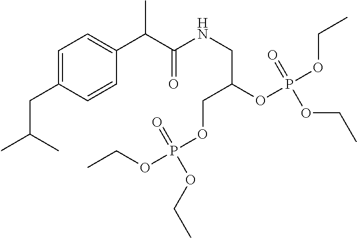 [SMILES: CCOP(=O)(OCC)OCC(CNC(=O)C(C)c1ccc(CC(C)C)cc1)OP(=O)(OCC)OCC]

### Tetraethyl 3-(2-(4-isobutylphenyl)propanamido)propane-1,2-diyl diphosphate

### Example 30

### Synthesis of Sulindac Butane Amide (Compound 95)

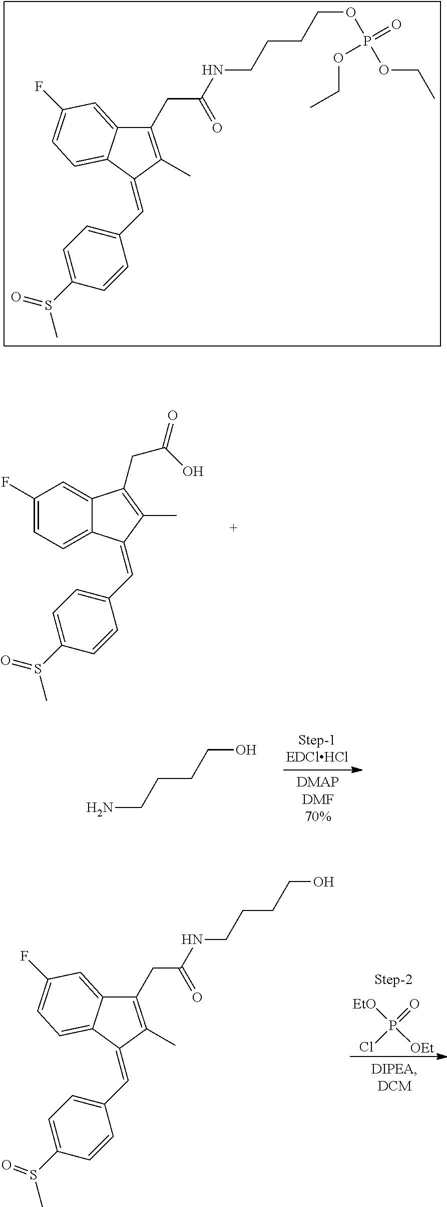 [SMILES: CCOP(=O)(OCC)OCCCCNC(=O)CC1=C(C)/C(=C/c2ccc(S(C)=O)cc2)c2ccc(F)cc21 ; CC1=C(CC(=O)O)c2cc(F)ccc2/C1=C\c1ccc(S(C)=O)cc1 ; NCCCCO ; CC1=C(CC(=O)NCCCCO)c2cc(F)ccc2/C1=C\c1ccc(S(C)=O)cc1 ; CCOP(=O)(Cl)OCC]

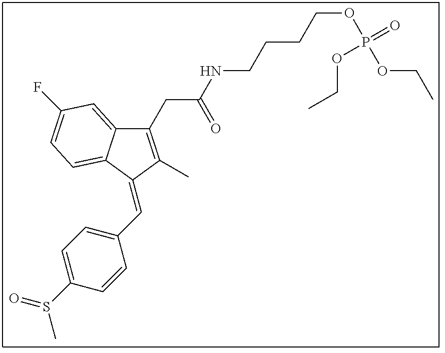 [SMILES: CCOP(=O)(OCC)OCCCCNC(=O)CC1=C(C)/C(=C/c2ccc(S(C)=O)cc2)c2ccc(F)cc21]

### Experimental Details

Step 1:

To a stirred solution of sulindac (25 g, 0.07022 mM) in DCM (250 ml), EDC.HCL(13.37 gm, 0.07022 mM) and DMAP (8.5 gm, 0.07022 mM) were added at 0° C. Then after 30 minutes 4-amino butanol (9.77 g, 0.070225 mM) was added. The reaction mixture was stirred at room temperature overnight. The reaction mixture was diluted with DCM and washed with water. The organic layer was separated and it was concentrated. The crude product was purified by flash column chromatography using 0-30% ethylacetate (Ea) in hexane as eluent. 29 g (72%) of product was obtained as color less liquid.

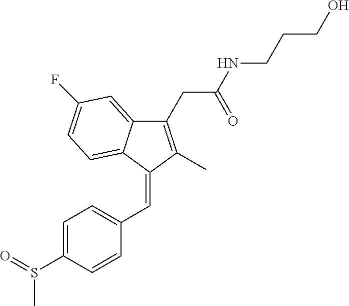 [SMILES: CC1=C(CC(=O)NCCCO)c2cc(F)ccc2/C1=C\c1ccc(S(C)=O)cc1]

### (Z)-2-(5-fluoro-2-methyl-1-(4-(methylsulfinyl)benzylidene)-1H-inden-3-yl)-N-(4-hydroxybutyl)acetamide

### Step 2:

To a stirred solution of SM (29 g, 0.16978 mM) in DCM (290 ml), DIPEA was added at −42° c. Then Diethyl chlorophoshate (24.54 g, 0.1697 mM) was added to the reaction mixture. The resulting reaction mixture was stirred at RT for 8 h. The reaction mixture was diluted with water and DCM. The organic layer was separated and dried over Na₂SO₄. It was concentrated. The crude compound was purified by flash silica gel column chromatography and further purification was done on COMBI FLASH (reverse phase). 15.0 g (42%) of required product was obtained as greenish yellow foamy solid.

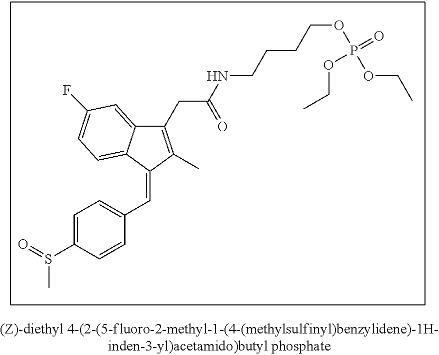 [SMILES: CCOP(=O)(OCC)OCCCCNC(=O)CC1=C(C)/C(=C/c2ccc(S(C)=O)cc2)c2ccc(F)cc21]

### Example 31

### Synthesis of Sulindac Butane Amide (Compound 99)

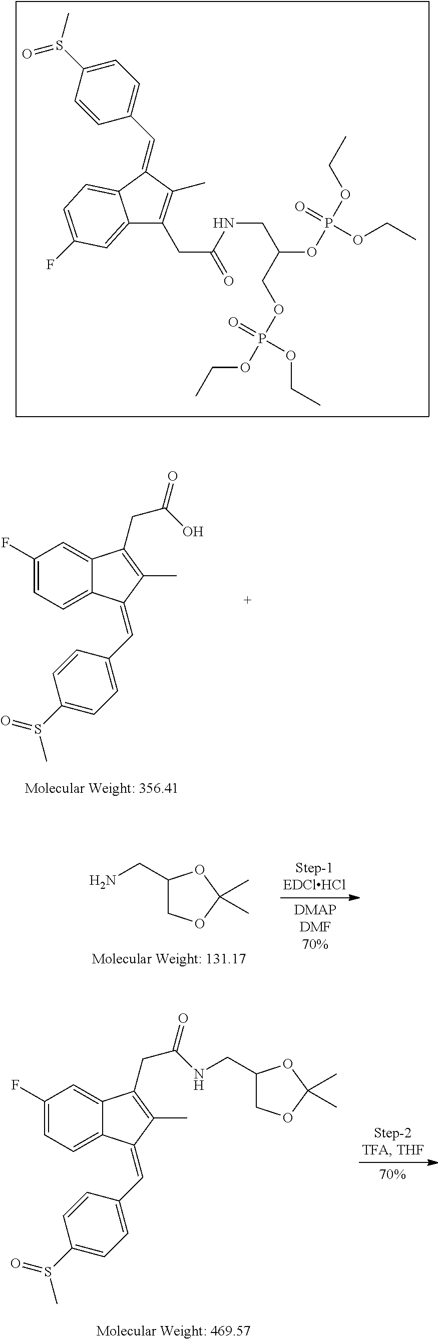

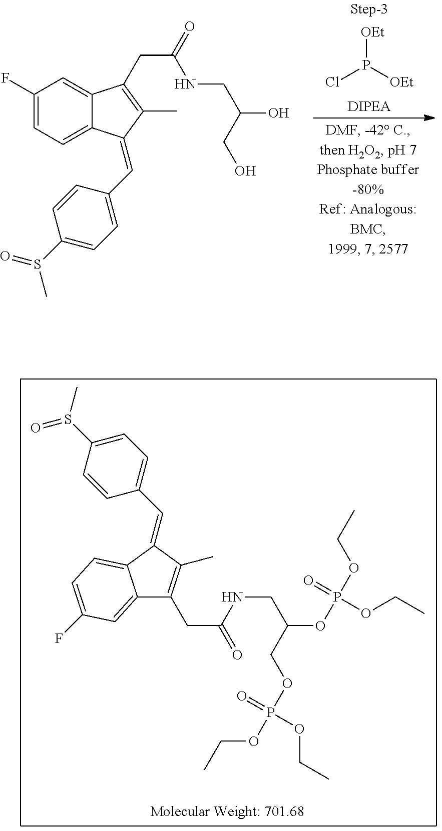 [SMILES: CC1=C(CC(=O)NCC(O)CO)c2cc(F)ccc2/C1=C\c1ccc(S(C)=O)cc1 ; CCOP(Cl)OCC ; CCOP(=O)(OCC)OCC(CNC(=O)CC1=C(C)/C(=C/c2ccc(S(C)=O)cc2)c2ccc(F)cc21)OP(=O)(OCC)OCC]

### Experimental Details

### Step 1:

To a stirred solution of sulindac (25 g, 0.07022 mM) in DCM (250 ml), EDC.HCL(16.81 g, 0.084269 mM) and DMAP (8.56 g, 0.07022 mM) were added at 0° C. Then after 30 minutes amine (9.91 g, 0.07022 mM) was added. The reaction mixture was stirred at room temperature overnight. The reaction mixture was diluted with DCM and washed with water. The organic layer was separated and it was concentrated. The crude product was purified by flash column chromatography using 0-30% Ea in hexane as eluent; 18 g (42%) of product was obtained as color less liquid.

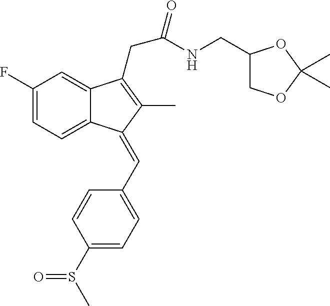 [SMILES: CC1=C(CC(=O)NCC2COC(C)(C)O2)c2cc(F)ccc2/C1=C\c1ccc(S(C)=O)cc1]

### N-((2,2-dimethyl-1,3-dioxolan-4-yl)methyl)-2-(4-isobutylphenyl) propanamide

Step 2:

To a stirred solution of SM (18 g, 0.03837 mM) in DCM, TFA was added slowly at 0° C. After 3 hours stirred at room temperature, the reaction mixture concentrated. The crude product was purified by flash column chromatography using 30% ethyl acetate in pet ether as eluent. 9.2 g (64%) was obtained as color less liquid.

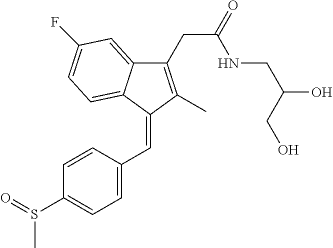 [SMILES: CC1=C(CC(=O)NCC(O)CO)c2cc(F)ccc2/C1=C\c1ccc(S(C)=O)cc1]

### (Z)-N-(2,3-dihydroxypropyl)-2-(5-fluoro-2-methyl-1-(4-(methylsulfinyl)benzylidene)-1H-inden-3-yl)acetamide

### Step 3:

To a solution of SM (8.2 g, 0.01911 mM) in DCM (80 ml), TEA was added at −42° C. Then Diethyl chlorophosphate (16.93 g, 0.097 mM) was added to the reaction mixture. The resulting reaction mixture was sonacated at RT for 3 hours. The reaction mixture was diluted with water and DCM. The organic layer was separated and dried over Na₂SO₄. The crude compound was purified by flash silica gel column chromatography and further purification was done on COMBI FLASH (reverse phase); 8.0 g (42%) of required product was obtained as greenish yellow foamy solid.

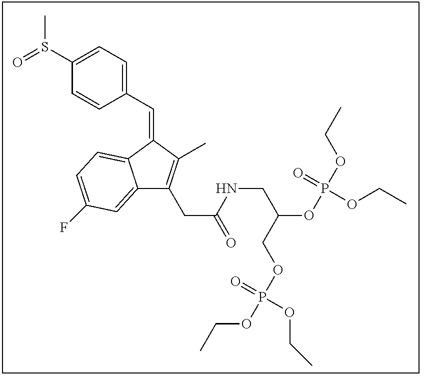 [SMILES: CCOP(=O)(OCC)OCC(CNC(=O)CC1=C(C)/C(=C/c2ccc(S(C)=O)cc2)c2ccc(F)cc21)OP(=O)(OCC)OCC]

### (Z)-tetraethyl 3-(2-(5-fluoro-2-methyl-1-(4-(methylsulfinyl)benzylidene)-1H-inden-3-yl)acetamido)propane-1,2-diyl diphosphate

* * * *

All publications, patent applications, and patents mentioned in this specification are herein incorporated by reference. While the invention has been described in connection with specific embodiments, it will be understood that these are representative, and the invention encompasses additional embodiments and is capable of further modifications. This application is intended to cover any variations, uses, or adaptations of the invention that follow, in general, the principles of the invention, including departures from the present disclosure that come within known or customary practice within the art. Other embodiments are within the claims.

What is claimed is:

1. A compound of Formula I 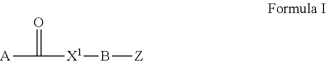
or an enantiomer, diastereomer, racemate, tautomer, salt or hydrate thereof, wherein
A is an optionally substituted aliphatic, heteroaliphatic, aromatic, heteroaromatic substituent or alkylaryl substituent having 1 to 100 carbon atoms;
X¹ is selected from the group consisting of —O—, —S— and —NR¹—, R¹ being hydrogen or C₁₋₁₀₀-alkyl;
B is selected from the group consisting of 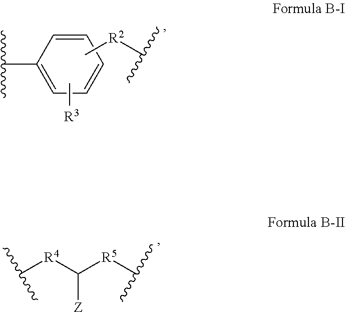
a single bond and an aliphatic group with 1 to 100, more preferred with 1 to 42 and particularly preferred with 1 to 22 carbon atoms,
R², R⁴ and R⁵ are the same or different C₁₋₃-alkylene,
R³ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)0-C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;
and Z is selected from the group consisting of 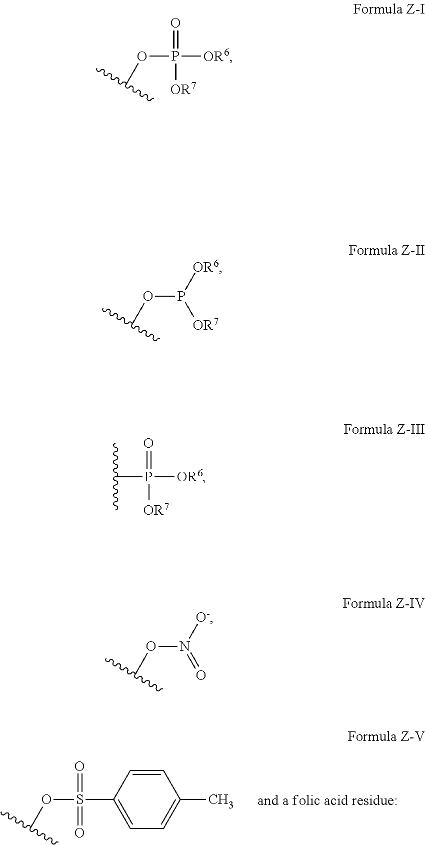
wherein R⁶ is independently selected from hydrogen, C₁₋₁₀₀-alkyl, preferably C₁₋₆-alkyl, and a polyethylene glycol residue,
R⁷ is independently selected from hydrogen, C₁₋₁₀₀-alkyl, preferably C₁₋₆-alkyl, and a polyethylene glycol residue; or
B together with Z forms a structure 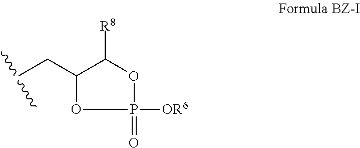
wherein R⁶ is defined as above, and R⁸ is independently selected from hydrogen, an aliphatic substituent with 1 to 22 carbon atoms, C₁₋₆-alkyl, and a polyethylene glycol residue.

2. A compound of claim 1, wherein A is selected from the group consisting of 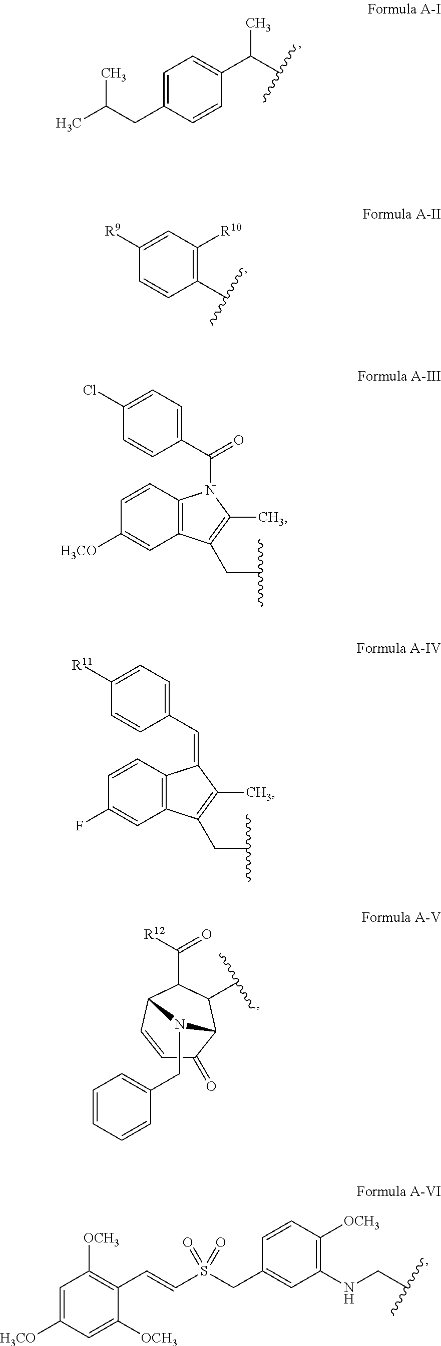 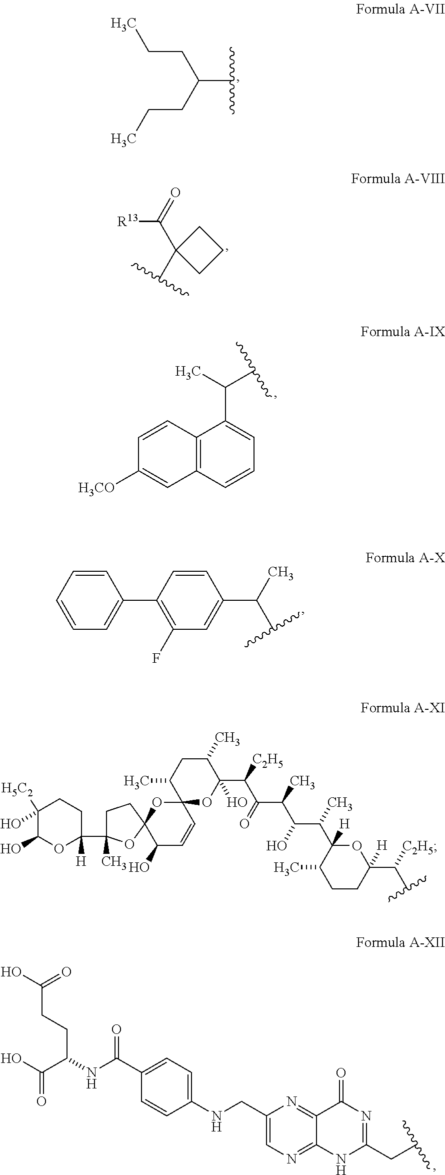 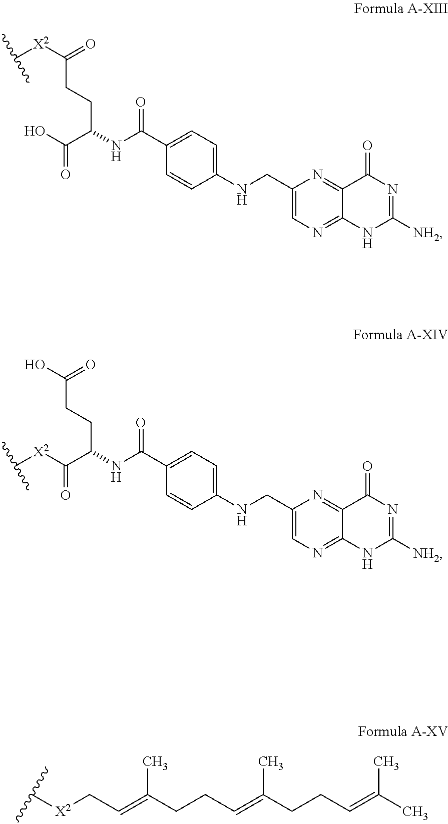
wherein:
R⁹ is selected from hydrogen and trifluoromethyl;
R¹⁰ is selected from —X²—C(O)—CH₃,
R¹¹ is selected from —SCH₃, —S(O)CH₃ and —S(O)₂CH₃;
R¹² is selected from hydroxy, —B—Z and Formula A-XII; and
X² is selected from the group consisting of —O—, —S— and —NR13—, R¹³ being hydrogen or C₁₋₆-alkyl.

3. A compound of claim 1, wherein Z is a folic acid residue selected from the group consisting of 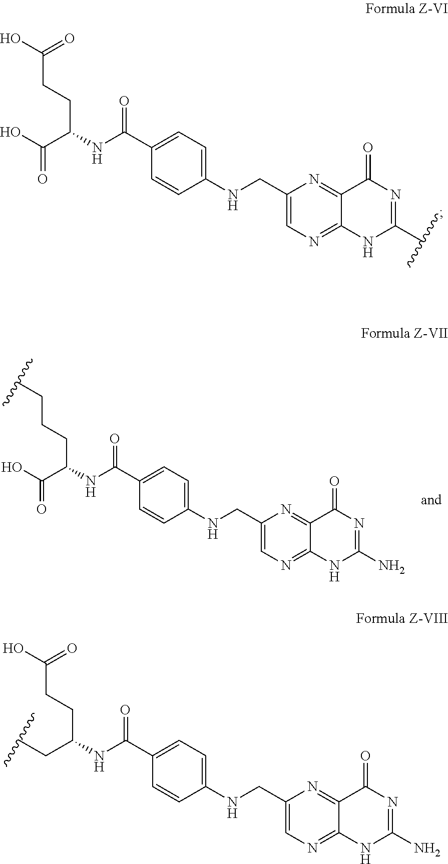 [SMILES: *c1nc(=O)c2nc(CNc3ccc(C(=O)N[C@@H](CCC(=O)O)C(=O)O)cc3)cnc2[nH]1 ; *CCC[C@H](NC(=O)c1ccc(NCc2cnc3[nH]c(N)nc(=O)c3n2)cc1)C(=O)O ; *C[C@H](CCC(=O)O)NC(=O)c1ccc(NCc2cnc3[nH]c(N)nc(=O)c3n2)cc1]
and B is selected from the group consisting of 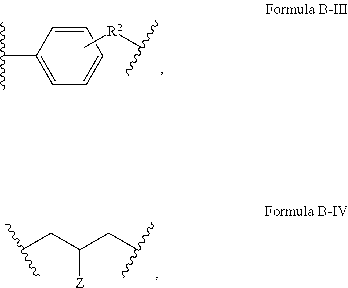
single bond, C₁₋₆-alkylene, C₂₋₆-alkenylene and C₂₋₆-alkynylene;
Z is represented by Formula Z-I,
wherein:
R⁶ is independently selected from hydrogen, C₁₋₃-alkyl and (OCH₂CH₂)ₙOCH₃,
R⁷ is independently selected from C₁₋₃-alkyl and (OCH₂CH₂)ₙOCH₃, whereby n is from 40 to 50.

4. A compound of claim 1, wherein X¹ is —NR¹—, R¹ is hydrogen; B is selected from the group consisting of C₁₋₄-alkylene and 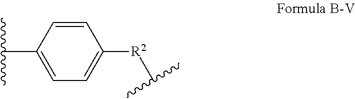
R² being methylene or ethylene; and Z is represented by Formula Z-I, wherein R⁶ and R⁷ are identical C₁₋₃-alkyl substituents.

5. A compound of claim 1, wherein X¹ is —NR¹—, R¹ is hydrogen; B is —(CH₂)₄—; and Z is represented by Formula Z-I, R⁶ and R⁷ being identical C₁₋₃-alkyl substituents.

6. A compound according to claim 1, wherein X¹ is —NH—; B is —(CH₂)₄—; and Z is represented by Formula Z-I, R⁶ and R⁷ being identical C₁₋₃ alkyl substituents.

7. A compound of Formula II 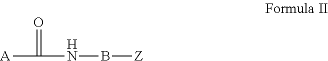
or an enantiomer, diastereomer, racemate, tautomer, salt or hydrate thereof, wherein A is an optionally substituted aliphatic, heteroaliphatic, aromatic, heteroaromatic substituent or alkylaryl substituent having 1 to 100 carbon atoms;
X¹ is selected from the group consisting of —O—, —S— and —NR¹—, R¹ being hydrogen or C₁₋₁₀₀-alkyl;
B is selected from the group consisting of 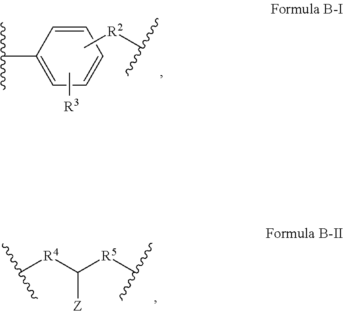
a single bond, and an aliphatic group with 1 to 100 carbon atoms, 1 to 42 carbon atoms, or 1 to 22 carbon atoms,
R², R⁴ and R⁵ are the same or different C₁₋₃-alkylene,
R³ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O-C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy;
and Z is selected from the group consisting of 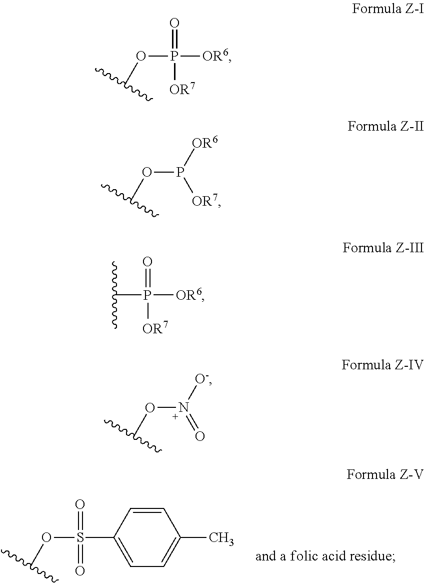
wherein R⁶ is independently selected from hydrogen, C₁₋₁₀₀-alkyl, preferably C₁₋₆-alkyl, and a polyethylene glycol residue,
R⁷ is independently selected from hydrogen, C₁₋₁₀₀-alkyl, preferably C₁₋₆-alkyl, and a polyethylene glycol residue;
or B together with Z forms a structure 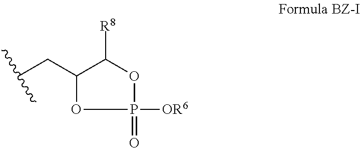
wherein R⁶ is defined as above, and
R⁸ is independently selected from hydrogen, an aliphatic substituent with 1 to 22 carbon atoms, more preferred C₁₋₆-alkyl and a polyethylene glycol residue.

8. A compound of claim 7, wherein A is selected from the group consisting of 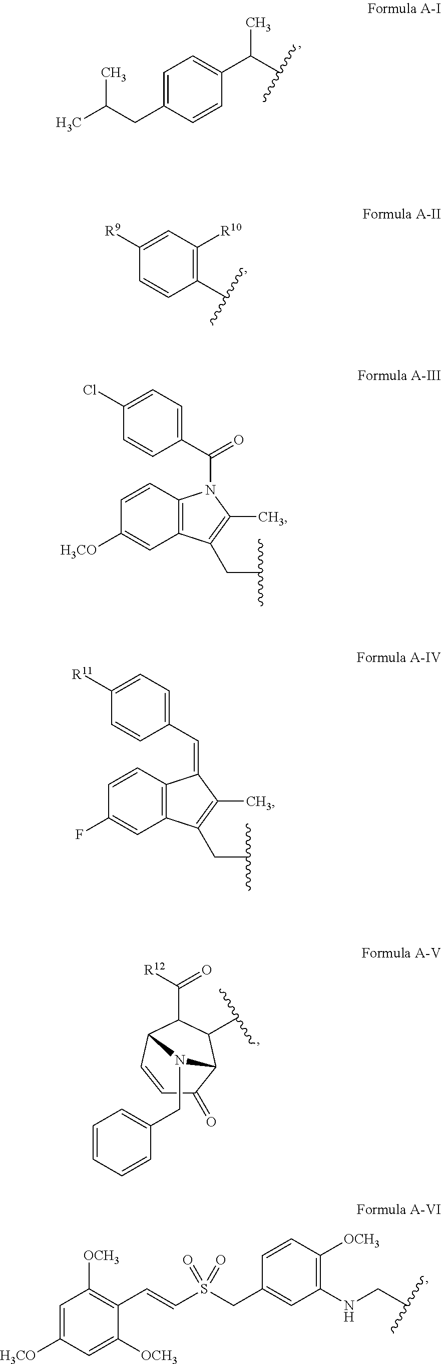 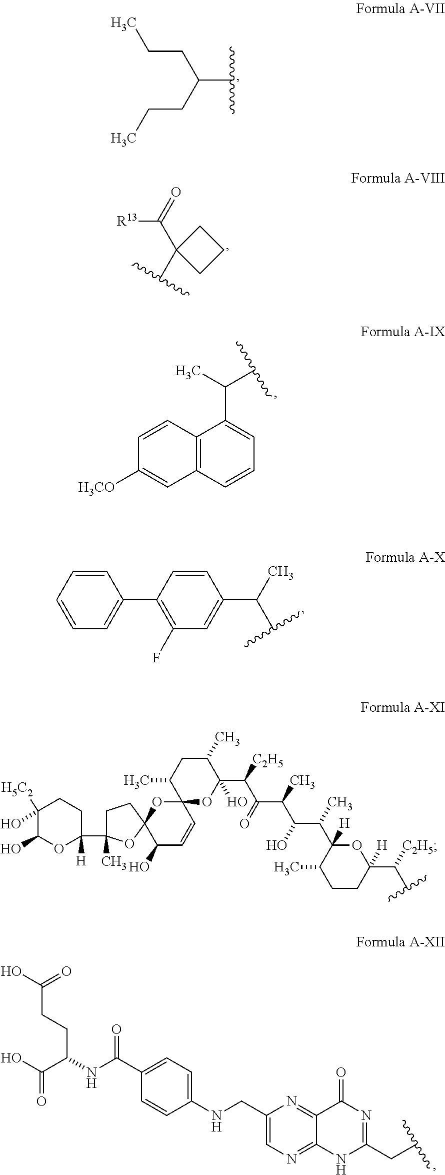 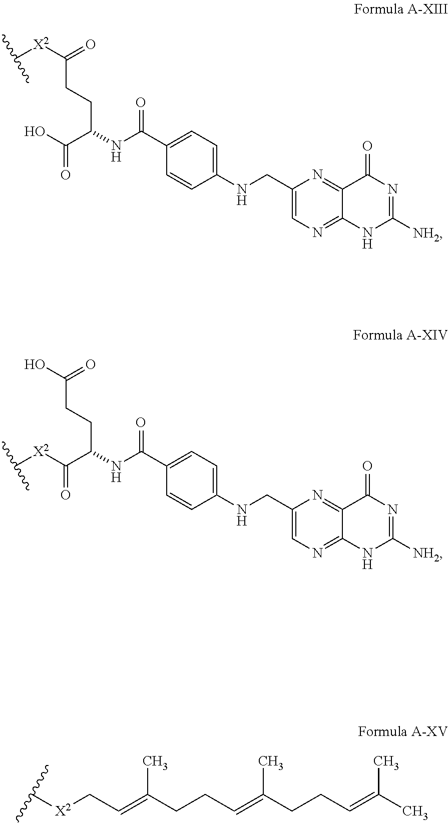
wherein R⁹ is selected from hydrogen and trifluoromethyl;
R¹⁰ is selected from —X²—C(O)—CH₃,
R¹¹ is selected from —SCH₃, —S(O)CH₃ and —S(O)₂CH₃;
R¹² is selected from hydroxy, —B—Z and Formula A-XII;
X² is selected from the group consisting of —O—, —S— and —NR¹³—, and
R¹³ is hydrogen or C₁₋₆-alkyl.

9. A compound of claim 7, wherein the folic acid residue is selected from the group consisting of 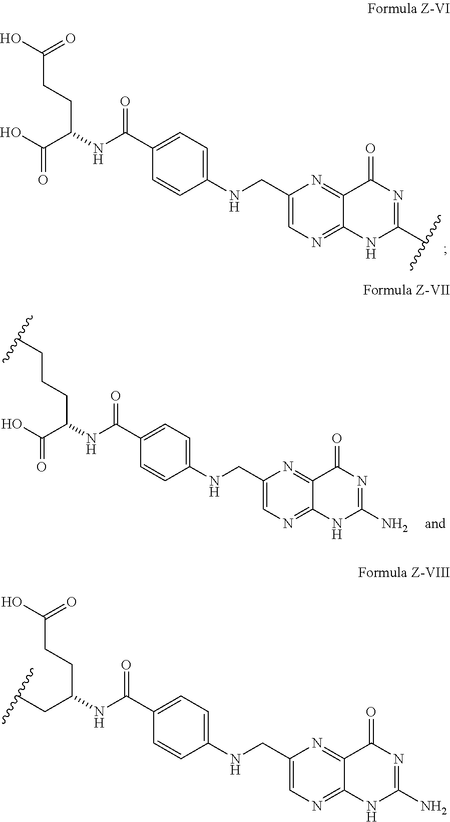
B is selected from the group consisting of 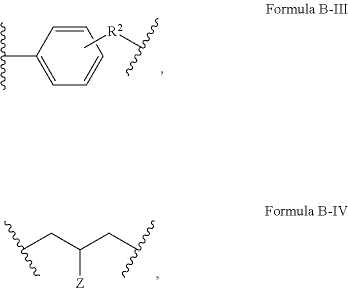
a single bond, C₁₋₆-alkylene, C₂₋₆-alkenylene and C₂₋₆-alkynylene; and
Z is represented by Formula Z-I, wherein
R⁶ is independently selected from hydrogen, C₁₋₃-alkyl and (OCH₂CH₂)ₙOCH₃,
R⁷ is independently selected from C₁₋₃-alkyl and (OCH₂CH₂)ₙOCH₃, and n is from 40 to 50.

10. A compound of claim 7, wherein X¹ is —NR¹—, R¹ is hydrogen; B is selected from the group consisting of C₁₋₄-alkylene and 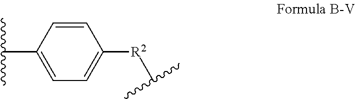
R² being methylene or ethylene; and
Z is represented by Formula Z-I, wherein R⁶ and R⁷ are identical C₁₋₃-alkyl substituents.

11. A compound of claim 7, wherein X¹ is —NR¹—, R¹ is hydrogen; B is —(CH₂)₄—; and Z is represented by Formula Z-I, R⁶ and R⁷ being identical C₁₋₃-alkyl substituents.

12. A compound according to claim 7, wherein X¹ is —NH—; B is —(CH₂)₄—; and Z is represented by Formula Z-I, R⁶ and R⁷ being identical C₁₋₃ alkyl substituents.

13. A compound of Formula IV 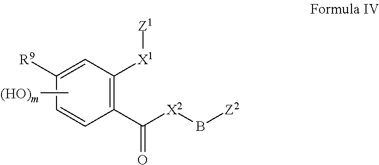
or an enantiomer, diastereomer, racemate, tautomer, salt or hydrate thereof, wherein
m=0 or 1;
X¹ and X² are independently selected from the group consisting of —O—, —S— and
—NR¹—, R¹ being hydrogen or C₁₋₆-alkyl;
B is an optionally substituted aliphatic, heteroaliphatic, aromatic, heteroaromatic or alkylaryl substituent having 1 to 40 carbon atoms;
Z¹ is selected from the group consisting of hydrogen, farnesyl and a folic acid residue;
Z² is selected from the group consisting of 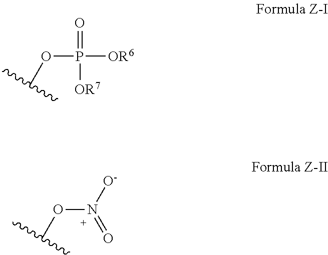
wherein R⁶ is independently selected from hydrogen, C₁₋₁₀₀-alkyl and a polyethylene glycol residue,
R⁷ is independently selected from hydrogen, C₁₋₁₀₀-alkyl and a polyethylene glycol residue;
or B together with Z² forms a structure 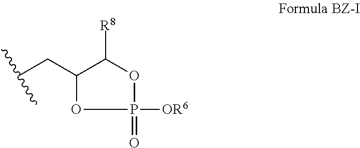
wherein R⁶ is defined as above,
R⁸ is independently selected from hydrogen, an aliphatic substituent with 1 to 22 carbon atoms, a C₁₋₆-alkyl group, and a polyethylene glycol residue and
R⁹ is hydrogen or trifluoromethyl.

14. A compound of claim 13, wherein the folic acid residue is selected from the group consisting of

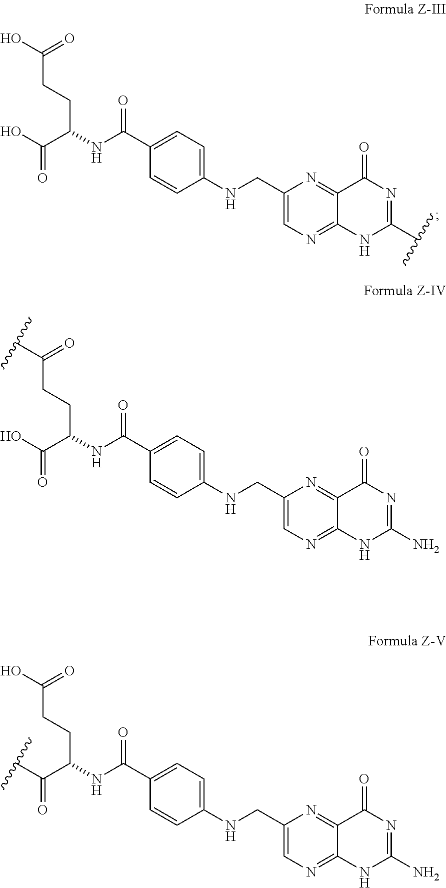

15. A compound of claim 14, wherein Z¹ is hydrogen.

16. A compound of claim 16, wherein Z¹ is farnesyl.

17. A compound of claim 1, wherein B is selected from the group consisting of 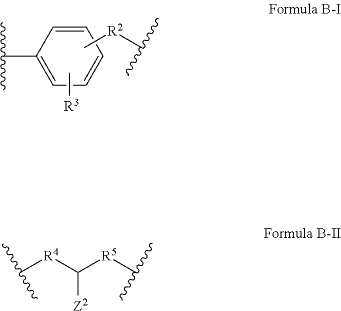
and an aliphatic substituent with 1 to 40 carbon atoms,
R², R⁴ and R⁵ are the same or different C₁₋₃-alkylene, and
R³ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy.

18. A compound of claim 7, wherein B is selected from the group consisting of 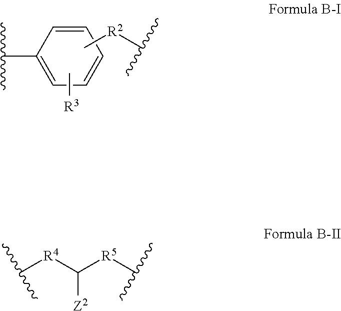
and an aliphatic substituent with 1 to 40 carbon atoms,
R², R⁴ and R⁵ are the same or different C₁₋₃-alkylene, and
R³ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —CO(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy.

19. A compound of claim 13, wherein B is selected from the group consisting of 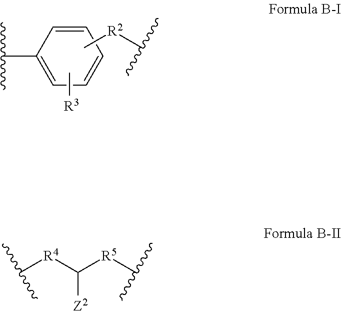
and an aliphatic substituent with 1 to 40 carbon atoms,
R², R⁴ and R⁵ are the same or different C₁₋₃-alkylene, and
R³ is hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy.

20. A method of treatment comprising administering a compound of claim 1 to a patient in need thereof.
